(19) United States
(12) Patent Application Publication
(10) Pub. No.: US 2021/0102876 A1
(43) Pub. Date: Apr. 8, 2021

(54) TESTING FOR PARTICULATES

(71) Applicant: HERO SCIENTIFIC LTD., Jerusalem (IL)

(72) Inventors: Lazar FRUCHTER, Efrat (IL); Arie Oscar Holtz, Jerusalem (IL); Robert Eric Levitz, Beit Shemesh (IL); Leah Forgosh, West Hartford, CT (US); Boaz Arieli, Mavaseret Tzion (IL); Zvi Feldman, Sde Zvi (IL); Maoz Cohen, Bat Yam (IL); Michael Librus, Netany (IL); Avihu Izhak Sivan, Petach Tikvah (IL); Raz Silberman, Holon (IL)

(73) Assignee: HERO SCIENTIFIC LTD., Jerusalem (IL)

(21) Appl. No.: 17/122,594

(22) Filed: Dec. 15, 2020

Related U.S. Application Data
(63) Continuation-in-part of application No. 16/489,853, filed on Aug. 29, 2019.
Application No. 16/489,853 is a continuation-in-part of application No. PCT/IL2019/050997, filed on Sep. 5, 2019.
(60) Provisional application No. 62/727,268, filed on Sep. 5, 2018.

(30) Foreign Application Priority Data
Mar. 2, 2017 (GB) 1703383.8

(51) Int. Cl. G01N 1/40

(52) U.S. Cl. CPC G01N 1/4077; B01L 2200/0668; B01L 2300/0681; G01N 2001/4088

(57) ABSTRACT

A method is provided that includes collecting, from a patient, gargled fluid that potentially contains a particulate selected from the group consisting of: a bacterium and a virus; passing the gargled fluid through a filter; and subsequently, testing for the presence of the particulate trapped by the filter. Other embodiments are also described.

### CROSS-REFERENCE TO RELATED APPLICATIONS

The present application is:

(a) a continuation-in-part of U.S. application Ser. No. 16/489,853, filed Aug. 29, 2019, which is the US national stage of International Application PCT/IL2018050225, filed Feb. 28, 2018, which claims foreign priority from UK Application GB1703383.8, filed Mar. 2, 2017, now abandoned, and

(b) a continuation-in-part of International Application PCT/IL2019/050997, filed Sep. 5, 2019, which claims priority from U.S. Provisional Application 62/727,268, filed Sep. 5, 2018.

All of the above-mentioned applications are assigned to the assignee of the present application and incorporated herein by reference.

### FIELD OF THE INVENTION

Applications of the present invention relate to testing for the presence of particulates, such as bacteria and viruses, in fluids.

### BACKGROUND

Streptococcal pharyngitis, streptococcal tonsillitis, or streptococcal sore throat (known colloquially as strep throat) is a type of pharyngitis caused by group A beta hemolytic streptococcus bacteria. Common symptoms include fever, sore throat, and enlarged cervical lymph nodes.

The rapid strep test is commonly used to test for the presence of group A streptococcus bacteria. In this test, a swab is streaked across the throat to collect bacteria, and is subsequently inserted into an extraction solution, e.g., a mixture of 2M sodium nitrite (hereinbelow, “solution A”), and 0.2M acetic acid (hereinbelow, “solution B”). (Hereinbelow, this mixture is sometimes referred to as “A and B solution.”) The extraction solution extracts strep A carbohydrate antigen from the bacteria. A dipstick containing an antibody specific to strep A carbohydrate antigen is inserted into the mixture containing the antigen. The mixture migrates up the dipstick and reacts with the antibody, thus generating a line on the dipstick. The presence of this line indicates a positive test result.

Other clinical situations also call for testing for presence of a particulate. For example, a physician may wish to test a patient's blood for the presence of a virus, or a stool specimen for the presence of a pathogen.

### SUMMARY OF THE INVENTION

Some applications of the present invention provide apparatus for testing a fluid for presence of a particulate such as a microorganism, a spore, a virus, or other biological entity, e.g., a biological cell. The apparatus includes a tube and a plunger sized and shaped to be advanceable within the tube while sealably contacting the tube. First, the fluid that potentially contains the particulate is collected in the tube. Subsequently, the plunger is used to push the fluid through a filter. The filter may be disposed at the distal end of the tube, the fluid being pushed through the filter and out of the tube. Alternatively, the filter is disposed on the distal end of the plunger, and the fluid is pushed through the filter and into one or more compartments in the plunger. Following the pushing of the fluid through the filter, and while the filter is inside the tube, the filter may be tested for presence of the particulate. For example, the tube and plunger may be turned upside down, and the rapid strep test may then be conducted via an opening at the distal end of the tube. Alternatively or additionally, a throat culture may be performed on the bacteria collected on the filter.

In some applications of the present invention, a testing device is provided for testing for presence of particulate in a liquid, such as group A streptococcus bacteria. The testing device typically comprises a liquid container for containing the liquid; a filter, disposed in or downstream of the liquid container; and a liquid-pressure source, such as a plunger, which is arranged to apply pressure to drive the liquid contained in the liquid container through the filter. For some applications, the liquid comprises gargled fluid, i.e., a gargle fluid that the patient has gargled in his or her mouth and spit out, perhaps along with some saliva. Alternatively, for some applications, the liquid comprises saliva not swabbed from the throat of a patient.

Typically, types of particulates that may be tested for include a microorganism (e.g., a parasite), a fungus, a bacteria, a spore (e.g., a pollen spore), a virus, a mite, a biological cell (e.g., a cancerous cell), a biological antigen, a protein, a protein antigen, and a carbohydrate antigen.

There is therefore provided, in accordance with an application of the present invention, a method including:

collecting, from a patient, gargled fluid that potentially contains a particulate selected from the group consisting of: a bacterium and a virus;

passing the gargled fluid through a filter; and

subsequently, testing for the presence of the particulate trapped by the filter.

For some applications, testing for the presence of the particulate trapped by the filter includes testing the filter for the presence of the particulate.

For some applications, the particulate is the bacterium, and testing for the presence of the particulate trapped by the filter includes testing for the presence of the bacterium trapped by the filter.

For some applications, the particulate is Streptococcus bacterium, and testing for the presence of the particulate trapped by the filter includes testing for the presence of the Streptococcus bacterium trapped by the filter.

For some applications, testing for the presence of the Streptococcus bacterium trapped by the filter includes testing the filter for the presence of the Streptococcus bacterium.

For some applications, testing for the presence of the Streptococcus bacterium trapped by the filter includes testing for the presence of Streptococcus antigen trapped by the filter.

For some applications, testing for the presence of the Streptococcus antigen trapped by the filter includes adding a detergent to the gargled fluid.

For some applications, adding the detergent to the gargled fluid includes adding polysorbate to the gargled fluid.

For some applications, testing for the presence of the Streptococcus antigen trapped by the filter includes performing a rapid strep test using a dipstick.

For some applications, performing the rapid strep test includes performing the rapid strep test without first culturing the gargled fluid.

For some applications, the dipstick is a lateral flow immunoassay test strip.

For some applications, testing for the presence of the Streptococcus antigen trapped by the filter includes applying, to the filter, an extraction solution configured to extract the Streptococcus antigen from the Streptococcus bacterium.

For some applications, the extraction solution is configured to extract Strep A carbohydrate antigen from the Streptococcus bacterium, and testing for the presence of the Streptococcus antigen trapped by the filter includes testing for the presence of the Strep A carbohydrate antigen trapped by the filter.

For some applications, testing for the presence of the Streptococcus bacterium trapped by the filter further includes increasing a surface area of the filter that is exposed to the extraction solution before testing for the presence of the Streptococcus antigen trapped by the filter.

For some applications, testing for the presence of the Streptococcus bacterium trapped by the filter further includes mixing the filter after applying the extraction solution to the filter.

For some applications, the particulate is the virus, and testing for the presence of the particulate trapped by the filter includes testing for the presence of the virus trapped by the filter.

For some applications, testing for the presence of the particulate trapped by the filter further includes tearing the filter before testing for the presence of the particulate trapped by the filter.

For some applications, the method further includes puncturing the filter before testing for the presence of the particulate trapped by the filter.

For some applications, testing for the presence of the particulate trapped by the filter further includes crushing the filter before testing for the presence of the particulate trapped by the filter.

For some applications, testing for the presence of the particulate trapped by the filter further includes concentrating the filter into a more compact volume before testing for the presence of the particulate trapped by the filter.

For some applications, collecting the gargled fluid includes collecting a gargle fluid that the patient has gargled in his or her mouth and spit out, and the gargle fluid includes a detergent.

For some applications, the detergent includes polysorbate.

For some applications, passing the gargled fluid through the filter includes pushing the gargled fluid through the filter.

For some applications, the method further includes, before passing the gargled fluid through the filter, culturing the particulate using a culture medium.

For some applications, the method further includes, before passing the gargled fluid through the filter, preserving the particulate using a preserving medium.

For some applications, passing the gargled fluid through the filter includes passing the gargled fluid through the filter such that adhesive properties of the filter facilitate trapping of the particulate by the filter.

For some applications, passing the gargled fluid through the filter includes passing the gargled fluid through the filter such that the filter traps mucus.

For some applications, passing the gargled fluid through the filter includes passing the gargled fluid through the filter such that the mucus adheres to the filter.

For some applications, the filter includes first and second filters, and passing the gargled fluid through the filter includes passing the gargled fluid through the first and the second filters.

For some applications, testing for the presence of the particulate includes testing for the presence of the particulate captured by the first filter.

For some applications, testing for the presence of the particulate includes testing for the presence of the particulate captured by the first filter and testing for the presence of the particulate captured by the second filter.

For some applications:

the particulate is the bacterium, and testing for the presence of the particulate includes testing for the presence of the bacterium captured by the first filter, and

the method further includes testing for the presence of a virus captured by the second filter.

For some applications, passing the gargled fluid through the first and the second filters includes passing the gargled fluid through the first and the second filters while the first filter is disposed within a tube.

For some applications, passing the gargled fluid through the first and the second filters includes advancing a plunger within the tube.

For some applications, passing the gargled fluid through the first and the second filters includes passing the gargled fluid through the first filter and then through the second filter, and a pore size of the first filter is larger than a pore size of the second filter.

For some applications, the pore size of the second filter is no more than 20 microns.

For some applications, the pore size of the second filter is no more than 1 micron.

For some applications, the pore size of the second filter is between 0.1 and 1 micron.

For some applications, the pore size of the second filter is between 0.01-0.3 micron.

For some applications, the pore size of the first filter is between 0.5 and 100 microns.

For some applications, the pore size of the first filter is between 10 and 100 microns.

There is further provided, in accordance with an application of the present invention, a kit for testing gargled fluid for the presence of a particulate selected from the group consisting of: a bacterium and a virus, the kit including:

a filtering apparatus, which includes:
a tube having a proximal opening;
a first filter disposed within the tube, the first filter having a pore size of between 0.5 and 100 microns; and
a second filter having a pore size of no more than 20 microns, and less than the pore size of the first filter,
wherein the second filter is disposed distally to the first filter, such that the gargled fluid, when in the tube, passes through the first filter and then through the second filter; and

a lateral flow immunoassay test strip, which is configured to detect the presence of the particulate.

For some applications, the second filter has a pore size of no more than 1 micron.

For some applications, the second filter has a pore size of between 0.1 and 20 microns.

For some applications, the second filter has a pore size of between 0.1 microns and 1 micron.

For some applications, the second filter has a pore size of between 0.01 microns and 0.3 micron.

For some applications, the second filter has a pore size of between 1 micron and microns.

For some applications, the first filter has a pore size of between 0.5 and 20 microns.

For some applications, the first filter has a pore size of between 10 and 100 microns.

For some applications, the first filter has a pore size of between 5 and 25 microns.

For some applications, the filtering apparatus further includes a fluid-collection compartment distal to the first and the second filters.

For some applications, a wall of the fluid-collection compartment is shaped so as to define a pressure-release hole, such that air pressure in the fluid-collection compartment is released through the pressure-release hole.

For some applications, the filtering apparatus further includes a plunger sized and shaped to be distally advanceable through the proximal opening of the tube and within the tube.

For some applications, the second filter is disposed within the tube.

There is still further provided, in accordance with an application of the present invention, a kit for testing gargled fluid for the presence of a particulate selected from the group consisting of: a bacterium and a virus, the kit including:

a filtering apparatus, which includes:
a tube having a proximal opening;
a first filter disposed within the tube, the first filter having a pore size of between 0.5 and 100 microns; and
a second filter having a pore size of no more than 20 microns, and less than the pore size of the first filter,
wherein the second filter is disposed distally to the first filter, such that the gargled fluid, when in the tube, passes through the first filter and then through the second filter;

a vial; and

a liquid for bathing the particulate in the vial, the liquid selected from the group consisting of: a lysis buffer, saline solution, and transport medium.

For some applications, the first filter has a pore size of between 0.5 and 20 microns.

For some applications, the first filter has a pore size of between 10 and 100 microns.

For some applications, the vial contains the liquid.

There is still further provided in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

using a plunger, pushing the fluid through a filter disposed at a location selected from the group consisting of: a distal portion of the tube, and a distal end of the plunger; and

subsequently, while the filter is inside the tube, ascertaining if any of the particulate was trapped by the filter by applying a particulate-presence-testing-facilitation solution to the filter.

For some applications, ascertaining includes ascertaining using a first protocol, and if no particulate is found to be present, ascertaining using a second protocol.

For some applications, ascertaining using the first protocol includes applying the particulate-presence-testing-facilitation solution to the filter, the method further including taking a sample from the filter prior to applying the particulate-presence-testing-facilitation solution to the filter.

For some applications, the method further includes culturing the sample taken from the filter for 2-48 hours, and ascertaining using the second protocol includes ascertaining if any of the particulate is present in the sample after the 2-48 hours.

For some applications, collecting the fluid in the tube includes collecting gargled fluid in the tube.

For some applications, a temperature of the gargled fluid is 1-38 degrees Celsius.

For some applications,

the particulate includes a microorganism,

the particulate-presence-testing-facilitation solution includes a releasing agent configured to release an antigen from the microorganism, and

testing for presence of the particulate includes testing for presence of the particulate by testing for presence of the antigen.

For some applications, using the plunger to push the fluid through the filter includes advancing the plunger at least until the plunger contacts the filter.

For some applications, the method further includes puncturing the filter before testing for presence of the particulate.

For some applications, testing for presence of the particulate includes testing for presence of the particulate while a distal opening of the tube is above a proximal opening of the tube, and the plunger and tube are resting on a horizontal surface, on a proximal end of the plunger.

For some applications, testing for presence of the particulate includes testing for presence of the particulate while a distal opening of the tube is above a proximal opening of the tube, and the plunger and tube are resting on a horizontal surface, on a proximal end of the tube.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube having a filter, fluid that potentially contains the particulate;

passing the fluid through the filter; and

subsequently, while the filter is inside the tube, testing for presence of the particulate, by ascertaining if any of the particulate was trapped by the filter by applying a particulate-presence-testing-facilitation solution to the filter.

For some applications, ascertaining includes ascertaining using a first protocol, and if no particulate is found to be present, ascertaining using a second protocol.

For some applications, ascertaining using the first protocol includes applying the particulate-presence-testing-facilitation solution to the filter, the method further including taking a sample from the filter prior to applying the particulate-presence-testing-facilitation solution to the filter.

For some applications, the method further includes culturing the sample taken from the filter for 2-48 hours, and ascertaining using the second protocol includes ascertaining if any of the particulate is present in the sample after the 2-48 hours.

For some applications, collecting the fluid in the tube includes collecting gargled fluid in the tube.

For some applications, a temperature of the gargled fluid is 1-38 degrees Celsius.

There is further provided, in accordance with some applications of the present invention, apparatus including:

a tube having a funnel-shaped proximal opening, a proximal-most diameter of the funnel-shaped proximal opening being at least 20% greater than a diameter of the tube;

a plunger sized and shaped to be advanceable within the tube while sealably contacting the tube; and

a filter disposed at a location selected from the group consisting of: a distal portion of the tube, and a distal end of the plunger.

For some applications, the proximal-most diameter of the funnel-shaped proximal opening is at least 30% greater than the diameter of the tube.

There is therefore provided, in accordance with some applications of the present invention, apparatus including:

a tube, closed at a distal end thereof;

a filter disposed within the tube, the tube being shaped to define a fluid-collection compartment distal to the filter; and

a plunger sized and shaped to be advanceable within the tube while sealably contacting the tube, the plunger being arranged to push a fluid through the filter and into the fluid-collection compartment.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

pushing the fluid through a filter, disposed within the tube, into a fluid-collection compartment distal to the filter; and

subsequently, while the filter is inside the tube, testing for presence of the particulate, by ascertaining if any of the particulate was trapped by the filter.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

pushing the fluid through a filter disposed within a distal portion of the tube;

tearing the filter while the filter is inside the tube; and

subsequently, while the filter is inside the tube, testing for presence of the particulate, by ascertaining if any of the particulate was trapped by the filter.

There is further provided, in accordance with some applications of the present invention, apparatus including:

a tube;

a plunger sized and shaped to be advanceable within the tube, a ratio of (a) a diameter of a proximal end of the plunger, to (b) a length of the plunger, being at least 1; and

a filter disposed at a location selected from the group consisting of: a distal portion of the tube, and a distal end of the plunger.

There is further provided, in accordance with some applications of the present invention, apparatus including:

a tube; and

a plunger sized and shaped to be advanceable within the tube while sealably contacting the tube, a proximally-facing surface at a distal end of the tube inhibiting advancement of the plunger;

the plunger and tube being shaped to provide an empty volume of 0.03-5 mL proximal to the proximally-facing surface, when the plunger is maximally advanced within the tube.

For some applications, the empty volume is 0.03-1 mL.

For some applications, the apparatus further includes a filter disposed within a distal portion of the tube.

For some applications, the apparatus further includes a puncturing element protruding from a distal end of the plunger, the puncturing element being configured to puncture the filter upon the plunger being advanced to the filter.

For some applications, the apparatus further includes (a) a kit in which the plunger and tube are disposed, and (b) a puncturing element disposed within the kit,

the puncturing element being sized and shaped to be passable through an opening at a distal end of the tube and configured to puncture the filter by being longer than a distance from (i) the opening at the distal end of the tube to (ii) the filter.

For some applications, a distal end of the plunger is shaped to define a distally-facing cavity therein, the cavity providing at least part of the empty volume.

For some applications, a volume of the cavity is between 0.03 and 5 mL.

For some applications, the volume of the cavity is 0.03-1 mL.

For some applications, the volume of the cavity is at least 0.15 mL.

For some applications, the volume of the cavity is at least 0.25 mL.

For some applications, the volume of the cavity is at least 0.4 mL.

For some applications, the tube does not include a Luer lock.

For some applications, the tube does not include a needle-coupling mechanism.

For some applications, the apparatus further includes a kit in which the plunger and tube are disposed, the plunger being disposed entirely outside of the tube.

For some applications, a distal end of the plunger is not convex.

For some applications, a volume of the tube is between 1 and 70 mL.

For some applications, the volume of the tube is between 1 and 8 mL.

For some applications, the volume of the tube is between 8 and 15 mL.

For some applications, the volume of the tube is between 15 and 30 mL.

For some applications, the volume of the tube is between 30 and 70 mL.

For some applications, the apparatus further includes (a) a kit in which the plunger and tube are disposed, and (b) a particulate-presence-testing-facilitation solution disposed within the kit, the particulate selected from the group consisting of: a microorganism, a fungus, a bacteria, a spore, a virus, a mite, a biological cell, a biological antigen, a protein, a protein antigen, and a carbohydrate antigen.

For some applications, a distal end of the plunger is shaped to define at least one enclosed cavity containing a particulate-presence-testing-facilitation solution,

the enclosed cavity being configured to open upon the plunger being moved within the tube, and

the particulate selected from the group consisting of: a microorganism, a fungus, a bacteria, a spore, a virus, a mite, a biological cell, a biological antigen, a protein, a protein antigen, and a carbohydrate antigen.

For some applications, a distal end of the plunger is shaped to define at least one enclosed cavity containing a particulate-presence-testing-facilitation solution,

the enclosed cavity being configured to open while the plunger is inside the tube, and

the particulate selected from the group consisting of: a microorganism, a fungus, a bacteria, a spore, a virus, a mite, a biological cell, a biological antigen, a protein, a protein antigen, and a carbohydrate antigen.

For some applications, a wall of the tube is shaped to define at least one enclosed cavity containing a particulate-presence-testing-facilitation solution,

a wall of the enclosed cavity being configured to open upon the plunger being moved within the tube, and

the particulate selected from the group consisting of: a microorganism, a fungus, a bacteria, a spore, a virus, a mite, a biological cell, a biological antigen, a protein, a protein antigen, and a carbohydrate antigen.

For some applications, a wall of the tube is shaped to define at least one enclosed cavity containing a particulate-presence-testing-facilitation solution,

a wall of the enclosed cavity being configured to open, and

the particulate selected from the group consisting of: a microorganism, a fungus, a bacteria, a spore, a virus, a mite, a biological cell, a biological antigen, a protein, a protein antigen, and a carbohydrate antigen.

For some applications, the enclosed cavity further contains a gas above atmospheric pressure, such that the particulate-presence-testing-facilitation solution is forced out upon the opening of the wall of the cavity.

For some applications, the plunger is shaped to define at least one plunger lumen containing a particulate-presence-testing-facilitation solution,

the particulate selected from the group consisting of: a microorganism, a fungus, a bacteria, a spore, a virus, a mite, a biological cell, a biological antigen, a protein, a protein antigen, and a carbohydrate antigen.

For some applications, the plunger lumen is configured to open upon the plunger being moved within the tube.

For some applications, the plunger lumen is configured to open while the plunger is inside the tube.

For some applications, the plunger lumen further contains a gas above atmospheric pressure, such that the particulate-presence-testing-facilitation solution is forced out of the plunger lumen upon opening of the plunger lumen.

For some applications, the apparatus further includes at least one sub-plunger configured to be slidably disposed within the plunger lumen and to deploy the particulate-presence-testing-facilitation solution.

For some applications, a proximal end of the tube is shaped to define a funnel.

For some applications, a distal end of the tube is shaped to define a conduit.

For some applications, a distal end of the tube is funnel-shaped.

For some applications, a ratio of a diameter of a proximal opening of the tube to a diameter of a distal opening of the tube is at least 13.

For some applications, a proximal end of the plunger is not proximal to a proximal end of the tube, when the plunger is maximally advanced within the tube.

For some applications,

the plunger is shaped to define a plunger lumen, and

the apparatus further includes a shaft shaped to be slidably disposed within the plunger lumen, a distal end of the shaft including a puncturing element.

There is further provided, in accordance with some applications of the present invention, apparatus including:

a tube;

a plunger sized and shaped to be advanceable within the tube while sealably contacting the tube, a distal end of the plunger shaped to define one or more passageways therethrough; and

a filter coupled to the distal end of the plunger.

For some applications, the plunger is shaped to define one or more compartments in fluid communication with the passageways.

For some applications, the apparatus further includes (a) a first sealing ring surrounding the plunger proximally to the compartments, and (b) a second sealing ring surrounding the plunger distally to the compartments.

For some applications, a total volume of the compartments is between 0.5 and 60 mL.

For some applications, the total volume is between 5 and 30 mL.

For some applications, the total volume is between 8 and 20 mL.

For some applications, the plunger is shaped to define a disk proximal to the compartments, the disk being configured to inhibit passage of liquid from the compartments to a proximal side of the disk, when the disk is inside the tube.

For some applications, the tube includes a distal cylindrical portion, a length of the plunger distal to the disk being within 10 mm of a height of the cylindrical portion.

For some applications, the tube further includes a proximal funnel portion coupled to the cylindrical portion.

For some applications, a length of the plunger proximal to the disk is not greater than a height of the funnel portion.

For some applications, a distal end of the tube is shaped to define an openable seal.

For some applications, a distal end of the tube does not have an opening.

For some applications, the apparatus further includes a stopper configured to close a distal opening of the tube.

For some applications, the stopper is disposed over the distal opening of the tube.

For some applications, a distal end of the tube is shaped to define a conduit.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

using a plunger, pushing the fluid through a filter disposed at a location selected from the group consisting of: a distal portion of the tube, and a distal end of the plunger, and

subsequently, while the filter is inside the tube, testing for presence of the particulate, by ascertaining if any of the particulate was trapped by the filter.

For some applications, the selected location is the distal portion of the tube, and pushing the fluid includes pushing the fluid out of the tube through a conduit disposed at the distal end of the tube.

For some applications, the plunger is shaped to define at least one compartment, the selected location is the distal end of the plunger, and pushing the fluid includes pushing the fluid into the at least one compartment.

For some applications, the apparatus further includes puncturing the filter before testing for presence of the particulate.

For some applications, the selected location is the distal end of the tube, and puncturing the filter includes using a puncturing element protruding from a distal end of the plunger to puncture the filter.

For some applications, the selected location is the distal end of the plunger and puncturing the filter includes using a puncturing element protruding, in a proximal direction, from the distal end of the tube to puncture the filter.

For some applications, the selected location is the distal end of the tube, and the plunger is configured to rotate with respect to the tube when inside the tube, at least one puncturing element protruding from a distal end of the plunger.

For some applications, the selected location is the distal end of the plunger, and the plunger is configured to rotate with respect to the tube when inside the tube, at least one puncturing element protruding, in a proximal direction, from the distal end of the tube.

For some applications, testing for presence of the particulate includes testing for presence of the particulate while a distal opening of the tube is above a proximal opening of the tube.

For some applications, testing for presence of the particulate includes testing for presence of the particulate while the plunger and tube are resting on a horizontal surface, on a proximal end of the plunger.

For some applications, testing for presence of the particulate includes testing for presence of the particulate while the plunger and tube are resting on a horizontal surface, on a proximal end of the tube.

For some applications, testing for presence of the particulate includes testing for presence of the particulate via a passageway passing through the plunger from a proximal end of the plunger to the distal end of the plunger.

For some applications, testing for presence of the particulate includes applying a particulate-presence-testing-facilitation solution to the filter.

For some applications, the selected location is the distal portion of the tube, the filter is a first filter, a second filter is disposed in the distal end of the tube, and applying the particulate-presence-testing-facilitation solution to the filter includes applying the particulate-presence-testing-facilitation solution to the first filter but not to the second filter, the two filters being separated by (a) a barrier extending in a proximal direction disposed within the tube or (b) a recess defined in the distal end of the tube.

For some applications, the selected location is the distal end of the plunger, the filter is a first filter, a second filter is disposed in the distal end of the plunger, and applying the particulate-presence-testing-facilitation solution to the filter includes applying the particulate-presence-testing-facilitation solution to the first filter but not to the second filter, the two filters being separated by (a) a recess defined in the distal end of the plunger or (b) a barrier protruding in a distal direction from the distal end of the plunger.

For some applications, applying the particulate-presence-testing-facilitation solution to the filter includes applying the particulate-presence-testing-facilitation solution to the filter by passing the particulate-presence-testing-facilitation solution through a conduit at a distal end of the tube.

For some applications, the distal surface of the plunger is disposed at a slant with respect to a longitudinal axis of the plunger, and passing the particulate-presence-testing-facilitation solution through a conduit at a distal end of the tube includes passing the particulate-presence-testing-facilitation solution through a conduit disposed over a higher end of the slanted distal surface of the plunger when a proximal end of the tube or a proximal end of the plunger is resting on a horizontal surface.

For some applications, the plunger is shaped to define at least one plunger lumen, and applying the particulate-presence-testing-facilitation solution to the filter includes applying the particulate-presence-testing-facilitation solution to the filter by passing the particulate-presence-testing-facilitation solution out of the plunger lumen.

For some applications, a proximally-facing distal surface of the tube is disposed at a slant with respect to a lateral wall of the tube, and passing the particulate-presence-testing-facilitation solution out of the plunger lumen includes passing the particulate-presence-testing-facilitation solution out of a plunger lumen disposed over a higher end of the slanted distal surface of the tube, when a distally-facing distal surface of the tube is resting on a horizontal surface.

For some applications,

the particulate includes a microorganism,

the particulate-presence-testing-facilitation solution includes a releasing agent configured to release an antigen from the microorganism, and

testing for presence of the particulate includes testing for presence of the particulate by testing for presence of the antigen.

For some applications, applying the particulate-presence-testing-facilitation solution to the filter includes releasing the particulate-presence-testing-facilitation solution inside the tube by using the plunger to open an enclosed cavity inside of which the particulate-presence-testing-facilitation solution is disposed.

For some applications, applying the particulate-presence-testing-facilitation solution to the filter includes releasing the particulate-presence-testing-facilitation solution inside the tube by opening an enclosed cavity inside of which the particulate-presence-testing-facilitation solution is disposed.

For some applications, using the plunger to push the fluid through the filter includes advancing the plunger at least until the plunger contacts the filter.

For some applications, collecting the fluid in the tube includes collecting gargled fluid in the tube.

For some applications, the gargled fluid includes an element selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid includes a plurality of elements selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid is carbonated.

For some applications, a temperature of the gargled fluid is 1-38 degrees Celsius.

For some applications, collecting the fluid in the tube includes collecting biological fluid in the tube.

For some applications, collecting the biological fluid in the tube includes collecting saliva in the tube.

For some applications, collecting the biological fluid in the tube includes collecting blood in the tube.

For some applications, collecting the biological fluid in the tube includes collecting urine in the tube.

For some applications, collecting the biological fluid in the tube includes collecting stool in the tube.

For some applications, collecting the biological fluid in the tube includes collecting gastrointestinal fluid in the tube.

For some applications, collecting the biological fluid in the tube includes collecting bronchoalveolar lavage fluid in the tube.

For some applications, testing for presence of the particulate includes testing for presence of a bacteria.

For some applications, testing for presence of a bacteria includes testing for presence of a streptococcus bacteria.

For some applications, testing for presence of the particulate includes testing for presence of a virus.

For some applications, testing for presence of the particulate includes testing for presence of a biological cell.

For some applications, testing for presence of the biological cell includes testing for presence of a cancerous cell.

For some applications, testing for presence of the particulate includes testing for presence of a pollen spore.

For some applications, testing for presence of the particulate includes testing for presence of a fungus.

For some applications, testing for presence of the particulate includes testing for presence of a mite.

For some applications, the method further includes, before testing for presence of the particulate, using a culture medium to culture the particulate.

For some applications, the method further includes, before testing for presence of the particulate, using a preserving medium to preserve the particulate.

For some applications, ascertaining includes ascertaining using a first protocol, and if no particulate is found to be present, ascertaining using a second protocol.

For some applications, ascertaining using the first protocol includes applying a particulate-presence-testing-facilitation solution to the filter.

For some applications, the selected location is the distal portion of the tube, the method further includes removing the plunger from the tube and subsequently transferring a sample from the distal end of the plunger to a culture media surface, and ascertaining using the second protocol includes ascertaining whether the particulate is on the culture media surface.

For some applications, the method further includes taking a sample from the filter prior to applying the particulate-presence-testing-facilitation solution to the filter.

For some applications, taking the sample from the filter includes swabbing the filter.

For some applications, the selected location is the distal portion of the tube, and swabbing the filter includes swabbing the filter from a proximal end of the plunger through a plunger lumen of the plunger, while the plunger is inside the tube.

For some applications, the selected location is the distal portion of the tube, and taking the sample includes removing the plunger from the tube, and subsequently swabbing the filter from a proximal end of the tube.

For some applications, the selected location is the distal portion of the tube, and swabbing the filter includes swabbing the filter through a conduit in a distal end of the tube.

For some applications, the selected location is the distal end of the plunger, and swabbing the filter includes swabbing the filter from a proximal end of the plunger through a plunger lumen of the plunger.

For some applications, the selected location is the distal end of the plunger, and swabbing the filter includes swabbing the filter through a conduit defined in the distal end of the tube, while the plunger is inside the tube.

For some applications, the method further includes culturing the sample taken from the filter for 2-48 hours, and ascertaining using the second protocol includes ascertaining if any of the particulate is present in the sample after the 2-48 hours.

For some applications, ascertaining if any of the particulate is present in the sample includes applying a particulate-presence-testing-facilitation solution to the sample after the 2-48 hours.

For some applications, culturing the sample includes plating the sample on a culture media surface, and ascertaining if any of the particulate is present in the sample includes ascertaining whether the particulate is on the culture media surface after the 2-48 hours.

There is further provided, in accordance with some applications of the present invention, apparatus including:

a tube;

a plunger sized and shaped to be advanceable within the tube while sealably contacting the tube;

an insert disposed within a distal portion of the tube and not fixed to the plunger; and

a filter coupled to a proximally-facing surface of the insert.

For some applications, the insert is shaped to define (a) an at least partially distally-facing opening therein, and (b) a passage from the proximally-facing surface of the insert to the at least partially distally-facing opening.

For some applications, the insert is further shaped to define a plurality of grooves in the proximally-facing surface of the insert, respective spaces within the grooves being in fluid communication with the passage.

There is further provided, in accordance with some applications of the present invention, apparatus including:

a tube shaped to define a plurality of openings at a distal end thereof;

a plunger sized and shaped to be advanceable within the tube while sealably contacting the tube; and

a filter disposed within a distal portion of the tube, proximal to the plurality of openings.

For some applications, a total area of the plurality of openings is between 10/and 90% of a cross-sectional area of the distal end of the tube.

For some applications, the total area of the plurality of openings is between 10% and 80% of the cross-sectional area of the distal end of the tube.

For some applications, the total area of the plurality of openings is between 10% and 70% of the cross-sectional area of the distal end of the tube.

For some applications, the total area of the plurality of openings is between 20% and 70% of the cross-sectional area of the distal end of the tube.

There is further provided, in accordance with some applications of the present invention, apparatus including:

a tube containing a medium selected from the group consisting of a culture medium configured to culture a microorganism, a culture medium configured to culture a fungus, a culture medium configured to culture a bacteria, a culture medium configured to culture a spore, a culture medium configured to culture a mite, a culture medium configured to culture a biological cell, a culture medium configured to culture a virus, a releasing medium configured to release an antigen from a microorganism, a releasing medium configured to release an antigen from a protein, a releasing medium configured to release an antigen from a carbohydrate, a heating medium configured to undergo an exothermic reaction, a salt, a preserving medium configured to preserve a microorganism, a preserving medium configured to preserve a fungus, a preserving medium configured to preserve a bacteria, a preserving medium configured to preserve a biological cell, a preserving medium configured to preserve a mite, a preserving medium configured to preserve a spore, and a preserving medium configured to preserve a virus;

a filter disposed within a distal portion of the tube; and

a plunger sized and shaped to be advanceable within the tube while sealably contacting the tube.

For some applications, the medium is disposed proximally to the filter.

For some applications, the medium is disposed distally to the filter.

For some applications, the apparatus further includes:

a heating element configured to heat the tube; and

a kit in which the heating element and tube are disposed.

For some applications, the heating element includes a chemical heating element.

For some applications, the heating element includes an electric heating element.

There is further provided, in accordance with some applications of the present invention, a method including:

placing a fluid into a tube containing a medium selected from the group consisting of: a culture medium configured to culture a microorganism, a culture medium configured to culture a fungus, a culture medium configured to culture a bacteria, a culture medium configured to culture a spore, a culture medium configured to culture a mite, a culture medium configured to culture a biological cell, a culture medium configured to culture a virus, a releasing medium configured to release an antigen from a microorganism, a releasing medium configured to release an antigen from a protein, a releasing medium configured to release an antigen from a carbohydrate, a heating medium configured to undergo an exothermic reaction, a salt, a preserving medium configured to preserve a microorganism, a preserving medium configured to preserve a fungus, a preserving medium configured to preserve a bacteria, a preserving medium configured to preserve a biological cell, a preserving medium configured to preserve a mite, a preserving medium configured to preserve a spore, and a preserving medium configured to preserve a virus; and

using a plunger to push the fluid through a filter disposed at a location selected from the group consisting of: a distal portion of the tube, and a distal end of the plunger.

For some applications, the method further including heating the tube.

For some applications, the method further including, following the pushing of the fluid through the filter and while the filter is inside the tube, testing for presence of a particulate by ascertaining if any of the particulate was trapped by the filter, the particulate being selected from the group consisting of: a microorganism, a fungus, a bacteria, a spore, a virus, a mite, a biological cell, a biological antigen, a protein, a protein antigen, and a carbohydrate antigen.

There is further provided, in accordance with some applications of the present invention, apparatus including:

a tube;

a plunger sized and shaped to be advanceable within the tube;

a first filter disposed within a distal portion of the tube, the first filter having a pore size of between 0.5 and 100 microns; and

a second filter having a pore size of between 0.1 and 20 microns.

For some applications, the pore size of the first filter is larger than the pore size of the second filter.

For some applications, the second filter is disposed distally to the first filter.

For some applications, the first filter has a pore size of between 0.5 and 20 microns, and the second filter has a pore size of between 0.1 microns and 1 micron.

For some applications, the first filter has a pore size of between 10 and 100 microns, and the second filter has a pore size of between 1 micron and 10 microns.

For some applications, the first filter has a pore size of between 5 and 25 microns, and the second filter has a pore size of between 1 micron and 20 microns.

There is further provided, in accordance with some applications of the present invention, apparatus including:

a tube having a funnel-shaped proximal opening; and

a plunger sized and shaped to be advanceable within the tube while sealably contacting the tube, the tube and plunger being configured such that, following the plunger being maximally advanced within the tube, the plunger is withdrawable from the tube only by use of a tool or by breaking a portion of the apparatus.

For some applications, a proximal end of the plunger is not proximal to a proximal end of the tube, when the plunger is maximally advanced within the tube.

For some applications, the apparatus further includes a locking mechanism configured to lock the plunger inside the tube following the plunger being maximally advanced within the tube.

There is further provided, in accordance with some applications of the present invention, apparatus including:

a tube; and

a plunger sized and shaped to be advanceable within the tube while sealably contacting the tube,
a distal end of the plunger being shaped to define at least one enclosed cavity containing a particulate-presence-testing-facilitation solution,
the enclosed cavity being configured to open upon the plunger being moved within the tube, and
the particulate selected from the group consisting of: a microorganism, a spore, a virus, a mite, a biological cell, a biological antigen, a protein, a protein antigen, and a carbohydrate antigen.

There is further provided, in accordance with some applications of the present invention, apparatus including:

a tube; and

a plunger sized and shaped to be advanceable within the tube while sealably contacting the tube,
a distal end of the plunger being shaped to define at least one enclosed cavity containing a particulate-presence-testing-facilitation solution,
the enclosed cavity being configured to open while the plunger is inside the tube, and
the particulate selected from the group consisting of: a microorganism, a spore, a virus, a mite, a biological cell, a biological antigen, a protein, a protein antigen, and a carbohydrate antigen.

For some applications, in accordance with some applications of the present invention, apparatus including:

a tube; and

a plunger sized and shaped to be advanceable within the tube while sealably contacting the tube,
a wall of the tube being shaped to define at least one enclosed cavity containing a particulate-presence-testing-facilitation solution,
the enclosed cavity being configured to open upon the plunger being moved within the tube, and
the particulate selected from the group consisting of: a microorganism, a fungus, a bacteria, a spore, a virus, a mite, a biological cell, a biological antigen, a protein, a protein antigen, and a carbohydrate antigen.

There is further provided, in accordance with some applications of the present invention, apparatus including:

a tube; and

a plunger sized and shaped to be advanceable within the tube while sealably contacting the tube,
a wall of the tube being shaped to define at least one enclosed cavity containing a particulate-presence-testing-facilitation solution,
the enclosed cavity being configured to open, and
the particulate selected from the group consisting of: a microorganism, a fungus, a bacteria, a spore, a virus, a mite, a biological cell, a biological antigen, a protein, a protein antigen, and a carbohydrate antigen.

There is further provided, in accordance with some applications of the present invention, apparatus including:

a tube; and

a plunger sized and shaped to be advanceable within the tube while sealably contacting the tube,
the plunger being shaped to define at least one plunger lumen containing a particulate-presence-testing-facilitation solution,
the particulate selected from the group consisting of: a microorganism, a fungus, a bacteria, a spore, a virus, a mite, a biological cell, a biological antigen, a protein, a protein antigen, and a carbohydrate antigen.

There is further provided, in accordance with some applications of the present invention, apparatus including:

a tube;

a filter disposed within a distal portion of the tube;

a plunger sized and shaped to be advanceable within the tube while sealably contacting the tube; and

a puncturing element protruding from a distal end of the plunger, the puncturing element being configured to puncture the filter upon the plunger being advanced to the filter.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube having a filter, fluid that potentially contains the particulate;

passing the fluid through the filter; and

subsequently, while the filter is inside the tube, testing for presence of the particulate, by ascertaining if any of the particulate was trapped by the filter.

For some applications, ascertaining includes ascertaining using a first protocol, and if no particulate is found to be present, ascertaining using a second protocol.

For some applications, ascertaining using the first protocol includes applying a particulate-presence-testing-facilitation solution to the filter.

For some applications, the method further includes taking a sample from the filter prior to applying the particulate-presence-testing-facilitation solution to the filter.

For some applications, taking the sample from the filter includes swabbing the filter.

For some applications, swabbing the filter includes swabbing the filter from a proximal end of the tube.

For some applications, swabbing the filter includes swabbing the filter through a conduit in a distal end of the tube.

For some applications, the method further includes culturing the sample taken from the filter for 2-48 hours, and wherein ascertaining using the second protocol includes ascertaining if any of the particulate is present in the sample after the 2-48 hours.

For some applications, ascertaining if any of the particulate is present in the sample includes applying a particulate-presence-testing-facilitation solution to the sample after the 2-48 hours.

For some applications, culturing the sample includes plating the sample on a culture media surface, and ascertaining if any of the particulate is present in the sample includes ascertaining whether any of the particulate is on the culture media surface after the 2-48 hours.

For some applications, collecting the fluid in the tube includes collecting gargled fluid in the tube.

For some applications, the gargled fluid includes an element selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid includes a plurality of elements selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid is carbonated.

For some applications, a temperature of the gargled fluid is 1-38 degrees Celsius.

There is further provided, in accordance with some applications of the present invention, apparatus including:

a tube having proximal and distal ends;

a barrier extending in a proximal direction, disposed within the tube;

a plunger sized and shaped to be advanceable within the tube while sealably contacting the tube, a distal end of the plunger shaped to define a recess into which the barrier fits upon the plunger being advanced to the barrier; and two filters disposed at a location selected from the group consisting of: the distal end of the tube, and a distal end of the plunger.

For some applications, the selected location is the distal end of the tube, and the two filters are separated by the barrier.

For some applications, the selected location is the distal end of the plunger, and the two filters are separated by the recess defined in the distal end of the plunger.

For some applications, the selected location is the distal end of the tube, and the apparatus further includes at least two puncturing elements protruding from the distal end of the plunger, the puncturing elements being configured to puncture the two filters, respectively, upon the plunger being advanced to the filters.

For some applications, the selected location is the distal end of the plunger, and the apparatus further includes at least two puncturing elements protruding in a proximal direction from the distal end of the tube, the puncturing elements being configured to puncture the two filters respectively upon the plunger being advanced to the barrier.

For some applications, the selected location is the distal end of the tube, and the distal end of the tube is shaped to define at least two conduits, the conduits being configured to align with the two filters respectively.

For some applications, the selected location is the distal end of the plunger, and the distal end of the tube is shaped to define at least two conduits, the conduits being configured to align with the two filters respectively when the plunger is inside the tube.

For some applications, the plunger is shaped to define a plunger lumen containing a particulate-presence-testing-facilitation solution, an opening of the plunger lumen being arranged to align with one of the filters and not to simultaneously align with the other filter, such that the particulate-presence-testing-facilitation solution is applied to only the one of the filters.

For some applications, one of the two filters is at least 25% larger than the other.

For some applications, a culture medium is disposed on at least one of the filters.

For some applications, no culture medium is disposed on at least one of the filters.

For some applications, the plunger, once maximally advanced to the barrier, is configured to prevent a particulate-presence-testing-facilitation solution that is applied to one filter from contacting the other filter.

For some applications, a height of the barrier extending in a distal to proximal direction is less than 90% of a height of the tube.

For some applications, the height of the barrier is less than a height of the tube that corresponds to a volume of 10 cc in the tube, measured from the distal end of the tube.

For some applications, the height of the barrier is less than a height of the tube that corresponds to a volume of 5 cc in the tube, measured from the distal end of the tube.

For some applications, the height of the barrier is less than a height of the tube that corresponds to a volume of 1 cc in the tube, measured from the distal end of the tube

For some applications, a distal portion of the tube is (a) shaped to define at least one enclosed cavity containing a particulate-presence-testing-facilitation solution, and (b) configured such that the particulate-presence-testing-facilitation solution in the cavity is applied to only one filter.

For some applications, a wall of the enclosed cavity is configured to open and release the particulate-presence-testing-facilitation solution to the only one filter.

For some applications, a wall of the enclosed cavity is configured to open and release the particulate-presence-testing-facilitation solution to the only one filter following initiation of distal motion of the plunger in the tube.

For some applications, the two filters are a first filter and a second filter, the barrier is a first barrier, the recess is a first recess, and the first filter is separated from the second filter by the first barrier or by the first recess,

(a) the apparatus further including a second barrier extending in a proximal direction, disposed within the tube,

(b) the distal end of the plunger being further shaped to define a second recess into which the second barrier fits upon the plunger being advanced to the barriers, and

(c) the apparatus further including a third filter disposed at a location selected from the group consisting of: the distal end of the tube, and the distal end of the plunger,
the third filter separated from the second filter by the second barrier or by the second recess.

For some applications, a culture medium is disposed on at least one of the filters.

For some applications, the plunger, once maximally advanced to the barriers, is configured to prevent a particulate-presence-testing-facilitation solution that is applied to one filter from contacting any other filter.

For some applications, at least one of the filters is at least 25% larger than at least one other filter.

For some applications, the selected location is the distal end of the tube, and the apparatus further includes at least three puncturing elements protruding from a distal end of the plunger, the puncturing elements being configured to puncture the respective filters, upon the plunger being advanced to the filters.

For some applications, the selected location is the distal end of the plunger, and the apparatus further includes at least three puncturing elements protruding in a proximal direction from the distal end of the tube, the puncturing elements being configured to puncture the respective filters, upon the plunger being advanced to the barriers.

For some applications, the selected location is the distal end of the tube, and the distal end of the tube is shaped to define at least three conduits, the conduits being configured to align with the respective filters.

For some applications, the selected location is the distal end of the plunger, and the distal end of the tube is shaped to define at least three conduits, the conduits being configured to align with the respective filters, when the plunger is in the tube.

There is further provided, in accordance with some applications of the present invention, apparatus including:

a tube having proximal and distal ends, the distal end of the tube shaped to define a recess;

a plunger sized and shaped to be advanceable within the tube while sealably contacting the tube;

a barrier protruding in a distal direction from a distal end of the plunger, configured to fit into the recess of the tube upon the plunger being advanced to the recess; and

two filters disposed at a location selected from the group consisting of: the distal end of the tube, and the distal end of the plunger.

For some applications, the selected location is the distal end of the tube, and the two filters are separated by the recess defined in the distal end of the tube.

For some applications, the selected location is the distal end of the plunger, and the two filters are separated by the barrier protruding from the distal end of the plunger.

For some applications, the selected location is the distal end of the tube, and the apparatus further includes at least two puncturing elements protruding from the distal end of the plunger, the puncturing elements being configured to puncture the two filters, respectively, upon the plunger being advanced to the filters.

For some applications, the selected location is the distal end of the plunger, and the apparatus further includes at least two puncturing elements protruding in a proximal direction from the distal end of the tube, the puncturing elements being configured to puncture the two filters respectively upon the plunger being advanced to the recess.

For some applications, the selected location is the distal end of the tube, and the distal end of the tube is shaped to define at least two conduits, the conduits being configured to align with the two filters respectively.

For some applications, the selected location is the distal end of the plunger, and the distal end of the tube is shaped to define at least two conduits, the conduits being configured to align with the two filters respectively when the plunger is inside the tube.

For some applications, the plunger is shaped to define at least one plunger lumen containing a particulate-presence-testing-facilitation solution, an opening of the plunger lumen being arranged to align with one of the filters and not to simultaneously align with the other filter, such that the particulate-presence-testing-facilitation solution is applied to only the one of the filters.

For some applications, one of the two filters is at least 25% larger than the other.

For some applications, a culture medium is disposed on at least one of the filters.

For some applications, no culture medium is disposed on at least one of the filters.

For some applications, the plunger, once maximally advanced to the recess, is configured to prevent a particulate-presence-testing-facilitation solution that is applied to one filter from contacting the other filter.

For some applications, a distal portion of the tube is (a) shaped to define at least one enclosed cavity containing a particulate-presence-testing-facilitation solution, and (b) configured such that the particulate-presence-testing-facilitation solution in the cavity is applied to only one filter.

For some applications, a wall of the enclosed cavity is configured to open and release the particulate-presence-testing-facilitation solution to the only one filter.

For some applications, a wall of the enclosed cavity is configured to open and release the particulate-presence-testing-facilitation solution to the only one filter following initiation of distal motion of the plunger in the tube.

For some applications, the two filters are a first filter and a second filter, the barrier is a first barrier, the recess is a first recess, and the first filter separated from the second filter by the first recess or by the first barrier,

(a) the apparatus further including a second barrier protruding in a distal direction from the distal end of the plunger,

(b) the distal end of the tube being further shaped to define a second recess into which the second barrier fits upon the plunger being advanced to the recess, and

(c) the apparatus further including a third filter disposed at a location selected from the group consisting of: the distal end of the tube, and the distal end of the plunger,
the third filter being separated from the second filter by the second recess or by the second protrusion.

For some applications, a culture medium is disposed on at least one of the filters.

For some applications, the plunger, once maximally advanced to the recesses, is configured to prevent a particulate-presence-testing-facilitation solution that is applied to one filter from contacting any other filter.

For some applications, at least one of the filters is at least 25% larger than at least one other filter.

For some applications, the selected location is the distal end of the tube, and the apparatus further includes at least three puncturing elements protruding from a distal end of the plunger, the puncturing elements being configured to puncture the respective filters, upon the plunger being advanced to the filters.

For some applications, the selected location is the distal end of the plunger, and the apparatus further includes at least three puncturing elements protruding in a proximal direction from the distal end of the tube, the puncturing elements being configured to puncture the respective filters, upon the plunger being advanced to the recesses.

For some applications, the selected location is the distal end of the tube, and the distal end of the tube is shaped to define at least three conduits, the conduits being configured to align with the respective filters.

For some applications, the selected location is the distal end of the plunger, and the distal end of the tube is shaped to define at least three conduits, the conduits being configured to align with the respective filters, when the plunger is in the tube.

There is further provided, in accordance with some applications of the present invention apparatus including:

a tube, a distal surface of the tube being oriented at a slant with respect to a lateral wall of the tube;

a plunger sized and shaped to be advanceable within the tube while sealably contacting the tube, a distal surface of the plunger being oriented at a slant with respect to a longitudinal axis of the plunger;

the tube and plunger being configured such that the slant of the distal surface of the plunger aligns with the slant of the distal surface of the tube at at least one rotational orientation of the tube with respect to the plunger; and

a filter disposed at a location selected from the group consisting of: a distal end of the tube, and a distal end of the plunger.

For some applications, the distal surface of the tube is shaped to define a cone.

For some applications, a distal end of the tube is shaped to define at least two conduits disposed at a higher end of the slant of the tube and at a lower end of the slant of the tube, respectively, when a proximal end of the tube or a proximal end of the plunger is resting on a horizontal surface.

For some applications, a proximally-facing distal surface of the tube is oriented at a slant with respect to a lateral wall of the tube, and the plunger is shaped to define at least two plunger lumens disposed over a higher end of the slant of the tube and over a lower end of the slant of the tube, respectively, when a distally-facing distal end of the tube is resting on a horizontal surface.

For some applications, the tube and plunger are shaped to have rotational asymmetry, such that during at least a portion of the advancement of the plunger within the tube, the plunger is advanceable within the tube in only a single orientation of the plunger with respect to the tube.

For some applications, the tube and plunger include corresponding interlockable pieces such that the plunger is advanceable within the tube in only a single orientation of the plunger with respect to the tube.

There is further provided, in accordance with some applications of the present invention, apparatus including:

a plunger; and

a tube,
the plunger being sized and shaped to be advanceable within the tube while sealably contacting the tube, and
a wall of the tube being shaped to define at least one enclosed cavity containing a particulate-presence-testing-facilitation solution and a gas above atmospheric pressure.

For some applications, the apparatus further includes a filter disposed at a location selected from the group consisting of: a distal end of the tube, and a distal end of the plunger.

For some applications, the selected location is the distal end of the tube, and the enclosed cavity is configured to open such that the particulate-presence-testing-facilitation solution is forced out of the enclosed cavity and applied to the filter.

For some applications, the selected location is the distal end of the tube, and the enclosed cavity is configured to open following initiation of distal motion of the plunger in the tube, such that the particulate-presence-testing-facilitation solution is forced out of the enclosed cavity and applied to the filter.

There is further provided, in accordance with some applications of the present invention, apparatus including:

a plunger,
a wall of the plunger being shaped to define at least one enclosed cavity containing a particulate-present-testing-facilitation solution and a gas above atmospheric pressure; and

a tube,
the plunger being sized and shaped to be advanceable within the tube while sealably contacting the tube.

For some applications, the apparatus further includes a filter disposed at a location selected from the group consisting of a distal end of the tube, and a distal end of the plunger.

For some applications, the selected location is the distal end of the plunger and the enclosed cavity is configured to open following initiation of distal motion of the plunger in the tube, such that the particulate-presence-testing-facilitation solution is forced out of the enclosed cavity and applied to the filter.

There is further provided, in accordance with some applications of the present invention, apparatus including:

a tube, closed at a distal end thereof;

a filter disposed within the tube, the tube being shaped to define a fluid-collection compartment distal to the filter; and

a plunger sized and shaped to be advanceable within the tube while sealably contacting the tube, the plunger being arranged to push a fluid through the filter and into the fluid-collection compartment; and

a support which is (a) shaped to define one or more openings, (b) disposed within the tube, and (c) in contact with the filter.

For some applications, the support is disposed proximal to the filter within the tube, and is configured to support the filter during withdrawal of the plunger in a proximal direction.

For some applications, the support is disposed distal to the filter within the tube, and is configured to support the filter during the pushing of the fluid through the filter.

For some applications, the support is positioned to inhibit distal advancement of the plunger past the filter.

For some applications, a wall of the compartment is shaped to define a pressure-release hole, such that air pressure in the compartment generated by advancing the plunger is released through the pressure-release hole.

For some applications, a diameter of the pressure-release hole is 50-1500 microns.

For some applications, the pressure-release hole is disposed above a volume of 2 cc of the compartment when the distal end of the tube is resting on a horizontal surface.

For some applications, the tube is shaped to define a flat external, surface-contact portion which is shaped to contact a horizontal surface when the distal end of the tube is resting on the horizontal surface, the surface-contact portion having a diameter at least equal to a diameter of the filter.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate; using a plunger, pushing the fluid through a filter, disposed within the tube, into a fluid-collection compartment distal to the filter; and

subsequently, while the filter is inside the tube, testing for presence of the particulate, by ascertaining if any of the particulate was trapped by the filter.

For some applications, collecting the fluid in the tube includes collecting gargled fluid in the tube.

For some applications, the gargled fluid includes an element selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid includes a plurality of elements selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid is carbonated.

For some applications, a temperature of the gargled fluid is 1-38 degrees Celsius.

There is further provided, in accordance with some applications of the present invention, apparatus including:

a tube;

a plunger sized and shaped to be advanceable within the tube while sealably contacting the tube;

a filter; and

at least one puncturing element,
the filter and puncturing element being disposed such that:
   (a) the filter is disposed in a distal portion of the tube, and the puncturing element protrudes in a distal direction from a distal end of the plunger, or
   (b) the filter is disposed at a distal end of the plunger, and the puncturing element protrudes in a proximal direction from a distal end of the tube.

For some applications, the at least one puncturing element is configured to puncture the filter upon the plunger being maximally advanced within the tube.

For some applications, the at least one puncturing element is configured to tear the filter upon rotation of the plunger, when the plunger is maximally advanced within the tube.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

pushing the fluid through a filter disposed within a distal portion of the tube;

tearing the filter while the filter is inside the tube; and

subsequently, while the filter is inside the tube, testing for presence of the particulate, by ascertaining if any of the particulate was trapped by the filter by applying a particulate-presence-testing-facilitation solution to the filter.

For some applications, pushing the fluid includes pushing the fluid using a plunger in the tube.

For some applications, tearing the filter includes rotating the plunger with respect to the tube when the plunger is maximally advanced within the tube, at least one puncturing element protruding from a distal end of the plunger.

For some applications, tearing the filter includes rotating the plunger with respect with respect to the tube when the plunger is maximally advanced within the tube.

For some applications, the filter is disposed on a distal end of the plunger and tearing the filter includes rotating the plunger with respect to the tube when the plunger is maximally advanced within the tube, at least one puncturing element protruding in a proximal direction from a distal end of the tube.

For some applications, collecting the fluid in the tube includes collecting gargled fluid in the tube.

For some applications, the gargled fluid includes an element selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid includes a plurality of elements selected from the group consisting of: carbonated water, phosphate buffered saline, pelargonium sidoides extract, tannic acid, balloon flower platycodon grandiflorus, berberine sulfate, S-carboxymethylcysteine, and curcumin.

For some applications, the gargled fluid is carbonated.

For some applications, a temperature of the gargled fluid is 1-38 degrees Celsius.

There is further provided, in accordance with some applications of the present invention, apparatus including:

a plunger; and

a tube,
the plunger being sized and shaped to be advanceable within the tube while sealably contacting the tube, and
the tube and plunger being shaped to have rotational asymmetry such that during at least a portion of the advancement of the plunger within the tube, the plunger is advanceable within the tube in only a single rotational orientation of the plunger with respect to the tube.

There is further provided, in accordance with some applications of the present invention, apparatus including:

a plunger; and

a tube,
the tube and plunger including corresponding interlockable pieces such that the plunger is advanceable within the tube in only a single rotational orientation of the plunger with respect to the tube.

There is further provided, in accordance with some applications of the present invention, apparatus including:

a tube;

a plunger;

a protrusion; and

threading,
the protrusion and threading being disposed such that:
   (a) the threading is disposed on the inside of at least a portion of the tube and the protrusion protrudes outwards from a wall of the plunger, or
   (b) the threading is disposed on the outside of at least a portion of the plunger and the protrusion protrudes inwards from a wall of the tube,
the protrusion being configured to slidably engage the threading such that the plunger is advanceable within the tube by rotation of the plunger with respect to the tube.

There is further provided, in accordance with some applications of the present invention, apparatus including:

a tube;

a plunger;

a protrusion; and

threading,
the protrusion and threading being disposed such that:
   (a) the threading is disposed on the inside of at least a portion of the tube and the protrusion protrudes outwards from a wall of the plunger, or
   (b) the threading is disposed on the outside of at least a portion of the plunger and the protrusion protrudes inwards from a wall of the tube,
a pitch of the threading at a first location being different from the pitch of the threading at a second location.

For some applications, the pitch of the threading at the second location is greater than the pitch of the threading at the first location, the second location being distal to the first location.

For some applications, the pitch of the threading at the second location is less than the pitch of the threading at the first location, the second location being distal to the first location.

There is further provided, in accordance with some applications of the present invention, apparatus including:

a tube;

a plunger;

a protrusion; and

threading,
the protrusion and threading being disposed such that:
   (a) the threading is disposed on the inside of at least a portion of the tube and the protrusion protrudes outwards from a wall of the plunger, or
   (b) the threading is disposed on the outside of at least a portion of the plunger and the protrusion protrudes inwards from a wall of the tube, and
   (c) a portion of the threading closest to a distal end of the tube or plunger is perpendicular to a line, wherein the line is parallel to a longitudinal axis of the tube,
wherein:
(a) the protrusion is configured to slidably engage the threading such that the plunger is advanceable within the tube by rotation of the plunger with respect to the tube, and
(b) the protrusion is configured to engage the perpendicular portion of the threading when the plunger is maximally advanced within the tube, such that the plunger can rotate with respect to the tube without further inhibition by the threading.

There is further provided, in accordance with some applications of the present invention, a method for testing for presence of a particulate selected from the group consisting of: a microorganism, a fungus, a bacteria, a spore, a virus, a mite, a biological cell, a biological antigen, a protein, a protein antigen, and a carbohydrate antigen, the method including:

collecting, in a tube, fluid that potentially contains the particulate;

using a plunger, pushing the fluid through a filter disposed at a location selected from the group consisting of: a distal portion of the tube, and a distal end of the plunger;

removing the plunger from the tube and transferring a sample from the distal end of the plunger to a culture media surface; and

subsequently, ascertaining if any of the particulate is on the culture media surface.

There is further provided, in accordance with some applications of the present invention, apparatus including:

a tube;

a plunger sized and shaped to be advanceable within the tube while sealably contacting the tube; and

a filter disposed at a location selected from the group consisting of: a distal portion of the tube, and a distal end of the plunger,

the distal end of the plunger and the distal end of the tube being configured to tear the filter upon the plunger being maximally advanced within the tube and rotated with respect to the tube.

There is therefore provided, in accordance with an Inventive Concept 1 of the present invention, apparatus including a testing device for testing for the presence of particulate in a liquid, the testing device including:

a liquid container for containing the liquid;

a filter, disposed in or downstream of the liquid container:

a liquid-pressure source, which is arranged to apply pressure to drive the liquid contained in the liquid container through the filter; and

a filter chamber that is (a) disposed downstream of the liquid container, (b) shaped so as to define an inlet, and (c) in fluid communication with the filter.

Inventive Concept 2. The apparatus according to Inventive Concept 1, wherein the inlet of the filter chamber has an inlet area that equals between 4% and 40% of a filter area of the filter.

Inventive Concept 3. The apparatus according to Inventive Concept 1, wherein the inlet of the filter chamber has an inlet area that is less than a greatest cross-sectional area of the filter chamber, the inlet area and the greatest cross-sectional area measured in respective planes parallel to each other.

Inventive Concept 4. The apparatus according to Inventive Concept 1, wherein the filter chamber has an internal volume of between 0.5 and 12 ml.

Inventive Concept 5. The apparatus according to Inventive Concept 4, wherein the internal volume is between 0.5 and 4 ml.

Inventive Concept 6. The apparatus according to Inventive Concept 4, wherein the internal volume is between 1 and 5 ml.

Inventive Concept 7. The apparatus according to Inventive Concept 1, wherein the filter chamber has an internal surface area that equals between 10% and 150% of a filter surface area of an upstream side of the filter.

Inventive Concept 8. The apparatus according to Inventive Concept 1, wherein the filter chamber has an internal length of between 0.5 and 10 cm.
Inventive Concept 9. The apparatus according to Inventive Concept 1, wherein the filter chamber has an internal length equal to between 50% and 2000% of a greatest internal width of the filter chamber.
Inventive Concept 10. The apparatus according to Inventive Concept 1, wherein the filter chamber is nipple-shaped.
Inventive Concept 11. The apparatus according to Inventive Concept 1, wherein the filter chamber includes one or more pressure-activated valves, not disposed at the inlet of the filter chamber.
Inventive Concept 12. The apparatus according to Inventive Concept 1, wherein the liquid-pressure source includes a vacuum pump disposed downstream of filter.
Inventive Concept 13. The apparatus according to Inventive Concept 1, wherein the liquid-pressure source includes a positive-pressure pump disposed upstream of the filter.
Inventive Concept 14. The apparatus according to Inventive Concept 1, wherein the filter has a filter surface area of an upstream side of the filter that equals between 0.3 and 100 cm2.
Inventive Concept 15. The apparatus according to Inventive Concept 14, wherein the filter surface area equals between 0.3 and 30 cm2.
Inventive Concept 16. The apparatus according to Inventive Concept 1, wherein the filter is configured to trap at least 40% of group A streptococcus bacteria and allow passage of the liquid.
Inventive Concept 17. The apparatus according to Inventive Concept 1, wherein the filter is configured to trap at least 40% of the particulate to be tested and allow passage of the liquid.
Inventive Concept 18. The apparatus according to any one of Inventive Concepts 1-17, wherein the testing device further includes a waste liquid receptacle, which is coupled to the liquid container downstream of the filter, and wherein the liquid-pressure source is arranged to apply pressure to drive the liquid contained in the liquid container through the filter and then into the waste liquid receptacle.
Inventive Concept 19. The apparatus according to Inventive Concept 18,

wherein the liquid-pressure source includes a plunger, which includes a plunger head that is shaped so as to be insertable into the liquid container, and

wherein the plunger is shaped so as to define the waste liquid receptacle.

Inventive Concept 20. The apparatus according to Inventive Concept 18, wherein the waste liquid receptacle contains an antibacterial agent.
Inventive Concept 21. The apparatus according to Inventive Concept 18, wherein the filter chamber is laterally surrounded by at least a portion of the waste liquid receptacle.
Inventive Concept 22. The apparatus according to Inventive Concept 18, wherein the filter chamber is disposed within the waste liquid receptacle.
Inventive Concept 23. The apparatus according to any one of Inventive Concepts 1-17, wherein the filter is removably disposed upstream of the filter chamber with the filter partially covering the inlet of the filter chamber.
Inventive Concept 24. The apparatus according to Inventive Concept 23, wherein the inlet of the filter chamber has an inlet centroid that is disposed within a distance of a filter centroid, the distance equal to 50% of a greatest dimension of the filter, when the filter is removably disposed upstream of the filter chamber with the filter partially covering the inlet of the filter chamber.
Inventive Concept 25. The apparatus according to Inventive Concept 23, wherein the testing device further includes a support for the filter, disposed at least partially between the inlet of the filter chamber and the filter.
Inventive Concept 26. The apparatus according to Inventive Concept 23, wherein the apparatus further includes an elongate member configured to push at least a portion of the filter into the filter chamber.
Inventive Concept 27. The apparatus according to Inventive Concept 23,

wherein the liquid-pressure source includes a plunger, which includes a plunger head that is shaped so as to be insertable into the liquid container, and

wherein the plunger head is configured to push at least a portion of the filter into the filter chamber.

Inventive Concept 28. The apparatus according to Inventive Concept 23, wherein the testing device further includes a frangible seal that removably blocks liquid flow into the inlet of the filter chamber.
Inventive Concept 29. The apparatus according to Inventive Concept 23, wherein the filter chamber includes one or more valves, not disposed at the inlet of the filter chamber.
Inventive Concept 30. The apparatus according to Inventive Concept 29, wherein the one or more valves include one or more pressure-activated valves.
Inventive Concept 31. The apparatus according to Inventive Concept 30, wherein the one or more valves include one or more non-pressure-activated valves.
32. The apparatus according to Inventive Concept 29,

wherein the liquid container is shaped so as to define one or more openings through a wall of the liquid container,

wherein the one or more openings are downstream of the filter when the filter is removably disposed upstream of the filter chamber with the filter partially covering the inlet of the filter chamber, and

wherein the filter chamber is not disposed so as to receive the liquid that is driven through the one or more openings.

Inventive Concept 33. The apparatus according to any one of Inventive Concepts 1-17, wherein the filter is disposed at least partially within the filter chamber.
Inventive Concept 34. The apparatus according to Inventive Concept 33, wherein the filter is disposed entirely within the filter chamber.
Inventive Concept 35. The apparatus according to Inventive Concept 33, wherein the filter is shaped as a receptacle.
Inventive Concept 36. The apparatus according to any one of Inventive Concepts 1-17, wherein the liquid-pressure source includes a plunger, which includes a plunger head that is shaped so as to be insertable into the liquid container.
Inventive Concept 37. The apparatus according to Inventive Concept 36, wherein the plunger is shaped so as to define the filter chamber.
Inventive Concept 38. The apparatus according to any one of Inventive Concepts 1-17, wherein the testing device further includes one or more heating elements which are configured to heat the filter at a generally constant temperature, the temperature in the range of 20 and 50 degrees C.
Inventive Concept 39. The apparatus according to Inventive Concept 38, wherein the temperature is in the range of 30 to 40 degrees C.
Inventive Concept 40. The apparatus according to Inventive Concept 38, wherein the liquid container includes, upstream of the filter, a frangible dividing waterproof or water-resistant membrane that isolates the filter from the liquid in the liquid container.
Inventive Concept 41. The apparatus according to Inventive Concept 38,
wherein the liquid-pressure source includes a plunger, which includes a plunger head that is shaped so as to be insertable into the liquid container, and
wherein the one or more heating elements are disposed in the plunger.
   Inventive Concept 42. The apparatus according to any one of Inventive Concepts 1-17, wherein the liquid container is shaped so as to define upstream and downstream openings, and wherein an area of the upstream opening is greater than the area of the downstream opening.
   Inventive Concept 43. The apparatus according to Inventive Concept 42, wherein the liquid container includes an upstream end portion that includes the upstream opening, and wherein the upstream end portion is conical.
   Inventive Concept 44. The apparatus according to Inventive Concept 43, wherein a diameter of the upstream opening is at least 20% greater than a diameter of the downstream opening.
   Inventive Concept 45. The apparatus according to any one of Inventive Concepts 1-17, wherein the apparatus further includes sterile packaging, in which at least the liquid container, the filter chamber, and the filter are removably disposed.
   Inventive Concept 46. The apparatus according to any one of Inventive Concepts 1-17, wherein the apparatus further includes at least one container including an extraction reagent.
   Inventive Concept 47. The apparatus according to Inventive Concept 46, wherein the apparatus further includes a test strip.

There is further provided, in accordance with an Inventive Concept 48 of the present invention, a method including:

applying pressure to drive liquid contained in a liquid container of a testing device through a filter of the testing device, wherein the filter is disposed in or downstream of the liquid container, and wherein the liquid includes at least one substance selected from the group of substances consisting of gargled fluid, saliva not swabbed from a throat of a patient, and an incubated culture medium containing a biological sample; and

thereafter, testing, within a filter chamber of the testing device, for the presence of particulate trapped by the filter while the filter is disposed at least partially in the filter chamber, wherein the filter chamber is (a) disposed downstream of the liquid container, (b) shaped so as to define an inlet, and (c) in fluid communication with the filter.

Inventive Concept 49. The method according to Inventive Concept 48, wherein testing includes applying an extraction reagent to the filter while the filter is in the filter chamber.
Inventive Concept 50. The method according to Inventive Concept 49, wherein testing further includes after applying the extraction reagent, inserting a test strip into the filter chamber and examining the test strip to test for the presence of the particulate.
Inventive Concept 51. The method according to Inventive Concept 48, further including taking a sample from the filter, and testing the sample, outside the testing device, for the presence of the particulate.
Inventive Concept 52. The method according to Inventive Concept 51, wherein testing the sample outside the testing device includes testing the sample outside the testing device without first incubating the sample.
Inventive Concept 53. The method according to Inventive Concept 52, wherein testing the sample outside the testing device includes performing a technique selected from the group consisting of: a nucleic acid amplification rapid strep test (RST) technique and real-time quantitative polymerase chain reaction (qPCR) assaying.
Inventive Concept 54. The method according to Inventive Concept 51, wherein testing the sample outside the testing device includes incubating the sample outside the testing device and subsequently testing the sample outside the testing device.
Inventive Concept 55. The method according to Inventive Concept 54, wherein testing the sample outside the testing device includes performing a technique selected from the group consisting of: lateral flow immunoassaying, an ELISA-based rapid strep test (RST), an antibody-coated-beads-based RST, a nucleic-acid-based RST, and a fluorescent immunoassaying (FIA).
Inventive Concept 56. The method according to Inventive Concept 48, wherein the liquid includes the gargled fluid.
Inventive Concept 57. The method according to Inventive Concept 48, wherein the liquid includes the saliva not swabbed from the throat of the patient.
Inventive Concept 58. The method according to Inventive Concept 57, wherein the saliva not swabbed from the throat of the patient is saliva spit by the patient.
Inventive Concept 59. The method according to Inventive Concept 48, wherein the liquid includes the incubated culture medium containing the biological sample.
Inventive Concept 60. The method according to Inventive Concept 48, wherein applying the pressure includes pushing a plunger including a plunger head inserted into the liquid container.
Inventive Concept 61. The method according to Inventive Concept 60, wherein the plunger is shaped so as to define the filter chamber.
Inventive Concept 62. The method according to Inventive Concept 48, wherein applying the pressure includes applying positive pressure using a positive-pressure pump disposed upstream of the filter.
Inventive Concept 63. The method according to Inventive Concept 48, wherein applying the pressure includes applying negative pressure using a vacuum pump disposed downstream of the filter.
Inventive Concept 64. The method according to Inventive Concept 48,

wherein applying the pressure includes applying the pressure while the filter is removably disposed upstream of the filter chamber with the filter partially covering the inlet of the filter chamber, and

wherein testing includes pushing at least a portion of the filter into the filter chamber.

Inventive Concept 65. The method according to Inventive Concept 64, wherein pushing includes pushing the at least a portion of the filter into the filter chamber using an elongate member.
Inventive Concept 66. The method according to Inventive Concept 65, wherein pushing the at least a portion of the filter into the filter chamber includes taking a sample from the filter using the elongate member, and testing the sample, outside the testing device, for the presence of the particulate.
Inventive Concept 67. The method according to Inventive Concept 64,

wherein the testing device further includes a frangible seal that removably blocks liquid flow into the inlet of the filter chamber, and

wherein the method further includes, after applying the pressure and before testing for the presence of the particulate trapped by the filter, breaking the frangible seal.

Inventive Concept 68. The method according to Inventive Concept 64, wherein the filter chamber includes one or more valves, not disposed at the inlet of the filter chamber.
Inventive Concept 69. The method according to Inventive Concept 68, wherein the one or more valves include one or more pressure-activated valves.
Inventive Concept 70. The method according to Inventive Concept 69, wherein the one or more valves include one or more non-pressure-activated valves.
Inventive Concept 71. The method according to Inventive Concept 48, wherein applying the pressure includes applying the pressure while the filter is disposed at least partially within the filter chamber.
Inventive Concept 72. The method according to Inventive Concept 71, wherein applying the pressure includes applying the pressure while the filter is disposed entirely within the filter chamber.
Inventive Concept 73. The method according to Inventive Concept 71, wherein the filter is shaped as a receptacle.
Inventive Concept 74. The method according to Inventive Concept 48, further including, before applying the pressure, taking a sample of the liquid, and testing the sample, outside the testing device, for the presence of the particulate.

There is still further provided, in accordance with an Inventive Concept 75 of the present invention, a method including:

applying pressure to drive liquid contained in a liquid container of a testing device (a) through a filter of the testing device and (b) then through one or more valves of the testing device, wherein the filter is disposed in or downstream of the liquid container, wherein the one or more valves are disposed downstream of the filter, and wherein the liquid includes at least one substance selected from the group of substances consisting of gargled fluid, saliva not swabbed from a throat of a patient, and an incubated culture medium containing a biological sample; and

thereafer, testing, within the testing device, for the presence of particulate trapped by the filter while the one or more valves are closed and the filter is disposed in the testing device.

Inventive Concept 76. The method according to Inventive Concept 75, wherein testing includes applying an extraction reagent to the filter.
Inventive Concept 77. The method according to Inventive Concept 76, wherein testing further includes after applying the extraction reagent, inserting a test strip into the testing device and examining the test strip to test for the presence of the particulate.
Inventive Concept 78. The method according to Inventive Concept 75, wherein the liquid includes the gargled fluid.
Inventive Concept 79. The method according to Inventive Concept 75, wherein the liquid includes the saliva not swabbed from the throat of the patient.
Inventive Concept 80. The method according to Inventive Concept 79, wherein the saliva not swabbed from the throat of the patient is saliva spit by the patient.
Inventive Concept 81. The method according to Inventive Concept 75, wherein the liquid includes the incubated culture medium containing the biological sample.
Inventive Concept 82. The method according to Inventive Concept 75, wherein the liquid container has an internal volume of between 0.5 and 500 ml.
Inventive Concept 83. The method according to Inventive Concept 75, wherein applying the pressure includes pushing a plunger including a plunger head inserted into the liquid container.
Inventive Concept 84. The method according to Inventive Concept 75, wherein applying the pressure includes applying positive pressure using a positive-pressure pump disposed upstream of the filter.
Inventive Concept 85. The method according to Inventive Concept 75, wherein applying the pressure includes applying negative pressure using a vacuum pump disposed downstream of the one or more valves.
Inventive Concept 86. The method according to Inventive Concept 75, wherein the one or more valves include one or more pressure-activated valves.
Inventive Concept 87. The method according to Inventive Concept 75, wherein the one or more valves include one or more non-pressure-activated valves.
Inventive Concept 88. The method according to Inventive Concept 87, wherein the testing device is configured to automatically close the one or more non-pressure-activated valves after the pressure is applied to drive the liquid through the filter and then through the one or more non-pressure-activated valves.
Inventive Concept 89. The method according to Inventive Concept 87, wherein the one or more non-pressure-activated valves include two discs that are shaped so as to define respective sets of openings, and wherein the one or more non-pressure-activated valves are configured to assume open and closed states when the two sets of openings are aligned and non-aligned with each other.
Inventive Concept 90. The method according to Inventive Concept 87,

wherein applying the pressure includes pushing a plunger including a plunger head inserted into the liquid container, and

wherein the testing device is configured to automatically close the one or more non-pressure-activated valves after the plunger applies the pressure to drive the liquid contained in the liquid container through the filter and then through the one or more non-pressure-activated valves.

Inventive Concept 91. The method according to Inventive Concept 90, wherein the testing device is configured such that motion of the plunger automatically closes the one or more non-pressure-activated valves after the plunger applies the pressure to drive the liquid contained in the liquid container through the filter and then through the one or more non-pressure-activated valves.
Inventive Concept 92. The method according to Inventive Concept 91,

wherein pushing the plunger include rotating the plunger, and

wherein the testing device is configured such that rotational motion of the plunger automatically closes the one or more non-pressure-activated valves after the plunger applies the pressure to drive the liquid contained in the liquid container through the filter and then through the one or more non-pressure-activated valves.

Inventive Concept 93. The method according to Inventive Concept 92, wherein the plunger is shaped so as to define one or more plunger threads, and wherein an internal wall of the liquid container is shaped so as to define one or more liquid-container threads that engage the one or more plunger threads such that rotation of the plunger advances the plunger in a downstream direction within the liquid container.
Inventive Concept 94. The method according to Inventive Concept 92,

wherein the one or more non-pressure-activated valves include two discs that are shaped so as to define respective sets of openings, and wherein the one or more non-pressure-activated valves are configured to assume open and closed states when the two sets of openings are aligned and non-aligned with each other,

wherein pushing the plunger include rotating the plunger, and

wherein the testing device is configured such that rotational motion of the plunger automatically closes the one or more non-pressure-activated valves by rotating at least one of the two discs with respect to the other of the discs, after the plunger applies the pressure to drive the liquid contained in the liquid container through the filter and then through the one or more non-pressure-activated valves.

Inventive Concept 95. The method according to Inventive Concept 75, wherein applying the pressure includes applying the pressure to drive the liquid contained in the liquid container through the filter, then through the one or more valves, and then into a waste liquid receptacle of the testing device, wherein the waste liquid receptacle is coupled to the liquid container downstream of the one or more valves.
Inventive Concept 96. The method according to Inventive Concept 95,

wherein applying the pressure includes pushing a plunger including a plunger head inserted into the liquid container, and

wherein the plunger is shaped so as to define the waste liquid receptacle.

Inventive Concept 97. The method according to Inventive Concept 95, wherein the waste liquid receptacle contains an antibacterial agent.
Inventive Concept 98. The method according to Inventive Concept 75, wherein the testing device further includes a filter chamber that is (a) disposed downstream of the liquid container, (b) shaped so as to define an inlet, and (c) in fluid communication with filter.
Inventive Concept 99. The method according to Inventive Concept 98,

wherein applying the pressure includes pushing a plunger including a plunger head inserted into the liquid container, and

wherein the plunger is shaped so as to define the filter chamber.

Inventive Concept 100. The method according to Inventive Concept 98,

wherein the liquid-pressure source is arranged to apply pressure to drive the liquid contained in the liquid container through the filter, then through the one or more valves, and then into a waste liquid receptacle of the testing device, wherein the waste liquid receptacle is coupled to the liquid container downstream of the one or more valves, and

wherein the filter chamber is laterally surrounded by at least a portion of the waste liquid receptacle.

Inventive Concept 101. The method according to Inventive Concept 98,

wherein the liquid-pressure source is arranged to apply pressure to drive the liquid contained in the liquid container through the filter, then through the one or more valves, and then into a waste liquid receptacle of the testing device, wherein the waste liquid receptacle is coupled to the liquid container downstream of the one or more valves, and

wherein the filter chamber is disposed within the waste liquid receptacle.

Inventive Concept 102. The method according to Inventive Concept 98, wherein the inlet of the filter chamber has an inlet area that is less than a greatest cross-sectional area of the filter chamber, the inlet area and the greatest cross-sectional area measured in respective planes parallel to each other.
Inventive Concept 103. The method according to Inventive Concept 98, wherein the filter chamber is nipple-shaped.
Inventive Concept 104. The method according to Inventive Concept 98, wherein the filter chamber includes at least one of the one or more valves, not disposed at the inlet of the filter chamber.
Inventive Concept 105. The method according to Inventive Concept 98, wherein applying the pressure includes applying the pressure while the filter is removably disposed upstream of the filter chamber with the filter partially covering the inlet of the filter chamber.
Inventive Concept 106. The method according to Inventive Concept 105, wherein the method further includes, after applying the pressure and before testing for the presence of the particulate trapped by the filter, pushing at least a portion of the filter into the filter chamber.
Inventive Concept 107. The method according to Inventive Concept 106,

wherein applying the pressure includes pushing a plunger including a plunger head inserted into the liquid container, and

wherein pushing the at least a portion of the filter into the filter chamber includes pushing the at least a portion of the filter into the filter chamber using the plunger head.

Inventive Concept 108. The method according to Inventive Concept 106, wherein pushing includes pushing the at least a portion of the filter into the filter chamber using an elongate member.
Inventive Concept 109. The method according to Inventive Concept 108, wherein pushing the at least a portion of the filter into the filter chamber includes taking a sample from the filter using the elongate member, and testing the sample, outside the testing device, for the presence of the particulate.
Inventive Concept 110. The method according to Inventive Concept 105, wherein the inlet of the filter chamber has an inlet centroid that is disposed less than a distance from a filter centroid, the distance equal to 50% of a greatest dimension of the filter.
Inventive Concept 111. The method according to Inventive Concept 105,

wherein the testing device further includes a frangible seal that removably blocks liquid flow into the inlet of the filter chamber, and

wherein the method further includes, after applying the pressure and before testing for the presence of the particulate trapped by the filter, breaking the frangible seal.

Inventive Concept 112. The method according to Inventive Concept 98, wherein the filter chamber is not disposed so as to receive the liquid that is driven through at least one of the one or more valves.
Inventive Concept 113. The method according to Inventive Concept 98, wherein the filter chamber includes at least one of the one or more valves, not disposed at the inlet of the filter chamber.
Inventive Concept 114. The method according to Inventive Concept 113,

wherein the liquid container is shaped so as to define one or more openings through a wall of the liquid container,

wherein the one or more openings are downstream of the filter when the filter is removably disposed upstream of the filter chamber with the filter partially covering the inlet of the filter chamber,

wherein the filter chamber is not disposed so as to receive the liquid that is driven through the one or more openings, and

wherein applying the pressure includes applying the pressure to drive the liquid (i) partially through (a) the filter and (b) one or more of the one or more valves of the testing device and (ii) partially through the one or more openings.

Inventive Concept 115. The method according to Inventive Concept 98, wherein the filter is disposed at least partially within the filter chamber.
Inventive Concept 116. The method according to Inventive Concept 115, wherein the filter is disposed entirely within the filter chamber.
Inventive Concept 117. The method according to Inventive Concept 115, wherein the filter is shaped as a receptacle.
Inventive Concept 118. The method according to Inventive Concept 75, wherein the filter is configured to trap at least 40% of group A streptococcus bacteria and allow passage of the liquid.
Inventive Concept 119. The method according to Inventive Concept 75, wherein the filter is configured to trap at least 40% of the particulate.
Inventive Concept 120. The method according to Inventive Concept 75, wherein the method further includes, before testing for the presence of the particulate trapped by the filter, activating one or more heating elements which are configured to heat the filter at a generally constant temperature, the temperature in the range of 20 and 50 degrees C.
Inventive Concept 121. The method according to Inventive Concept 120, wherein the temperature is in the range of 30 to 40 degrees C.
Inventive Concept 122. The method according to Inventive Concept 120, wherein the one or more heating elements are disposed within the testing device.
Inventive Concept 123. The method according to Inventive Concept 122,

wherein applying the pressure includes pushing a plunger including a plunger head inserted into the liquid container, and

wherein the one or more heating elements are disposed in the plunger.

Inventive Concept 124. The method according to Inventive Concept 75, wherein the method further includes, before most of the liquid initially contained in the testing device has been driven through the filter, activating one or more heating elements which are configured to heat the filter at a generally constant temperature, the temperature in the range of 20 and 50 degrees C.
Inventive Concept 125. The method according to Inventive Concept 124, wherein the temperature is in the range of 30 to 40 degrees C.
Inventive Concept 126. The method according to Inventive Concept 124, wherein activating the one or more heating elements includes activating the one or more heating elements before any of the liquid initially contained in the testing device has been driven through the filter.
Inventive Concept 127. The method according to Inventive Concept 126,

wherein the liquid container includes, upstream of the filter, a frangible dividing waterproof or water-resistant membrane that isolates the filter from the liquid in the liquid container, and

wherein the method further includes breaking the frangible dividing waterproof or water-resistant membrane before driving the liquid through the filter.

Inventive Concept 128. The method according to Inventive Concept 126, wherein activating the one or more heating elements includes orienting the testing device with the filter above the liquid, such that the liquid is not in contact with the filter during heating.
Inventive Concept 129. The method according to Inventive Concept 124, wherein the one or more heating elements are disposed within the testing device.
Inventive Concept 130. The method according to Inventive Concept 75,

wherein the one or more valves are one or more first valves, and

wherein the testing device further includes one or more second pressure relief valves, which are in fluid communication with the liquid container and are disposed upstream of the filter.

Inventive Concept 131. The method according to Inventive Concept 130,

wherein the liquid-pressure source is arranged to apply pressure to drive the liquid contained in the liquid container through the filter, then through the one or more valves, and then into a waste liquid receptacle of the testing device, wherein the waste liquid receptacle is coupled to the liquid container downstream of the one or more valves, and

wherein the one or more second pressure relief valves are in fluid communication with the waste liquid receptacle not via the filter.

Inventive Concept 132. The method according to Inventive Concept 130,

wherein the liquid-pressure source includes a plunger, which includes (a) a plunger shaft and (b) a plunger head that is disposed at a downstream end portion of the plunger shaft and shaped so as to be insertable into the liquid container,

wherein the testing device includes one or more unfiltered liquid receptacles,

wherein the one or more second pressure relief valves are in fluid communication with the one or more unfiltered liquid receptacles, and

wherein the method further includes, after applying the pressure, taking a sample of the liquid in the one or more unfiltered liquid receptacles, and testing the sample, outside the testing device, for the presence of the particulate.

Inventive Concept 133. The method according to Inventive Concept 132, wherein the one or more unfiltered liquid receptacles are disposed along the plunger shaft.
Inventive Concept 134. The method according to Inventive Concept 133, wherein the one or more unfiltered liquid receptacles are removably coupled to the plunger.
Inventive Concept 135. The method according to Inventive Concept 130,

wherein the one or more first valves include one or more first pressure-activated valves configured to open upon exposure to a first pressure gradient across the one or more first pressure-activated valves, and

wherein the one or more second pressure relief valves are configured to open upon exposure to a second pressure gradient across the one or more second pressure relief valves, the second pressure gradient greater than the first pressure gradient.

Inventive Concept 136. The method according to Inventive Concept 75, wherein the method further includes, before applying the pressure, removing the liquid container, the one or more valves, and the filter from sterile packaging.
Inventive Concept 137. The method according to Inventive Concept 75, wherein the particulate includes biological particulate.
Inventive Concept 138. The method according to Inventive Concept 137, wherein the biological particulate is selected from the group consisting of: a microorganism, a fungus, a bacterium, a spore, a virus, a mite, a biological cell, a biological antigen, a protein, a protein antigen, and a carbohydrate antigen.
Inventive Concept 139. The method according to Inventive Concept 75, wherein testing for the presence of the particulate includes applying an extraction reagent to the filter after applying the pressure.
Inventive Concept 140. The method according to Inventive Concept 139, wherein testing for the presence of the particulate includes using a test strip.
Inventive Concept 141. The method according to Inventive Concept 75, further including, after applying the pressure, taking a sample from the filter, and testing the sample, outside the testing device, for the presence of the particulate trapped by the filter.
Inventive Concept 142. The method according to Inventive Concept 75, further including, before applying the pressure, taking a sample of the liquid, and testing the sample, outside the testing device, for the presence of the particulate.
Inventive Concept 143. The method according to Inventive Concept 75, further including taking a sample from the filter, and testing the sample, outside the testing device, for the presence of the particulate.
Inventive Concept 144. The method according to Inventive Concept 143, wherein testing the sample outside the testing device includes testing the sample outside the testing device without first incubating the sample.
Inventive Concept 145. The method according to Inventive Concept 144, wherein testing the sample outside the testing device includes performing a technique selected from the group consisting of: a nucleic acid amplification rapid strep test (RST) technique and real-time quantitative polymerase chain reaction (qPCR) assaying.
Inventive Concept 146. The method according to Inventive Concept 143, wherein testing the sample outside the testing device includes incubating the sample outside the testing device and subsequently testing the sample outside the testing device.
Inventive Concept 147. The method according to Inventive Concept 146, wherein testing the sample outside the testing device includes performing a technique selected from the group consisting of: lateral flow immunoassaying, an ELISA-based rapid strep test (RST), an antibody-coated-beads-based RST, a nucleic-acid-based RST, and a fluorescent immunoassaying (FIA).

There is additionally provided, in accordance with an Inventive Concept 148 of the present invention, apparatus including a testing device for testing for the presence of particulate in a liquid, the testing device including:

a liquid container for containing the liquid, the liquid container shaped so as to define upstream and downstream openings;

a filter, removably disposed in the liquid container; and

a plunger head that (a) is shaped so as to be insertable into the liquid container so as to form a movable seal with a wall of the liquid container, and (b) is arranged such that when pushed, the plunger head applies pressure to drive the liquid contained in the liquid container through the filter and then through the downstream opening,

wherein the testing device is configured such that rotation of the plunger head radially compresses the filter toward a central longitudinal axis of the plunger head.

Inventive Concept 149. The apparatus according to Inventive Concept 148, wherein the testing device is configured such that the rotation of the plunger head crushes the filter.
Inventive Concept 150. The apparatus according to Inventive Concept 149, wherein the plunger head includes a protrusion, and wherein the testing device is configured such that the rotation of the plunger head causes the protrusion to move radially toward the central longitudinal axis of the plunger head.
Inventive Concept 151. The apparatus according to Inventive Concept 150,

wherein the liquid container is shaped so as to define a filter-support surface surrounding the downstream opening,

wherein the filter-support surface supports a radial portion of the filter excluding a central portion of the filter,

wherein the filter-support surface is shaped so as to define a spiral groove,

wherein the protrusion is configured to engage the spiral groove through the filter, and

wherein the testing device is configured such that the rotation of the plunger head causes the spiral groove to guide the protrusion radially toward the central longitudinal axis of the plunger head.

There is yet additionally provided, in accordance with an Inventive Concept 152 of the present invention, a method including:

inserting a plunger head into a liquid container of a testing device so as to form a movable seal with a wall of the liquid container;

pushing the plunger head to apply pressure to drive liquid contained in the liquid container through a filter of the testing device and then through a downstream opening of the liquid container, which also has an upstream opening, wherein the filter is removably disposed in the liquid container; and

rotating the plunger head to radially crush the filter toward a central longitudinal axis of the plunger head.

Inventive Concept 153. The method according to Inventive Concept 152, further including, after rotating the plunger head, testing the filter for the presence of particulate trapped by the filter.
Inventive Concept 154. The method according to Inventive Concept 152, wherein rotating the plunger head crushes the filter.
Inventive Concept 155. The method according to Inventive Concept 154, wherein the plunger head includes a protrusion, and wherein rotating the plunger head causes the protrusion to move radially toward the central longitudinal axis of the plunger head.
Inventive Concept 156. The method according to Inventive Concept 155,

wherein the liquid container is shaped so as to define a filter-support surface surrounding the downstream opening,

wherein the filter-support surface supports a radial portion of the filter excluding a central portion of the filter,

wherein the filter-support surface is shaped so as to define a spiral groove,

wherein the protrusion is configured to engage the spiral groove through the filter, and

wherein rotating the plunger head causes the spiral groove to guide the protrusion radially toward the central longitudinal axis of the plunger head.

Inventive Concept There is further provided, in accordance with an Inventive Concept 157 of the present invention, apparatus including a testing device for testing for the presence of particulate in a liquid, the testing device including:

a liquid container for containing the liquid, wherein the liquid container has an internal volume of between 0.5 and 500 ml; one or more valves;

a filter, disposed in or downstream of the liquid container and upstream of the one or more valves; and

a plunger, which (a) includes a plunger head that is shaped so as to be insertable into the liquid container, and (b) is arranged to apply pressure to drive the liquid contained in the liquid container through the filter and then through the one or more valves.

Inventive Concept 158. The apparatus according to Inventive Concept 157, wherein the one or more valves include one or more pressure-activated valves.
Inventive Concept 159. The apparatus according to Inventive Concept 157, wherein the one or more valves include one or more non-pressure-activated valves.
Inventive Concept 160. The apparatus according to Inventive Concept 159, wherein the one or more non-pressure-activated valves include two discs that are shaped so as to define respective sets of openings, and wherein the one or more non-pressure-activated valves are configured to assume open and closed states when the two sets of openings are aligned and non-aligned with each other.
Inventive Concept 161. The apparatus according to Inventive Concept 159, wherein the testing device is configured to automatically close the one or more non-pressure-activated valves after the plunger applies the pressure to drive the liquid contained in the liquid container through the filter and then through the one or more non-pressure-activated valves.
Inventive Concept 162. The apparatus according to Inventive Concept 161, wherein the testing device is configured such that motion of the plunger automatically closes the one or more non-pressure-activated valves after the plunger applies the pressure to drive the liquid contained in the liquid container through the filter and then through the one or more non-pressure-activated valves.
Inventive Concept 163. The apparatus according to Inventive Concept 162, wherein the testing device is configured such that rotational motion of the plunger automatically closes the one or more non-pressure-activated valves after the plunger applies the pressure to drive the liquid contained in the liquid container through the filter and then through the one or more non-pressure-activated valves.
Inventive Concept 164. The apparatus according to Inventive Concept 163, wherein the plunger is shaped so as to define one or more plunger threads, and wherein an internal wall of the liquid container is shaped so as to define one or more liquid-container threads that engage the one or more plunger threads such that rotation of the plunger advances the plunger in a downstream direction within the liquid container.
Inventive Concept 165. The apparatus according to Inventive Concept 163,

wherein the one or more non-pressure-activated valves include two discs that are shaped so as to define respective sets of openings, and wherein the one or more non-pressure-activated valves are configured to assume open and closed states when the two sets of openings are aligned and non-aligned with each other, and

wherein the testing device is configured such that rotational motion of the plunger automatically closes the one or more non-pressure-activated valves by rotating at least one of the two discs with respect to the other of the discs, after the plunger applies the pressure to drive the liquid contained in the liquid container through the filter and then through the one or more non-pressure-activated valves.

Inventive Concept 166. The apparatus according to Inventive Concept 157, wherein the filter has a filter surface are of an upstream side of the filter that equals between 0.3 and 100 cm2.
Inventive Concept 167. The apparatus according to Inventive Concept 166, wherein the filter surface area equals between 0.3 and 30 cm2.
Inventive Concept 168. The apparatus according to Inventive Concept 157, wherein the filter is configured to trap at least 40% of group A streptococcus bacteria and allow passage of the liquid.
Inventive Concept 169. The apparatus according to Inventive Concept 157, wherein the filter is configured to trap at least 40% of the particulate to be tested and allow passage of the liquid.
Inventive Concept 170. The apparatus according to any one of Inventive Concepts 157-169, wherein the testing device further includes a waste liquid receptacle, which is coupled to the liquid container downstream of the one or more valves, and wherein the plunger is arranged to apply pressure to drive the liquid contained in the liquid container through the filter, then through the one or more valves, and then into the waste liquid receptacle.
Inventive Concept 171. The apparatus according to Inventive Concept 170, wherein the plunger is shaped so as to define the waste liquid receptacle.
Inventive Concept 172. The apparatus according to Inventive Concept 170, wherein the waste liquid receptacle contains an antibacterial agent.
Inventive Concept 173. The apparatus according to any one of Inventive Concepts 157-169, wherein the testing device further includes a filter chamber that is (a) disposed downstream of the liquid container, (b) shaped so as to define an inlet, and (c) in fluid communication with the filter.
Inventive Concept 174. The apparatus according to Inventive Concept 173, wherein the plunger is shaped so as to define the filter chamber.
Inventive Concept 175. The apparatus according to Inventive Concept 173,

wherein the testing device further includes a waste liquid receptacle, which is coupled to the liquid container downstream of the filter,

wherein the plunger is arranged to apply pressure to drive the liquid contained in the liquid container through the filter and then into the waste liquid receptacle, and

wherein the filter chamber is laterally surrounded by at least a portion of the waste liquid receptacle.

Inventive Concept 176. The apparatus according to Inventive Concept 173,

wherein the testing device further includes a waste liquid receptacle, which is coupled to the liquid container downstream of the filter,

wherein the plunger is arranged to apply pressure to drive the liquid contained in the liquid container through the filter and then into the waste liquid receptacle, and

wherein the filter chamber is disposed within the waste liquid receptacle.

Inventive Concept 177. The apparatus according to Inventive Concept 173, wherein the inlet of the filter chamber has an inlet area that equals between 4% and 40% of a filter surface area of an upstream side of the filter.
Inventive Concept 178. The apparatus according to Inventive Concept 173, wherein the inlet of the filter chamber has an inlet area that is less than a greatest cross-sectional area of the filter chamber, the inlet area and the greatest cross-sectional area measured in respective planes parallel to each other.
Inventive Concept 179. The apparatus according to Inventive Concept 173, wherein the filter chamber has an internal volume of between 0.5 and 12 ml.
Inventive Concept 180. The apparatus according to Inventive Concept 179, wherein the internal volume is between 0.5 and 4 ml.
Inventive Concept 181. The apparatus according to Inventive Concept 179, wherein the internal volume is between 1 and 5 ml.
Inventive Concept 182. The apparatus according to Inventive Concept 173, wherein the filter chamber has an internal surface area that equals between 10% and 150% of a filter surface area of an upstream side of the filter.
Inventive Concept 183. The apparatus according to Inventive Concept 173, wherein the filter chamber has an internal length of between 0.5 and 10 cm.
Inventive Concept 184. The apparatus according to Inventive Concept 173, wherein the filter chamber has an internal length equal to between 50% and 2000% of a greatest internal width of the filter chamber.
Inventive Concept 185. The apparatus according to Inventive Concept 173, wherein the filter chamber is nipple-shaped.
Inventive Concept 186. The apparatus according to Inventive Concept 173, wherein the filter chamber includes at least one of the one or more valves, not disposed at the inlet of the filter chamber.
Inventive Concept 187. The apparatus according to Inventive Concept 173, wherein the filter is removably disposed upstream of the filter chamber with the filter partially covering the inlet of the filter chamber.
Inventive Concept 188. The apparatus according to Inventive Concept 187, wherein the inlet of the filter chamber has an inlet centroid that is disposed less than a distance from a filter centroid, the distance equal to 50% of a greatest dimension of the filter, when the filter is removably disposed upstream of the filter chamber with the filter partially covering the inlet of the filter chamber.
Inventive Concept 189. The apparatus according to Inventive Concept 187, wherein the testing device further includes a support for the filter, disposed at least partially between the inlet of the filter chamber and the filter.
Inventive Concept 190. The apparatus according to Inventive Concept 187, wherein the apparatus further includes an elongate member configured to push at least a portion of the filter into the filter chamber.
Inventive Concept 191. The apparatus according to Inventive Concept 187, wherein the plunger head is configured to push at least a portion of the filter into the filter chamber.
Inventive Concept 192. The apparatus according to Inventive Concept 187, wherein the testing device further includes a frangible seal that removably blocks liquid flow into the inlet of the filter chamber.
Inventive Concept 193. The apparatus according to Inventive Concept 187, wherein the filter chamber is not disposed so as to receive the liquid that is driven through at least one of the one or more valves.
Inventive Concept 194. The apparatus according to Inventive Concept 187, wherein the filter chamber includes at least one of the one or more valves, not disposed at the inlet of the filter chamber.
Inventive Concept 195. The apparatus according to Inventive Concept 194,

wherein the liquid container is shaped so as to define one or more openings through a wall of the liquid container,

wherein the one or more openings are downstream of the filter when the filter is removably disposed upstream of the filter chamber with the filter partially covering the inlet of the filter chamber, and

wherein the filter chamber is not disposed so as to receive the liquid that is driven through the one or more openings.

Inventive Concept 196. The apparatus according to Inventive Concept 173, wherein the filter is disposed at least partially within the filter chamber.
Inventive Concept 197. The apparatus according to Inventive Concept 196, wherein the filter is disposed entirely within the filter chamber.
Inventive Concept 198. The apparatus according to Inventive Concept 196, wherein the filter is shaped as a receptacle.
Inventive Concept 199. The apparatus according to any one of Inventive Concepts 157-169, wherein the testing device further includes one or more heating elements which are configured to heat the filter at a generally constant temperature, the temperature in the range of 20 and 50 degrees C.
Inventive Concept 200. The apparatus according to Inventive Concept 199, wherein the temperature is in the range of 30 to 40 degrees C.
Inventive Concept 201. The apparatus according to Inventive Concept 199, wherein the liquid container includes, upstream of the filter, a frangible dividing waterproof or water-resistant membrane that isolates the filter from the liquid in the liquid container.
Inventive Concept 202. The apparatus according to Inventive Concept 199, wherein the one or more heating elements are disposed in the plunger.
Inventive Concept 203. The apparatus according to any one of Inventive Concepts 157-169,

wherein the one or more valves are one or more first valves, and

wherein the testing device further includes one or more second pressure relief valves, which are in fluid communication with the liquid container and are disposed upstream of the filter.

Inventive Concept 204. The apparatus according to Inventive Concept 203,

wherein the testing device further includes a waste liquid receptacle, which is coupled to the liquid container downstream of the filter,

wherein the plunger is arranged to apply pressure to drive the liquid contained in the liquid container through the filter and then into the waste liquid receptacle, and

wherein the one or more second pressure relief valves are in fluid communication with the waste liquid receptacle not via the filter.

Inventive Concept 205. The apparatus according to Inventive Concept 203,

wherein the plunger includes a plunger shaft, and the plunger head is disposed at a downstream end portion of the plunger shaft,

wherein the testing device includes one or more unfiltered liquid receptacles, and

wherein the one or more second pressure relief valves are in fluid communication with the one or more unfiltered liquid receptacles.

Inventive Concept 206. The apparatus according to Inventive Concept 205, wherein the one or more unfiltered liquid receptacles are disposed along the plunger shaft.
Inventive Concept 207. The apparatus according to Inventive Concept 206, wherein the one or more unfiltered liquid receptacles are removably coupled to the plunger.
Inventive Concept 208. The apparatus according to Inventive Concept 203,

wherein the one or more first valves include one or more first pressure-activated valves configured to open upon exposure to a first pressure gradient across the one or more first pressure-activated valves, and

wherein the one or more second pressure relief valves are configured to open upon exposure to a second pressure gradient across the one or more second pressure relief valves, the second pressure gradient greater than the first pressure gradient.

Inventive Concept 209. The apparatus according to any one of Inventive Concepts 157-169, wherein the liquid container is shaped so as to define upstream and downstream openings, and wherein an area of the upstream opening is greater than the area of the downstream opening.
Inventive Concept 210. The apparatus according to Inventive Concept 209, wherein the liquid container includes an upstream end portion that includes the upstream opening, and wherein the upstream end portion is conical.
Inventive Concept 211. The apparatus according to Inventive Concept 210, wherein a diameter of the upstream opening is at least 10% greater than a diameter of the downstream opening.
Inventive Concept 212. The apparatus according to any one of Inventive Concepts 157-169, wherein the apparatus further includes sterile packaging, in which at least the at least the liquid container, the one or more valves, and the filter are removably disposed.
Inventive Concept 213. The apparatus according to any one of Inventive Concepts 157-169, wherein the apparatus further includes at least one container containing an extraction reagent.
Inventive Concept 214. The apparatus according to Inventive Concept 213, wherein the apparatus further includes a test strip.
Inventive Concept 215. The apparatus according to any one of Inventive Concepts 157-169, wherein the apparatus further includes a container containing a solution for use in a detecting a pathogen.

There is further provided, in accordance with an Inventive Concept 216 of the present invention, apparatus including a testing device for testing for the presence of particulate in a liquid, the testing device including:

a liquid container for containing the liquid, wherein the liquid container has an internal volume of between 0.5 and 500 ml;

one or more non-pressure-activated valves;

a filter, disposed in or downstream of the liquid container and upstream of the one or more valves; and

a liquid-pressure source, which is arranged to apply pressure to drive the liquid contained in the liquid container through the filter and then through the one or more valves,

wherein the testing device is configured to automatically close the one or more non-pressure-activated valves after the liquid-pressure source applies the pressure to drive the liquid contained in the liquid container through the filter and then through the one or more non-pressure-activated valves.

Inventive Concept 217. The apparatus according to Inventive Concept 216, wherein the testing device is configured such that motion of the liquid-pressure source automatically closes the one or more non-pressure-activated valves after the liquid-pressure source applies the pressure to drive the liquid contained in the liquid container through the filter and then through the one or more non-pressure-activated valves.
Inventive Concept 218. The apparatus according to Inventive Concept 217, wherein the testing device is configured such that rotational motion of the liquid-pressure source automatically closes the one or more non-pressure-activated valves after the liquid-pressure source applies the pressure to drive the liquid contained in the liquid container through the filter and then through the one or more non-pressure-activated valves.
Inventive Concept 219. The apparatus according to Inventive Concept 218,

wherein the one or more non-pressure-activated valves include two discs that are shaped so as to define respective sets of openings, and wherein the one or more non-pressure-activated valves are configured to assume open and closed states when the two sets of openings are aligned and non-aligned with each other, and

wherein the testing device is configured such that rotational motion of the liquid-pressure source automatically closes the one or more non-pressure-activated valves by rotating at least one of the two discs with respect to the other of the discs, after the liquid-pressure source applies the pressure to drive the liquid contained in the liquid container through the filter and then through the one or more non-pressure-activated valves.

There is further provided, in accordance with an Inventive Concept 220 of the present invention, a method including:

incubating gargled fluid for between 12 and 75 hours in a container that contains a liquid growth medium, a dehydrated growth medium, or a gel growth medium; and

thereafter, performing a strep test using a rapid strep test (RST) technique on the gargled fluid and growth medium.

Inventive Concept 221. The method according to Inventive Concept 220, wherein performing the strep test using the RST technique includes performing a lateral flow test.
Inventive Concept 222. The method according to Inventive Concept 220, wherein performing the strep test using the RST technique includes performing an RST technique selected from the group consisting of: an ELISA-based RST, an antibody-coated-beads-based RST, a nucleic-acid-based RST, and a fluorescent immunoassaying (FIA) RST.
Inventive Concept 223. The method according to Inventive Concept 220, wherein performing the strep test using the RST technique includes performing the RST technique on the gargled fluid and the growth medium while the gargled fluid and the growth medium are in the container.
Inventive Concept 224. The method according to Inventive Concept 220, wherein performing the strep test using the RST technique includes transferring at least a portion of the gargled fluid and the growth medium to another container and performing the RST technique while the at least a portion of the gargled fluid and the growth medium are in the other container.
Inventive Concept 225. The method according to Inventive Concept 224, wherein transferring the at least a portion of the gargled fluid and the growth medium to the other container includes:

inserting an absorbent element into the gargled fluid and growth medium; and

thereafter, placing the absorbent element into the other container.

Inventive Concept 226. The method according to Inventive Concept 225, wherein the absorbent element is a swab.
Inventive Concept 227. The method according to Inventive Concept 226, wherein the swab is a flocked swab.
Inventive Concept 228. The method according to Inventive Concept 224, wherein transferring the at least a portion of the gargled fluid and the growth medium includes transferring at least 0.05 ml of the gargled fluid and the growth medium.
Inventive Concept 229. The method according to Inventive Concept 220, wherein performing the strep test using the RST technique further includes filtering the gargled fluid and the growth medium after incubating, and performing the strep test using the RST technique on the filter.
Inventive Concept 230. The method according to Inventive Concept 229, wherein filtering the gargled fluid and the growth medium after incubating includes:

placing the gargled fluid and the growth medium in a liquid container of a testing device; and

applying pressure to drive the gargled fluid and the growth medium contained in the liquid container (a) through a filter of the testing device and (b) then through one or more valves of the testing device, wherein the filter is disposed in or downstream of the liquid container, and wherein the one or more valves are disposed downstream of the filter.

Inventive Concept 231. The method according to Inventive Concept 229, wherein filtering the gargled fluid and the growth medium after incubating includes:

placing the gargled fluid and the growth medium in a liquid container of a testing device;

applying pressure to drive the gargled fluid and the growth medium contained in the liquid container through a filter of the testing device, wherein the filter is disposed in or downstream of the liquid container; and

thereafter, testing, within a filter chamber of the testing device, for the presence of particulate trapped by the filter while the filter is disposed at least partially in the filter chamber, wherein the filter chamber is (a) disposed downstream of the liquid container, (b) shaped so as to define an inlet, and (c) in fluid communication with the filter.

There is further provided, in accordance with an Inventive Concept 232 of the present invention, a method for testing for the presence of particulate in gargled fluid, the method including:

incubating the gargled fluid for between 12 and 75 hours in a container that contains a liquid growth medium, a dehydrated growth medium, or a gel growth medium; and

thereafter, performing a test for the particulate using a rapid test technique on the gargled fluid and growth medium.

Inventive Concept 233. The method according to Inventive Concept 232, wherein performing the test using the rapid test technique includes performing a lateral flow test.
Inventive Concept 234. The method according to Inventive Concept 232, wherein performing the test using the rapid test technique includes performing a rapid test technique selected from the group consisting of: an ELISA-based rapid test, an antibody-coated-beads-based rapid test, a nucleic-acid-based rapid test, and a fluorescent immunoassaying (FIA) rapid test.

There is further provided, in accordance with an Inventive Concept 235 of the present invention, a method for testing for the presence of particulate in gargled fluid, the method including:

incubating the gargled fluid for between 12 and 75 hours in a container that contains a liquid growth medium, a dehydrated growth medium, or a gel growth medium; and

thereafter, performing a lateral flow test for the particulate on the gargled fluid and growth medium.

Inventive Concept 236. The method according to Inventive Concept 235, wherein the particulate is strep, and wherein performing the lateral flow test includes performing the lateral flow test for the strep.
Inventive Concept 237. The method according to any one of Inventive Concepts 232 and 235, wherein performing the test includes performing the test on the gargled fluid and the growth medium while the gargled fluid and the growth medium are in the container.
Inventive Concept 238. The method according to any one of Inventive Concepts 232 and 235, wherein performing the test includes transferring at least a portion of the gargled fluid and the growth medium to another container and performing the test while the at least a portion of the gargled fluid and the growth medium are in the other container.
Inventive Concept 239. The method according to any one of Inventive Concepts 220, 232, and 235, wherein the container does not contain agar.
Inventive Concept 240. The method according to any one of Inventive Concepts 220, 232, and 235, further including mixing the gargled fluid with the growth medium before incubating.

There is further provided, in accordance with an Inventive Concept 241 of the present invention, a method including:

incubating saliva not swabbed from a patient's throat for between 12 and 75 hours in a container that contains a liquid growth medium, a dehydrated growth medium, or a gel growth medium; and

thereafter, performing a strep test using a rapid strep test (RST) technique on the saliva and growth medium.

Inventive Concept 242. The method according to Inventive Concept 241, wherein the saliva not swabbed from the throat of the patient is saliva spit by the patient.
Inventive Concept 243. The method according to Inventive Concept 241, wherein the container does not contain agar.
Inventive Concept 244. The method according to Inventive Concept 241, further including mixing the saliva with the growth medium before incubating.
Inventive Concept 245. The method according to Inventive Concept 241, wherein performing the strep test using the RST technique includes performing a lateral flow test.
Inventive Concept 246. The method according to Inventive Concept 241, wherein performing the strep test using the RST technique includes performing an RST technique selected from the group consisting of: an ELISA-based RST, an antibody-coated-beads-based RST, a nucleic-acid-based RST, and a fluorescent immunoassaying (FIA) RST.
Inventive Concept 247. The method according to Inventive Concept 241, wherein incubating includes:

receiving, on an absorbent element, saliva from the patient's mouth; and

thereafter, placing the absorbent element into the container that contains the liquid growth medium, dehydrated growth medium, or gel growth medium.

Inventive Concept 248. The method according to Inventive Concept 247, wherein the absorbent element is a swab.
Inventive Concept 249. The method according to Inventive Concept 248, wherein the swab is a flocked swab.
Inventive Concept 250. The method according to Inventive Concept 247, wherein performing the strep test using the RST technique includes performing the RST technique on the saliva and the growth medium while the saliva and the growth medium are in the container.
Inventive Concept 251. The method according to Inventive Concept 247, wherein performing the strep test using the RST technique includes transferring at least a portion of the saliva and the growth medium to another container and performing the RST technique while the at least a portion of the saliva and the growth medium are in the other container.
Inventive Concept 252. The method according to Inventive Concept 251, wherein transferring the at least a portion of the saliva and the growth medium to the other container includes:

removing the swab from the container that contains the liquid growth medium, dehydrated growth medium, or gel growth medium; and

thereafter, placing the swab into the other container.

Inventive Concept 253. The method according to Inventive Concept 251, wherein transferring the at least a portion of the saliva and the growth medium includes transferring at least 0.05 ml of the saliva and the growth medium.
Inventive Concept 254. The method according to Inventive Concept 247, wherein performing the strep test using the RST technique further includes filtering the at least a portion of the saliva and the growth medium after incubating, and performing the strep test using the RST technique on the filter.
Inventive Concept 255. The method according to Inventive Concept 254, wherein filtering the saliva and the growth medium after incubating includes:

placing the saliva and the growth medium in a liquid container of a testing device; and

applying pressure to drive the saliva and the growth medium contained in the liquid container (a) through a filter of the testing device and (b) then through one or more valves of the testing device, wherein the filter is disposed in or downstream of the liquid container, and wherein the one or more valves are disposed downstream of the filter.

Inventive Concept 256. The method according to Inventive Concept 254, wherein filtering the saliva and the growth medium after incubating includes:

placing the saliva and the growth medium in a liquid container of a testing device;

applying pressure to drive the saliva and the growth medium contained in the liquid container through a filter of the testing device, wherein the filter is disposed in or downstream of the liquid container; and

thereafter, testing, within a filter chamber of the testing device, for the presence of particulate trapped by the filter while the filter is disposed at least partially in the filter chamber, wherein the filter chamber is (a) disposed downstream of the liquid container, (b) shaped so as to define an inlet, and (c) in fluid communication with the filter.

There is further provided, in accordance with an Inventive Concept 257 of the present invention, a system including:

(a) a liquid including at least one substance selected from the group of substances consisting of gargled fluid, saliva not swabbed from a throat of a patient, and an incubated culture medium containing a biological sample; and

(b) a testing device, which includes:
a liquid container containing the liquid;
one or more valves;
a filter, disposed in or downstream of the liquid container and upstream of the one or more valves; and
a liquid-pressure source, which is arranged to apply pressure to drive the liquid contained in the liquid container through the filter and then through the one or more valves.
   Inventive Concept 258. The system according to Inventive Concept 257, wherein the liquid includes the gargled fluid.
   Inventive Concept 259. The system according to Inventive Concept 257, wherein the liquid includes the saliva not swabbed from the throat of the patient.
   Inventive Concept 260. The system according to Inventive Concept 257, wherein the liquid includes the incubated culture medium containing the biological sample.
   Inventive Concept 261. The system according to Inventive Concept 257, wherein the liquid container has an internal volume of between 0.5 and 500 ml.
   Inventive Concept 262. The system according to Inventive Concept 257, wherein the liquid-pressure source includes a plunger, which includes a plunger head that is shaped so as to be insertable into the liquid container.
   Inventive Concept 263. The system according to Inventive Concept 257, wherein the liquid-pressure source includes a vacuum pump disposed downstream of the one or more valves.
   Inventive Concept 264. The system according to Inventive Concept 257, wherein the liquid-pressure source includes a positive-pressure pump disposed upstream of the filter.
   Inventive Concept 265. The system according to Inventive Concept 257, wherein the one or more valves include one or more pressure-activated valves.
   Inventive Concept 266. The system according to Inventive Concept 257, wherein the one or more valves include one or more non-pressure-activated valves.
   Inventive Concept 267. The system according to Inventive Concept 257, wherein the testing device further includes a waste liquid receptacle, which is coupled to the liquid container downstream of the one or more valves, and wherein the liquid-pressure source is arranged to apply pressure to drive the liquid contained in the liquid container through the filter, then through the one or more valves, and then into the waste liquid receptacle.
   Inventive Concept 268. The system according to Inventive Concept 257, wherein the testing device further includes a filter chamber that is (a) disposed downstream of the liquid container, (b) shaped so as to define an inlet, and (c) in fluid communication with the filter.

There is further provided, in accordance with an Inventive Concept 268 of the present invention, apparatus including a testing device for testing for the presence of particulate in a liquid, the testing device including:

(a) an upstream component, which includes:
(i) a plunger housing, which is shaped so as to define upstream and downstream openings; and
(ii) a plunger, which includes a downstream plunger head that is shaped so as to be insertable into the plunger housing so as to form a movable seal with a wall of the plunger housing, wherein an area of a downstream surface of the downstream plunger head equals between 80% and 100% of an area of the downstream opening; and

(b) a downstream component, which includes:
(i) a filter, which has a filter surface area of an upstream side of the filter equal to at least 80% of the area of the downstream surface of the downstream plunger head; and
(ii) a waste liquid receptacle, disposed downstream of the filter,

wherein the testing device is shaped so as to define a liquid container for containing the liquid, and

wherein the upstream component and the downstream component are configured to be removably coupled together so as to form a liquid-impermeable seal.

Inventive Concept 270. The apparatus according to Inventive Concept 269, wherein the upstream component and the downstream component are configured to be removably coupled together so as to form the liquid-impermeable seal, such that the upstream component and the downstream component partially overlap each other at an axial overlap region that at least partially defines the liquid container.
Inventive Concept 271. The apparatus according to Inventive Concept 269, wherein the testing device is configured such that at least 800% of the surface area of an upstream side of the filter is exposed to outside the testing device when the upstream component and the downstream component are decoupled from each other.
Inventive Concept 272. The apparatus according to Inventive Concept 269, wherein the upstream component and the downstream component are configured to be removably coupled together by click-fitting together.
Inventive Concept 273. The apparatus according to Inventive Concept 269, wherein the upstream component and the downstream component are configured to be removably coupled together by friction-fitting together.
Inventive Concept 274. The apparatus according to Inventive Concept 269, wherein the upstream component and the downstream component are configured to be removably coupled together by twist-and-lock fitting together.
Inventive Concept 275. The apparatus according to any one of Inventive Concepts 269-274, wherein an area of the upstream opening is greater than the area of the downstream opening.
Inventive Concept 276. The apparatus according to Inventive Concept 275, wherein the plunger housing includes an upstream end portion that includes the upstream opening, and wherein the upstream end portion is conical.
Inventive Concept 277. The apparatus according to Inventive Concept 276, wherein a diameter of the upstream opening is at least 10% greater than a diameter of the downstream opening.

There is further provided, in accordance with an Inventive Concept 278 of the present invention, a method including:

decoupling an upstream component of a testing device from a downstream component of the testing device so as to expose a filter of the testing device; and

testing for particulate trapped in the filter,

wherein the upstream component includes:
(i) a plunger housing, which is shaped so as to define upstream and downstream openings; and
(ii) a plunger, which includes a downstream plunger head that is shaped so as to be insertable into the plunger housing so as to form a movable seal with a wall of the plunger housing, wherein an area of a downstream surface of the downstream plunger head equals between 80% and 100% of an area of the downstream opening,

wherein the downstream component includes:
(i) the filter, wherein the filter has a filter surface area of an upstream side of the filter equal to at least 80% of the area of the downstream surface of the downstream plunger head; and
(ii) a waste liquid receptacle, disposed downstream of the filter,

wherein the testing device is shaped so as to define a liquid container for containing a liquid that includes at least one substance selected from the group of substances consisting of gargled fluid, saliva not swabbed from a throat of a patient, and an incubated culture medium containing a biological sample, and

wherein the upstream component and the downstream component are configured to be removably coupled together so as to form a liquid-impermeable seal.

Inventive Concept 279. The method according to Inventive Concept 278, wherein the upstream component and the downstream component are configured to be removably coupled together so as to form the liquid-impermeable seal, such that the upstream component and the downstream component partially overlap each other at an axial overlap region that at least partially defines the liquid container.
Inventive Concept 280. The method according to Inventive Concept 278, wherein the testing device is configured such that at least 80% of the surface area of an upstream side of the filter is exposed to outside the testing device when the upstream component and the downstream component are decoupled from each other.
Inventive Concept 281. The method according to Inventive Concept 278, wherein an area of the upstream opening is greater than the area of the downstream opening.
Inventive Concept 282. The method according to Inventive Concept 281, wherein the plunger housing includes an upstream end portion that includes the upstream opening, and wherein the upstream end portion is conical.
Inventive Concept 283. The method according to Inventive Concept 282, wherein a diameter of the upstream opening is at least 10% greater than a diameter of the downstream opening.
Inventive Concept 284. The method according to Inventive Concept 278, wherein the upstream component and the downstream component are configured to be removably coupled together by click-fitting together.
Inventive Concept 285. The method according to Inventive Concept 278, wherein the upstream component and the downstream component are configured to be removably coupled together by friction-fitting together.
Inventive Concept 286. The method according to Inventive Concept 278, wherein the upstream component and the downstream component are configured to be removably coupled together by twist-and-lock fitting together.

There is further provided, in accordance with an Inventive Concept 286 of the present invention, apparatus including a testing device for testing for the presence of particulate in a liquid, the testing device including:

a liquid container for containing the liquid;

a filter, disposed in or downstream of the liquid container; and

a plunger head, which (a) is shaped so as to be insertable into the liquid container, (b) is configured to apply pressure to drive the liquid from the liquid container through the filter, and (c) has a downstream surface that is at least partially coated with a solid or semi-solid growth medium.

Inventive Concept 288. The apparatus according to Inventive Concept 287, wherein an area of the downstream surface of the plunger head is between 0.3 and 100 cm2.
Inventive Concept 289. The apparatus according to Inventive Concept 287, wherein the plunger head is shaped so as to be insertable into the liquid container so as to form a movable seal with a wall of the liquid container.
Inventive Concept 290. The apparatus according to Inventive Concept 287, wherein the testing device further includes a plunger shaft, and the plunger head is disposed at a downstream end portion of the plunger shaft.
Inventive Concept 291. The apparatus according to Inventive Concept 287, wherein the testing device further includes a waste liquid receptacle, coupled to the liquid container downstream of the filter.
Inventive Concept 292. The apparatus according to any one of Inventive Concepts 287-291, wherein the growth medium includes agar.
Inventive Concept 293. The apparatus according to any one of Inventive Concepts 287-291, wherein the growth medium is solid.
Inventive Concept 294. The apparatus according to Inventive Concept 293, wherein the solid growth medium is dehydrated.
Inventive Concept 295. The apparatus according to Inventive Concept 293, wherein the solid growth medium includes powdered solid growth medium.
Inventive Concept 296. The apparatus according to any one of Inventive Concepts 287-291, further including a cap that is configured to be coupled to and fully cover the growth medium on the downstream surface of the plunger head.
Inventive Concept 297. The apparatus according to Inventive Concept 296, wherein the cap is transparent.

There is further provided, in accordance with an Inventive Concept 297 of the present invention, a method including:

pushing a plunger head to apply pressure to drive liquid from a liquid container of a testing device through a filter of the testing device, wherein the plunger head has a downstream surface that is at least partially coated with a solid or semi-solid growth medium;

touching the downstream surface of the plunger head to the filter; and

assessing the downstream surface of the plunger head for biological growth.

Inventive Concept 299. The method according to Inventive Concept 298, wherein the liquid includes at least one substance selected from the group of substances consisting of gargled fluid, saliva not swabbed from a throat of a patient, and an incubated culture medium containing a biological sample.
Inventive Concept 300. The method according to Inventive Concept 298, wherein assessing includes assessing the downstream surface of the plunger head for biological growth of a biological particulate selected from the group consisting of: a microorganism, a fungus, a bacterium, a spore, a virus, a mite, a biological cell, a biological antigen, a protein, a protein antigen, and a carbohydrate antigen.
Inventive Concept 301. The method according to Inventive Concept 298, wherein the growth medium includes agar.
Inventive Concept 302. The method according to Inventive Concept 298, wherein the method further includes heating the plunger head before assessing the downstream surface of the plunger head for biological growth.
Inventive Concept 303. The method according to Inventive Concept 298, wherein the growth medium is solid.
Inventive Concept 304. The method according to Inventive Concept 303, wherein the solid growth medium is dehydrated.
Inventive Concept 305. The method according to Inventive Concept 303, wherein the solid growth medium includes powdered solid growth medium.
Inventive Concept 306. The method according to Inventive Concept 298, further including coupling a cap the plunger head such that the cap fully covers the growth medium on the downstream surface of the plunger head.
Inventive Concept 307. The method according to Inventive Concept 306, wherein the cap is transparent.

There is further provided, in accordance with an Inventive Concept 307 of the present invention, a method including:

pushing a plunger head to apply pressure to drive liquid from a liquid container of a testing device through a filter of the testing device;

touching a downstream surface of the plunger head to the filter,

thereafter, touching the downstream surface of the plunger head to culture medium contained in a culture-medium container;

heating the culture-medium container; and

assessing the culture-medium container for biological growth.

Inventive Concept 309. The method according to Inventive Concept 308, wherein the culture medium includes agar.
Inventive Concept 310. The method according to Inventive Concept 308, wherein the liquid includes at least one substance selected from the group of substances consisting of gargled fluid, saliva not swabbed from a throat of a patient, and an incubated culture medium containing a biological sample.
Inventive Concept 311. The method according to Inventive Concept 308, wherein assessing includes assessing the culture-medium container for biological growth of a biological particulate selected from the group consisting of: a microorganism, a fungus, a bacterium, a spore, a virus, a mite, a biological cell, a biological antigen, a protein, a protein antigen, and a carbohydrate antigen.
Inventive Concept 312. The method according to Inventive Concept 308, wherein the downstream surface of the plunger head is rough.
Inventive Concept 313. The method according to Inventive Concept 312, wherein touching the downstream surface of the plunger head to the filter includes grinding the filter with the rough downstream surface.
Inventive Concept 314. The method according to Inventive Concept 308, further including testing, within the testing device, for the presence of a biological particulate trapped by the filter.

There is further provided, in accordance with an Inventive Concept 314 of the present invention, a testing kit for testing for the presence of particulate in a liquid, the testing kit including:

a liquid container for containing the liquid, the liquid container shaped so as to define upstream and downstream openings;

a filter, disposed in or downstream of the liquid container; and

a plunger head that (a) is shaped so as to be insertable into the liquid container so as to form a movable seal with a wall of the liquid container, and (b) is arranged such that when pushed, the plunger head applies pressure to drive the liquid contained in the liquid container through the filter and then through the downstream opening,

wherein the testing kit does not include a plunger shaft.

Inventive Concept 316. The testing kit according to Inventive Concept 315, wherein the filter is configured to trap at least 40% of group A streptococcus bacteria and allow passage of the liquid.
Inventive Concept 317. The testing kit according to Inventive Concept 315, wherein the filter is configured to trap at least 40% of a particulate to be tested and allow passage of the liquid.
Inventive Concept 318. The testing kit according to Inventive Concept 315, further including sterile packaging, in which at least the liquid container, plunger head, and the filter are removably disposed.
Inventive Concept 319. The testing kit according to Inventive Concept 315, wherein the liquid container includes a liquid-tight seal disposed downstream of the filter, and

wherein the testing kit is arranged such that when the plunger head is pushed, the plunger head applies the pressure to break or open the seal and drive the liquid through the filter and then through the downstream opening.

There is further provided, in accordance with an Inventive Concept 319 of the present invention, a method including:

receiving a testing kit including (a) a liquid container, the liquid container shaped so as to define upstream and downstream openings, (b) a filter, disposed in or

downstream of the liquid container, (c) and a plunger head;

coupling the plunger head to a plunger shaft;

receiving a liquid in the liquid container; inserting the plunger head into the liquid container so as to form a movable seal with a wall of the liquid container; and

using the plunger shaft, pushing the plunger head to apply pressure to drive the liquid contained in the liquid container through the filter and then through the downstream opening,

wherein the testing kit does not include the plunger shaft.

Inventive Concept 321. The method according to Inventive Concept 320, further including, after pushing the plunger head, testing for the presence of particulate trapped by the filter.

There is further provided, in accordance with an Inventive Concept 322 of the present invention, a method for testing for the presence of group A streptococcus bacteria in a sample of oral fluid obtained from a patient, the method including:

generating a biological product by incubating the sample of oral fluid for between 12 and 50 hours in a container that contains a liquid growth medium, the liquid growth medium having (a) a total nitrogen source concentration between 75 and 300 g/L and (b) a total solids concentration between 92.5 and 370 g/L; and

thereafter, performing a strep test using a rapid strep test (RST) technique on the biological product.

Inventive Concept 323. The method according to Inventive Concept 322, wherein incubating includes incubating for between 16 and 50 hours.
Inventive Concept 324. The method according to Inventive Concept 322, wherein the container does not contain agar.
Inventive Concept 325. The method according to Inventive Concept 322, wherein performing the strep test using the RST technique includes performing a lateral flow test.
Inventive Concept 326. The method according to Inventive Concept 322, wherein performing the strep test using the RST technique includes performing an RST technique selected from the group consisting of: an ELISA-based RST, an antibody-coated-beads-based RST, a nucleic-acid-based RST, and a fluorescent immunoassaying (FIA) RST.
Inventive Concept 327. The method according to Inventive Concept 322, wherein the liquid growth medium has a pH of between 6 and 8.3.
Inventive Concept 328. The method according to Inventive Concept 322, wherein incubating includes incubating for between 12 and 36 hours.
Inventive Concept 329. The method according to any one of Inventive Concepts 322-328, wherein the sample of oral fluid is selected from the group consisting of: gargled fluid gargled by the patient, and saliva not swabbed from a throat of the patient.
Inventive Concept 330. The method according to Inventive Concept 329, wherein the sample of oral fluid is the gargled fluid.
Inventive Concept 331. The method according to Inventive Concept 329, wherein the sample of oral fluid is the saliva not swabbed from the throat of the patient.
Inventive Concept 332. The method according to Inventive Concept 331, wherein the saliva not swabbed from the throat of the patient is saliva spit by the patient.
Inventive Concept 333. The method according to any one of Inventive Concepts 322-328, wherein the sample of oral fluid is saliva swabbed from a tonsil of the patient.
Inventive Concept 334. The method according to any one of Inventive Concepts 322-328, wherein the liquid growth medium has a total sugar concentration of between 7 g/L and 20 g/L.
Inventive Concept 335. The method according to Inventive Concept 334, wherein the total sugar concentration is between 7 g/L and 14 g/L.
Inventive Concept 336. The method according to Inventive Concept 334, wherein the liquid growth medium has a glucose concentration of between 8 g/L and 12 g/L.
Inventive Concept 337. The method according to Inventive Concept 336, wherein the glucose concentration is between 8.5 g/L and 9.5 g/L.
Inventive Concept 338. The method according to any one of Inventive Concepts 322-328, wherein the total nitrogen source concentration is between 105 and 180 g/L.
Inventive Concept 339. The method according to Inventive Concept 338, wherein the total nitrogen source concentration is between 120 and 160 g/L.
Inventive Concept 340. The method according to any one of Inventive Concepts 322-328, wherein the total solids concentration is between 130 and 222 g/L.
Inventive Concept 341. The method according to Inventive Concept 340, wherein the total solids concentration is between 148 and 193 g/L.
Inventive Concept 342. The method according to any one of Inventive Concepts 322-328, wherein performing the strep test includes applying one or more extraction reagents to the biological product.
Inventive Concept 343. The method according to any one of Inventive Concepts 322-328, wherein generating the biological product further includes filtering the sample of oral fluid and the liquid growth medium after incubating.
Inventive Concept 344. The method according to Inventive Concept 343, wherein performing the strep test using the RST technique includes performing the strep test using the RST technique on the filter.
Inventive Concept 345. The method according to Inventive Concept 343, wherein performing the strep test includes applying one or more extraction reagents to the filtered biological product.

There is further provided, in accordance with an Inventive Concept 345 of the present invention, a liquid growth medium having (a) a total nitrogen source concentration between 75 and 300 g/L and (b) a total solids concentration between 92.5 and 370 g/L.

Inventive Concept 347. The liquid growth medium according to Inventive Concept 346, wherein the liquid growth medium has a pH of between 6 and 8.3.
Inventive Concept 348. The liquid growth medium according to Inventive Concept 346, wherein the liquid growth medium has a total sugar concentration of between 7 g/L and 20 g/L.
Inventive Concept 349. The liquid growth medium according to Inventive Concept 348, wherein the total sugar concentration is between 7 g/L and 14 g/L.
Inventive Concept 350. The liquid growth medium according to Inventive Concept 348, wherein the liquid growth medium has a glucose concentration of between 8 g/L and 12 g/L.
Inventive Concept 351. The liquid growth medium according to Inventive Concept 350, wherein the glucose concentration is between 8.5 g/L and 9.5 g/L
352. The liquid growth medium according to Inventive Concept 346, wherein the total nitrogen source concentration is between 105 and 180 g/L.
Inventive Concept 353. The liquid growth medium according to Inventive Concept 352, wherein the total nitrogen source concentration is between 120 and 160 g/L.
Inventive Concept 354. The liquid growth medium according to Inventive Concept 346, wherein the total solids concentration is between 130 and 222 g/L.
Inventive Concept 355. The liquid growth medium according to Inventive Concept 354, wherein the total solids concentration is between 148 and 193 g/L.
Inventive Concept 356. An assembly including the liquid growth medium according to any one of Inventive Concepts 346-355, the assembly further including a sealed sterile container that contains the liquid growth medium.
Inventive Concept 357. An assembly including the liquid growth medium according to any one of Inventive Concepts 346-355, the assembly further including a container that contains the liquid growth medium and a sample of oral fluid obtained from a patient.
Inventive Concept 358. A kit including the liquid growth medium according to any one of Inventive Concepts 346-355, the kit further including a lateral flow strep test strip.
Inventive Concept 359. A kit including the liquid growth medium according to any one of Inventive Concepts 346-355, the kit further including one or more extraction reagents.
Inventive Concept 360. A kit including the liquid growth medium according to any one of Inventive Concepts 346-355, the kit further including a filter.

There is further provided, in accordance with an Inventive Concept 361 of the present invention, a method of preparing a liquid growth medium, the method including:

adding a quantity of powdered growth medium to a volume of distilled water; and

stirring until the powdered growth medium is dissolved in the distilled water to produce the liquid growth medium,

wherein the quantity of powdered growth medium and the volume of the distilled water are selected such that the liquid growth medium has (a) a total nitrogen source concentration between 75 and 300 g/L and (b) a total solids concentration between 92.5 and 370 g/L.

Inventive Concept 362. The method according to Inventive Concept 361, wherein the liquid growth medium has a pH of between 6 and 8.3.
Inventive Concept 363. The method according to Inventive Concept 361, wherein the quantity of powdered growth medium and the volume of the distilled water are selected such that the liquid growth medium has a total sugar concentration of between 7 g/L and 20 g/L.
Inventive Concept 364. The method according to Inventive Concept 363, wherein the quantity of powdered growth medium and the volume of the distilled water are selected such that the total sugar concentration is between 7 g/L and 14 g.
Inventive Concept 365. The method according to Inventive Concept 363, wherein the quantity of powdered growth medium and the volume of the distilled water are selected such that the liquid growth medium has a glucose concentration of between 8 g/L and 12 g/L.
Inventive Concept 366. The method according to Inventive Concept 365, wherein the quantity of powdered growth medium and the volume of the distilled water are selected such that the glucose concentration is between 8.5 g/L and 9.5 g/L.
Inventive Concept 367. The method according to Inventive Concept 361, wherein the quantity of powdered growth medium and the volume of the distilled water are selected such that the total nitrogen source concentration is between 105 and 180 g/L.
Inventive Concept 368. The method according to Inventive Concept 367, wherein the quantity of powdered growth medium and the volume of the distilled water are selected such that the total nitrogen source concentration is between 120 and 160 g/L.
Inventive Concept 369. The method according to Inventive Concept 361, wherein the quantity of powdered growth medium and the volume of the distilled water are selected such that the total solids concentration is between 130 and 222 g/IL.
Inventive Concept 370. The method according to Inventive Concept 369, wherein the quantity of powdered growth medium and the volume of the distilled water are selected such that the total solids concentration is between 148 and 193 g/L.

The present invention will be more fully understood from the following detailed description of applications thereof, taken together with the drawings, in which:

### BRIEF DESCRIPTION OF THE DRAWINGS

FIGS. 1-7 are schematic illustrations of apparatus for testing for presence of a particulate in a fluid, in accordance with some applications of the present invention;

FIG. 8 is a schematic illustration of apparatus for collecting fluid, in accordance with some applications of the present invention;

FIGS. 9A-B are schematic illustrations of a configuration of the apparatus for testing for presence of a particulate in a fluid, in accordance with some applications of the present invention;

FIGS. 10A-B are schematic illustrations of a configuration of the apparatus for testing for presence of a particulate in a fluid, in accordance with some applications of the present invention;

FIGS. 11A-B are schematic illustrations of a configuration of the apparatus for testing for presence of a particulate in a fluid, in accordance with some applications of the present invention;

FIGS. 12A-E are schematic illustrations of various configurations of the apparatus for testing for presence of a particulate in a fluid, in accordance with some applications of the present invention;

FIGS. 13A-D are schematic illustrations of various configurations of the apparatus for testing for presence of a particulate in a fluid, in accordance with some applications of the present invention;

FIGS. 14A-D are schematic illustrations of a tube and a plunger having rotational asymmetry, in accordance with some applications of the present invention;

FIGS. 15A-B are schematic illustrations of a configuration of the apparatus for testing for presence of a particulate in a fluid, in accordance with some applications of the present invention;

FIGS. 16A-B are schematic illustrations of a configuration of the apparatus for testing for presence of a particulate in a fluid, in accordance with some applications of the present invention;

FIGS. 17A-B are schematic illustrations of a configuration of the apparatus for testing for presence of a particulate in a fluid, in accordance with some applications of the present invention;

FIGS. 18A-B are schematic illustrations of a threaded tube and a plunger configured to engage the threads, in accordance with some applications of the present invention;

FIGS. 19A-B are schematic illustrations of a threaded tube and a plunger configured to engage the threads, in accordance with some applications of the present invention;

FIG. 20 is a schematic illustration of a configuration of the apparatus for testing for presence of a particulate in a fluid, in accordance with some applications of the present invention;

FIGS. 21A-B are schematic illustrations of a configuration of the apparatus for testing for presence of a particulate in a fluid, in accordance with some applications of the present invention;

FIGS. 22A-H are schematic illustrations of a testing device for testing for presence of particulate in a liquid, in accordance with an application of the present invention;

FIG. 23 is a schematic illustration of another testing device for testing for presence of particulate in a liquid, in accordance with an application of the present invention;

FIGS. 24A-B are schematic illustrations of yet another testing device for testing for presence of particulate in a liquid, in accordance with an application of the present invention;

FIG. 24C is a schematic illustration of still another testing device for testing for presence of particulate in a liquid, in accordance with an application of the present invention;

FIGS. 25A-C are schematic illustrations of another testing device for testing for presence of particulate in a liquid, in accordance with an application of the present invention;

FIGS. 26A-B are schematic illustrations of additional testing devices for testing for presence of particulate in a liquid, in accordance with respective applications of the present invention;

FIGS. 27A-C are schematic illustrations of yet another testing device for testing for presence of particulate in a liquid, in accordance with an application of the present invention;

FIGS. 28A-C and 29A-E are schematic illustrations of still another testing device for testing for presence of particulate in a liquid, in accordance with an application of the present invention;

FIG. 30 is a schematic illustration of another testing device for testing for presence of particulate in a liquid, in accordance with an application of the present invention;

FIGS. 31A-C are schematic illustrations of yet another testing device for testing for presence of particulate in a liquid, in accordance with an application of the present invention;

FIGS. 31D-K are schematic illustrations of a testing device for testing for presence of particulate in a liquid, in accordance with an application of the present invention;

FIGS. 31L-M are schematic illustrations of a testing device for testing for presence of particulate in a liquid, in accordance with an application of the present invention;

FIGS. 31N-O are schematic illustrations of a testing device for testing for presence of particulate in a liquid, in accordance with an application of the present invention;

FIGS. 31P-Q are schematic illustrations of a testing device for testing for presence of particulate in a liquid, in accordance with an application of the present invention;

FIGS. 32A-E are schematic illustrations of the testing device of FIGS. 22A-H further comprising one or more heating elements, in accordance with respective applications of the present invention;

FIG. 33 is a schematic illustration of a method for performing a test, in accordance with an application of the present invention;

FIGS. 34A-B are schematic illustrations of a method for performing a backup test, in accordance with an application of the present invention;

FIG. 34C is a schematic illustration of another method for performing a backup test, in accordance with an application of the present invention;

FIG. 34D is a flowchart depicting a method for performing a backup strep test using rapid strep test (RST) techniques on gargled fluid after incubation, in accordance with some applications of the present invention;

FIG. 34E is a flowchart depicting a method for performing a backup strep test using rapid strep test (RST) techniques on saliva not swabbed from a patient's throat after incubation, in accordance with some applications of the present invention;

FIGS. 35A-C are schematic illustrations of another testing device for testing for presence of particulate in a liquid, in accordance with an application of the present invention;

FIGS. 36A-C are schematic illustrations of a method for using the testing device of FIGS. 35A-C for testing for presence of particulate in a liquid, in accordance with an application of the present invention;

FIGS. 37A-B are schematic illustrations of a testing system, in accordance with an application of the present invention;

FIG. 38 is a schematic exploded view of a testing device of the testing system of FIG. 37A-B, in accordance with an application of the present invention;

FIGS. 39A-F are schematic illustrations of a method for using the testing system of FIGS. 37A-B to test for the presence of the particulate in a liquid, in accordance with an application of the present invention;

FIGS. 40A-D, 41, 42, and 43 are tables that present results of an experiment conducted in accordance with an application of the present invention;

FIGS. 44, 45, 46, 47, and 48 are tables that present results of another experiment conducted in accordance with an application of the present invention; and

FIGS. 49 and 50 are graphs that present results of experiments conducted in accordance with respective applications of the present invention.

### DETAILED DESCRIPTION OF APPLICATIONS

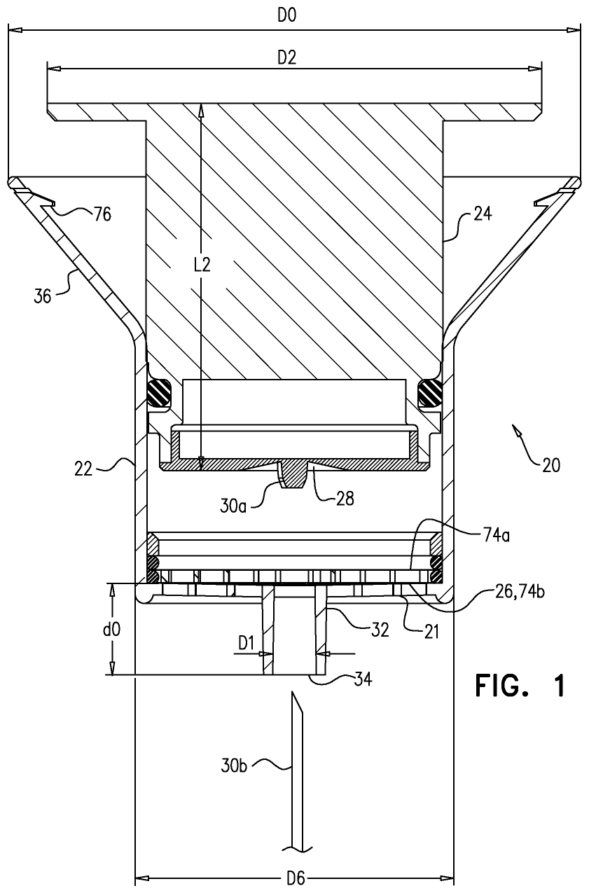

Reference is made to FIG. 1, which is a schematic illustration of apparatus 20 for testing for presence of a particulate in a fluid, in accordance with some applications of the present invention. Apparatus 20 comprises a tube 22 and a plunger 24, plunger 24 being sized and shaped to be advanceable within tube 22 while sealably contacting the tube. A proximally-facing surface 21 at a distal end of the tube inhibits advancement of the plunger.

Fluid that potentially contains the particulate is collected in the tube. Using the plunger, the fluid is pushed through a filter 26 disposed within a distal portion of the tube. (It is noted that in the context of the claims and specification of the present application, the term “proximal” refers to the top of the apparatus as depicted in FIG. 1, while the term “distal” refers to the bottom. For example, a user of apparatus 20 would place his thumb on the proximal end of plunger 24, and using the plunger, would push fluid out of the distal end of tube 22.) Typically, the plunger is advanced at least until the plunger contacts the filter. Filter 26 allows for passage of the fluid therethrough, but does not allow for passage of at least some (e.g., a substantial portion) of the particulate. Consequently, following the pushing of the fluid through the filter, the filter may be tested for presence of the particulate, i.e., the presence of the particulate may be tested for by ascertaining if any of the particulate was trapped by the filter. Typically, the filter is tested for presence of the particulate while the filter is inside the tube.

Types of fluid that may be collected in tube 22 include gargled fluid and/or biological fluid such as saliva. For example, a patient may gargle a saline fluid and subsequently spit the gargled fluid, perhaps along with some saliva, into the tube. (Alternatively, e.g., for juvenile patients who cannot gargle, saliva may be collected without any gargled fluid.) Other types of biological fluid that may be collected in tube 22 include blood (e.g., diluted blood), urine, stool (e.g., diluted stool), gastrointestinal (GI) fluid, and bronchoalveolar lavage fluid. Types of particulates that may be tested for include a microorganism (e.g., a parasite), a fungus, a bacteria, a spore (e.g., a pollen spore), a virus, a mite, a biological cell (e.g., a cancerous cell), a biological antigen, a protein, a protein antigen, and a carbohydrate antigen.

For example, using apparatus 20:

(a) Gargled fluid may be tested for presence of a streptococcus bacteria, as further described hereinbelow.

(b) Diluted blood may be tested for presence of an intracellular or extracellular pathogen (e.g. Plasmodium falciparum, a parasite causing malaria, or a blood-borne streptococcus bacteria), or cancerous cells.

(c) Urine may be tested for a urinary tract pathogen.

(d) Diluted stool may be tested for an enteric pathogen (e.g., salmonella).

(e) GI fluid (e.g., GI fluid obtained via a nasogastric or endoscopic tube) may be tested for a pathogen, e.g., giardia.

(f) Aspirated fluid may be tested for presence of cancerous cells.

Typically, the gargle fluid includes carbonated water, phosphate buffered saline, pelargonium sidoides extract, tannic acid, balloon flower platycodon grandiflorus, berberine sulfate, S-carboxymethylcysteine, curcumin, or any combination thereof. In some applications, the gargle fluid is carbonated. Typically, the temperature of the gargle fluid is 1-38 degrees Celsius.

Typically, a volume of the tube is at least 1 mL and/or less than 70 mL, e.g., between 1 and 8 mL, between 8 and 15 mL, between 15 and 30 mL, or between 30 and 70 mL. In some applications, the tube does not comprise a Luer lock or any other type of needle-coupling mechanism.

In some applications, the plunger and tube are shaped to provide an empty volume proximal to surface 21 of at least 0.03 and/or less than 5 mL (e.g., 0.03-1 mL) when the plunger is maximally advanced within the tube. For example, the distal end of the plunger may be shaped to define a distally-facing cavity 28 (e.g., a “dimple”) therein, cavity 28 providing at least part of the empty volume. The empty volume, which may be proximal and/or distal to the filter, facilitates the testing of the filter for the particulate, by providing a “testing area” in fluid continuity with the filter. For example, when conducting a rapid strep test, it is typically necessary to apply the A and B solution to the filter, i.e., place the A and B solution in contact with the filter, such that the strep A carbohydrate antigen may be drawn out from the trapped bacteria and into the solution. The empty volume provides an area in fluid continuity with the filter in which the A and B solution may collect, and into which the dipstick may be subsequently placed. Typically, a volume of the cavity is at least 0.03 mL and/or less than 5 mL (e.g., 0.03-1 mL). For example, the volume of the cavity may be at least 0.15 mL, e.g., at least 0.25 mL, e.g., at least 0.4 mL.

In some applications, apparatus 20 comprises a kit in which the plunger and tube are disposed. In some applications, the plunger is disposed entirely outside of the tube when contained in the kit, to allow for immediate use of the tube without first removing the plunger. In some applications, the kit further contains the particulate-presence-testing-facilitation solution (e.g., the A and B solution).

In some applications, apparatus 20 further comprises a puncturing element 30a protruding from a distal end of the plunger, puncturing element 30a being configured to puncture the filter upon the plunger being advanced to the filter. In other applications, a disconnected puncturing element 30b is disposed within the kit that contains the plunger and tube. Puncturing element 30b is sized and shaped to be passable through an opening 34 at a distal end of the tube, and is configured to puncture the filter by being longer than a distance d0 from opening 34 to the filter. (Typically, the puncturing element is at least as long as the distance from opening 34 to the proximal side of the filter.) The puncturing of the filter facilitates the testing, by allowing the particulate-presence-testing-facilitation solution, which is typically passed into the tube from the distal end of the tube (as further described hereinbelow), to collect in cavity 28. Furthermore, the puncturing of the filter facilitates collection of the particulate for subsequent culturing, such as, for example, when a throat culture is performed alternatively or additionally to the rapid strep test. Typically, the distal end of plunger 24 is not convex; rather, the distal end of the plunger is generally flat. For example, as shown in FIG. 1, the distal end of the plunger, with the exception of cavity 28, is generally flat. In general, a plunger having a generally flat distal end is able to push more of the fluid through the filter, relative to a plunger having a convex distal end, since a greater portion of the distal end may be advanced all the way to the filter.

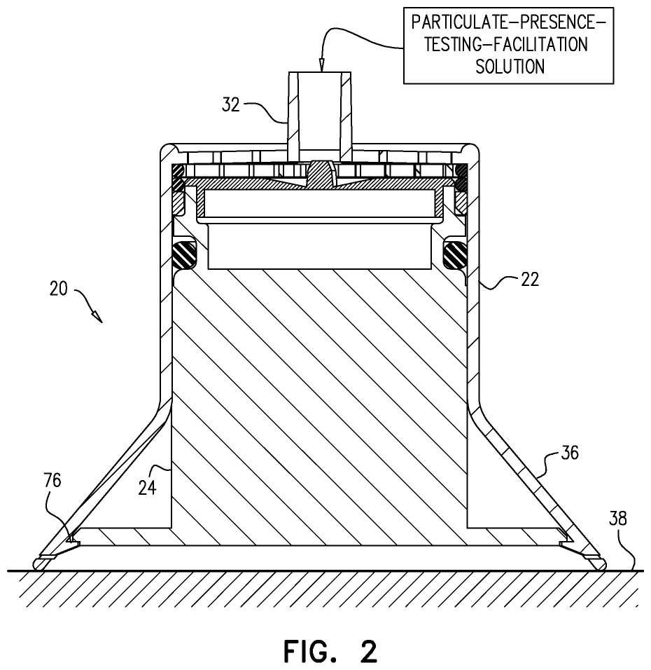

Typically, the proximal end of the tube is shaped to define a funnel-shaped proximal opening 36, which facilitates the collection of fluid in the tube. For some applications, to facilitate easily depositing gargled fluid directly from a subject's mouth into tube 22, a proximal-most diameter D0 of funnel-shaped proximal opening 36 is at least 20%, e.g., at least 25%, e.g., at least 30%, e.g., at least 40%, e.g., at least 50%, greater than a diameter D6 of tube 22, and is typically less than 300° %, e.g., less than 250%, e.g., less than 200% greater than diameter D6 of tube 22. In some applications, the distal end of the tube is shaped to define a conduit 32, such as, for example, by comprising a Luer lock. Conduit 32 facilitates testing for presence of the particulate, as further described hereinbelow with reference to FIG. 2.

Reference is now made to FIG. 2, which is a schematic illustration of apparatus 20, in accordance with some applications of the present invention. Typically, following the pushing of the fluid through filter 26 (FIG. 1), the plunger and tube are turned upside-down, such that the distal opening of the tube is above (with respect to gravity) the proximal opening of the tube. For example, the plunger and tube may be made to rest on a horizontal surface 38, on the proximal end of the tube (as shown in the figure) or the proximal end of the plunger. Subsequently, the particulate-presence-testing-facilitation solution is applied to the filter, i.e., the solution is placed into the tube (e.g., by being passed through conduit 32) such that the solution is in contact with the filter. In some applications, the distal end of the tube (e.g., conduit 32) is funnel-shaped; this shape facilitates the placing of the particulate-presence-testing-facilitation solution into the tube. Conduit 32 also facilitates a rapid strep test, by allowing passage of the dipstick and by holding the dipstick when the proximal end of the tube or plunger is resting on a horizontal surface.

In some applications, before testing the filter for presence of the particulate, a culture medium (e.g., tryptic soy broth) is used to culture the particulate, and/or a preserving medium is used to preserve the particulate in a viable or non-viable state. (For example, saline may be used to preserve the particulate in a viable state, while ethanol may be used to preserve the particulate in a non-viable state.) An advantage of culturing the particulate is that the testing sensitivity generally increases as the amount of particulate increases. An advantage of preserving the particulate is that the testing (e.g., a rapid strep test, or a throat culture to supplement the rapid strep test) may be performed even after some time has passed from the collection of the fluid.

In some applications, as noted above, apparatus 20 is used to test for presence of a microorganism, such as streptococcus bacteria. In such applications, the particulate-presence-testing-facilitation solution may include a releasing agent (e.g., the A and B solution), which, upon contacting the microorganism, releases an antigen from the microorganism. Subsequently, the area into which the antigen is released may be tested for presence of the antigen.

In some applications, the tube and plunger are configured such that, following the plunger being maximally advanced within the tube, the plunger is withdrawable from the tube only by use of a tool or by breaking a portion of the apparatus. For example, as shown in FIG. 2, the proximal end of the plunger may be not proximal to (i.e., distal to or flush with) the proximal end of the tube, when the plunger is maximally advanced within the tube, such that the plunger effectively becomes stuck in the tube. This generally serves to prevent the plunger from leaving the tube when the tube is handled, e.g., turned upside-down. Alternatively or additionally, the apparatus comprises a locking mechanism 76 (FIG. 1) that is configured to lock the plunger inside the tube following the plunger being maximally advanced within the tube. For example, as shown in FIG. 1, locking mechanism 76 may comprise tabs that can be pushed inward by the plunger as the plunger is advanced into the tube, but cannot be pushed outward, such that the plunger, following a maximal advancement thereof, is blocked from exiting the tube.

In some applications, there is no locking mechanism, and plunger 24 can easily be removed from tube 22 subsequently to plunger 24 being maximally advanced.

Reference is again made to FIG. 1. Typically, proximal-most diameter D0 of the proximal opening of the tube is relatively large, such as to facilitate (a) the collection of the fluid in the tube, and/or (b) the upside-down resting of the tube on horizontal surface 38 (FIG. 2). For example, a ratio of D0 to a diameter D1 of the distal opening of the tube may be at least 13. As noted above, in some applications, the tube and plunger rest on the proximal end of the plunger. To facilitate this, the proximal end of the plunger may be relatively wide; for example, a ratio of the diameter D2 of the proximal end of the plunger to the length L2 of the plunger may be at least 1.

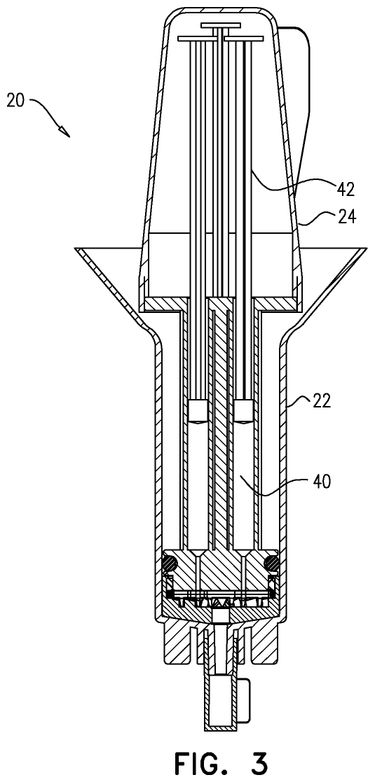

Reference is now made to FIG. 3, which is a schematic illustration of apparatus 20, in accordance with some applications of the present invention. In some applications, plunger 24 is shaped to define at least one plunger lumen 40 containing a particulate-presence-testing-facilitation solution. (FIG. 3 shows an application in which there are two plunger lumens, one containing the A solution, and the other containing the B solution.) The particulate-presence-testing-facilitation solution is deployed (i.e., placed into the tube) by being passed out of plunger lumen 40, e.g., via a sub-plunger 42 that is slidably disposed within the plunger lumen.

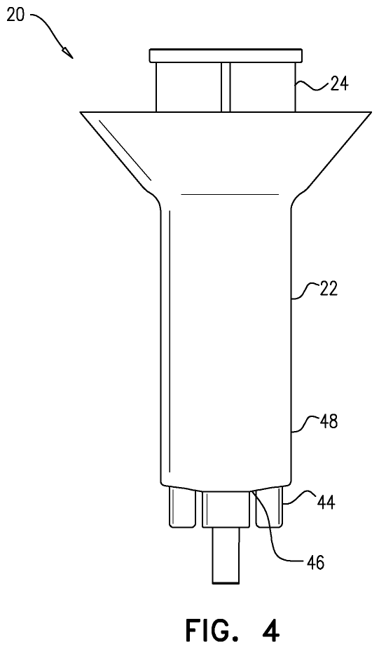

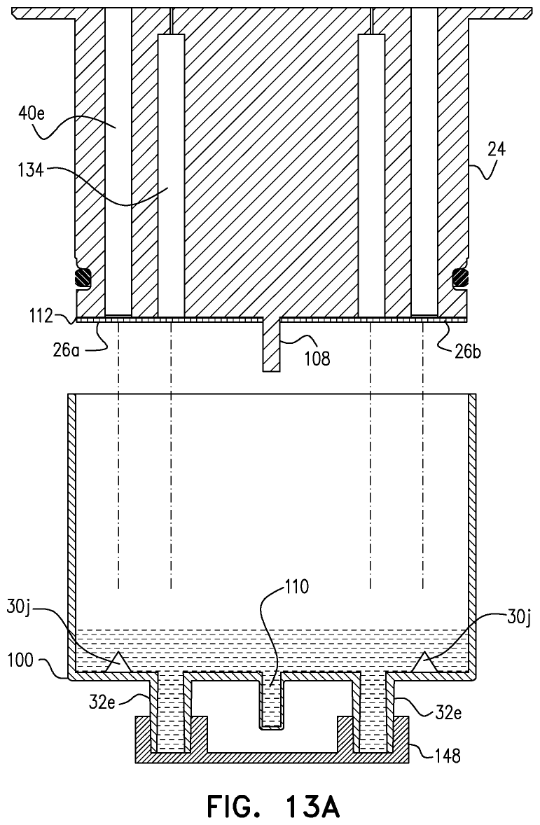

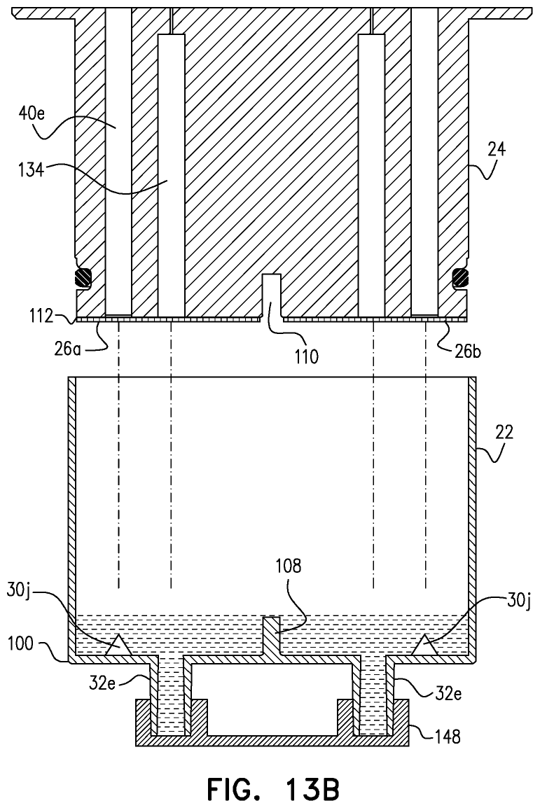

Reference is now made to FIG. 4, which is a schematic illustration of apparatus 20, in accordance with some applications of the present invention. In some applications, a wall of the tube is shaped to define at least one enclosed cavity 44 containing the particulate-presence-testing-facilitation solution. Enclosed cavity 44 is configured to open upon the plunger being moved within the tube. For example, the plunger may puncture a proximally-facing cover of the enclosed cavity as the plunger is advanced, thus opening the enclosed cavity. Alternatively, the enclosed cavity may be separated from the lumen of the tube by a one-way valve. Following maximal advancement of the plunger, the plunger is withdrawn slightly, thus creating a vacuum proximal to the valve that causes the valve to open. FIG. 4 shows an application in which the bottom wall 46 of the tube is shaped to define enclosed cavity 44. Alternatively or additionally, a distal portion of the lateral wall 48 of the tube, and/or the distal end of the plunger, may be shaped to define an enclosed cavity containing the particulate-presence-testing-facilitation solution. In some applications, cavity 44 further contains a gas above atmospheric pressure (e.g., as shown in FIG. 10A), such that the particulate-presence-testing-facilitation solution is forced out upon the opening of cavity 44. In general, for all applications described herein, the opening of cavity 44 can alternatively be done independently of plunger 24.

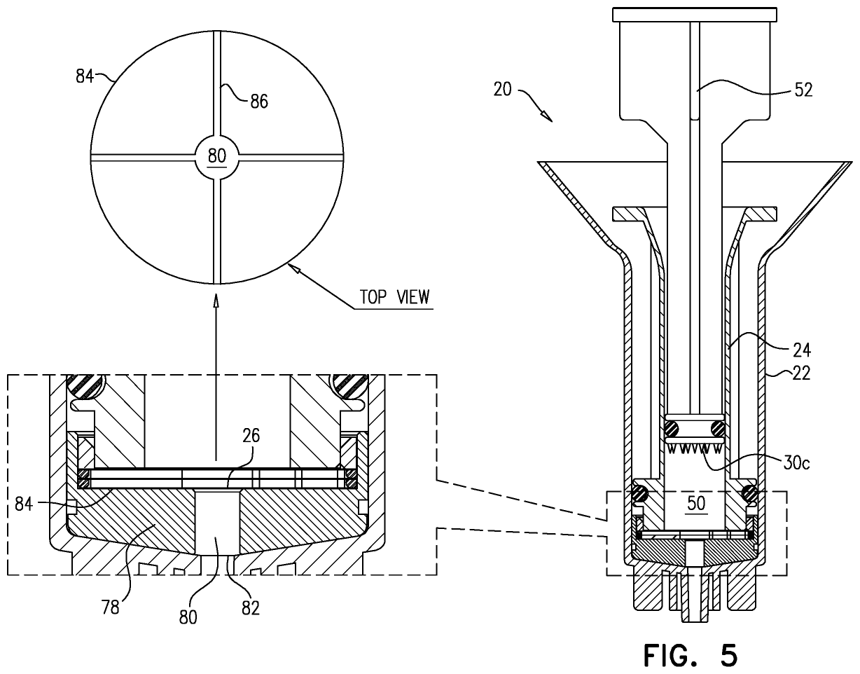

Reference is now made to FIG. 5, which is a schematic illustration of apparatus 20, in accordance with some applications of the present invention. In some applications, plunger 24 is shaped to define a plunger lumen 50. A shaft 52, which is shaped to be slidably disposed within plunger lumen 50, comprises a puncturing element 30c at a distal end thereof. Shaft 52 is advanced within the plunger lumen until puncturing element 30c punctures the filter. (As described above with reference to FIG. 1, the puncturing of the filter facilitates testing for the particulate.)

As also shown in FIG. 5, in some applications, apparatus 20 comprises an insert 78 disposed within a distal portion of the tube and not fixed to the plunger, filter 26 being coupled to a proximally-facing surface 84 of insert 78. One function of insert 78 is to provide a generally flat proximally-facing surface to support the filter, such that a particular tube may be used even if the tube has a convex distal end. Typically, insert 78 is shaped to define (a) an at least partially distally-facing opening 82 therein, and (b) a passage 80 from proximally-facing surface 84 to opening 82. Passage 80 provides for exit of fluid from the tube as the plunger is advanced. In some applications, as shown in the top (distally-facing) view of surface 84, the insert is further shaped to define a plurality of grooves 86 in surface 84, respective spaces within the grooves being in fluid communication with passage 80. Grooves 86 facilitate the flow of fluid through the passage and out of the tube.

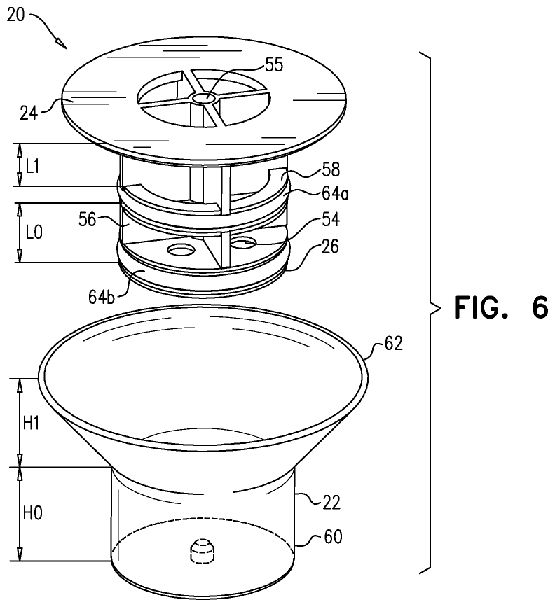

Reference is now made to FIG. 6, which is a schematic illustration of apparatus 20, in accordance with some applications of the present invention. In the application shown in FIG. 6, the distal end of plunger 24 is shaped to define one or more passageways 54 therethrough, and filter 26 is coupled to the distal end of the plunger. In some applications, the distal end of tube 22 does not have an opening, or has an opening that is permanently closed. In other applications, the distal end of tube 22 is shaped to define an openable seal (not shown), and/or apparatus 20 comprises a stopper (for example, as show in FIGS. 13A-D) configured to close the distal opening of the tube, e.g., by being disposed over the distal opening of the tube. In any case, while the plunger is advanced, the distal end of the tube remains closed, such that the fluid in the tube is pushed through the filter, through passageways 54, and into one or more (e.g., four) compartments 56 that are in fluid communication with the passageways. Typically, a total volume of compartments 56 is at least 0.5 mL and/or less than 60 mL, e.g., between 5 and 30 mL, e.g., between 8 and 20 mL.

The tube comprises a distal cylindrical portion 60, and/or a proximal funnel portion 62 coupled to cylindrical portion 60. Typically, the plunger is shaped to define a disk 58 that is proximal to compartments 56, disk 58 inhibiting passage of liquid from the compartments to a proximal side of the disk, when the disk is inside the tube. Length L0 of the plunger distal to disk 58 is approximately equal to (e.g., is within 10 mm of) the height H0 of the cylindrical portion. Thus, once the plunger has been maximally advanced within the tube, the fluid is trapped inside the tube, such the tube and plunger may be safely handled, e.g., turned upside-down (as shown in FIG. 2) for testing purposes. Typically, a first sealing ring 64a surrounds the plunger proximally to the compartments, and/or a second sealing ring 64b surrounds the plunger distally to the compartments. First sealing ring 64a facilitates the trapping of the fluid inside the compartments, while second sealing ring 64b inhibits fluid from passing between the plunger and the tube as the plunger is advanced.

Passageways 54 are typically many, well distributed, and/or large, to facilitate efficient passage of fluid therethrough. Typically, compartments 56 are not completely surrounded by a wall, such that air may escape the compartments while fluid flows in to the compartments. For example, each of the four compartments shown in FIG. 6 is entirely open. In some applications, the length L1 of the plunger proximal to the disk is not greater than height H1 of the funnel portion, such that the plunger becomes stuck in the tube upon being maximally advanced. Alternatively or additionally, locking mechanism 76 (FIG. 1) locks the plunger inside the tube.

In an alternative application, air escapes through one or more passageways (not shown) leading from the compartments to the proximal end of the plunger. (The passageways are closed subsequent to the plunger being maximally advanced within the tube.)

Following the plunger being maximally advanced within the tube, the filter may be tested for presence of the particulate, e.g., as described hereinabove with reference to FIG. 2. In some applications, the distal end of the tube is shaped to define a conduit, such as a Luer lock, that facilitates the testing for the particulate, as described hereinabove with reference to FIG. 2. For applications in which the distal end of the tube is permanently closed, the testing of the filter may be conducted via a passageway 55 passing through the plunger (but not through any of passageways 54) from the proximal end of the plunger to the distal end of the plunger. For example, a particulate-presence-testing-facilitation solution and/or a dipstick may be passed through passageway 55.

Reference is now made to FIG. 7, which is a schematic illustration of apparatus 20, in accordance with some applications of the present invention. In FIG. 7, tube 22 is shaped to define a plurality of openings 66 at a distal end thereof. The filter is disposed within a distal portion of the tube, proximal to the plurality of openings 66. The plurality of openings facilitates the pushing of the fluid through the filter and out of the tube by reducing the pressure that must be applied to the plunger, relative to if the tube were to have a single (relatively small) opening. The total area of the plurality of openings is at least 10% and/or less than 90% (e.g., 10%-80%, e.g., 10%-70%, e.g., 20%-70%) of the cross-sectional area of the distal end of the tube.

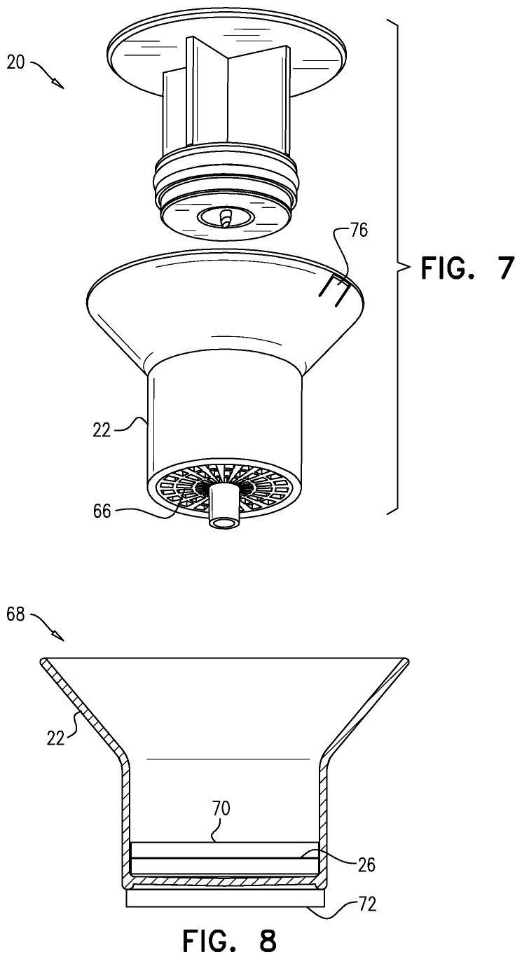

Reference is now made to FIG. 8, which is a schematic illustration of apparatus 68 for collecting fluid, in accordance with some applications of the present invention. Apparatus 68 comprises tube 22, which contains a medium 70 (e.g., in the form of a gel, powder, or coating) that facilitates testing the fluid for presence of the particulate. For example, medium 70 may include a culture medium (e.g., tryptic soy broth) configured to culture a microorganism, a spore, a virus, a mite, a biological cell, a biological antigen, a protein, a protein antigen, and/or a carbohydrate antigen. Alternatively or additionally, medium 70 may include a releasing medium (e.g., A and B solution) configured to release an antigen from a microorganism. Alternatively or additionally, medium 70 may include a heating medium (e.g., plaster and/or calcium chloride) configured to undergo an exothermic reaction, the heat from the exothermic reaction helping to preserve and/or culture the particulate. Alternatively or additionally, medium 70 may include a salt, and/or another preserving medium (e.g., formalin and/or ethanol alcohol) configured to preserve a microorganism, a spore, a virus, a mite, a biological cell, a biological antigen, a protein, a protein antigen, and/or a carbohydrate antigen in a viable or non-viable state. (One advantage of having a salt contained in the tube is that the subject need not gargle saline solution in order to preserve the bacteria; rather, the subject may gargle a more pleasant-tasting fluid, and subsequently spit the gargled fluid into the salt-containing tube.)

Filter 26 is disposed within a distal portion of the tube, the medium being disposed distally and/or proximally to the filter. Following the collection of the fluid in the tube, the plunger is used to push the fluid through the filter and out of the tube. (Alternatively, apparatus 68 may be used in combination with apparatus and techniques described with reference to FIG. 6; in such applications, filter 26 is disposed on the distal end of the plunger.)

In some applications, apparatus 68 further comprises a heating element 72 that is configured to heat the tube. For example, apparatus 68 may be contained in a kit in which heating element 72 and the tube are disposed. The heating element may comprise a chemical heating element (e.g., plaster and/or calcium chloride), and/or an electric heating element. The heating of the tube generally facilitates the culturing and/or preserving function of medium 70.

One manner in which apparatus 68 may be used will now be described. A subject at home experiences a sore throat, and decides that he would like to have a rapid strep test done. The subject therefore opens up his “home strep test kit” and pulls out the tube and plunger. The subject collects gargled fluid in the tube, uses the plunger as described hereinabove, and subsequently, brings the tube to the doctors office. From the time of collection until the subject arrives at the doctor's office, culture medium 70 (optionally, in combination with heat from heating element 72 and/or a heating medium) allows for the bacteria to multiply. At the doctor's office, the doctor conducts a rapid strep test. (Alternatively, the collection of fluid in the tube may be done at the doctor's office; in such cases, the doctor may optionally heat the tube for some time before performing the strep test, in order to boost the sensitivity of the strep test.)

Reference is again made to FIG. 1. In some applications, apparatus 20 comprises two filters disposed within the distal portion of the tube. The first filter 74a, which may act as a “pre-filter”, has a pore size of at least 0.5 microns and/or less than 100 microns, while the second filter 74b, which is typically disposed distally to first filter 74a, has a pore size of at least 0.1 microns and/or less than 20 microns. Alternatively, the second filter 74b has a pore size of at least 0.01 microns and/or less than 2 microns (e.g., for filtering viruses and/or bacteria, such as at least 0.01 microns and/or less than 0.3 microns (e.g., for filtering viruses). (Typically, the pore size of the first filter is larger than the pore size of the second filter. In some applications, however, the respective pore sizes may be equal.) It is hypothesized by the inventors that the second filter, in addition to capturing the particulate, may also facilitate the capturing of the particulate by the first filter, by providing additional resistance to the pushing of the fluid out of the filter.

In some applications, a method is provided that comprises testing for the presence of bacteria captured by the first filter, and testing for the presence of a virus captured by the second filter.

Filters 74a and 74b may also be disposed on the distal end of the plunger, e.g., in place of the single filter shown in FIG. 6. In such applications, the first filter (which, typically, has a larger pore size) is typically distal to the second filter.

The pore sizes of filters 74a and 74b vary, depending on the type of particulate being tested for. For example:

(a) For streptococcus bacteria, typical pore sizes are between 0.5 and 20 microns for the first filter, and between 0.1 microns and 1 micron for the second filter.

(b) For pollen spores, typical pore sizes are between 10 and 100 microns for the first filter, and between 1 micron and 10 microns for the second filter.

(c) For monocytes, typical pore sizes are between 5 and 25 microns for the first filter, and between 1 micron and 20 microns for the second filter.

In some applications, a kit is provided that comprises apparatus 20 comprising first filter 74a and second filter 74b; a vial; and a liquid for bathing the particulate in the vial, the liquid selected from the group consisting of: a lysis buffer, saline solution (e.g., phosphate buffered saline (PBS)), and transport medium (e.g., universal transport medium or a viral transport medium). Optionally, the vial contains the liquid. The kit may be used, for example, for filtering gargled fluid and transporting the one or both of first filter 74a and second filter 74b to a remote laboratory in the liquid in the vial, such as for performing Polymerase Chain Reaction (PCR) testing for the particulate trapped by one or both of the filters.

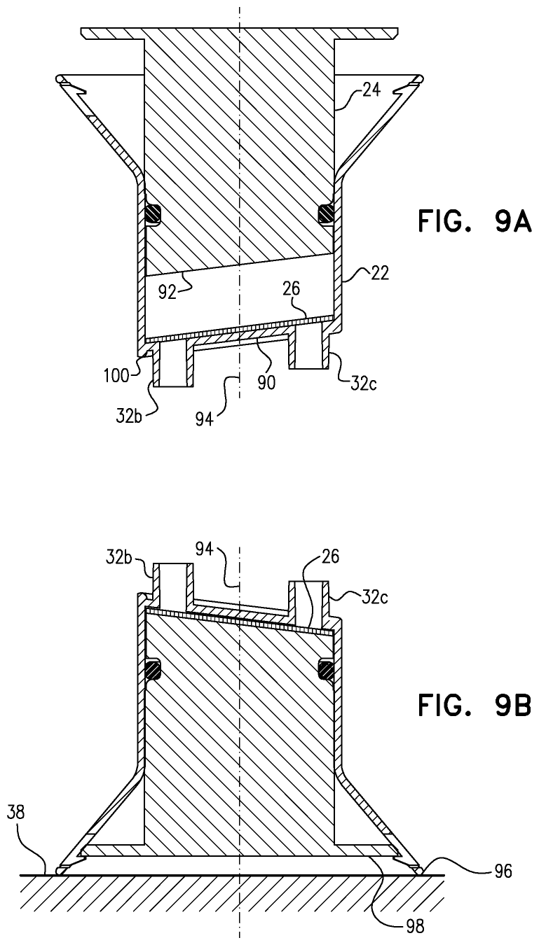

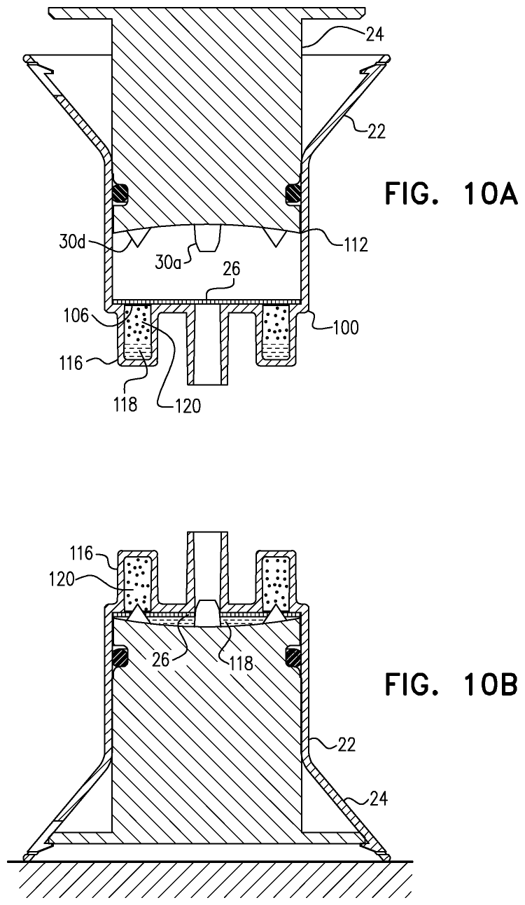

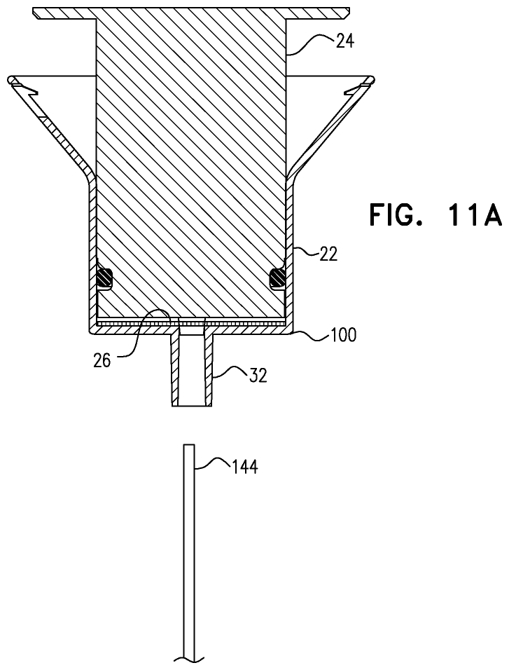

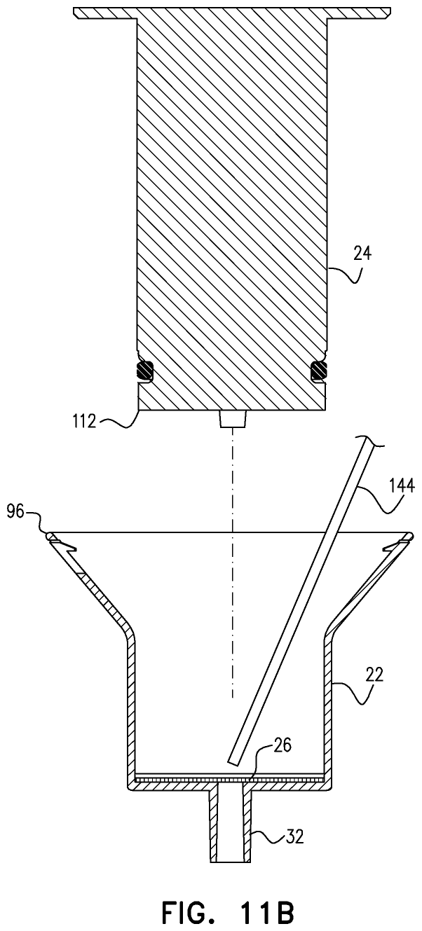

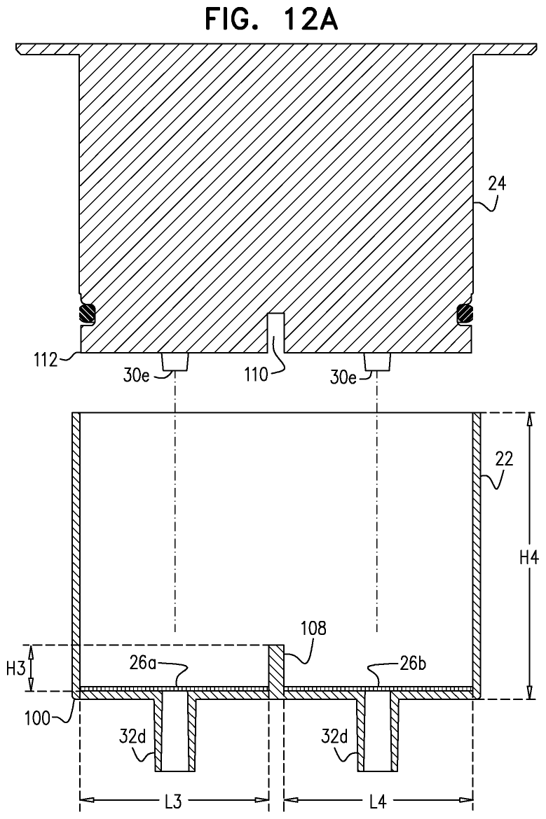

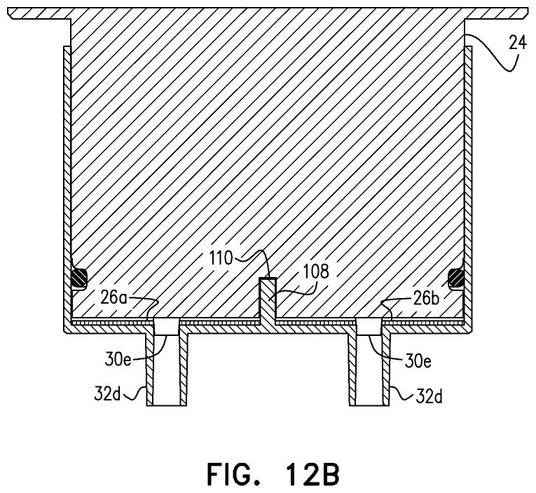

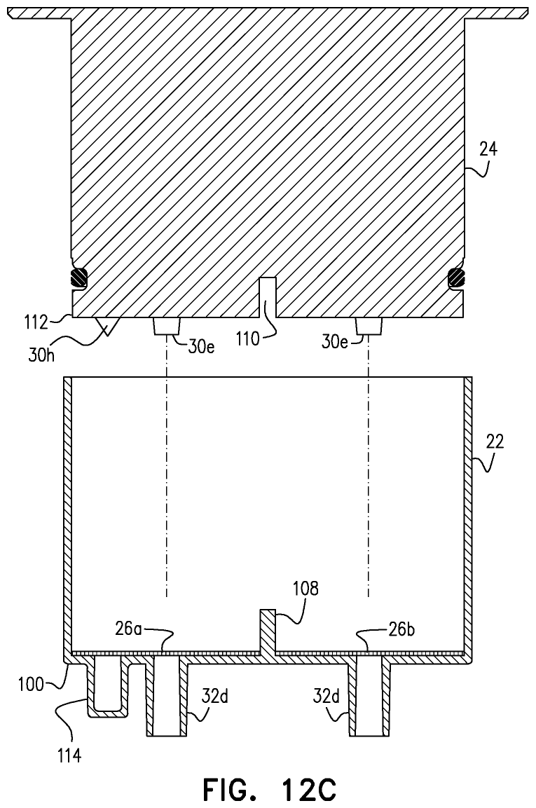

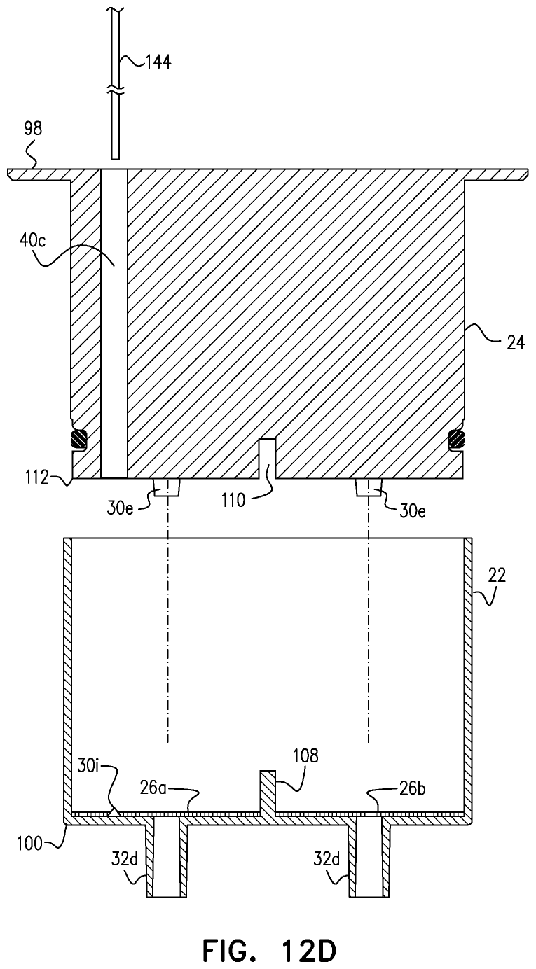

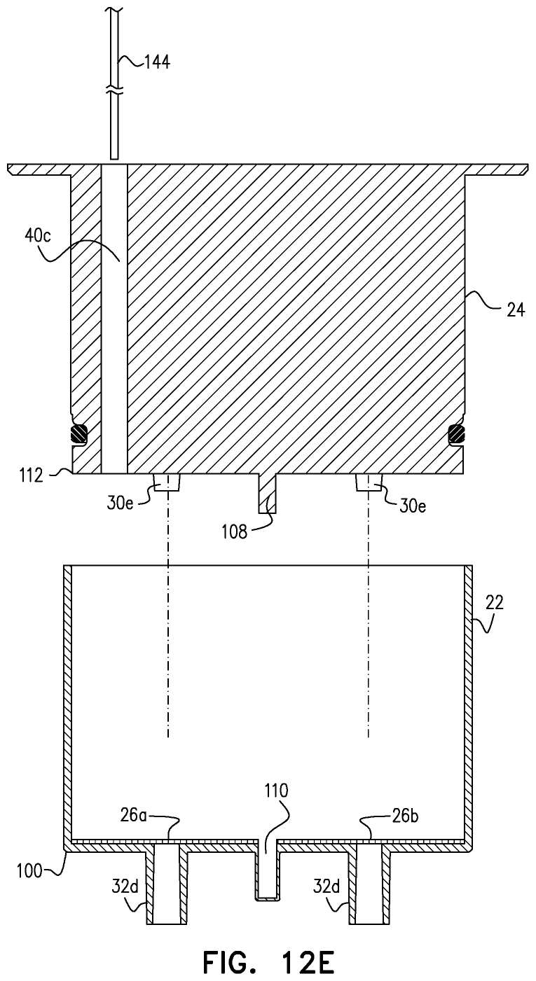

Reference is now made to FIGS. 9A-B, which are schematic illustrations of apparatus 20, in accordance with some applications of the present invention. In some applications, a distal surface 90 of tube 22 is oriented at a slant with respect to a lateral wall of tube 22, and a distal surface 92 of plunger 24 is oriented at a slant with respect to a longitudinal axis 94 of plunger 24. Typically, tube 22 and plunger 24 are configured such that the slant of distal surface 90 of tube 22 aligns with the slant of distal surface 92 of plunger 24 at at least one rotational orientation of tube 22 with respect to plunger 24. Filter 26 is disposed on the inside of slanted distal surface 90 of tube 22. Distal end 100 of tube 22 is shaped to define at least two conduits 32b and 32c, disposed such that conduit 32b is disposed at the higher end of the slant and conduit 32c is disposed at the lower end of the slant, when a proximal end 96 of tube 22 or a proximal end 98 of plunger 24 is resting on horizontal surface 38, as shown in FIG. 9B. Typically, the particulate-presence-testing-facilitation solution is passed through conduit 32b, disposed at the higher end of the slant, such that the particulate-presence-testing-facilitation solution flows down the slant along filter 26. Filter 26 may then be tested for presence of the particulate by inserting a dipstick through conduit 32c, disposed over the lower end of the slant.

In some applications, distal surface 90 of tube 22 is shaped to define a cone as is common in syringe plungers (configuration not shown).

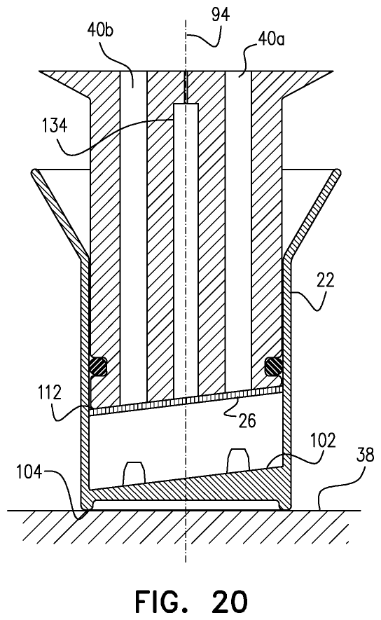

Reference is now made to FIG. 20, which is a schematic illustration of apparatus 20, in accordance with some applications of the present invention. In some applications, filter 26 is disposed on distal end 112 of plunger 24, such that the fluid is pushed through filter 26 into a compartment 134 in plunger 24. A proximally-facing distal surface 102 of tube 22 is oriented at a slant with respect to a lateral wall of tube 22, and distal end 112 of plunger 24 is oriented at a slant with respect to longitudinal axis 94 of plunger 24. Typically, tube 22 and plunger 24 are configured such that the slant of proximally-facing distal surface 102 of tube 22 aligns with the slant of distal end 112 of plunger 24 at at least one rotational orientation of tube 22 with respect to plunger 24. Plunger 24 is shaped to define at least two plunger lumens 40a and 40b, disposed such that plunger lumen 40a is disposed over the higher end of the slant and plunger lumen 40b is disposed over the lower end of the slant, when a distally-facing distal surface 104 of tube 22 is resting on horizontal surface 38. Typically, the particulate-presence-testing-facilitation solution is passed through plunger lumen 40a disposed over the higher end of the slant, such that the particulate-presence-testing-facilitation solution flows down the slant along filter 26. Filter 26 may then be tested for presence of the particulate by inserting a dipstick through plunger lumen 40b disposed over the lower end of the slant.

Reference is now made to FIGS. 10A-B, which are schematic illustrations of apparatus 20, in accordance with some applications of the present invention. In some applications, a wall of tube 22 is shaped to define at least one enclosed cavity 116 containing a particulate-presence-testing-facilitation solution 118 and a gas 120 above atmospheric pressure. As shown in FIG. 10A, cavity 16 may be in a distal wall of tube 22 and filter 26 disposed in distal end 100 of tube 22, proximal to cavity 116. Enclosed cavity 116 is closed with a seal 106 and configured to open following initiation of distal movement of plunger 24 in tube 22, such that particulate-presence-testing-facilitation solution 118 is forced out of enclosed cavity 116 and applied to filter 26. In some applications, at least one puncturing element 30d is disposed on distal end 112 of plunger 24, and configured to open enclosed cavity 116 by puncturing filter 26 and seal 106. In some applications, puncturing element 30a may be disposed on distal end 112 of plunger 24, and configured to puncture filter 26 upon plunger 24 being maximally advanced in tube 22.

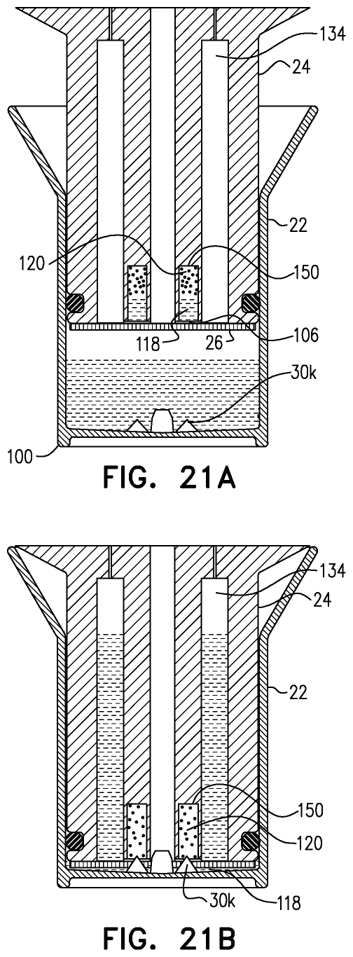

Reference is now made to FIGS. 21A-B, which are schematic illustrations of apparatus 20 in accordance with some applications of the present invention. In some applications, filter 26 is disposed on distal end 112 of plunger 24 and a wall of plunger 24 is shaped to define at least one enclosed cavity 150 containing particulate-presence-testing-facilitation solution 118 and gas 120 above atmospheric pressure (FIGS. 21A-B). Enclosed cavity 150 is closed with seal 106 disposed proximal to filter 26. At least one puncturing element 30k protrudes in a proximal direction from distal end 100 of tube 22 to puncture filter 26 and seal 106 (FIG. 21B).

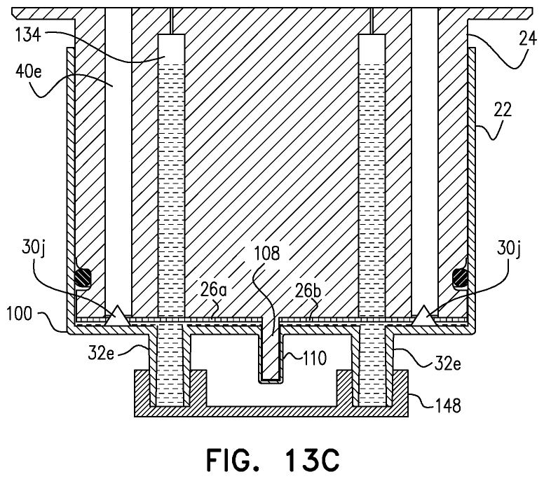

Reference is now made to FIGS. 11A-B, which are schematic illustrations of apparatus 20, in accordance with some applications of the present invention. In some applications, it would be advantageous to be able to test filter 26 for presence of the particulate in more than one way, for example by culturing (e.g., for 2-48 hours) the particulate collected on filter 26 as well as applying the particulate-presence-testing-facilitation solution for a rapid result. Therefore, in some applications, following the pushing of the fluid through filter 26, disposed in distal end 100 of tube 22, filter 26 is tested for presence of the particulate by (a) ascertaining if any of the particulate was trapped by the filter using a first protocol, and (b) if no particulate is found to be present, ascertaining using a second protocol. Typically, ascertaining using the first protocol comprises applying a particulate-presence-testing-facilitation solution to filter 26.

However, once the particulate-presence-testing-facilitation solution is applied to filter 26, the particulate present on filter 26 can no longer be cultured. Therefore, in some applications, a sample is taken prior to applying the particulate-presence-testing-facilitation solution to filter 26. In some applications, plunger 24 is removed from tube 22 (FIG. 118), and subsequently the sample is transferred from distal end 112 of plunger 24 to a culture media surface. Filter 26, while inside tube 22, is then tested, using the first protocol, for presence of the particulate and if no particulate is found then the sample taken from distal end 112 of plunger 24 can be tested using the second protocol by ascertaining whether the particulate is on the culture media surface after the sample has been cultured.

In some applications, the sample is taken by swabbing filter 26 with swab 144. Filter 26 may be swabbed through conduit 32 in distal end 100 of tube 22 (FIG. 11A), or from a proximal end 96 of tube 22 after removing plunger 24 from tube 22 (FIG. 11B).

In some applications, the sample taken from filter 26 is plated on a culture media surface and cultured (e.g., for 2-48 hours), and if no particulate is found when filter 26 is tested using the first protocol, then the sample taken from filter 26 is tested using the second protocol, by ascertaining if any of the particulate is present on the culture media surface after the sample has been cultured (e.g., for 2-48 hours). Typically, ascertaining if any of the particulate was present on the culture media surface comprises observing the culture media surface or applying a particulate-presence-testing-facilitation solution to the culture media surface.

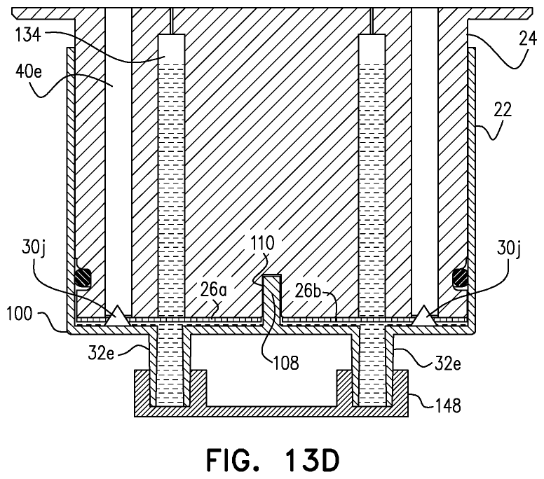

Reference is now made to FIGS. 12A-D, which are schematic illustrations of apparatus 20, in accordance with respective applications of the present invention. In some applications, a barrier 108 extends in a proximal direction from distal end 100 of tube 22, and plunger 24 is sized and shaped to define a recess 110 into which barrier 108 fits upon plunger 24 being advanced to barrier 108. This configuration allows for two filters, 26a and 26b, to be disposed in distal end 100 of tube 22 and separated by barrier 108. Having more than one filter provides the opportunity to simultaneously test for presence of the particulate using the first and second protocols, as well as the opportunity to test for the presence of more than one particulate without having to repeat the entire procedure.

Typically, plunger 24, once maximally advanced to barrier 108, is configured to prevent a particulate-presence-testing-facilitation solution that is applied to either one of filters 26a or 26b from contacting the other one of the filters.

Apparatus 20 may further comprise at least two puncturing elements 30e protruding from distal end 112 of plunger 24 and configured to puncture filters 26a and 26b, respectively, upon plunger 24 being advanced to filters 26a and 26b.

In some applications, distal end 100 of tube 22 is shaped to define at least two conduits 32d, configured to align with filters 26a and 26b, respectively. Following pushing the fluid through filters 26a and 26b, apparatus 20 may be turned upside-down and either one of filters 26a or 26b can be tested for presence of the particulate by passing the particulate-presence-testing-facilitation solution through a respective conduit 32d and subsequently inserting a dipstick through the respective conduit 32d. The second one of filters 26a or 26b can be left to culture inside tube 22 (e.g., 2-48 hours), or a sample may be taken from the second one filters 26a or 26b and cultured (e.g., for 2-48 hours).

In some applications, a distal portion of tube 22 is shaped to define at least one enclosed cavity 114 (FIG. 12C) containing the particulate-presence-testing-facilitation solution, and configured such that the particulate-presence-testing-facilitation solution in cavity 114 is applied to only filter 26a and not to filter 26b. A wall of cavity 114 is configured to open and release the particulate-presence-testing-facilitation solution. In some applications, at least one puncturing element 30h is disposed on distal end 112 of plunger 24, and configured to open enclosed cavity 114 by puncturing filter 26 the wall of cavity 114. Enclosed cavity 114 may contain both the particulate-presence-testing-facilitation solution and a gas above atmospheric pressure (for example, as shown in FIG. 10A), such that the particulate-presence-testing-facilitation solution is forced out upon opening of cavity 114. Filter 26a is then tested by inserting a dipstick through respective conduit 32d. Filter 26b may be left to culture (e.g., for 2-48 hours) inside tube 22, or a sample may be taken from filter 26b and cultured (e.g., for 2-48 hours).

In some applications, plunger 24 is shaped to define plunger lumen 40c (FIG. 12D), an opening of plunger lumen 40c being arranged to align with filter 26a and not to simultaneously align with filter 26b. A distal end of plunger lumen 40c is configured to open, upon plunger 24 being maximally advanced within tube 22, by a puncturing element 30i, protruding from distal end 100 of tube 22.

In some applications, plunger lumen 40c is closed at proximal end 98 of plunger 24 and contains the particulate-presence-testing-facilitation solution, and, upon opening of plunger lumen 40c, the particulate-presence-testing-facilitation solution is applied to only one filter 26a. In some applications, plunger lumen 40c further contains a gas above atmospheric pressure, such that the particulate-presence-testing-facilitation solution is forced out upon opening of plunger lumen 40c. Filter 26a is then tested by inserting a dipstick through respective conduit 32d. A sample may be taken from filter 26b and cultured (e.g., for 2-48 hours).

In other applications, plunger lumen 40c is initially empty and, following pushing the fluid through filters 26a and 26b, a sample can be taken from filter 26a by swabbing filter 26a with swab 144 from proximal end 98 of plunger 24 through plunger lumen 40c. The sample is cultured (e.g., for 2-48 hours) and after the sample has been taken, both filters 26a and 26b can then be tested by passing the particulate-presence-testing facilitation solution through respective conduits 32d and inserting dipsticks through respective conduits 32d. Filters 26a and 26b could be tested for presence of two different particulates respectively by passing different particulate-presence-testing-facilitation solutions through each respective conduit 32d.

In some applications, a length L3 (or corresponding area) of filter 26a(FIG. 12A) and a length L4 (or corresponding area) of filter 26b are equal to each other. In other applications, length L3 of filter 26a is at least 25% larger than length L4 of filter 26b.

Having filters 26a and 26b differ in size allows the particulate-presence-testing-facilitation solution to be applied to the larger of filters 26a or 26b, thereby increasing the chance of detecting the particulate with the initial rapid test. The smaller of filters 26a or 26b is typically cultured (e.g., for 2-48 hours) to increase the presence of the particulate.

In some applications, a culture medium is disposed on at least one of filters 26a or 26b, eliminating the need to swab the respective filter. Following the pushing of the fluid through filters 26a and 26b, one of filters 26a or 26b is simply left to culture and the particulate-presence-testing-facilitation solution is applied to the other one of filters 26a or 26b, on which no culture medium is disposed.

Height H3 of barrier 108 (FIG. 12A), extending from a distal to proximal direction, is typically low enough such that fluid collected in tube 22 is higher than the barrier (for example, as shown in FIG. 13B) and therefore evenly distributed over both filters 26a and 26b, yet high enough to prevent a particulate-presence-testing-facilitation solution that is applied to one of filters 26a or 26b after advancing of plunger 24 from contacting the other one of filters 26a or 26b. Typically, height H3 is less than 90% of a height H4 of tube 22. For example, height H3 of barrier 108 is less than a height of tube 22 that corresponds to a volume of 10 cc in tube 22, e.g. a height of tube 22 that corresponds to a volume of 5 cc, e.g. a height of tube 22 that corresponds to a volume of 1 cc, measured from distal end 100 of tube 22.

In some applications (configuration not shown), filters 26a and 26b are a first filter 26a and a second filter 26b, barrier 108 is a first barrier 108, and recess 110 is a first recess 110. First filter 26a is separated from second filter 26b by either first barrier 108 or first recess 110. Apparatus 20 further comprises a second barrier extending in a proximal direction, disposed within tube 22, and distal end 112 of plunger 24 may further be shaped to define a second recess into which the second barrier fits upon plunger 24 being advanced to the barriers. A third filter is disposed at either distal end 100 of tube 22 or distal end 112 of plunger 24, the third filter being separated from second filter 26b by either the second barrier or by the second recess. When apparatus includes three or more filters, as described hereinabove, apparatus 20 includes various combinations of the features described hereinabove with reference to FIGS. 12A-D. In general, the scope of the present invention includes using any number of filters, e.g., three or more.

Reference is now made to FIG. 12E, which is a schematic illustration of apparatus 20 in accordance with some applications of the present invention. In some applications, barrier 108 may protrude in a distal direction from distal end 112 of plunger 24, and distal end 100 of tube 22 may be shaped to define recess 110 into which barrier 108 fits upon plunger 24 being advanced to recess 110. When barrier 108 and recess 110 are configured as shown in FIG. 12E, apparatus 20 includes various combinations of the features described hereinabove with reference to FIGS. 12A-D.

Reference is now made to FIGS. 13A-D, which are schematic illustrations of apparatus 20 in accordance with some applications of the present invention. In some applications, filters 26a and 26b are disposed on distal end 112 of plunger 24 and filters 26a and 26b are separated by (a) recess 110 defined in distal end 112 of plunger 24 (FIGS. 13B and 13D) or (b) barrier 108 protruding in a distal direction from distal end 112 of plunger 24 (FIGS. 13A and 13C). Plunger 24 is shaped to define at least one compartment 134, and pushing the fluid through filters 26a and 26b pushes the fluid into compartment 134 (FIGS. 13C-D). In some applications, tube 22 comprises at least two puncturing elements 30j protruding in a proximal direction from distal end 100 of tube 22 and configured to puncture filters 26a and 26b, respectively, upon plunger 24 being advanced to barrier 108. In some applications, tube 22 does not comprise puncturing elements 30j. In some applications, plunger 24 is shaped to define at least one plunger lumen 40e, an opening of plunger lumen 40e being arranged to align with one of filters 26a or 26b, and not to simultaneously align with the other one of filters 26a or 26b. A distal end of plunger lumen 40e is configured to open upon plunger 24 being maximally advanced within tube 22 by puncturing element 30j, protruding from distal end 100 of tube 22.

In some applications, distal end 100 of tube 22 is shaped to define at least two conduits 32e, configured to align with filters 26a and 26b, respectively. A stopper 148 is initially disposed over the distal openings of conduits 32e. Following pushing the fluid through filters 26a and 26b, apparatus 20 may be turned upside-down such that the proximal end of tube 22 or plunger 24 can be rested on a horizontal surface, stopper 148 removed, and either one of filters 26a or 26b can be tested for presence of the particulate by passing the particulate-presence-testing-facilitation solution through a respective conduit 32e and subsequently inserting a dipstick through the respective conduit 32e.

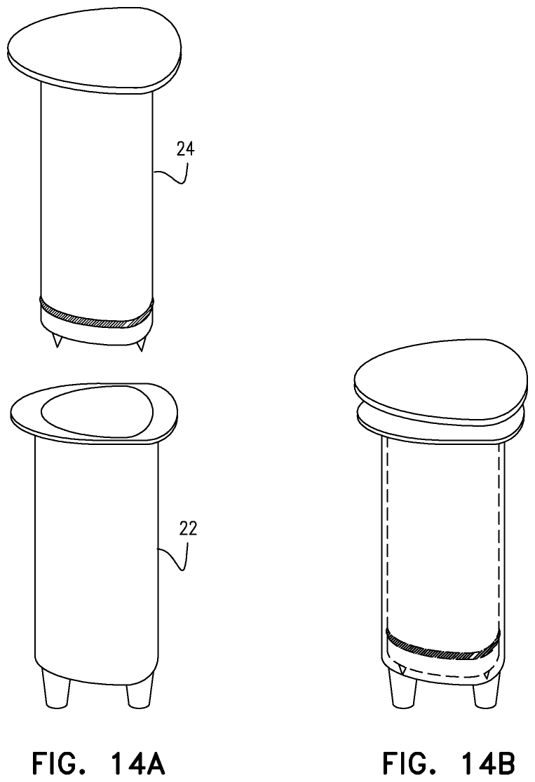

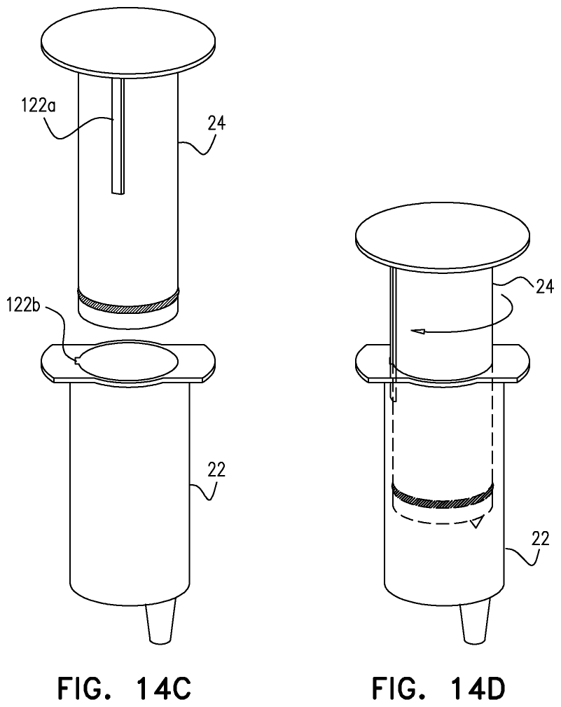

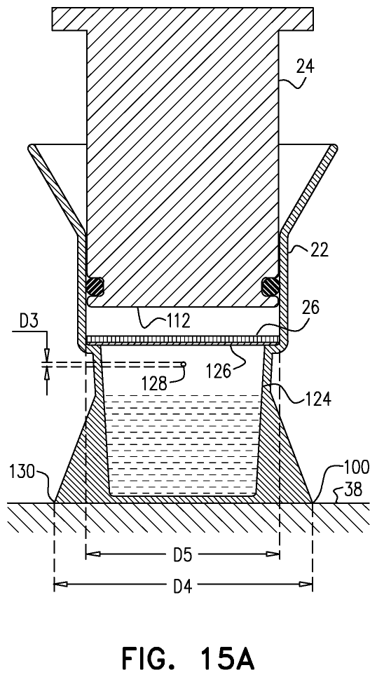

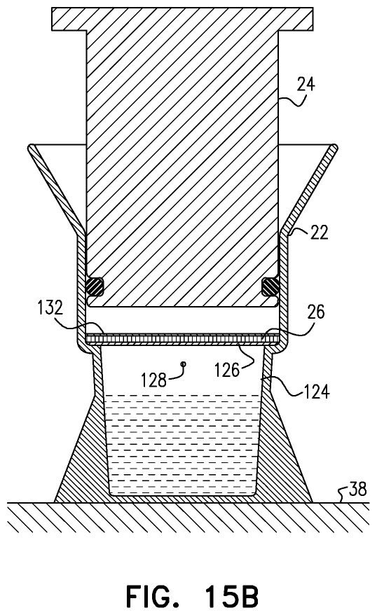

Reference is now made to FIGS. 14A-B, which are schematic illustrations of apparatus 20, in accordance with some applications of the present invention. In some applications, tube 22 and plunger 24 are shaped to have rotational asymmetry, such that during at least a portion of the advancement of plunger 24 within tube 22, plunger 24 is advanceable within tube 22 in only a single orientation of plunger 24 with respect to tube 22. The rotational asymmetry of plunger 24 and tube 22 facilitates, for example, in respective applications, the ability to easily align the slant of distal end 112 of plunger 24 (FIG. 20) with the slant of distal end 100 of tube 22, as well as the ability to easily align barrier 108 (FIG. 12A) with recess 110 as plunger 24 is advanced in tube 22.

Reference is now made to FIGS. 14C-D, which are schematic illustrations of apparatus 20, in accordance with some applications of the present invention. In some applications, the rotational asymmetry of tube 22 and plunger 24 is achieved by plunger 24 and tube 22 having corresponding interlockable pieces 122a and 122b, such that plunger 24 is advanceable within tube 22 in only a single orientation of plunger 24 with respect to tube 22. Interlockable piece 122a may be disposed on plunger 24 and interlockable piece 122b may be disposed in tube 22 (as shown in FIG. 14C), or they may be disposed in an opposite configuration wherein interlockable piece 122a is in tube 22 and interlockable piece 122b is on plunger 24.

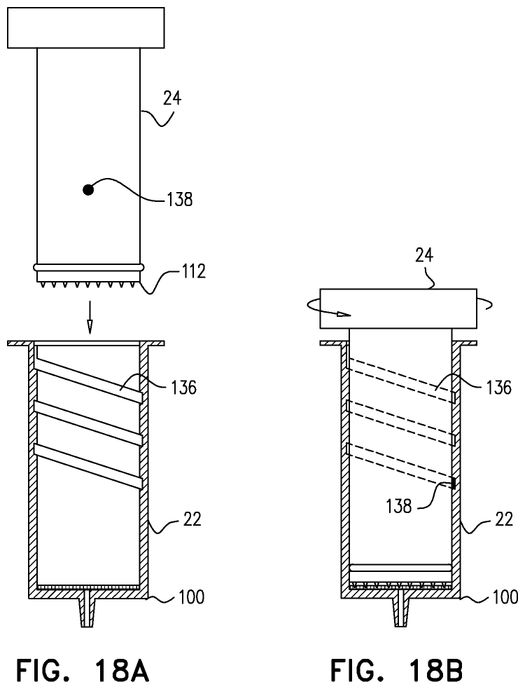

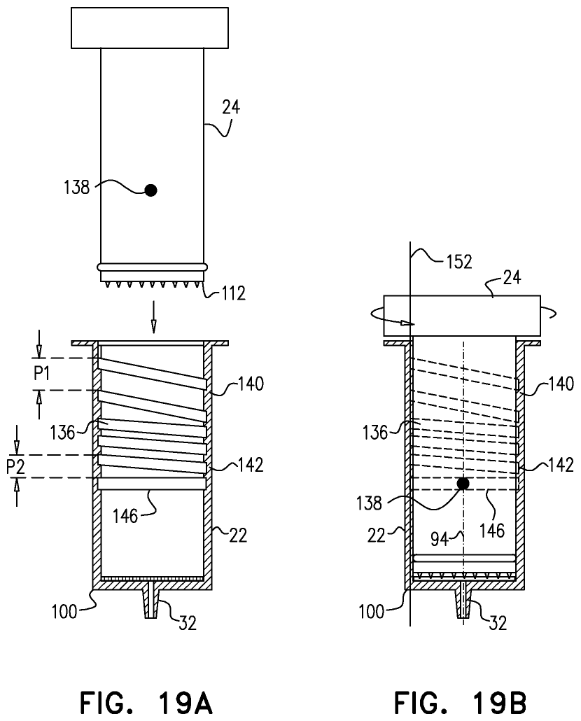

Reference is now made to FIGS. 16A-B, which is a schematic illustration of apparatus 20 in accordance with some applications of the present invention. In some applications, tube 22 is closed at a distal end thereof, filter 26 is disposed within tube 22, and tube 22 is shaped to define a fluid-collection compartment 124 distal to filter 26. Plunger 24 is arranged to push fluid through filter 26 and into fluid-collection compartment 124. In some applications, apparatus 20 further comprises a support 126, which is: (a) shaped to define one or more openings, (b) disposed distal to filter 26 within tube 22, (c) in contact with filter 26, and (d) configured to support filter 26 during the pushing of the fluid through filter 26. Typically, support 126 is positioned to inhibit distal movement of plunger 24 past filter 26. A wall of fluid-collection compartment 124 is shaped to define a pressure-release hole 128, such that air pressure in compartment 124, generated by advancing plunger 24, is released through pressure-release hole 128. Typically, a diameter D3 of pressure-release hole 128 is at least 50 microns and/or less than 1500 microns, such that it is small enough for air to easily pass through it but not for a liquid (such as the gargle fluid). Typically, pressure-release hole 128 is disposed above a volume of 2 cc of compartment 124 when distal end 100 of tube 22 is resting on horizontal surface 38. In some applications tube 22 is shaped to define a flat external, surface-contact portion 130 which is shaped to contact horizontal surface 38. Typically surface-contact portion 130 has a diameter D4 which is at least equal to a diameter D5 of filter 26, allowing apparatus 20 to stably balance on distal end 100 of tube 22.

In some applications, plunger 24 is configured to rotate with respect to tube 22 such that friction caused by the rotation of distal end 12 against filter 26 tears filter 26 upon plunger 24 being maximally advanced in tube 22 and subsequently rotated with respect to tube 22.

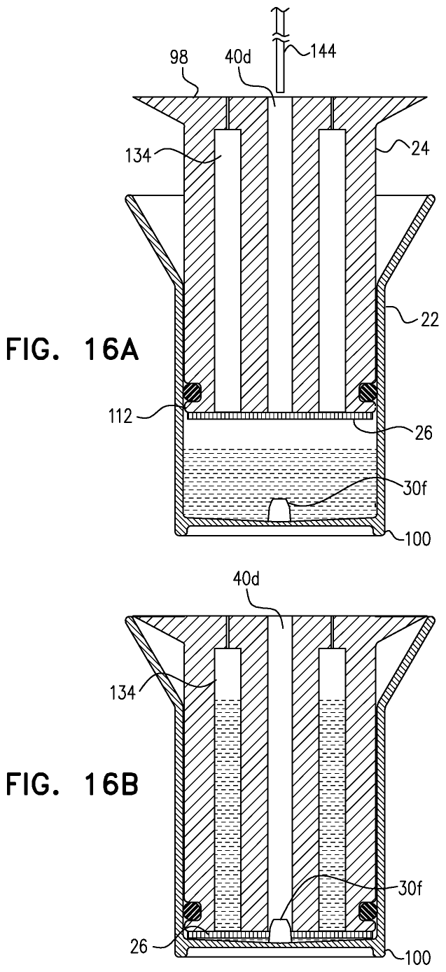

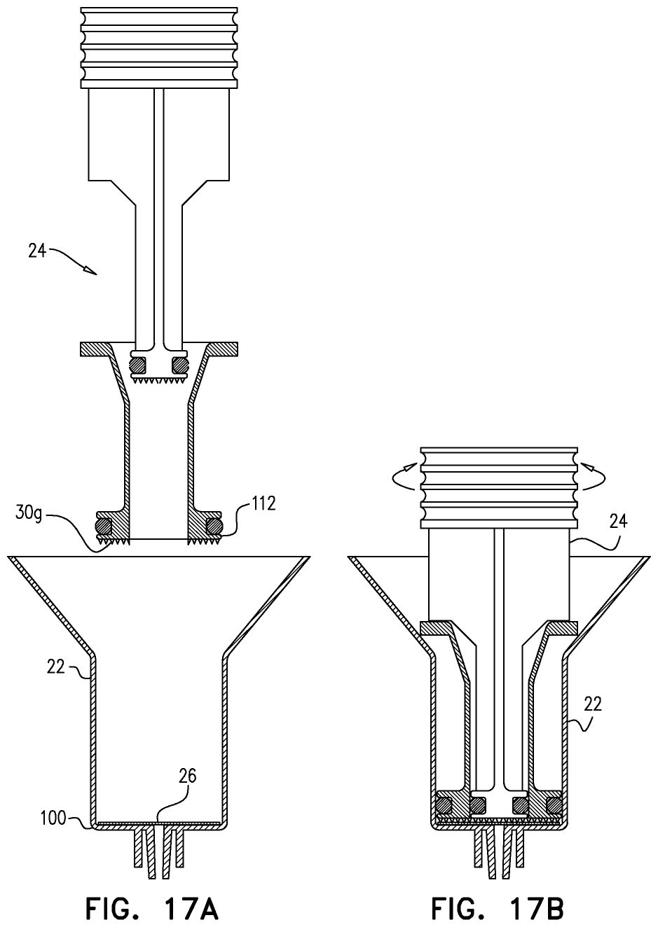

Reference is now made to FIG. 15B, which is a schematic illustration of apparatus 20 in accordance with some applications of the present invention. As plunger 24 is withdrawn from tube 22, a reverse-suction is created in tube 22 that may pull filter 26 in a proximal direction. To prevent this, in some applications, apparatus 20 further comprises a support 132, which is: (a) shaped to define one or more openings, (b) disposed proximal to filter 26 within tube 22, (c) in contact with filter 26, and (d) configured to support filter 26 during withdrawal of plunger 24 in a proximal direction.

Reference is now made to FIGS. 16A-B, which are schematic illustration of apparatus 20, in accordance with some applications of the present invention. In some applications, filter 26 is disposed on distal end 112 of plunger 24, and a puncturing element 30f protrudes in a proximal direction from distal end 100 of tube 22 and is configured to puncture filter 26 upon plunger 24 being maximally advanced in tube 22. In some applications, plunger 24 is configured to rotate with respect to tube 22 such that puncturing element 30f tears filter 26 upon plunger 24 being maximally advanced in tube 22 and subsequently rotated with respect to tube 22. Plunger 24 is shaped to define at least one compartment 134 and pushing the fluid through filter 26 pushes the fluid into compartment 134 (FIG. 16B). Plunger 24 is further shaped to define plunger lumen 40d, through which the particulate-presence-testing-facilitation solution may be passed and a dipstick inserted. A sample may be taken from filter 26 prior to filter 26 being tested for presence of the particulate by swabbing filter 26 with swab 144 from proximal end 98 of plunger 24 through plunger lumen 40d.

Reference is now made to FIGS. 17A-B, which are schematic illustrations of apparatus 20 in accordance with some applications of the present invention. In some applications, filter 26 is disposed in distal portion of tube 22, and at least one puncturing element 30g protrudes in a distal direction from distal end 112 of plunger 24. Puncturing element 30g is configured to puncture filter 26 upon plunger 24 being maximally advanced within tube 22. In some applications, plunger 24 is configured to rotate with respect to tube 22, when inside tube 22, such that puncturing element 30g tears filter 26 upon plunger 24 being maximally advanced to filter 26 and subsequently rotated with respect to tube 22. Tearing filter 26 further facilitates testing for presence of the particulate, as a larger surface area of filter 26 is exposed to the particulate-presence-testing-facilitation solution.

Reference is now made to FIGS. 18A-B, which are schematic illustrations of apparatus 20 in accordance with some applications of the present invention. In some applications, apparatus 20 comprises threading 136 and a protrusion 138 configured to slidably engage threading 136 such that plunger 24 is advanceable within tube 22 by rotation of plunger 24 with respect to tube 22. Advancing plunger 24 by rotation, guided by threading 136, helps control the speed of the advancement and helps maintain steady advancement against pressure in tube 22. In some applications, protrusion 138 and threading 136 are disposed such that threading 136 is on the inside of at least a portion of tube 22 and protrusion 138 protrudes outwards from a wall of plunger 24 (FIG. 18A). In some applications, protrusion 138 and threading 136 are disposed such that threading 136 is on the outside of at least a portion of plunger 24 and protrusion 138 protrudes inwards from a wall of tube 22 (configuration not shown).

Reference is now made to FIGS. 19A-B, which are schematic illustrations of apparatus 20 in accordance with some applications of the present invention. In some applications, a first pitch P1 of threading 136 at a first location 140 is different from a second pitch P2 of threading 136 at a second location 142. In some applications, second pitch P2 of threading 136 is less than first pitch P1 (FIG. 19A). A decreasing pitch, in a proximal to distal direction, is advantageous for example when filter 26 is disposed on distal end 112 of plunger 24 such that the fluid is being pushed proximally into a compartment in plunger 24, e.g., compartment 134 in FIG. 16A. Advancing plunger 24 through first location 140, corresponding to higher first pitch P1, requires a greater downward force on the proximal end of plunger 24, whereas advancing plunger 24 through second location 142, corresponding to lower second pitch P2, requires less downward force on the proximal end of plunger 24. Initially air (rather than the fluid) is pushed into the compartment, allowing plunger 24 to be pushed through first location 140 with relative ease, and subsequently the fluid is pushed into the compartment, at which point the pitch is decreased to lower pitch P2 to facilitate easier advancement of plunger 24 against the fluid. In addition, as the fluid is pushed through filter 26 the particulate collects on filter 26 such that further advancement of plunger 24 may become more difficult; the transition from higher first pitch P1 to lower second pitch P2 as plunger 24 is advanced helps decrease the force required to push the remaining fluid through filter 26.

In some applications, first pitch P1 of threading 136 at first location 140 is less than second pitch P2 of threading 136 at second location 142 (configuration not shown). An increasing pitch, in a proximal to distal direction, is advantageous for example when filter 26 is disposed in distal end 100 of tube 22, such that the fluid is being pushed distally out of a conduit in distal end 100 of tube 22, e.g., conduit 32. First pitch P1 is lower to facilitate easier advancement of plunger 24 while the fluid is initially pushed out of conduit 32, and subsequently, once the fluid has been pushed out, second pitch P2 is higher for the remaining advancement of plunger 24.

In some applications, a portion 146 of threading 136 that is closest to distal end 100 of tube 22 is perpendicular to a line 152 that is parallel to longitudinal axis 94 of tube 22. Protrusion 138 engages portion 146 of threading 136 when plunger 24 is maximally advanced within tube 22, such that plunger 24 can rotate with respect to tube 22 without further inhibition by threading 136. This uninhibited rotation of plunger 24 with respect to tube 22 facilitates, for example, tearing of filter 26 by rotation of plunger 24 once plunger 24 is maximally advanced within tube 22 and thereby testing for the particulate using the particulate-presence-testing-facilitation solution.

It is noted that apparatus 20 may include various combinations of features shown in FIGS. 1-21.

In general, the scope of the present invention includes using any number of filters, e.g., three or more. Furthermore, the scope of the present invention includes using adhesive properties of a filter to facilitate the trapping of the particulate. For example, mucus from the throat that contains the bacteria, and/or the cell walls of the bacteria, may adhere to the filter.

The scope of the present invention includes testing for various types of particulate matter, in addition to that which is delineated above. For example, apparatus and methods described herein may be used to test for parts of microscopic or macroscopic organisms, or for discharged matter (e.g., eggs) emanating from such organisms.

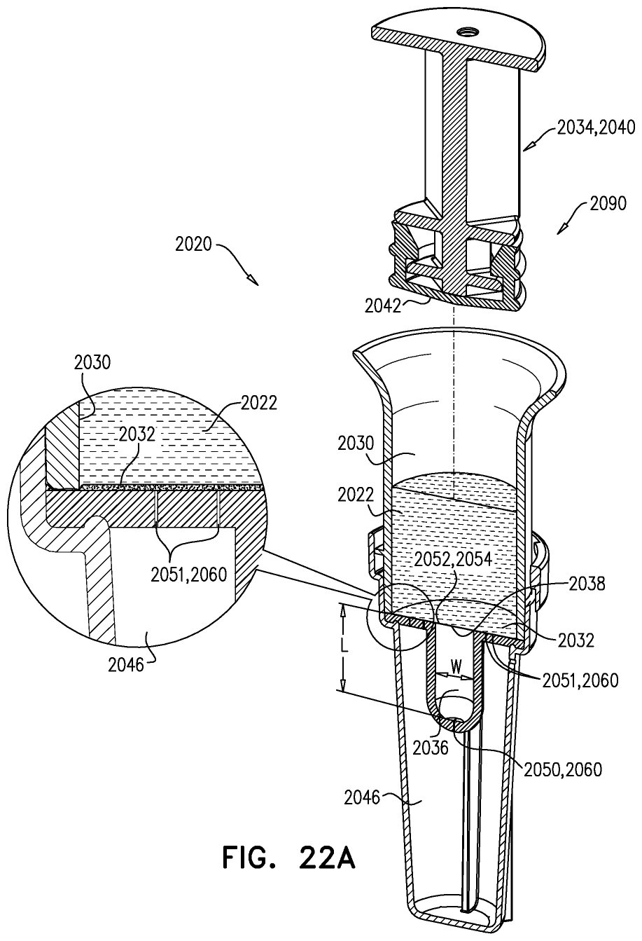

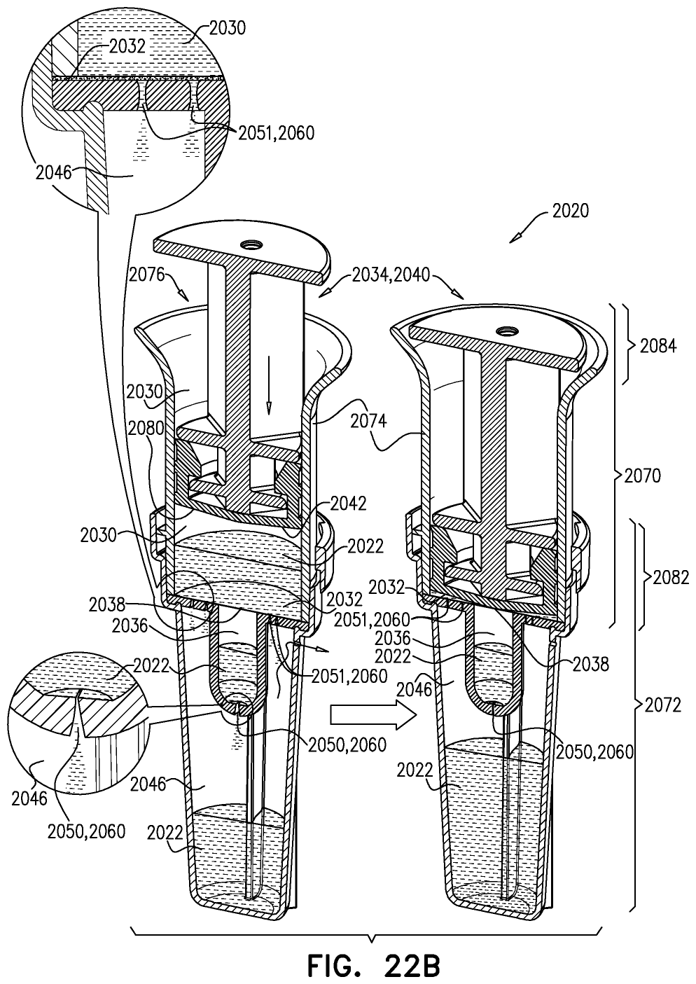

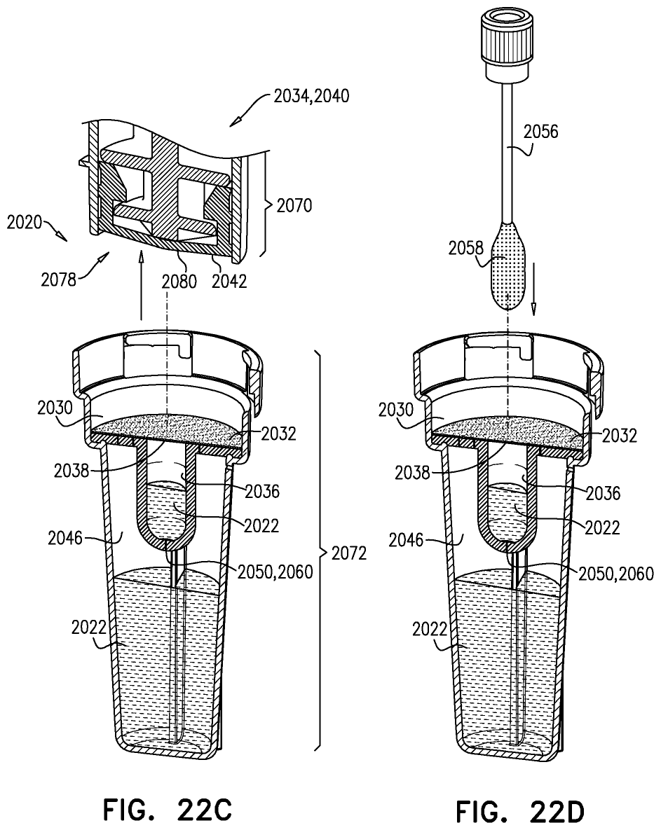

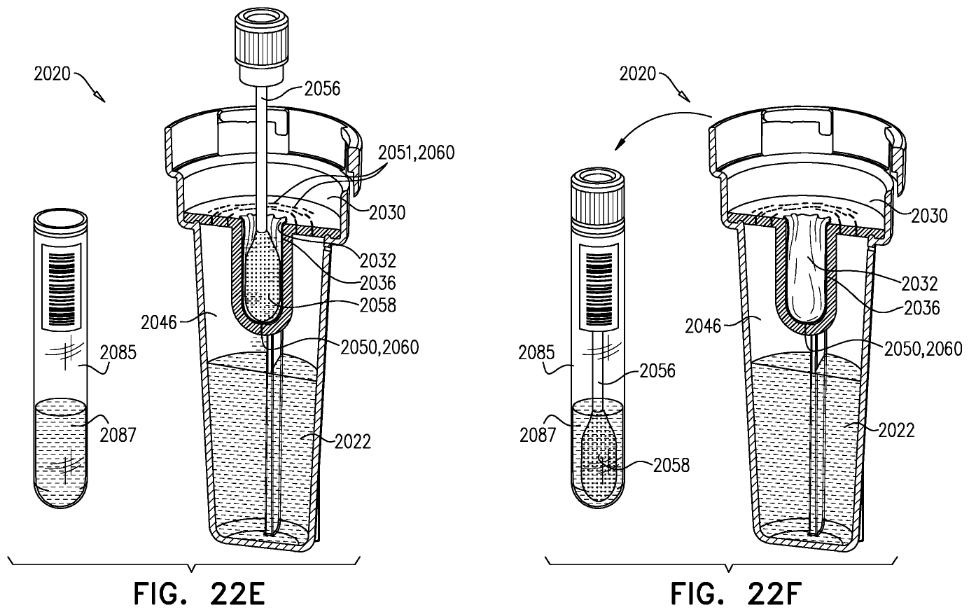

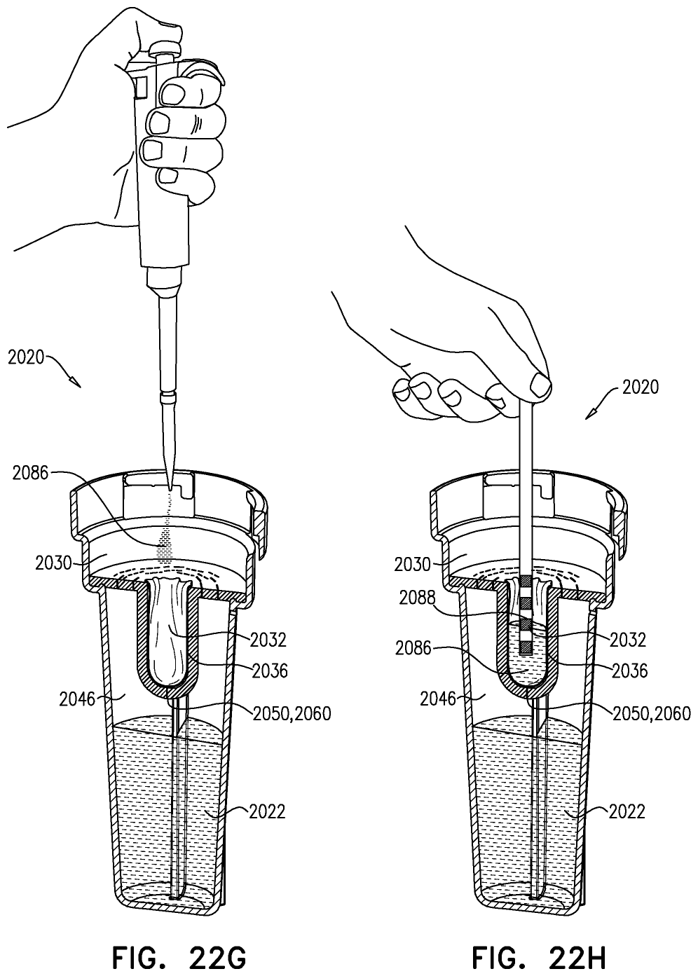

FIGS. 22A-H are schematic illustrations of a testing device 2020 for testing for presence of particulate in a liquid 2022, in accordance with an application of the present invention. For some applications, the particulate comprises biological particulate, for example, a microorganism, a fungus, a bacterium (e.g., a group A streptococcus bacterium), a spore, a virus, a mite, a biological cell, a biological antigen, a protein, a protein antigen, and a carbohydrate antigen. Alternatively, testing device 2020 is used for testing a non-particulate substance of interest, whether a biological or chemical substance, soluble, immiscible or an emulsion, an atom, a molecule, a polymer or a mixture of substances.

Testing device 2020 typically comprises:
a liquid container 2030 for containing liquid 2022; typically, liquid container 2030 has an internal volume of at least 0.5 ml (e.g., at least 1 ml, such as at least 5 ml), no more than 500 ml (e.g., no more than 70 ml), and/or between 0.5 ml (e.g., 1 ml or 5 ml) and 500 ml (e.g., 70 ml);
a filter 2032, disposed in or downstream of liquid container 2030; and
a liquid-pressure source 2034, which is arranged to apply pressure to drive liquid 2022 contained in liquid container 2030 through filter 2032.

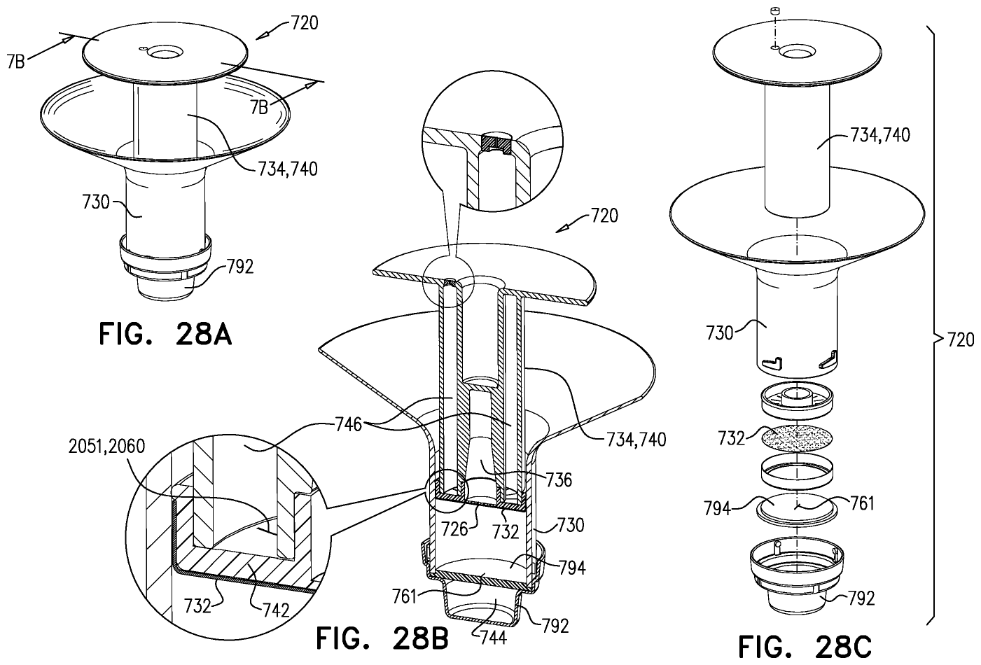

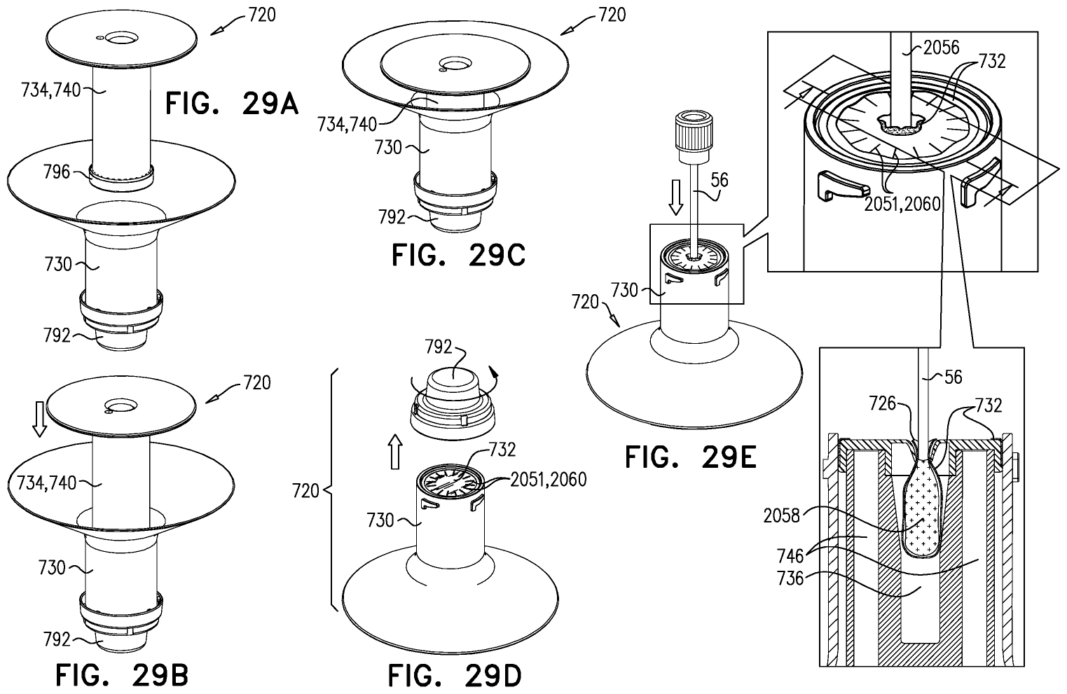

As used in the present application, including in the claims, “upstream” and “downstream” refer to the direction of fluid flow through testing device 2020, and not the orientation of the device with respect to the Earth. For example, filter chamber 736, described hereinbelow with reference to FIGS. 28A-8E, is downstream of liquid container 730, even though the filter chamber is physically above the liquid container when the testing device is oriented as shown in the figures.

Typically, liquid container 2030 does not comprise a Luer lock or any other type of needle-coupling mechanism.

Filter 2032 comprises synthetic or natural materials formed, for example, as a matrix, membrane, fabric, beads, or other configuration. For example, the inventors have tested the following filters manufactured by Sterlitech (Washington, USA):
Grade C glass microfiber filter media (Cat. No. C2500 & C3700)
GC-50 glass fiber membrane filters (Cat. No. GC5037100)
polyethersulfone (PES) membrane filters (Cat. No. PES0825100, PES0837100, PES1225100, PES1237100, PES06525100, PES4525100, PES4525100)
polycarbonate membrane filters (Cat. No. PCT0613100, PCT2025100, PCT0625100, PCT1025100, PCT0825100)
cellulose acetate membrane filters (Cat. No. CA0825100)
polyester membrane filters (Cat. No. PET0125100, PET0825100)

Typically, filter 2032 is configured to trap at least 40% (such as at least 95%, e.g., at least 99%) of the particulate to be tested and allow passage of liquid 2022. For example, for applications in which the particulate is group A streptococcus bacteria, the filter may be configured to trap at least 40% (such as at least 95%, e.g., at least 99%) of the group A streptococcus bacteria and allow passage of liquid 2022. For some applications, filter 2032 has a filter surface area of an upstream side of the filter equal to at least 0.3 cm2, no more than 100 cm2 (e.g., no more than 30 cm2), and/or between 0.3 cm2 and 100 cm2, such as between 0.3 and 30 cm2. For some applications, filter 2032 has a pore size of at least 0.01 microns, no more than 20 microns, and/or between 0.01 and 20 microns (such as for capturing viruses, bacteria, and/or cells), such as at least 0.01 microns and/or no more than 0.3 microns (e.g., for capturing viruses), at least 0.45 microns and/or no more than 2 microns (e.g., for capturing bacteria), at least 2 microns and/or no more than 20 microns (e.g., for capturing human cells), or at least 0.01 microns and/or no more than 20 microns (e.g. for capturing viruses and bacteria).

For some applications, liquid-pressure source 2034 comprises at least one of the following:
a plunger 2040, which comprises a plunger head 2042 that is shaped so as to be insertable into liquid container 2030 so as to form a movable seal with a wall of a plunger housing (optionally, all or a portion of liquid container 2030 defines the wall of the plunger housing);
a positive-pressure pump (e.g., a hydraulic pump, a syringe, or a motorized and/or electrical pump) disposed upstream of filter 2032 (configuration not shown); optionally, for some application, the positive-pressure pump comprises a chamber with one or more flexible walls, the squeezing of which pumps air and/or liquid 2022 itself out of the chamber; or
a vacuum pump disposed downstream of filter 2032 (and, if provided, of the one or more valves 2060, described hereinbelow)(configuration not shown).

For some applications, plunger 2040 further comprises a plunger shaft, and plunger head 2042 is disposed at a downstream end portion of the plunger shaft. Typically, but not necessarily, plunger 2040 has one of the following configurations:
the plunger head comprises a separate piece of material (e.g., comprising a polymer) that is coupled to the plunger shaft and is shaped so as to define the downstream surface of plunger head 2042 and optionally a lateral sealing surface, or
the distal surface of plunger head 2042 is defined by the end of the plunger shaft, and, for example, a separate sealing ring (e.g., comprising a polymer) may be provided to provide a lateral sealing surface.

For some applications, testing device 2020 further comprises a waste liquid receptacle 2046, which is coupled to liquid container 2030 downstream of filter 2032 (and, if provided, of the one or more valves 2060, described hereinbelow). Liquid-pressure source 2034 is arranged to apply pressure to drive liquid 2022 contained in liquid container 2030 through filter 2032 and then into waste liquid receptacle 2046.

For some applications, testing device 2020 further comprises a filter chamber 2036 that is (a) disposed downstream of liquid container 2030, (b) shaped so as to define an inlet 2038, and (c) in fluid communication with filter 2032. Filter chamber 2036 is shaped such that when filter 2032 is pushed into the filter chamber, such as described hereinbelow with reference to FIGS. 22D-E, the filter chamber collects filter 2032 into a relatively small volume, thereby increasing the consolidated sensitivity of rapid and backup tests for particulate trapped by filter 2032. If, by contrast, filter 2032 were flat in liquid container 2030, it would be difficult to collect a sample from a high percentage of the surface of the filter. In addition, filter chamber 2036 readily hosts at least one extraction reagent 2086 and a test strip 2088, as described hereinbelow with reference to FIGS. 22G-H.

Optionally, filter chamber 2036 is nipple-shaped. For some applications in which testing device 2020 comprises waste liquid receptacle 2046, filter chamber 2036 is laterally surrounded by at least a portion of waste liquid receptacle 2046, such as shown in FIGS. 22A-H. Alternatively or additionally, for some applications, filter chamber 2036 is disposed within waste liquid receptacle 2046, such as shown in FIGS. 22A-H.

For some applications, inlet 2038 of filter chamber 2036 has an inlet area that equals at least 4%, no more than 40%, and/or between 4% and 40% of a filter surface area of an upstream side of filter 2032, such as between 10% and 20%. Alternatively or additionally, for some applications, filter chamber 2036 has:
an internal volume of at least 0.5 ml, no more than 12 ml (e.g., no more than 4 ml), and/or between 0.5 and 12 ml (such as between 0.5 and 4 ml), such as at least 1 ml (e.g., at least 2 ml), no more than 5 ml, and/or between 1 and 5 ml, such as between 2 and 5 ml,
an internal surface area that equals at least 10%, no more than 150%, and/or between 10% and 150% of a filter surface area of an upstream side of filter 2032, such as between 70% and 130%,
an internal length L equal to between 0.5 and 10 cm, such as between 1.5 and 5 cm,
an internal width W equal to between 0.3 and 3 cm, such as between 0.5 and 1.5 cm,
an internal length L of at least 0.5 cm, no more than 10 cm (e.g., no more than 5 cm), and/or between 0.5 and 10 cm, such as between 0.5 cm and 5 cm, e.g., between 1 and 5 cm, and/or
an internal length L equal to at least 50%, no more than 2000%, and/or between 50% and 2000% of a greatest internal width W of filter chamber 2036, such as between 200% and 600%.

For some applications, such as shown in FIGS. 22A-H, filter chamber 2036 comprises one or more pressure-activated valves 2050, not disposed at inlet 2038. For applications in which testing device 2020 comprises waste liquid receptacle 2046, the one or more pressure-activated valves 2050 are typically disposed in fluid communication between filter chamber 2036 and waste liquid receptacle 2046. For some applications, liquid container 2030 is shaped so as to define one or more openings 2051 (typically, non-valved openings) through a wall of liquid container 2030, the one or more openings 2051 are downstream of filter 2032 when filter 2032 is removably disposed upstream of filter chamber 2036 with filter 2032 partially covering inlet 2038, and filter chamber 2036 is not disposed so as to receive liquid 2022 that is driven through the one or more openings 2051. The one or more openings 2051 allow liquid 2022 to pass, thereby drawing the liquid through filter 2032. (As described hereinabove with reference to FIGS. 22A-B, testing device 2020 may comprise an upstream component 2070 and a downstream component 2072 that are removably coupled together; in such configurations, the one or more openings 2051 defined by liquid container 2030 may optionally be defined by the portion of downstream component 2072 that helps define liquid container 2030.) For some applications, such as shown in FIGS. 22A-D, filter 2032 is removably disposed upstream of filter chamber 2036 with filter 2032 partially covering inlet 2038.

For some applications, inlet 2038 has an inlet centroid 2052 that is disposed within a distance of a filter centroid 2054, the distance equal to 50% of a greatest dimension of filter 2032, when filter 2032 is removably disposed upstream of filter chamber 2036 with filter 2032 partially covering inlet 2038. For example, filter 2032 may be centered upstream of inlet 2038.

For some applications, an elongate member 2056 is provided that configured to push at least a portion of filter 2032 into filter chamber 2036. Optionally, elongate member 2056 comprises a swab 2058 at a distal end of the elongate member. In applications in which filter chamber 2036 comprises one or more pressure-activated valves 2050, inserting elongate member 2056 into filter chamber may squeeze any liquid 2022 remaining in filter chamber 2036 through one or more pressure-activated valves 2050 and out of filter chamber 2036. For other applications in which liquid-pressure source 2034 comprises plunger 2040, plunger head 2042 is configured to push at least a portion of filter 2032 into filter chamber 2036 (configuration not shown). In applications in which filter chamber 2036 comprises one or more pressure-activated valves 2050, inserting plunger head 2042 into filter chamber may squeeze any liquid 2022 remaining in filter chamber 2036 through one or more pressure-activated valves 2050 and out of filter chamber 2036.

Reference is still made to FIGS. 22A-H. In an application of the present invention, testing device 2020 further comprises one or more valves 2060. For these applications, filter 2032 is typically disposed in or downstream of liquid container 2030 and upstream of the one or more valves 2060. Liquid-pressure source 2034 is arranged to apply pressure to drive liquid 2022 contained in liquid container 2030 through filter 2032 and then through the one or more valves 2060. For applications in which testing device 2020 comprises waste liquid receptacle 2046, waste liquid receptacle 2046 is typically coupled (removably or permanently) to liquid container 2030 downstream of the one or more valves 2060, and liquid-pressure source 2034 is arranged to apply pressure to drive liquid 2022 contained in liquid container 2030 through filter 2032, then through the one or more valves 2060, and then into waste liquid receptacle 2046. Typically filter chamber 2036 is not disposed so as to receive liquid 2022 that is driven through at least one of the one or more valves 2060.

For some applications, the one or more valves 2060 comprise one or more pressure-activated valves. For example, as mentioned above, filter chamber 2036 may comprise one or more pressure-activated valves 2050, not disposed at inlet 2038. For example, the pressure-activated valves may be formed from slits or flaps in an elastic material (such as silicone), or may comprise any small valves known in the valve art. The one or more pressure-activated valves are configured to open at the higher pressure applied by liquid-pressure source 2034, so as to allow liquid 2022 to pass through filter 2032, and to remain closed at the much lower pressure applied by at least one extraction reagent 2086, as described hereinbelow with reference to FIGS. 22G-H. Preventing the leakage of the at least one extraction reagent 2086 causes the at least one extraction reagent 2086 to bathe filter 2032, which is beneficial for optimal testing for particulate trapped by filter 2032 using a test strip 2088, also as described hereinbelow with reference to FIGS. 22G-H.

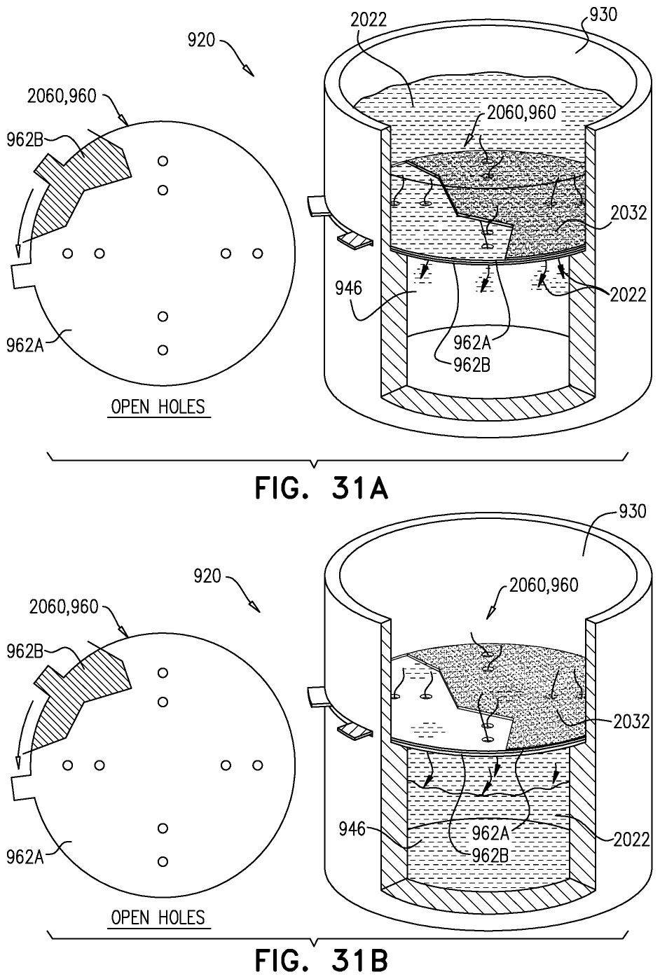

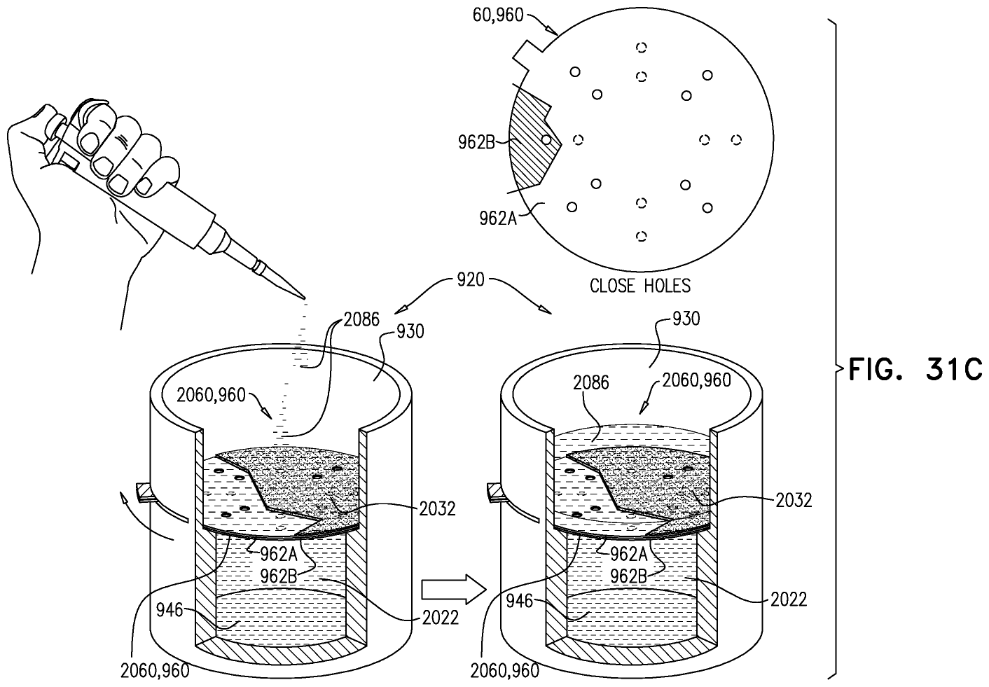

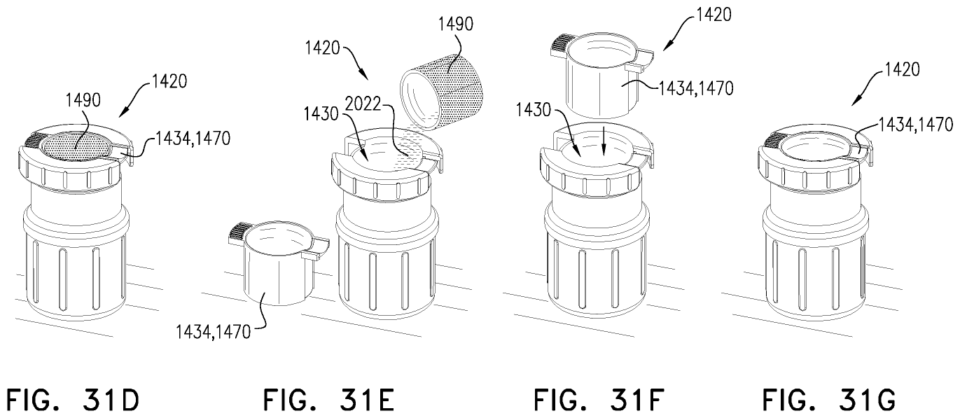

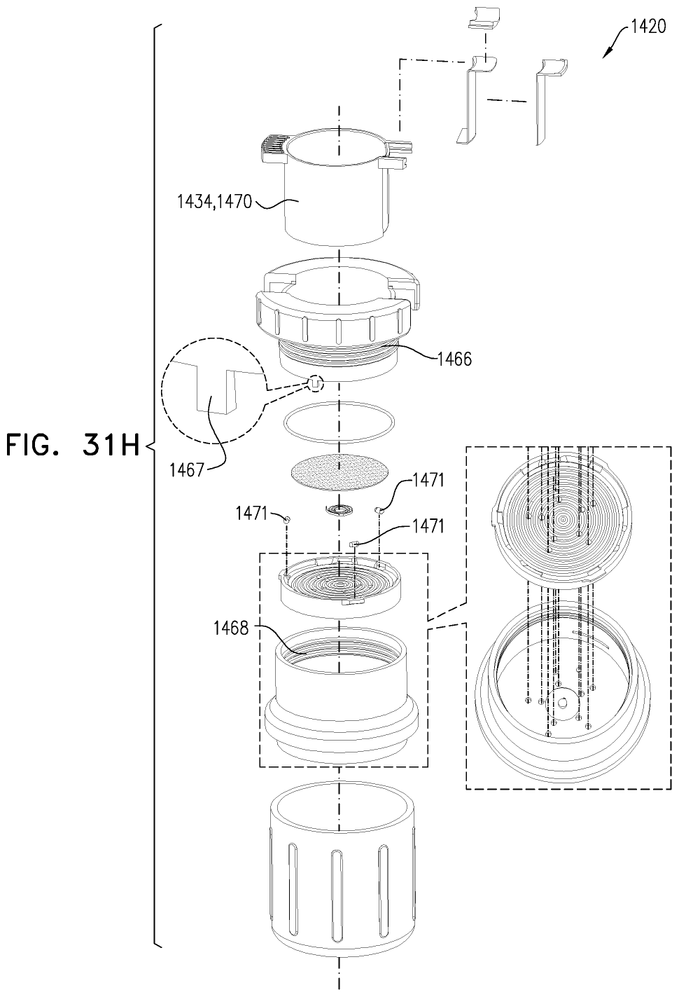

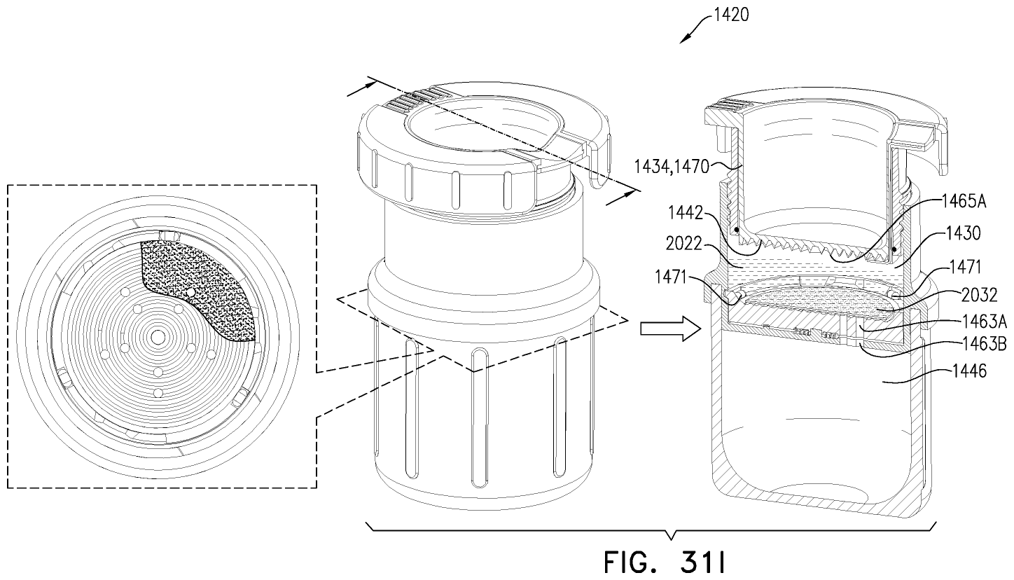

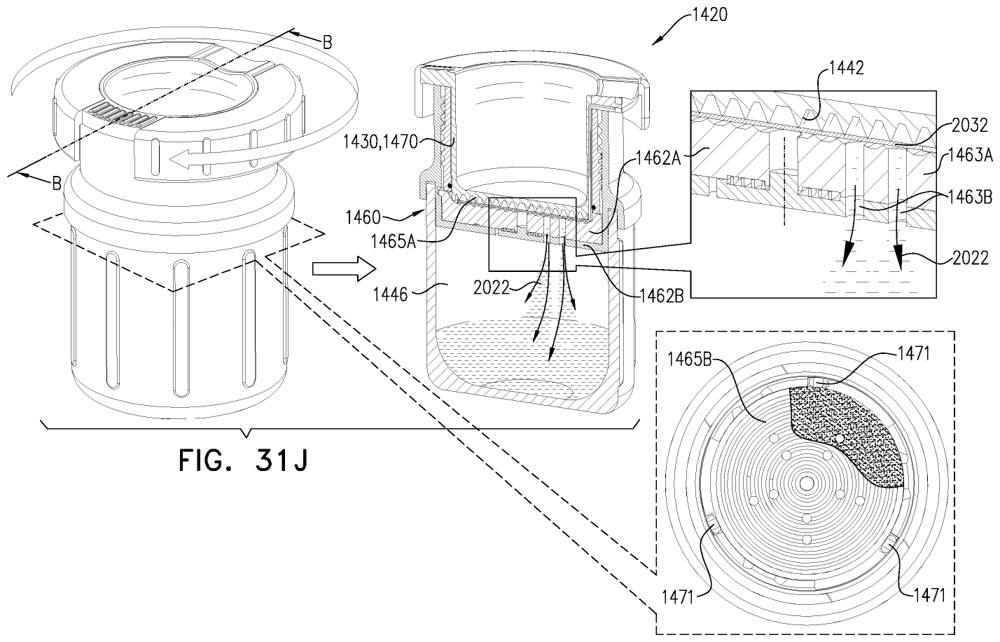

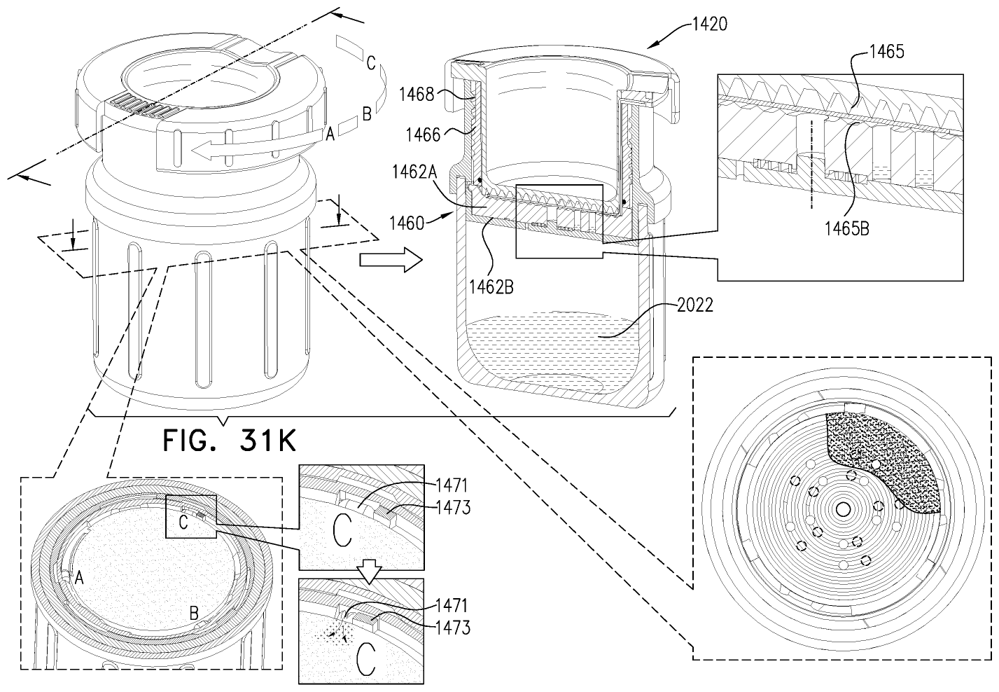

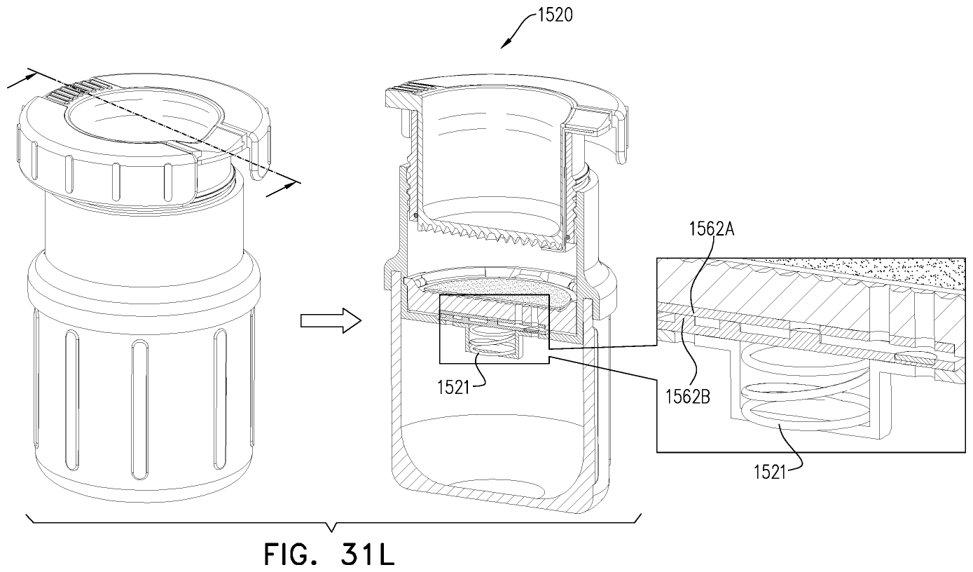

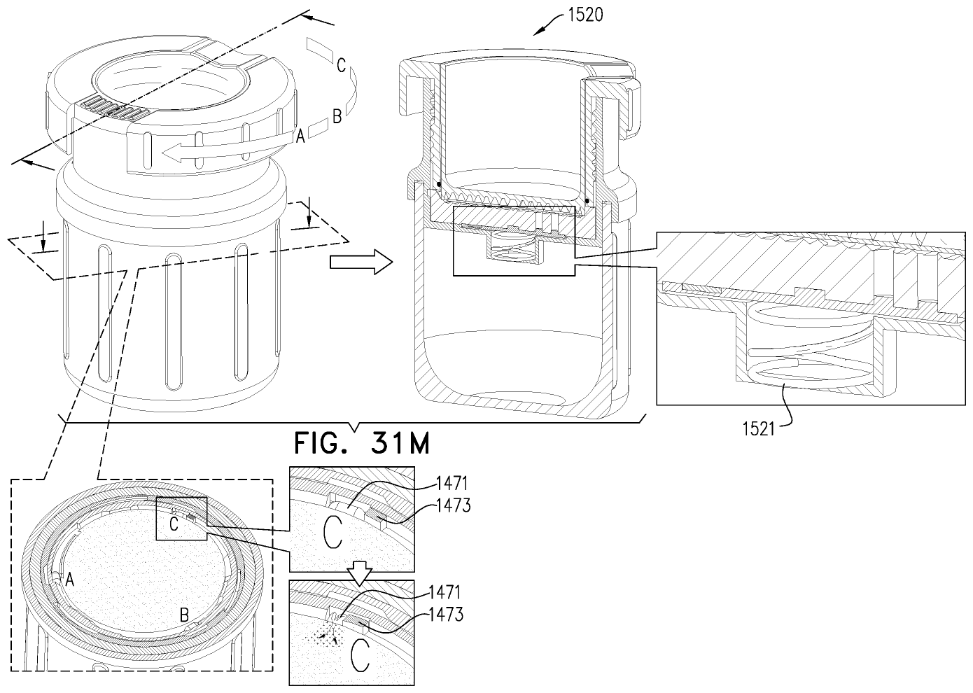

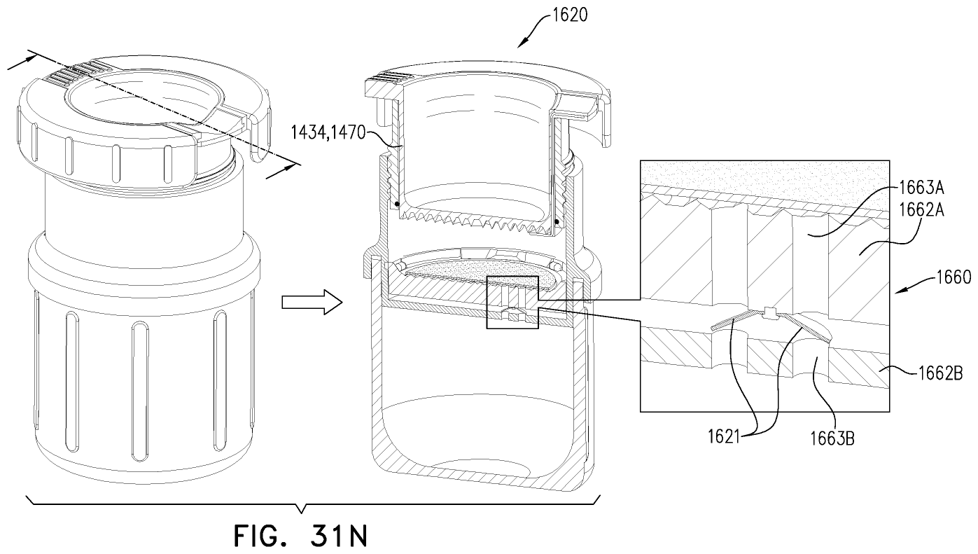

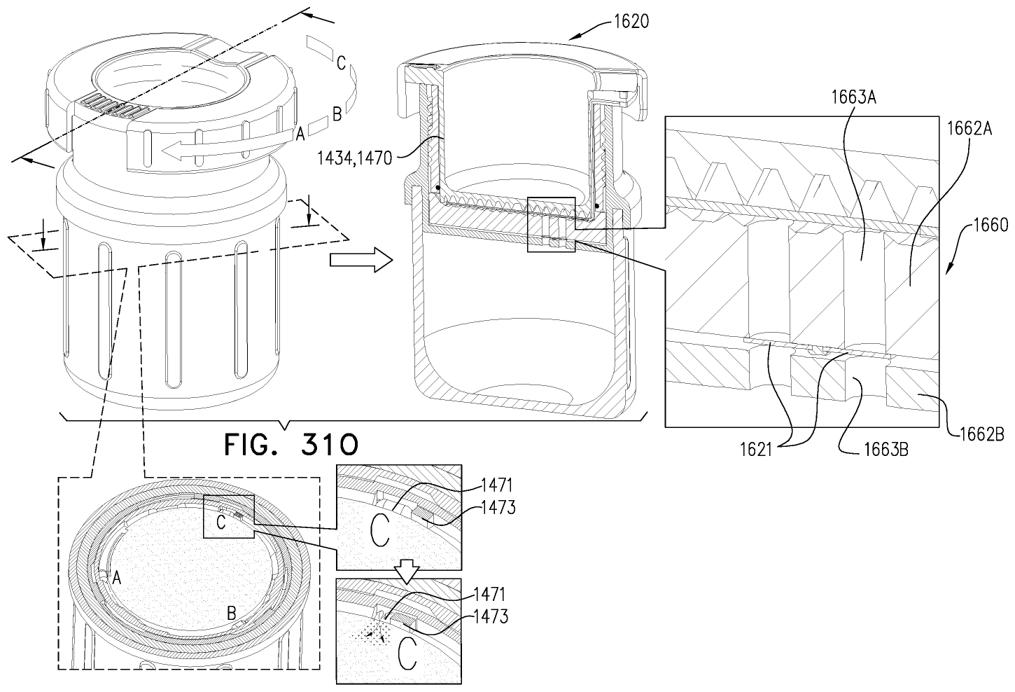

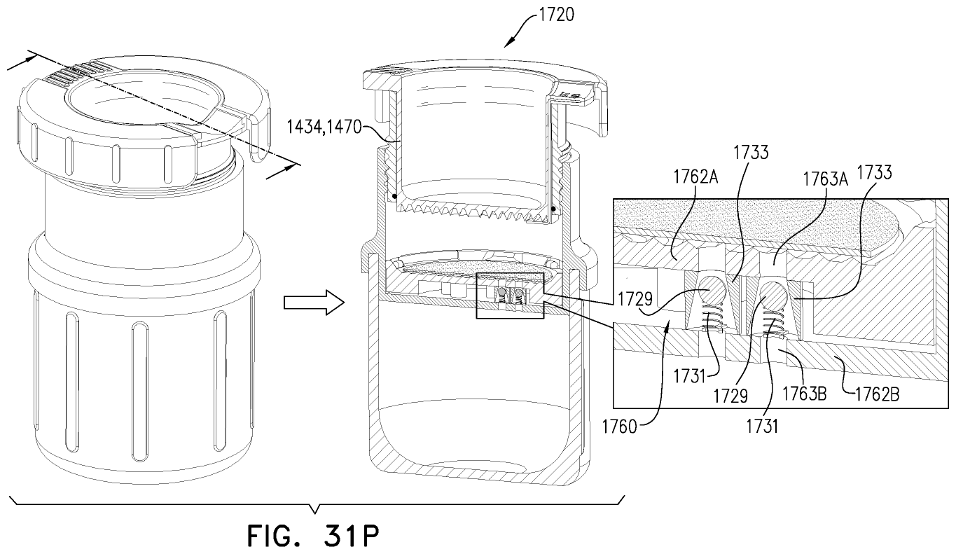

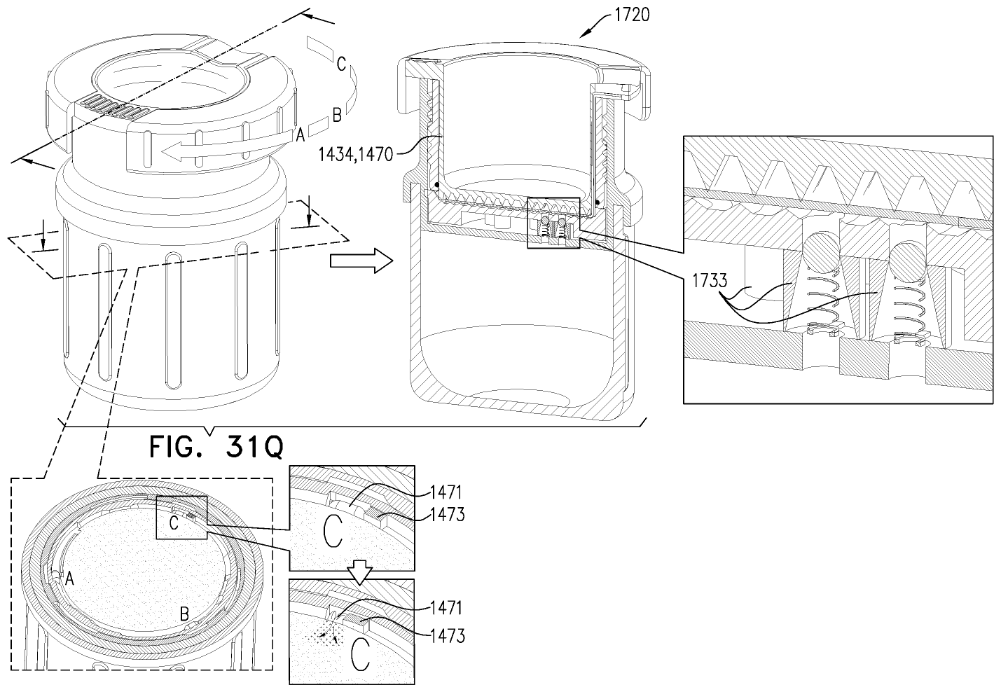

Alternatively or additionally, for some applications, the one or more valves 2060 comprise one or more non-pressure-activated valves, such as described hereinbelow with reference to FIGS. 31A-C, 31D-K, 31L-M, 31N-O, and/or 31P-Q.

For some applications, sterile packaging is provided, in which at least liquid container 2030, filter chamber 2036, the one or more valves 2060, and/or filter 2032 are removably disposed. The sterile packaging comprises one or more sterile packages; for example, each element may be removably disposed in a separate one of the packages, and/or more than one the elements may be disposed in a single one of the packages.

For some applications, at least one container comprising the at least one extraction reagent 2086 is provided. For example, the at least one extraction reagent 2086 may comprise 2M sodium nitrite and/or 0.2M acetic acid, and/or a releasing agent, which, upon contacting a microorganism, releases an antigen from the microorganism. For applications in which more than one extraction reagent 2086 is provided, and/or extraction reagent 2086 comprises a plurality of substances, each of the extraction reagents 2086 and/or substances may be provided in a separate container, and the extraction reagents 2086 and/or substances are combined prior to (e.g., immediately prior to) performing the assay. Alternatively or additionally, for some applications, a test strip 2088 is provided. Typically, test strip 2088 is a lateral flow test strip, such as a lateral flow immunoassay (e.g., chromatographic immunoassay) test strip, as is known in the art. For example, test strip 2088 may contain an antibody specific to strep A carbohydrate antigen, and the mixture migrates up the test strip and reacts with the antibody, thus generating a line on the test strip; the presence of this line indicates a positive test result. Alternatively or additionally, for some applications, a container is provided containing a solution for use in a detecting a pathogen.

Reference is still made to FIGS. 22A-H. In an application of the present invention, testing device 2020 comprises an upstream component 2070 and a downstream component 2072 (labeled in FIGS. 22B and 22C).

Upstream component 2070 typically comprises:
a plunger housing 2074, which is shaped so as to define an upstream opening 2076 (labeled in FIG. 22B) and a downstream opening 2078 (labeled in FIG. 22C); and
plunger 2040, which comprises a downstream plunger head 2042 that is shaped so as to be insertable into plunger housing 2074 so as to form a movable seal with a wall of plunger housing 2074; typically, an area of a downstream surface 2080 of downstream plunger head 2042 equals between 80% and 100% of an area of downstream opening 2078 (unlike in conventional syringes, in which the downstream surface of the plunger head is typically much larger than the narrow downstream opening of the syringe barrel).

Typically, plunger housing 2074 does not comprise a Luer lock or any other type of needle-coupling mechanism.

Downstream component 2072 typically comprises:
filter 2032, which has a filter surface area if an upstream side of the filter equal to at least 80% of the area of downstream surface 2080 of the downstream plunger head 2042;
waste liquid receptacle 2046, disposed downstream of filter 2032; and
for applications in which it is provide, filter chamber 2036.

Testing device 2020 is shaped so as to define liquid container 2030 for containing liquid 2022. Upstream component 2070 and downstream component 2072 are configured to be removably coupled together so as to form a liquid-impermeable seal, as shown in FIGS. 22A and 22B. FIG. 22C shows upstream component 2070 and downstream component 2072 after they have been decoupled from each other. For example, upstream component 2070 and downstream component 2072 may be configured to be removably coupled together by click-fitting together, by friction-fitting together, by twist-and-lock fitting together, or by magnetic coupling together.

For some applications, such as shown in FIGS. 22A-B, upstream component 2070 and downstream component 2072 are configured to be removably coupled together so as to form the liquid-impermeable seal, such that upstream component 2070 and downstream component 2072 partially overlap each other at an axial overlap region 2082 (labeled in FIG. 22B) that at least partially defines liquid container 2030. For other applications, upstream component 2070 and downstream component 2072 do not axially overlap (configuration not shown); in these other applications, liquid container 2030 is optionally defined only by downstream component 2072 and not by upstream component 2070. For some applications, as perhaps best shown in the blow-up in FIG. 22A, an outer edge of filter 2032 is squeezed directly or indirectly between upstream component 2070 and downstream component 2072 to hold the filter in place until upstream component 2070 is decoupled from downstream component 2072.

In general, in all of the configurations of testing devices described herein that comprise upstream and downstream components that are removably coupled together, the liquid container may be defined in part by the upstream component and in part by the downstream component. For example, a distal downstream wall of the liquid container that supports the filter may be defined by the downstream component, while the lateral wall of the liquid container may be defined by the upstream component or by the upstream and downstream components in combination.

For some applications, such as shown in FIGS. 22C-D, testing device 2020 is configured such that at least 80% of the surface area of an upstream side of filter 2032 is exposed to outside testing device 2020 when upstream component 2070 and downstream component 2072 are decoupled from each other.

For some applications, an area of upstream opening 2076 is greater than the area of downstream opening 2078. For example, a diameter of upstream opening 2076 may be at least 10% (e.g., 20%, such as 30%) greater than a diameter of downstream opening 2078. For some of these applications, plunger housing 2074 includes an upstream end portion 2084 (labeled in FIG. 22B) that includes upstream opening 2076, and upstream end portion 2084 is conical and/or funnel-shaped.

Reference is still made to FIGS. 22A-H. In an application of the present invention, a method is provided for testing liquid 2022 for the presence of the particulate. For some applications, the particulate comprises biological particulate, for example, a microorganism, a fungus, a bacterium, a spore, a virus, a mite, a biological cell, a biological antigen, a protein, a protein antigen, and a carbohydrate antigen.

For applications in which one or more components of testing device 2020 are removably disposed in sterile packing, the one or more components are removed from the sterile packaging.

As shown in FIG. 22A, the method comprises receiving, in liquid container 2030, liquid 2022 from a patient's mouth. For some applications, liquid 2022 comprises gargled fluid, i.e., a gargle fluid that the patient has gargled in his or her mouth and spit out, perhaps along with some saliva. In the present application, including in the claims, “gargled fluid” means “gargle fluid” that has been gargled by a patient. Typically, the gargle fluid includes water, carbonated water, saline (e.g., phosphate buffered saline), pelargonium sidoides extract, tannic acid, balloon flower platycodon grandiflorus, berberine sulfate, S-carboxymethylcysteine, curcumin, coloring, flavoring, a detergent (such as Polysorbate 20 (e.g., Tween® 20)), or any combination thereof. In some applications, the gargle fluid is carbonated. Alternatively or additionally, for some applications, a detergent, such as Polysorbate 20 (e.g., Tween® 20) is added to the gargled fluid after being gargled by the patient. Alternatively, liquid 2022 may comprise another type of biological fluid, such as blood (e.g., diluted blood), urine, stool (e.g., diluted stool), gastrointestinal (GI) fluid, or bronchoalveolar lavage fluid.

Alternatively, liquid 2022 comprises saliva not swabbed from the throat of a patient (i.e., the saliva was collected without swabbing the patient's throat). (The distinction between “swab” as a verb and as a noun is noted. A “swab” (as a noun) may be used to obtain saliva without “swabbing” (as a verb) the patient's throat. For example, the patient may suck on a swab, or a swab may be dipped in a container into which gargle fluid or saliva has been placed.) By contrast, in commonly-practiced techniques for testing for strep, the tonsils are swabbed. Further alternatively, liquid 2022 comprises liquid from a cultured medium containing a biological sample which had been incubated within the liquid container 2030 or incubated separately from the device and then added to liquid container 2030, for example for performing a backup test (e.g., a backup strep test) using rapid testing techniques, e.g., rapid strep testing techniques. As used in the present application, including in the claims, in the context of backup testing, “rapid” testing techniques (such as “rapid” strep testing techniques) refer to the type of test, rather than implying that the test is performed and provides results soon after the sample is obtained from the patient; indeed, for performing backup testing, the rapid testing techniques are typically performed well after the sample has been obtained from the patients, such as a number of hours thereafter, and typically include incubation of the sample.

liquid 2022 (e.g., saliva) may be spit directly by the patient into liquid container 2030 or transferred by a healthcare worker from another container into which the patient spit. Alternatively, in the case of saliva, the saliva may be collected from the patient's mouth by having the patient suck on a swab or other absorbent collecting element, such as flocked swabs or cotton rolls.

For applications in which testing device 2020 comprises plunger 2040 and plunger housing 2074, such as described above, liquid 2022 is typically received in liquid container 2030 before plunger 2040 has been inserted into plunger housing 2074 (or liquid container 2030).

As shown in FIG. 22B, pressure is applied to drive liquid 2022 contained in liquid container 2030 of testing device 2020 through filter 2032, such as using one or more of the techniques for applying pressure described hereinabove. For applications in which testing device 2020 comprises the one or more valves 2060, the pressure also drives liquid 2022 through the one or more valves 2060 after the liquid is driven through filter 2032. For applications in which liquid container 2030 is shaped so as to define the one or more openings 2051, as described hereinabove, the pressure drives liquid 2022 through the one or more openings 2051. Typically, toward the end of the application of the pressure, some air trapped in liquid container 2030 is blown through filter 2032, helping to expel most of liquid 2022 remaining in filter chamber 2036 and generally dry the filter chamber. For applications in which testing device 2020 comprises waste liquid receptacle 2046, applying the pressure drives liquid 2022 contained in liquid container 2030 through filter 2032, then through the one or more valves 2060, and then into waste liquid receptacle 2046. For some applications in which testing device 2020 comprises filter chamber 2036, applying the pressure also drives some of liquid 2022 into filter chamber 2036. For some applications, testing device 2020 further comprises a release button that pushes on filter chamber 2036 to extract any remaining gargled fluid upon completion of application of the pressure (configuration not shown).

As shown in FIG. 22C, for applications in which testing device 2020 comprises upstream component 2070 and downstream component 2072, upstream component 2070 is decoupled from downstream component 2072, in order to expose and provide access to filter 2032. Instead removing plunger 2040 from liquid container 2030 might cause some of liquid 2022 to spray out of liquid container.

As shown in FIG. 22A-D, for some application in which testing device 2020 comprises filter chamber 2036, the pressure is applied while filter 2032 is removably disposed upstream of filter chamber 2036 with filter 2032 partially covering inlet 2038. For some of these applications, after applying the pressure and before testing for the presence of the particulate trapped by filter 2032, at least a portion of filter 2032 is pushed into filter chamber 2036, such as shown in FIGS. 22D-F. For example, the at least a portion of filter 2032 may be pushed into filter chamber 2036 using an elongate member 2056, such as shown in FIG. 22D-E, using plunger head 2042 (configuration not shown), or using gas pressure and/or suction (configuration not shown). For applications in which filter chamber 2036 comprises one or more pressure-activated valves 2050, such as described hereinabove, the elongate member 2056 (e.g., swab 2058 thereof) drives liquid 2022 in filter chamber 2036 out of filter chamber 2036 through the one or more pressure-activated valves 2050, into waste liquid receptacle 2046 if provided, such as shown in FIG. 22E.

As shown in FIGS. 22E-F, for some applications, a sample is taken from filter 2032 (either from a surface of the filter or of the filter itself, such as a small part of the filter) using elongate member 2056 (e.g., swab 2058 thereof), and the sample is tested, outside testing device 2020, for the presence of the particulate. This testing may, for example, be an overnight backup test, e.g., an overnight backup strep test. The backup test may be performed by placing the sample (optionally while still on swab 2058) into a test tube 2085 containing growth medium 2087 (e.g., Todd Hewitt broth, tryptic soy broth, Columbia Broth, Nutrient Broth, or Thioglycollate broth), capping the test tube, and incubating the test tube, as is known in the art. Optionally, growth medium 2087 has the properties of the high-concentration liquid growth medium described in detail hereinbelow.

Alternatively, for some applications, the entire filter 2032 is removed from testing device 2020 and tested, outside testing device 2020, for the presence of the particulate. If such testing is a rapid strep test, the method may conclude with this test, and not continue with the performance of a test in testing device 2020, as described hereinbelow with reference to FIGS. 22G-H. For example, the rapid strep test may use any of the testing techniques described hereinbelow with reference to FIG. 33 regarding external analysis device 1010, with or without first culturing.

As shown in FIGS. 22G-H, the method further comprises testing, within testing device 2020, for the presence of particulate trapped by filter 2032 while filter 2032 is disposed in testing device 2020. For applications in which testing device 2020 comprises the one or more valves 2060, the testing is performed while the one or more valves 2060 are closed. Alternatively or additionally, for applications in which testing device 2020 comprises filter chamber 2036, the testing is performed within filter chamber 2036 while filter 2032 is disposed at least partially in the filter chamber.

For some applications, the testing is performed by:
applying an extraction reagent 2086 to filter 2032, such as shown in FIG. 22G; for applications in which testing device 2020 comprises filter chamber 2036, the extraction reagent 2086 is typically applied to filter 2032 while filter 2032 is in filter chamber 2036; as mentioned above, for applications in which testing device 2020 comprises the one or more valves 2060, the testing is performed while the one or more valves 2060 are closed so that extraction reagent 2086 is retained by filter 2032 rather than passing through the filter, and
after applying extraction reagent 2086, inserting a test strip 2088 into testing device 2020 (e.g., into filter chamber 2036) and examining the test strip to test for the presence of the particulate, such as shown in FIG. 22H; optionally, filter 2032 is mixed after application of extraction reagent 2086 but before insertion of test strip 2088.

Reference is still made to FIGS. 22A-H. In an application of the present invention, a system 2090 (labeled in FIG. 22A) is provided that comprises:
a liquid 2022 including at least one substance selected from the group of substances consisting of gargled fluid, saliva not swabbed from the throat of a patient, and an incubated culture medium containing a biological sample; and
testing device 2020, which comprises (a) liquid container 2030 containing the liquid 2022, (b) the one or more valves 2060, (c) filter 2032, disposed in or downstream of liquid container 2030 and upstream of the one or more valves 2060, and (d) liquid-pressure source 2034, which is arranged to apply pressure to drive liquid 2022 contained in liquid container 2030 through filter 2032 and then through the one or more valves 2060.

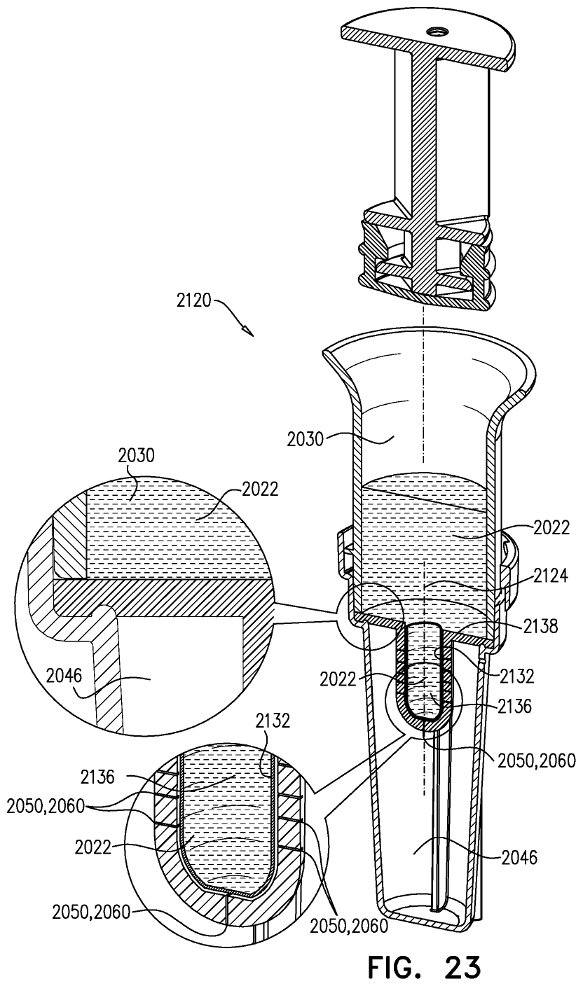

Reference is now made to FIG. 23, which is a schematic illustration of a testing device 2120 for testing for presence of particulate in liquid 2022, in accordance with an application of the present invention. Other than as described below, testing device 2120 is similar to testing device 2020, described hereinabove with reference to FIGS. 22A-H, and may implement any of the features thereof.

Testing device 2120 comprises a filter 2132 that is disposed at least partially, e.g., entirely, within a filter chamber 2136. By contrast, filter 2032 of testing device 2020 is removably disposed upstream of filter chamber 2036 with filter 2032 partially covering inlet 2038. Other than this feature, filter 2132 may have any of the features of filter 2032 described hereinabove with reference to FIGS. 22A-H, including material properties and dimensions.

Filter chamber 2136 may implement any of the features of filter chamber 2036, described hereinabove with reference to FIGS. 22A-H. Typically, filter chamber 2136 comprises one or more pressure-activated valves 2050 (such as a plurality, as shown in FIG. 23), not disposed at an inlet 2138. Inlet 2138 may implement any of the features of inlet 2038 described hereinabove with reference to FIGS. 22A-H. In applications in which a plurality of pressure-activated valves 2050 are provided, the bottom-most valve may serve to allow the flushing of the remaining liquid 2022.

Testing device 2120 may be used as described hereinabove with reference to FIGS. 22A-H. The pressure described hereinabove with reference to FIG. 22B is applied while filter 2132 is disposed at least partially (e.g., entirely) within filter chamber 2136.

The pressure drives liquid 2022 from liquid container 2030 to filter chamber 2136, then through filter 2132, and then through the one or more pressure-activated valves 2050, and optionally into waste liquid receptacle 2046, if provided. Typically, liquid container 2030 is not shaped so as to define the one or more openings 2051 described hereinabove with reference to FIGS. 22A-H.

For some applications, filter 2132 is disposed surrounding at least 270 degrees, typically 360 degrees, of a central longitudinal axis 2124 of filter chamber 2136, such that all or substantially all of liquid 2022 that passes out of filter chamber 2136 must pass through filter 2132. For some applications, filter 2132 covers all of the one or more pressure-activated valves 2050. For some applications, filter 2132 covers at least 80%, such as 100%, of the internal surface of filter chamber 2136. For some applications, such as shown in FIG. 23, filter 2132 is shaped as a receptacle.

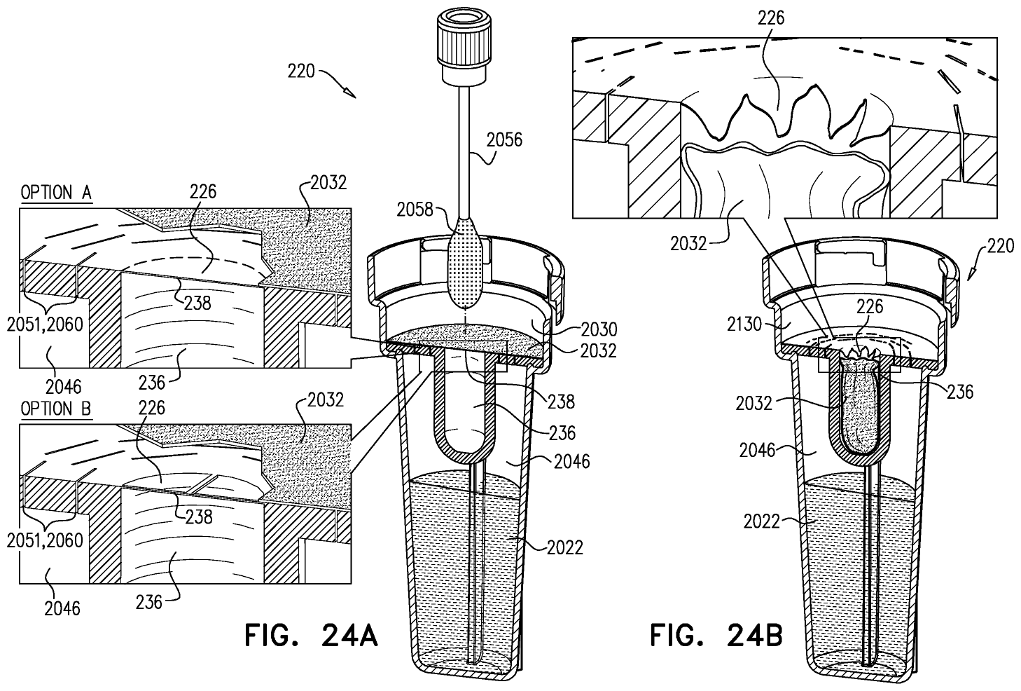

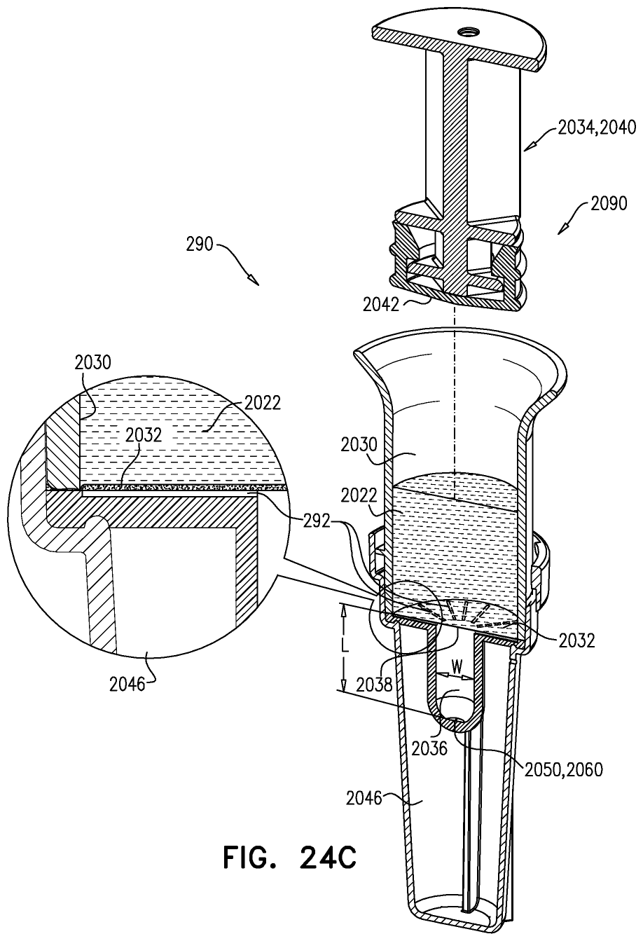

Reference is now made to FIGS. 24A-B, which are schematic illustrations of a testing device 220 for testing for presence of particulate in liquid 2022, in accordance with an application of the present invention. Other than as described below, testing device 220 is similar to testing device 2020, described hereinabove with reference to FIGS. 22A-H, and may implement any of the features thereof.

Testing device 220 further comprises a frangible seal 226 that removably blocks liquid flow into an inlet 238 of a filter chamber 236. For example, frangible seal 226 may comprise a pliable material (such as silicone) that is easily torn, such as shown in Option A in FIG. 24A, or a rigid material that is easily broken (e.g., shaped so as define slits to aid in breaking), such as shown in Option B in FIG. 24A. Typically, filter 2032 of testing device 220, like filter 2032 of testing device 2020, is removably disposed upstream of filter chamber 236 with filter 2032 partially covering inlet 238 of filter chamber 236. When pressure is applied, as described hereinabove with reference to FIG. 22B, substantially all of liquid 2022 is driven out of liquid container 2030, either the one or more openings 2051 or valves 2060 described herein. Thereafter, before testing for the presence of the particulate trapped by filter 2032, frangible seal 226 is broken, such as using elongate member 2056 (e.g., swab 2058 thereof), as shown in FIG. 24A, or using plunger head 2042 (configuration not shown), and at least a portion of (e.g., the entirely of) filter 2032 is pushed into filter chamber 236. Because liquid container 2030 is substantially empty of liquid 2022, only a minimal amount of liquid 2022 enters filter chamber 236. Therefore, filter chamber 236 typically does not comprise any pressure-activated valves 2050 not disposed at an inlet 238, because drainage of liquid is not required.

For some applications, filter chamber 236, before frangible seal 226 is broken, contains a material, such as a rapid test solution (e.g., a rapid strep test) in liquid or solid (e.g., powdered) form. This may simplify the use of the testing device because the material is not flushed during the application of pressure, and thus does not need to be added during use after applying the pressure.

For some applications, testing device 2020 further comprises a support for filter 2032 (e.g., the configuration of frangible seal 226 shown in Option B in FIG. 24A), disposed at least partially between inlet 2038 and filter 2032. During application of pressure, as described hereinbelow with reference to FIG. 22B, the support helps prevent filter 2032 from entering filter chamber 2036, which might occur if inlet 2038 is relative wide. The support is easily breakable or flexible such that filter 2032 can still readily be pushed into filter chamber 2036. For example, the support may comprise a very thin plastic sheet (like a plastic bag) that has holes such that the support provides enough support for the filter to rest on and not puncture, and also very flexible so that it can easily be pushed into filter chamber 2036 together with filter 2032. Alternatively, the support may comprise a harder or firmer material that is easily breakable, e.g., may comprise slits to enable easy breaking. Optionally, plunger head 2042 is configured to break the support (configuration not shown). The support may comprise an elastomer and/or be perforated with openings, e.g., having an average diameter of between 0.2 and 5 mm.

Reference is now made to FIG. 24C, which is a schematic illustration of a testing device 290 for testing for presence of particulate in liquid 2022, in accordance with an application of the present invention. Other than as described below, testing device 290 is similar to testing device 2020, described hereinabove with reference to FIGS. 22A-H, and may implement any of the features thereof. Liquid container 2030 is not shaped so as to define the one or more openings 2051 or the one or more valves 2060 described hereinabove with reference to FIGS. 22A-H. Instead, in order to allow liquid 2022 to pass through the peripheral portion of filter 2032 not disposed over inlet 2038, the downstream surface of liquid container 2030 is shaped so as define a plurality of elongate indentations 292 that extend radially inward to the edge of inlet 2038. The liquid 2022 that is driven through the peripheral portion of filter 2032 enters the indentations and drains from the indentations into filter chamber 2036. This configuration allows for the entire filter upstream surface area to be utilized while allowing the air trapped in liquid container 2030 to blow out most of remaining liquid 2022 from filter chamber 2036 to leave the filter chamber generally dry.

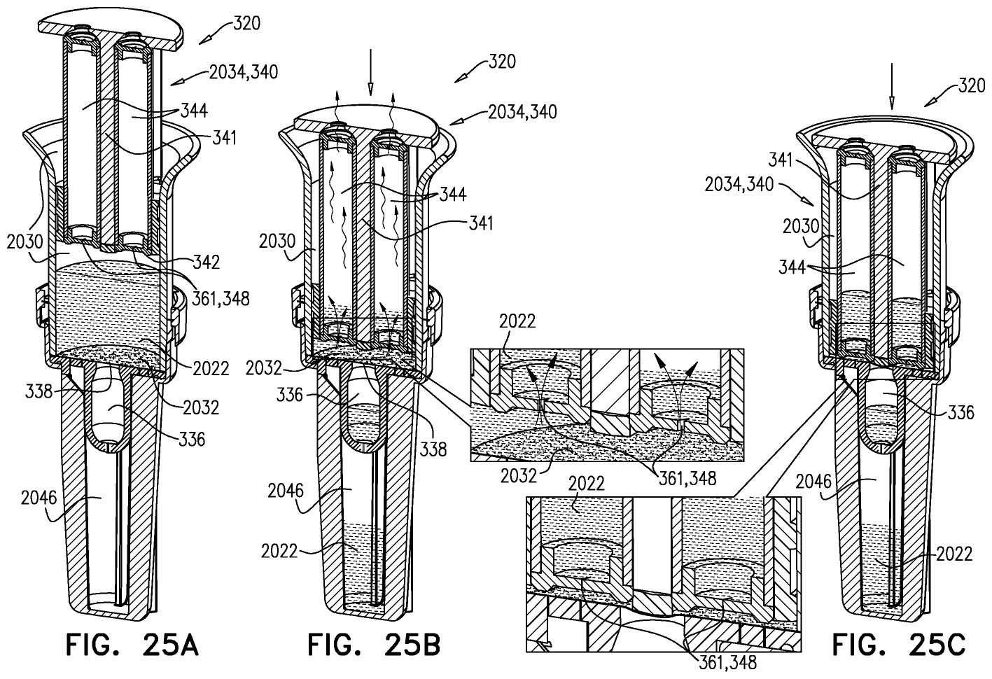

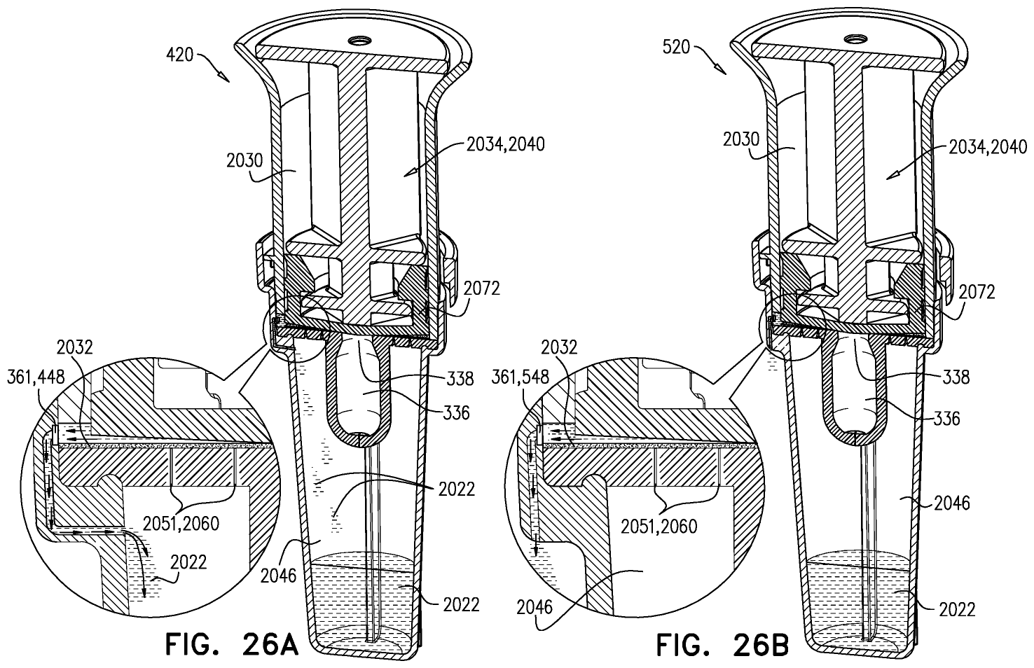

Reference is now made to FIGS. 25A-C, which are schematic illustrations of a testing device 320 for testing for presence of particulate in liquid 2022, in accordance with an application of the present invention. Reference is also made to FIGS. 26A-B, which are schematic illustrations of a testing device 420 and a testing device 520, respectively, for testing for presence of particulate in liquid 2022, in accordance with respective applications of the present invention. Other than as described below, testing devices 320, 420, and 520 are similar to testing device 2020, described hereinabove with reference to FIGS. 22A-H, and may implement any of the features thereof.

For applications in which the one or more valves 2060 are provided, such as shown in FIGS. 25A-C and 26A-B, the one or more valves 2060 are one or more first valves 2060, and testing devices 320, 420, and 520 further comprise one or more second pressure relief valves 361, which are in fluid communication with liquid container 2030 and are disposed upstream of filter 2032. For some applications, the one or more first valves 2060 comprise one or more first pressure-activated valves 2060 configured to open upon exposure to a first pressure gradient across the one or more first pressure-activated valves 2060, and the one or more second pressure relief valves 361 are configured to open upon exposure to a second pressure gradient across the one or more second pressure relief valves 361, the second pressure gradient greater than the first pressure gradient. Alternatively, the one or more valves 2060 are not provided; for example, the wall of liquid container 2030 may be shaped so as to define the one or more openings 2051 described hereinabove with reference to FIGS. 22A-H.

The one or more second pressure relief valves 361 allow drainage of liquid 2022 if excess pressure occurs in liquid container 2030, such as if filter 2032 becomes clogged during the application of pressure described hereinabove with reference to FIG. 22B.

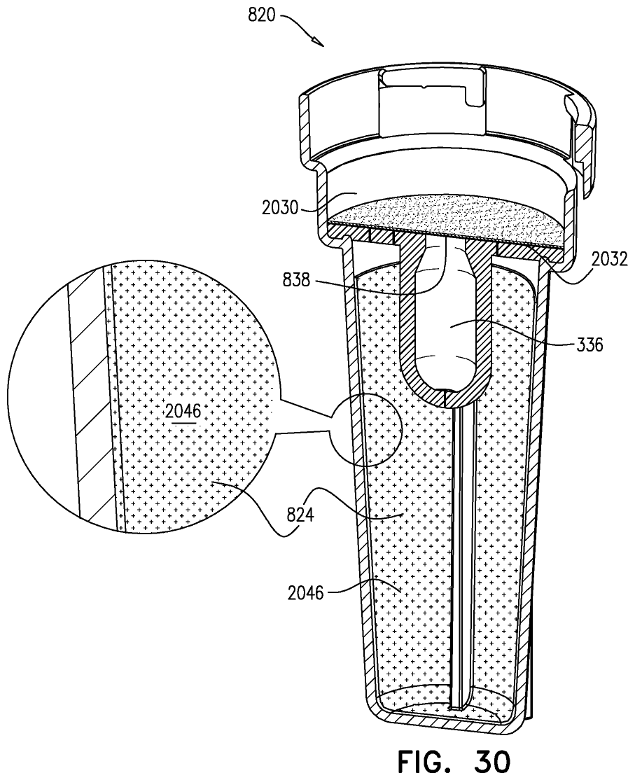

Reference is made to FIGS. 25A-C. For some applications, liquid-pressure source 2034 comprises a plunger 340, which comprises (a) a plunger shaft 341 and (b) a plunger head 342 disposed at a downstream end portion of plunger shaft 341 and shaped so as to be insertable into liquid container 2030. Plunger 340 (including plunger head 342 and plunger shaft 341) may implement any of the configures of plunger 2040 described hereinabove with reference to FIGS. 22A-H. Testing device 320 comprises one or more unfiltered liquid receptacles 344 (e.g., vials). The one or more second pressure relief valves 361 comprise one or more second pressure relief valves 348 that are in fluid communication with the one or more unfiltered liquid receptacles 344. For some applications, the one or more unfiltered liquid receptacles 344 are disposed along plunger shaft 341, such as shown. Optionally, the one or more unfiltered liquid receptacles 344 are removably coupled to plunger 340. Typically, the one or more unfiltered liquid receptacles 344 are shaped so as to define vents to allow the escape of air as liquid 2022 enters. Optionally, the one or more unfiltered liquid receptacles 344 contain an antibacterial agent, such as described hereinbelow with reference to FIG. 30 regarding waste liquid receptacle 2046.

Reference is made to FIG. 26A. For some applications, testing device 420 further comprises waste liquid receptacle 2046, which is coupled to liquid container 2030 downstream of filter 2032 (and of the one or more valves 2060, if provided). Liquid-pressure source 2034 is arranged to apply pressure to drive liquid 2022 contained in liquid container 2030 through filter 2032, then through the one or more openings 2051 or the one or more valves 2060, if provided, and then into waste liquid receptacle 2046. The one or more second pressure relief valves 361 comprise one or more second pressure relief valves 448 that are in fluid communication with waste liquid receptacle 2046 not via filter 2032.

Reference is made to FIG. 26B. For some applications, the one or more second pressure relief valves 361 comprise one or more second pressure relief valves 548 that are in fluid communication with outside testing device 520.

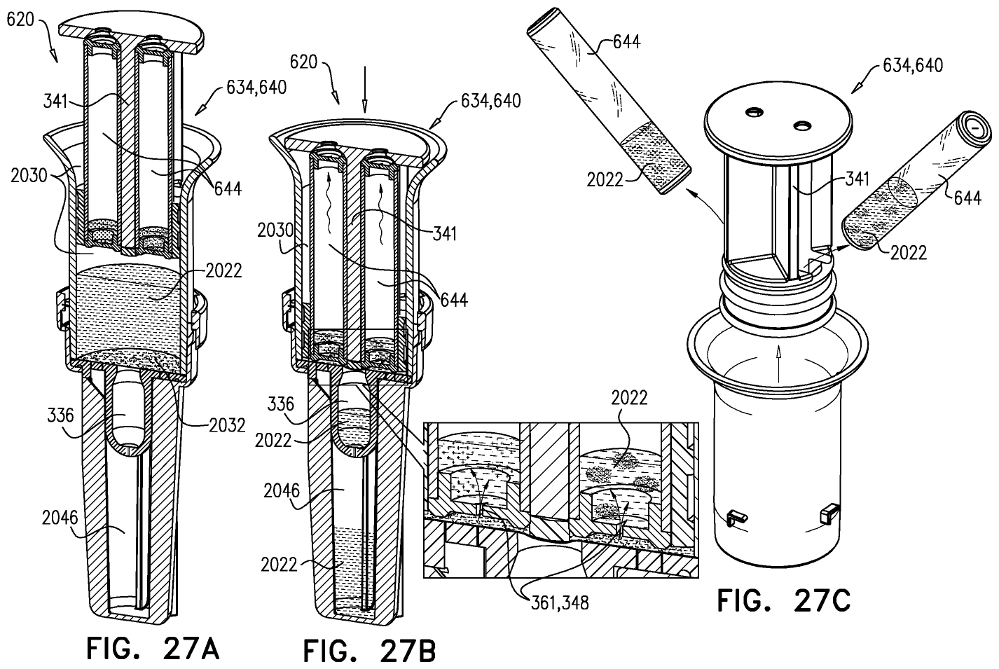

Reference is made to FIGS. 27A-C, which are schematic illustrations of a testing device 620 for testing for presence of particulate in liquid 2022, in accordance with an application of the present invention. Other than as described below, testing device 620 is similar to testing device 320, described hereinabove with reference to FIGS. 25A-C and may implement any of the features thereof. In addition, other than as described below, testing device 620 is similar to testing device 2020, described hereinabove with reference to FIGS. 22A-H, and may implement any of the features thereof.

Testing device 620 comprises one or more unfiltered liquid receptacles 644 (e.g., vials). The one or more second pressure relief valves 361, 348 are in fluid communication with the one or more unfiltered liquid receptacles 644, such that when pressure is applied, as described hereinabove with reference to FIG. 22B, a portion of the unfiltered liquid 2022 is driven into the one or more unfiltered liquid receptacles 644. After applying the pressure, a sample of liquid 2022 in the one or more unfiltered liquid receptacles 644 is taken, and the sample is tested, outside testing device 2020, for the presence of the particulate, using any overnight or rapid test (e.g., rapid strep test) including or not including incubation. This may simplify the process of taking a sample for backup test, e.g., a backup strep test (and may even get a better sample of bacteria). Optionally, the one or more unfiltered liquid receptacles 644 contain culture media, e.g., including red blood cells.

Reference is now made to FIGS. 28A-C and 29A-E, which are schematic illustrations of a testing device 720 for testing for presence of particulate in liquid 2022, in accordance with an application of the present invention. Other than as described below, testing device 720 is similar to the testing devices described hereinabove with reference to FIGS. 22A-6C, and may implement any of the features thereof; testing device is particularly similar to testing device 220, described hereinabove with reference to FIGS. 24A-B. Unless otherwise described, reference numerals valves 2060 in FIGS. 28A-C and 29A-E refer to like parts as reference numerals in FIGS. 22A-H based on the last two digits.

Testing device 720 comprises a liquid-pressure source 734, which comprises a plunger 740, which comprises a plunger head 742 that is shaped so as to be insertable into a liquid container 730. Plunger 740 is shaped so as to define a waste liquid receptacle 746.

Plunger 740 is also shaped so as to define a filter chamber 736. Filter chamber 736 typically does not comprise any pressure-activated valves 2050. Testing device 720 further comprises a frangible seal 726 that removably blocks liquid flow into an inlet 738 of filter chamber 736. Frangible seal 726 may implement any of the features of frangible seal 226 described hereinabove with reference to FIGS. 24A-B.

For some applications, testing device 720 comprises a cap 792, which is removably coupled to a distal end of liquid container 730 (i.e., to the end opposite the end into which plunger 740 is inserted). A proximal wall 794 of cap 792 defines a distal wall of liquid container 730. For some applications, cap 792 is shaped so as to define an unfiltered liquid receptacle 744, and proximal wall 794 of cap 792 comprises one or more second pressure relief valves 761 that (a) are in fluid communication with unfiltered liquid receptacle 744 and, when cap 792 is coupled to liquid container 730, with liquid container 730, and (b) are disposed upstream of filter 732.

Before use (e.g., during manufacture), cap 792 is removably coupled to liquid container 730, such as by twisting the cap onto liquid container 730, as shown in FIG. 29A. Also before use, plunger 740 is not coupled to liquid container 730. Optionally, plunger 740 has a distal protective cover 796, which is removed before use.

liquid 2022 (such as gargled fluid, saliva not swabbed from the throat of a patient, or an incubated culture medium containing a biological sample) is received in liquid container 730.

As shown in FIG. 29B, plunger 740 is inserted into liquid container 730 after removing cap 792.

As shown in FIG. 29C, plunger 740 is pushed until the distal end of the plunger reaches proximal wall 794 of cap 792. This pushing applies pressure to liquid 2022, such as described hereinabove with reference to FIG. 22B.

As shown in FIG. 29D, cap 792 is removed from liquid container 730, such as by twisting the cap, and, optionally, further pushing the plunger until the plunger pushes off the cap, in order to expose a filter 732. The cap is disposed.

As shown in FIG. 29E, at least a portion (e.g., the entirely) of filter 732 is pushed into filter chamber 736, such as using elongate member 2056, which also breaks frangible seal 726. (The filter may tear, as shown, leaving a portion of the filter outside filter chamber 736, e.g., connected at a periphery of the filter to liquid container 730, as shown.)

The use of testing device 720 may continue as described hereinabove with reference to FIGS. 22E-H, mutatis mutandis.

Reference is now made to FIG. 30, which is a schematic illustration of a testing device 820 for testing for presence of particulate in liquid 2022, in accordance with an application of the present invention. Other than as described below, testing device 820 is similar to the testing devices described hereinabove with reference to FIGS. 22A-8E, and may implement any of the features thereof. Similarly, any of testing devices described herein may implement the features of FIG. 30, mutatis mulandis.

Testing device 820 comprises waste liquid receptacle 2046, which contains an antibacterial agent 824, such as a detergent, thiomersal, bleach, or iodine (1/Kl) to kill any bacteria that passes through filter 2032, to reduce the risk of contamination upon accidental exposure to the liquid in waste liquid receptacle 2046.

For some applications, an inlet 838 of a filter chamber 336 of testing device 820 has an inlet area that is less than a greatest cross-sectional area of filter chamber 336, the inlet area and the greatest cross-sectional area measured in respective planes parallel to each other. For example, the inlet area may be no more than 95%, such as no more than 90%, e.g., no more than 80% of the greatest cross-sectional area of filter chamber 336. Providing this narrowing of filter chamber 336 at inlet 838 may help retain filter 2032 in filter chamber 336 during withdrawal of elongate member 2056, as described hereinabove with reference to FIG. 22F. Testing devices 320, 420, 520, 620, 1020, and 1120, described herein with reference to FIGS. 25A-C, 26A, 26B, 27A-C, 35B, and 3613, respectively, are also shown comprising filter chamber 336; these testing devices may alternatively comprise filter chambers 2036, 1136, 236, or 736, mutatis mutandis.

Reference is now made to FIGS. 31A-C, which are schematic illustrations of a testing device 920 for testing for presence of particulate in liquid 2022, in accordance with an application of the present invention. (Filter 2032 is shown in partial cut-away view to show the one or more non-pressure-activated valves 960 described below.) Other than as described below, testing device 920 is similar to the testing devices described hereinabove with reference to FIGS. 22A-9, and may implement any of the features thereof.

The one or more valves 2060 of testing device 920 comprise one or more non-pressure-activated valves 960. For example, the one or more non-pressure-activated valves 960 may be opened and closed by aligning and non-aligning, respectively, sets of openings in two discs 962A and 962B of the one or more non-pressure-activated valves 960, either manually or automatically by the testing device, such as described hereinbelow. Other manual and automated configurations will be readily apparent to those skilled in the art who have read the present application.

During use, liquid 2022 is received in a liquid container 930, as shown in FIG. 31A, typically while the one or more non-pressure-activated valves 960 are in an opened state (e.g., with the openings in disc 962A aligned with the openings in disc 962B), thereby allowing liquid 2022 to pass through the one or more valves and the filter, optionally into a waste liquid receptacle 946 if provided, as shown in FIGS. 31A-B. Typically, while the one or more non-pressure-activated valves 960 are open, pressure is applied using a liquid-pressure source such as those described herein.

Thereafter, as partially shown in FIG. 31C, the one or more non-pressure-activated valves 960 are closed (e.g., by rotating at least one of discs 962A and 962B so that their respective openings are not aligned with one another) and filter 2032 is tested for the presence of particulate trapped by filter 2032, such as described hereinabove with reference to FIGS. 22G-H, mutatis mutandis. The closed one or more valves retain extraction reagent 2086 in filter 2032 by preventing the extraction agent from passing through the filter.

As described hereinabove, for some applications, the testing devices described herein comprise a liquid-pressure source that is arranged to apply pressure to drive liquid contained in the liquid container through the filter and, optionally, then into the waste liquid receptacle. For some of these applications, the testing device is configured to automatically (typically, non-electrically) close one or more non-pressure-activated valves of the testing device after the plunger applies the pressure to drive the liquid contained in the liquid container through the filter and then through the one or more non-pressure-activated valves. For some of these applications, the testing device is configured such that motion of the plunger automatically (typically, non-electrically) closes the one or more non-pressure-activated valves after the plunger applies the pressure to drive the liquid contained in the liquid container through the filter and then through the one or more non-pressure-activated valves. Although the testing device is described in this and the following configurations as non-electrically closing the one or more non-pressure-activated valves, the testing device may alternatively electrically close the one or more non-pressure-activated valves, such as using a motor.

Reference is now made to FIGS. 31D-K, which are schematic illustrations of a testing device 1420 for testing for presence of particulate in liquid 2022, in accordance with an application of the present invention. Other than as described below, testing device 1420 is similar to the testing devices described hereinabove with reference to FIGS. 22A-9 and 31A-C, and may implement any of the features thereof, mutatis mutandis. Testing device 1420 comprises a liquid-pressure source 1434, which comprises a plunger 1440, which comprises a plunger head 1442 that is shaped so as to be insertable into a liquid container 1430. Testing device 1420 typically further comprises a waste liquid receptacle 1446, which is coupled to liquid container 1430 downstream of filter 2032. Plunger 1440 is arranged to apply pressure to drive liquid 2022 contained in liquid container 1430 through filter 2032 and then through one or more non-pressure-activated valves 1460 of testing device 1420, and into waste liquid receptacle 1446, if provided, as shown in FIGS. 31I-J.

As shown in FIG. 31K, testing device 1420 is configured such that rotational motion of plunger 1440 automatically (typically, non-electrically) closes the one or more non-pressure-activated valves 1460 of testing device 1420 after plunger 1440 applies the pressure to drive liquid 2022 contained in liquid container 1430 through filter 2032 and then through the one or more non-pressure-activated valves 1460. For example, the last turn of plunger 1440, or a fraction of the last turn (which may or may not include the last portion of the last turn), may automatically close the one or more non-pressure-activated valves 1460.

For some applications, plunger 1440 is shaped so as to define one or more plunger threads 1466, and an internal wall of liquid container 1430 is shaped so as to define one or more liquid-container threads 1468 that engage the one or more plunger threads 1466 such that rotation of plunger 1440 advances plunger 1440 in a downstream direction within liquid container 1430. Advancing plunger 1440 helps control the speed of the advancement and helps maintain steady advancement against pressure in liquid container 1430.

For some applications, the one or more non-pressure-activated valves 1460 comprise two discs 1462A and 1462B, which are shaped so as to define respective sets of openings 1463A and 1463B, for example as described hereinabove with reference to FIGS. 31A-C. For these applications, testing device 1420 is configured such that rotational motion of plunger 1440 automatically closes the one or more non-pressure-activated valves 1460 by rotating at least one of the two discs 1462A and 1462B with respect to the other of the discs, after plunger 1440 applies the pressure to drive liquid 2022 contained in liquid container 1430 through filter 2032 and then through the one or more non-pressure-activated valves 1460. For example, the last turn of plunger 1440, or a fraction of the last turn (which may or may not include the last portion of the last turn), may automatically rotate the at least one of the discs. For example, ridges 1465A on plunger head 1442 may engage, via filter 2032, corresponding ridges 1465B on an upstream surface of disc 1462A after plunger 1440 has been advanced in a downstream direction into contact with disc 1462A. Alternatively or additionally, for some applications, testing device 1420 comprises one or more tabs 1467 that rotate the upper disc and/or break the capsules described hereinbelow.

For some applications, testing device 1420 comprises one or more reagent containers 1471, such as capsules, that contain one or more extraction reagents 2086 (either the same type of extraction reagents or differing extraction reagents). Reagent containers 1471 are disposed at least partially in liquid container 1430, such that upon opening of the containers, such as by crushing, tearing, or breaking, extraction reagents 2086 are released into liquid container 1430, typically near filter 2032. For example, testing device 1420 may configured such that rotational motion of plunger 1440 automatically opens reagents containers 1471, such as by bringing one or more respective protrusions 1473 into contact with the reagent containers. For example, a fraction of the last turn (or the last turn), may automatically open reagents containers 1471. Typically, a fraction of last turn (may or may not include the last portion of the last turn) opens reagents containers 1471, and the fraction occurs after the fraction of the last turn that closes the one or more non-pressure-activated valves 1460, such that the one or more valves are closed before the reagents are released.

Reference is made to FIGS. 31D-G, which illustrate a portion of a method for using testing device 1420 for testing liquid 2022 for the presence of the particulate. This method is optional, and testing device 1420 is not necessarily used in this manner and thus does not necessarily comprise the elements necessary for use in this manner. These techniques may be also be practiced in combination with any of the testing devices described herein for which they are applicable, mutatis mulandis.

In this portion of the method, the user typically receives testing device 1420 with the elements thereof removably coupled together, as shown in FIG. 31D. The user removes plunger 1440 of liquid-pressure source 1434 and a container 1490 from the body of testing device 1420, as shown in FIG. 31E; for example, plunger 1440 may be removed from liquid container 1430 and container 1490 may be removed from a cavity defined by plunger 1440. Liquid 2022 is received from the patient into container 1490 (step not shown). Liquid 2022 is poured from container 1490 into liquid container 1430, as shown in FIG. 31E. Plunger 1440 is reinserted into liquid container 1430, as shown in FIG. 31G.

Reference is now made to FIGS. 31L-M, which are schematic illustrations of a testing device 1520 for testing for presence of particulate in liquid 2022, in accordance with an application of the present invention. Other than as described below, testing device 1520 is similar to testing device 1420 described hereinabove with reference to FIGS. 31D-K. and may implement any of the features thereof, mutatis mutandis. Testing device 1520 comprises a spring 1521, which is biased to hold slightly separated discs 1562A and 1562B of one or more non-pressure-activated valves 1560 of testing device 1520, thereby creating a fluid flow path through the openings of the discs, as shown in FIG. 31L The downstream advancing of the plunger pushes the upper disc 1562A downstream and thus the discs together (and compresses the spring), as shown in FIG. 31M, thereby blocking fluid flow through the openings. The discs typically do not rotate with respect to one another in this configuration.

Reference is now made to FIGS. 31N-O, which are schematic illustrations of a testing device 1620 for testing for presence of particulate in liquid 2022, in accordance with an application of the present invention. Other than as described below, testing device 1620 is similar to testing devices 1420 and 1520 described hereinabove with reference to FIGS. 31D-K and FIGS. 31L-M, respectively, and may implement any of the features thereof, mutatis mutandis. Testing device 1620 comprises one or more flaps 1621, which, in an initial configuration, do not block openings 1663A and 1663B defined by discs 1662A and 1662B, respectively, of one or more non-pressure-activated valves 1660 of testing device 1620, as shown in FIG. 31N. Typically, flaps 1621 are somewhat springy and biased to hold slightly separated discs 1562A and 1562B of one or more non-pressure-activated valves 1560 of testing device 1520. As shown in FIG. 31O, when plunger 1470 is advanced in a downstream direction into contact with disc 1662A, which in turn pushes upstream disc 1662A closer to downstream disc 1662B, thereby causing the one or more flaps 1621 to block openings 1663A and 1663B (such as by displacing or deforming the flaps). The discs typically do not rotate with respect to one another in this configuration.

Reference is now made to FIGS. 31P-Q, which are schematic illustrations of a testing device 1720 for testing for presence of particulate in liquid 2022, in accordance with an application of the present invention. Other than as described below, testing device 1720 is similar to testing devices 1420, 1520, and 1620 described hereinabove with reference to FIGS. 31D-K, FIGS. 31L-M, and FIGS. 31N-O, respectively, and may implement any of the features thereof, mutatis mulandis. Testing device 1720 comprises one or more compressible spacers 1733, which hold slightly separated discs 1762A and 1762B of one or more non-pressure-activated valves 1760 of testing device 1720, thereby creating a fluid flow path through the openings of the discs, as shown in FIG. 31P, by holding the discs at a sufficient distance from each other such that one or more plugs 1729 (e.g., spherical plugs) do not plug openings 1763A of upper disc 1762A. The downstream advancing of the plunger pushes upper disc 1762A downstream and thus the discs together (and compresses the compressible spacers 1733), as shown in FIG. 31Q, thereby causing the one or more plugs 1729 to block openings 1763A. One or more springs 1731 may be provided to push the one ore more plugs 1729 against openings 1763A. Alternatively, a spring similar to spring 1521 of testing device 1520 may be provided instead of or in addition to compressible spacers 1733. The discs typically do not rotate with respect to one another in this configuration.

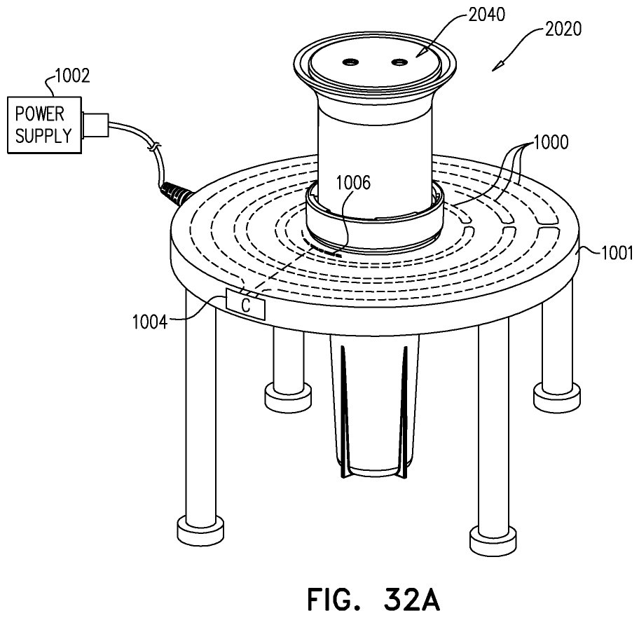

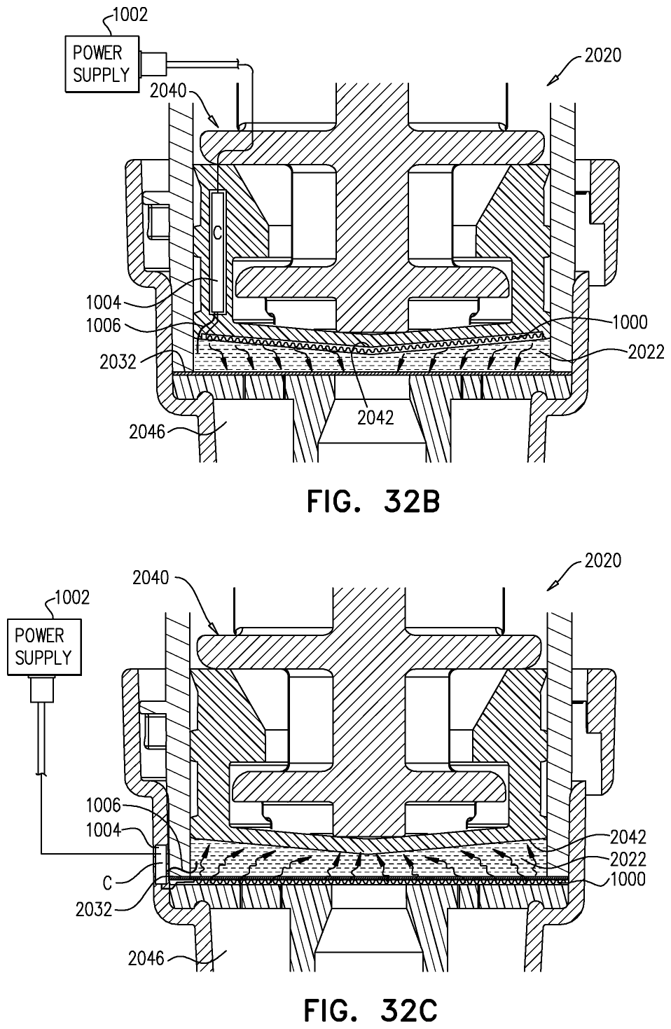

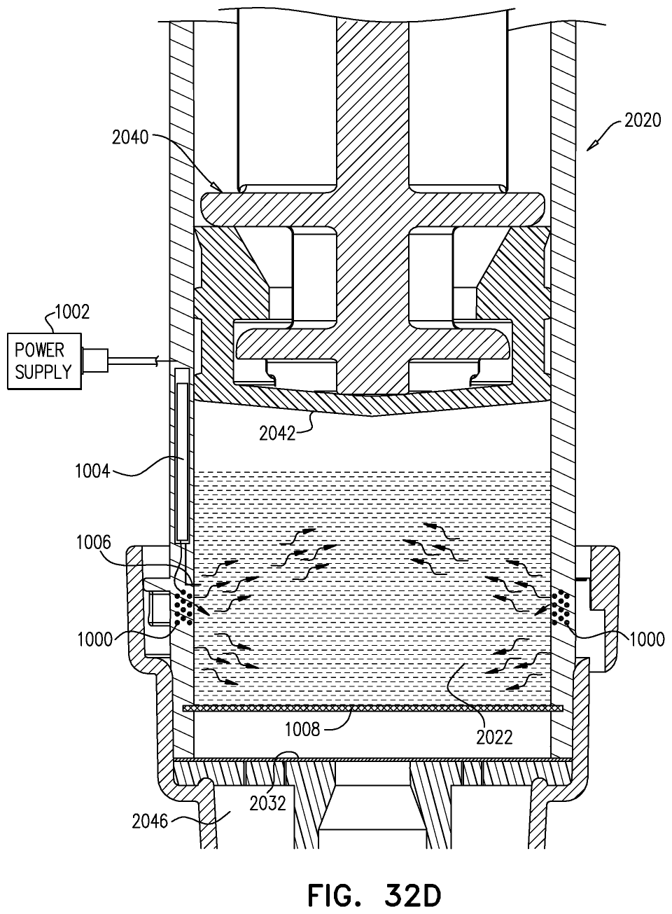

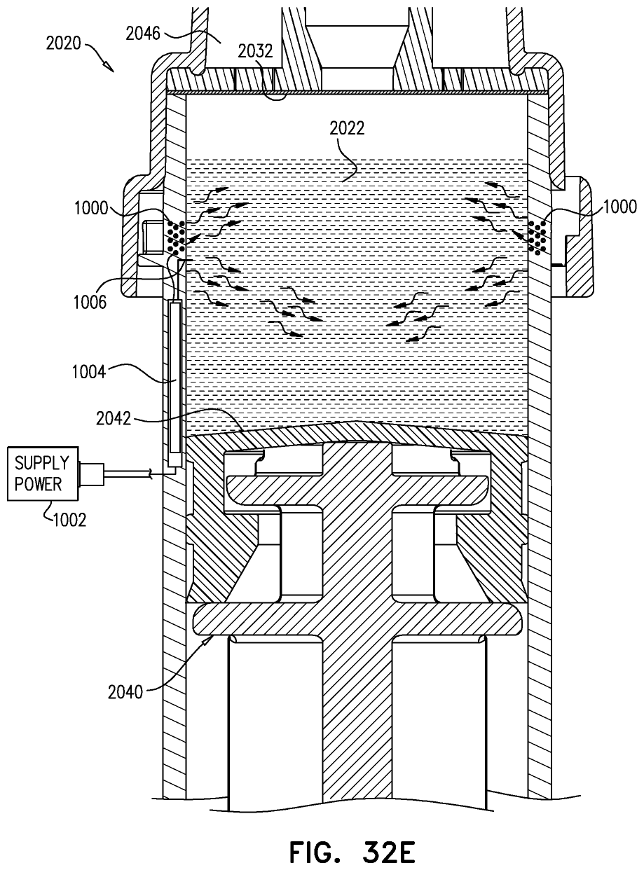

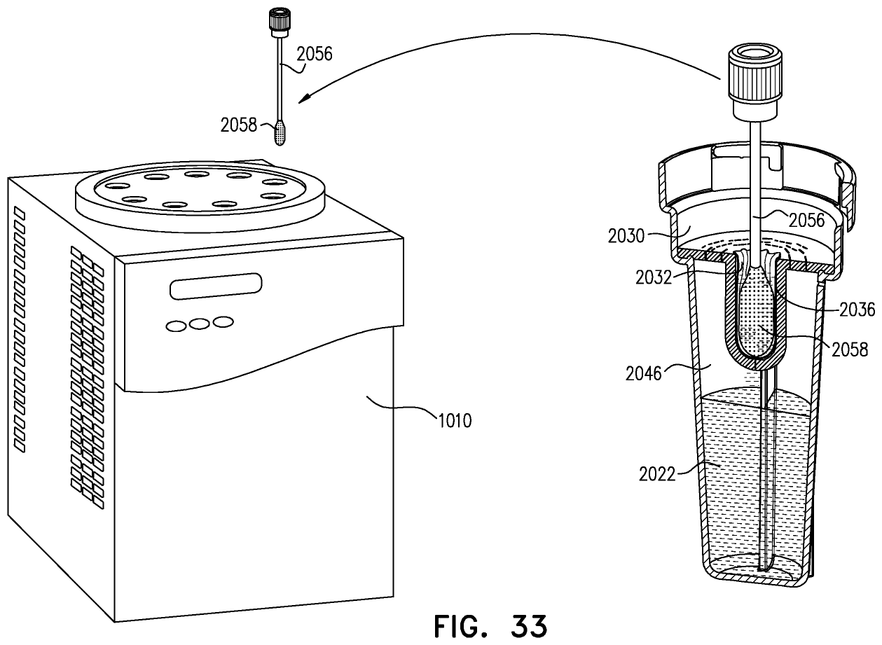

Reference is now made to FIGS. 32A-E, which are schematic illustrations of testing device 2020 further comprising one or more heating elements 1000, in accordance with respective applications of the present invention. Although these configurations are illustrated with respect to testing device 2020, they may also be combined with the other testing devices described herein, mutatis mutandis. These configurations enable incubation of liquid 2022 within testing device 2020.

In these configurations, testing device 2020 further comprises one or more heating elements 1000 that are configured to heat filter 2032 and/or liquid 2022 in liquid container 2030 at a generally constant temperature, typically in the range of 20 and 50 degrees C., such as in the range of 30 to 40 degrees C. It is noted that the temperature is considered “generally constant” even if the temperature varies somewhat, such as because of cycling on and off of the one or more heating elements 1000.

Heating elements 1000 may comprise, for example, electrical heating elements or chemical heating elements (e.g., a heating bag). For applications in which heating elements 1000 are electrical, they are coupled in electrical communication with a power supply 1002, such as an external power supply (e.g., the power grid) or an external or internal battery. For example, the coupling may be done using a conventional electrical plug or USB interface. For some applications, testing device comprises control circuitry 1004 and a heat sensor 1006 (e.g., a thermocouple or other thermostat), and control circuitry 1004 is configured to drive heating elements 1000 responsively to a temperature sensed using heat sensor 1006 in order to maintain the generally constant temperature mentioned above.

For some applications, heating elements 1000 are disposed external to main body of testing device 2020, such as supported by a stand 1001, such as shown in FIG. 32A.

For other applications, such as shown in FIG. 32B, in which liquid-pressure source 2034 comprises plunger 2040 that comprises plunger head 2042 that is shaped so as to be insertable into liquid container 2030, such as described hereinabove, the one or more heating elements 1000 are disposed in the plunger 2040, such as in plunger head 2042, e.g., separated from the distal end of the plunger head by a layer of material such that liquid 2022 does not interfere with the electrical current.

For still other applications, such as shown in FIG. 32C, the one or more heating elements 1000 are disposed downstream of filter 2032 (as shown) or upstream of filter 2032 (configuration not shown).

For other applications, such as shown in FIGS. 32D-E, the one or more heating elements 1000 are disposed around liquid container 2030.

For some applications, the one or more heating elements 1000 are configured to heat filter 2032 and/or liquid 2022 in liquid container 2030 after most or nearly all (e.g., at least 90%) of liquid 2022 has been driven out of liquid container 2030 and the particulate has been trapped by filter 2032, such as shown in FIGS. 32B and 32C (the configuration shown in FIG. 32D can alternatively be used with the plunger pushed farther down than illustrated, and the configuration shown in FIG. 32A can also be used). Typically, growth medium 2087 (e.g., Todd Hewitt broth or tryptic soy broth) is placed in testing device 2020 (e.g., in liquid container 2030, filter 2032, or the distal downstream surface of plunger head 2042) before the heating is performed, in order to incubate the particulate in liquid 2022 and/or filter 2032. For example, such heating may allow a rapid test (e.g., a rapid strep test) to be performed after incubation of the particulate trapped by the filter 2032, for example for between 1 and 24 hours, in order to achieve more accurate results. Optionally, growth medium 2087 has the properties of the high-concentration liquid growth medium described in detail hereinbelow.

For other applications, the one or more heating elements 1000 are configured to heat filter 2032 and/or liquid 2022 in liquid container 2030 while most (e.g., at least 90%) or all of liquid 2022 remains in liquid container 2030 before being driven out of liquid container 2030 and through filter 2032, e.g., by pushing with plunger head 2042, such as shown in FIGS. 32D and 32E (the configurations shown in FIGS. 32B and 32C can also be used with plunger pushed down less than illustrated, and the configuration shown in FIG. 32A can also be used). Typically, growth medium 2087 (e.g., Todd Hewitt broth or tryptic soy broth) is placed in testing device 2020 (e.g., in liquid container 2030, filter 2032, or the distal downstream surface of plunger head 2042) before the heating is performed, in order to incubate the particulate in liquid 2022. Thereafter, after waiting, for example for between 1 and 24 hours, liquid 2022 is driven through filter 2032, and the filter is tested for particulate, for example using a rapid test (e.g., a rapid strep test) performed within or outside of testing device 2020. Such incubation may achieve more accurate results. Optionally, growth medium 2087 has the properties of the high-concentration liquid growth medium described in detail hereinbelow.

Depending on the characteristics of the particular type of filter 2032 used, the filter may be damaged (e.g., degraded) by immersion in heated liquid 2022 for 1 to 24 hours. Therefore, in order to prevent such possible damage, testing device 2020 may be oriented with filter 2032 above liquid 2022 and liquid-pressure source 2034 (e.g., plunger 2040) below filter 2032, such that liquid 2022 is not in contact with filter 2032 during the heating, such as shown in FIG. 32E. Other configurations may also be used, such the configuration shown in FIG. 32A or FIG. 32B, mutatis mulandis.

Alternatively, in order to prevent the above-mentioned possible damage, for some applications, such as shown in FIG. 32D, liquid container 2030 comprises a frangible dividing waterproof or water-resistant membrane 1008 upstream of filter 2032 (e.g., spaced at least 1 mm, such as at least 3 mm, at least 5 mm, or at least 10 mm, from the filter), which isolates filter 2032 from liquid 2022 in liquid container 2030. For some applications, membrane 1008 is elastic, which among other things, may allow insertion of plunger head 2042 into liquid container 2030. After completion of incubation, pushing plunger 2040 causes the plunger to break (e.g., tear) membrane 1008 and allow liquid 2022 to come in contact with filter 2032 for passage therethrough. Other configurations may also be used, such the configuration shown in FIG. 32A, mutatis mulandis.

Reference is now made to FIG. 33, which is a schematic illustration of a method for performing a test, e.g., a backup strep test, in accordance with an application of the present invention. A sample is taken from filter 2032 (either from a surface of the filter or of the filter itself, such as a small part of the filter), e.g., using elongate member 2056 (e.g., swab 2058 thereof), such as described hereinabove with reference to FIGS. 22E-F. The sample is analyzed using an external analysis device 1010, such as a nucleic acid amplification RST technique, such as isothermal amplification, e.g., using Alere™ i (Abbott Laboratories, Waltham, Mass., USA), or real-time quantitative polymerase chain reaction (qPCR) assaying, typically without first incubating the sample. Alternatively, the sample is incubated (either before placing the sample in external analysis device 1010 or inside device 1010 by device 1010) and external analysis device 1010 tests the sample using a technique such as lateral flow immunoassaying, an ELISA-based RST, an antibody-coated-beads-based RST, a nucleic-acid-based RST, or a fluorescent immunoassaying (FIA).

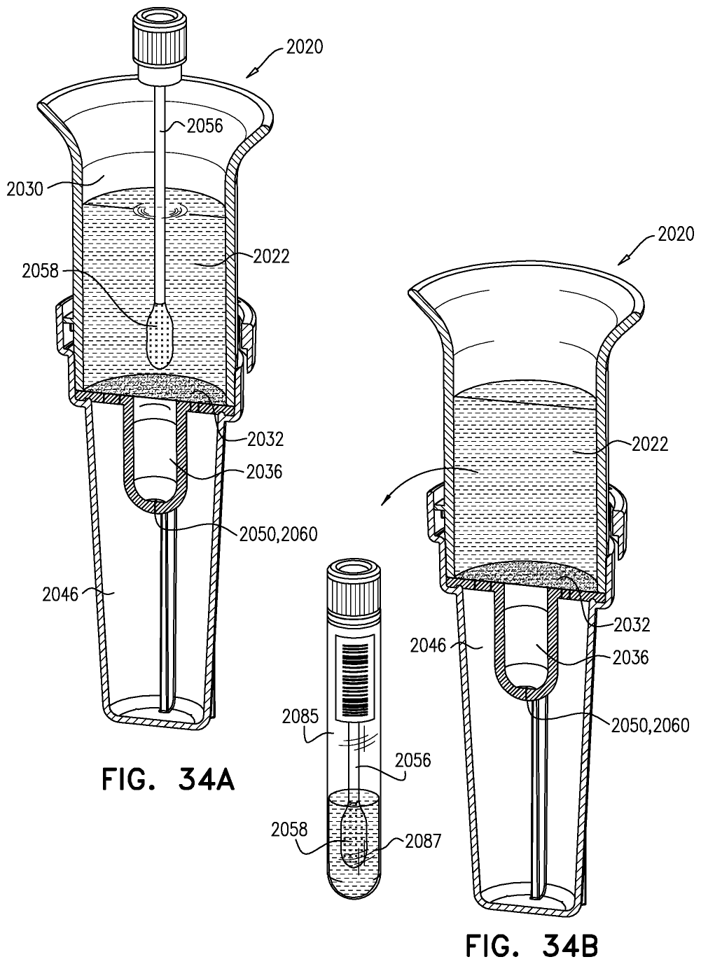

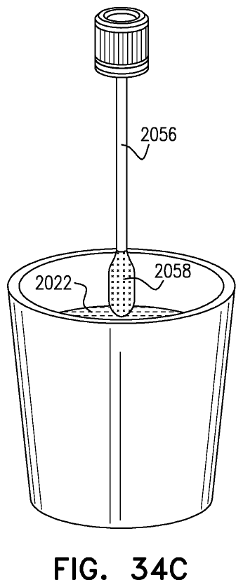

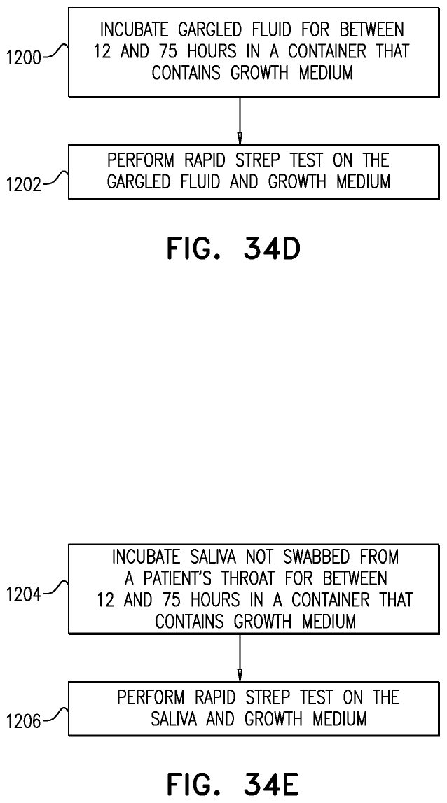

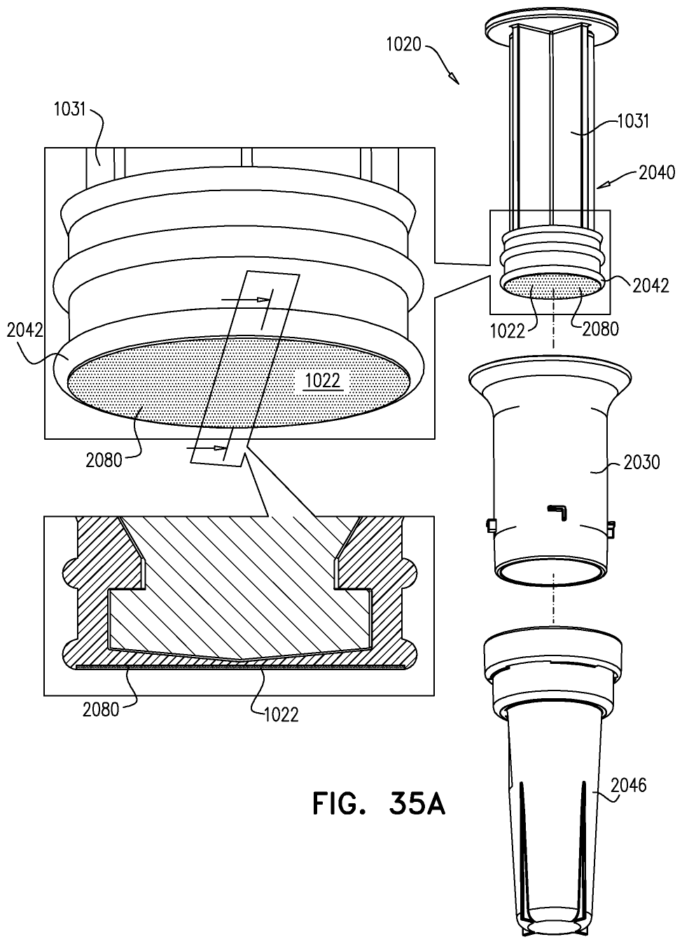

Reference is now made to FIGS. 34A-C, which are schematic illustrations of methods for performing a backup test, e.g., a backup strep test, in accordance with respective applications of the present invention. Although these configurations are illustrated with respect to testing device 2020, they may also be combined with the other testing devices described herein, mutatis mutandis. Prior to liquid 2022 being passed through filter 2032, some of liquid 2022, e.g., gargled fluid or saliva not swabbed from the patient's throat, is removed as a sample for a backup test. FIG. 34A shows an absorbent element, e.g., a swab 2058, e.g., a flocked swab, a cotton swab, or a polyester swab, being inserted into liquid 2022 and then placed into test tube 2085 containing growth medium 2087. For some applications, liquid 2022, or a portion of liquid 2022, is transferred into the container, e.g., test tube 2085, by other means, such as for example, pouring, using a syringe, using a pipette, or a pump. Growth medium 2087 may be a liquid growth medium, a dehydrated growth medium, or a gel growth medium. Optionally, growth medium 2087 has the properties of the high-concentration liquid growth medium described in detail hereinbelow. Typically, test tube 2085 does not contain agar.

Reference is now made to FIG. 34D, which is a flowchart depicting a method for performing a backup strep test using rapid strep test (RST) techniques on gargled fluid after incubation, in accordance with some applications of the present invention. Examples of the method of FIG. 34D are provided in the experimental data set forth hereinbelow in the section entitled, “Measuring Group A Beta-Hemolytic Streptococcus Bacteria in Throat Gargle: Results of Overnight Growth in Liquid Media, Assayed by Rapid Strep Test.” In step 1200 the gargled fluid is incubated for at least 12 hours and/or less than 75 hours in a container, e.g., test tube 2085, that contains a liquid growth medium, a dehydrated growth medium, or a gel growth medium. As illustrated by the experimental data, the total volume of gargled fluid and growth medium is typically at least 0.45 ml and/or less than 3.6 ml. In some applications, the gargled fluid is mixed with the growth medium before incubation. Typically, the container containing the growth medium does not contain agar.

In step 1202, after incubation, an RST, e.g., a lateral flow test, is performed on the mixture of gargled fluid and growth medium. For some applications, the RST may be one of the following: an ELISA-based RST, an antibody-coated-beads-based RST, a nucleic-acid-based RST, and a fluorescent immunoassaying (FIA) RST. As supported by the experimental data set forth hereinbelow in the section entitled, “Measuring Group A Beta-Hemolytic Streptococcus Bacteria in Throat Gargle: Results of Overnight Growth in Liquid Media, Assayed by Rapid Strep Test,”, a number of methods used for performing the RST yield usable results, as follows:
RST is performed on the mixture of gargled fluid and growth medium while the gargled fluid and growth medium are in the container. This method of RST is referred to as “whole tube RST” in the experimental data.
At least a portion, e.g., at least 0.05 ml, e.g., 0.1 ml, of the mixture of gargled fluid and growth medium is transferred to another container, and the RST is performed on the portion of the gargled fluid and growth medium in the other container. This method of RST is referred to as “sample RST” in the experimental data. For some applications, the portion of the mixture is transferred by inserting an absorbent element, e.g., a swab, e.g., a flocked, cotton, or polyester swab, into the mixture of gargled fluid and growth medium and then placing the swab into the other container. Alternatively, if an absorbent element, e.g., a swab, was used to transfer liquid 2022, e.g., the gargled fluid, into the container with growth medium, then that same absorbent element, e.g., swab may be removed and used to transfer the portion of the mixture into the other container for “sample RST.” For some applications, the portion of the mixture is transferred into the container, e.g., test tube 2085, by other means, such as for example, pouring, using a syringe, using a pipette, or a pump.
At least a portion of the mixture of gargled fluid and growth medium, after incubation, is filtered, e.g., passed through a filtration membrane (optionally, using any of the filtering devices described herein), and the RST is performed on the filter. This method of RST is referred to as “filter RST” in the experimental data.

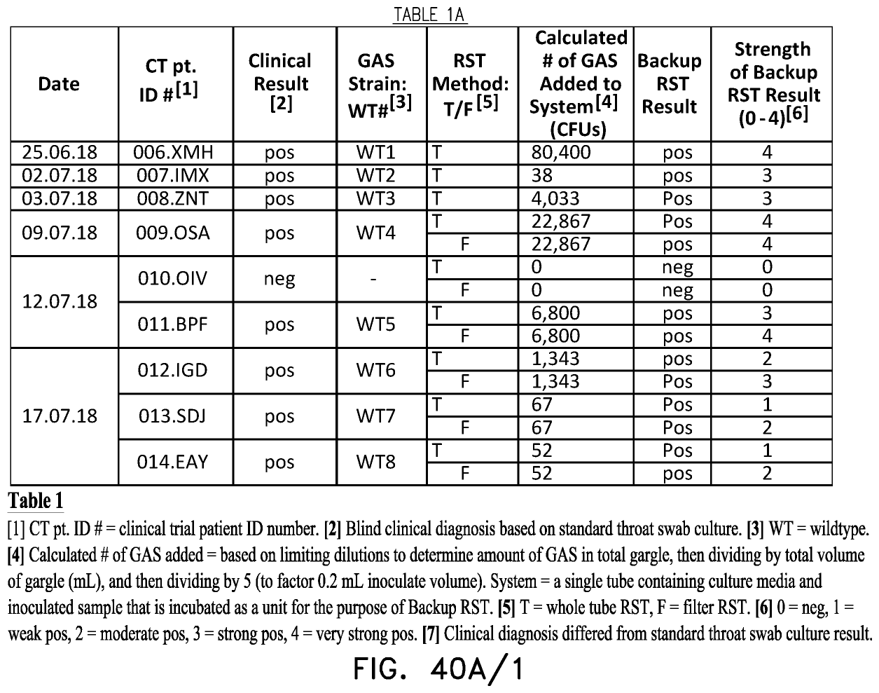

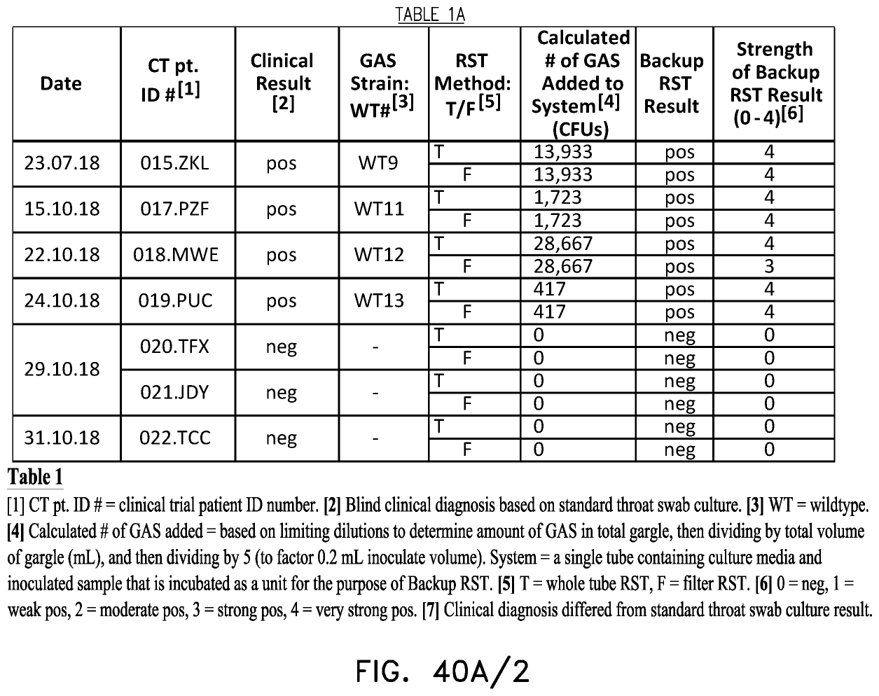

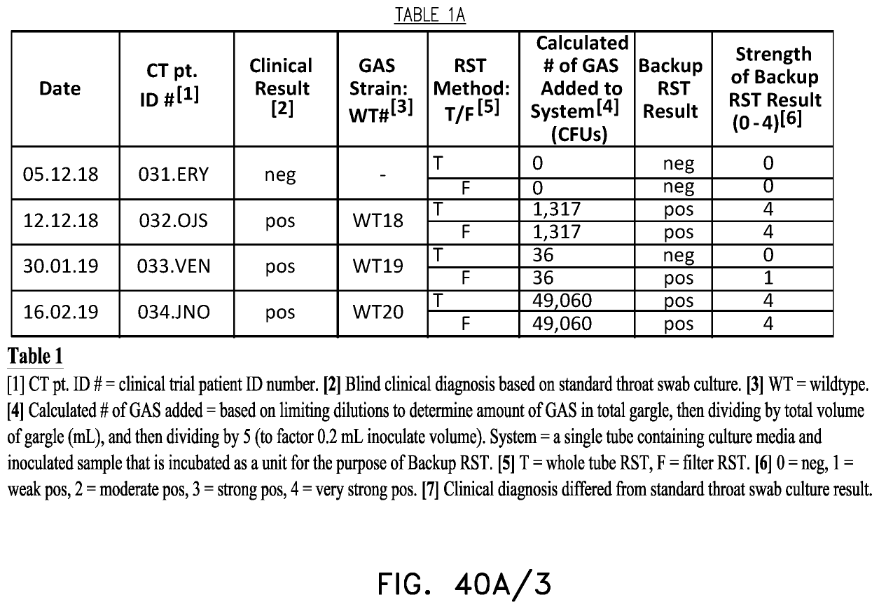

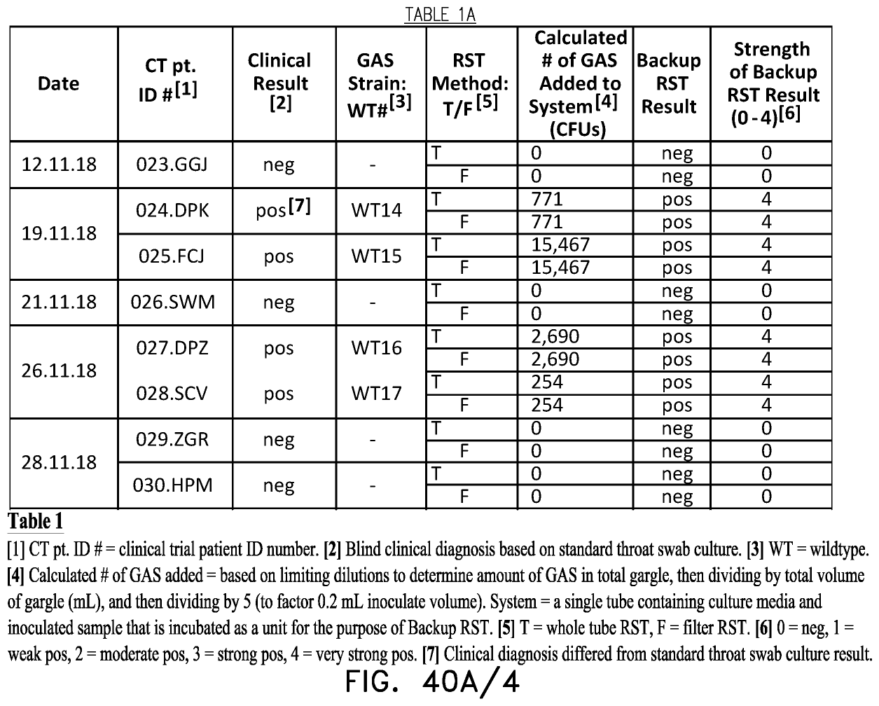

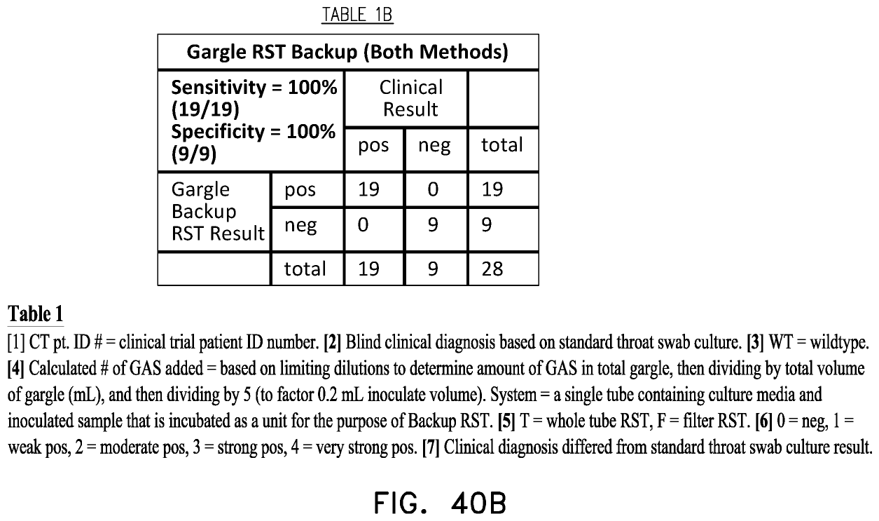

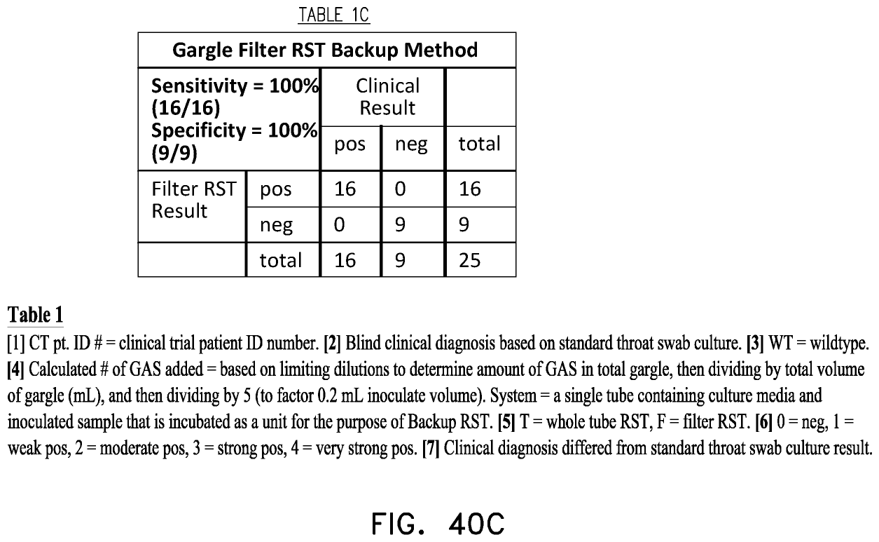

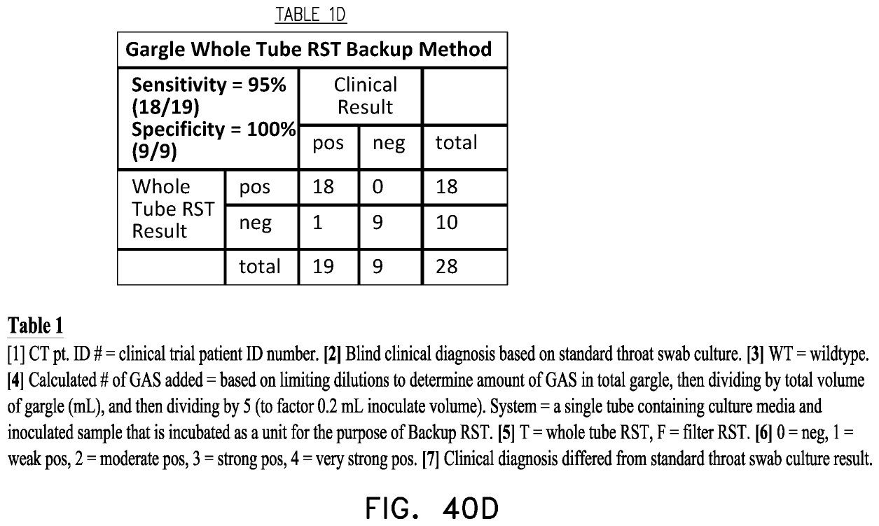

Results of a clinical trial performed by the inventors, including twenty-eight patients, are shown in Tables 1A-1D of FIGS. 40A-D, respectively, of the experimental data set forth hereinbelow in the section entitled, “Measuring Group A Beta-Hemolytic Streptococcus Bacteria in Throat Gargle: Results of Overnight Growth in Liquid Media, Assayed by Rapid Strep Test.” Gargled fluid was collected from each of the 28 patients and tested using “whole tube RST” and/or “filter RST” after incubation for at least 21 hours and/or less than 25 hours (further details regarding parameters of the tested systems are set forth in the experimental data). As shown in Tables 1A-1D, 19 systems yielded true positive RST results, which corresponded to 19 of the patients who were clinically positive for GAS pharyngitis (true positive), and nine systems yielded true negative results, which corresponded to nine patients who were clinically negative for GAS pharyngitis (true negative). See Table 2 of FIG. 41 in the experimental data for parameters from 78 additional experimental systems that yielded true positive RST results. These additional experimental systems each contained either (a) gargled fluid which was spiked with Group A Streptococcal (“GAS”) bacteria, or (b) a GAS bacteria suspension in a pure buffer.

Reference is now made to FIG. 34E, which is a flowchart depicting a method for performing a backup strep test using rapid strep test (RST) techniques on saliva not swabbed from a patient's throat after incubation, in accordance with some applications of the present invention. Examples of this method are provided in the experimental data set forth hereinbelow in the section entitled, “Measuring Group A Beta-Hemolytic Streptococcus Bacteria in Saliva Sample: Results of Overnight Growth in Liquid Media, Assayed by Rapid Strep Test.” In step 1204 the saliva swab is incubated for at least 12 hours and/or less than 75 hours in a container, e.g., test tube 2085, that contains a liquid growth medium, a dehydrated growth medium, or a gel growth medium. In some applications, the saliva is mixed with the growth medium before incubation.

Typically, the container containing the growth medium does not contain agar. For some applications, the patient sucks on the swab, or the swab is rubbed on the patient's tongue and/or cheek. In this manner, the saliva is received on the swab, e.g., a flocked swab, a cotton swab, or a polyester swab, from the patient's mouth, and the swab is then placed directly into the container that contains the growth medium. Alternatively, the patient spits saliva into the container.

In step 1206, after incubation, an RST, e.g., a lateral flow test, is performed on the mixture of saliva and growth medium. For some applications, the RST may be one of the following: an ELSA-based RST, an antibody-coated-beads-based RST, a nucleic-acid-based RST, and a fluorescent immunoassaying (FIA) RST. As supported by the experimental data set forth hereinbelow in the section entitled, “Measuring Group A Beta-Hemolytic Streptococcus Bacteria in Saliva Sample: Results of Overnight Growth in Liquid Media, Assayed by Rapid Strep Test,” and similarly to as described hereinabove with reference to FIG. 34D, a number of methods were used for performing the RST, as follows:
“Whole tube RST,” as described hereinabove, mutatis mulandis.
“Sample RST,” as described hereinabove, mutatis mulandis.
“Filter RST,” as described hereinabove, mutatis mutandis.
After incubation, at least a portion of the mixture of saliva and growth medium is transferred to another container by removing the swab from the container containing the growth medium and placing the swab into the other container, and the RST is performed on the swab in the other container. This method of RST is referred to as “swab RST” in the experimental data.

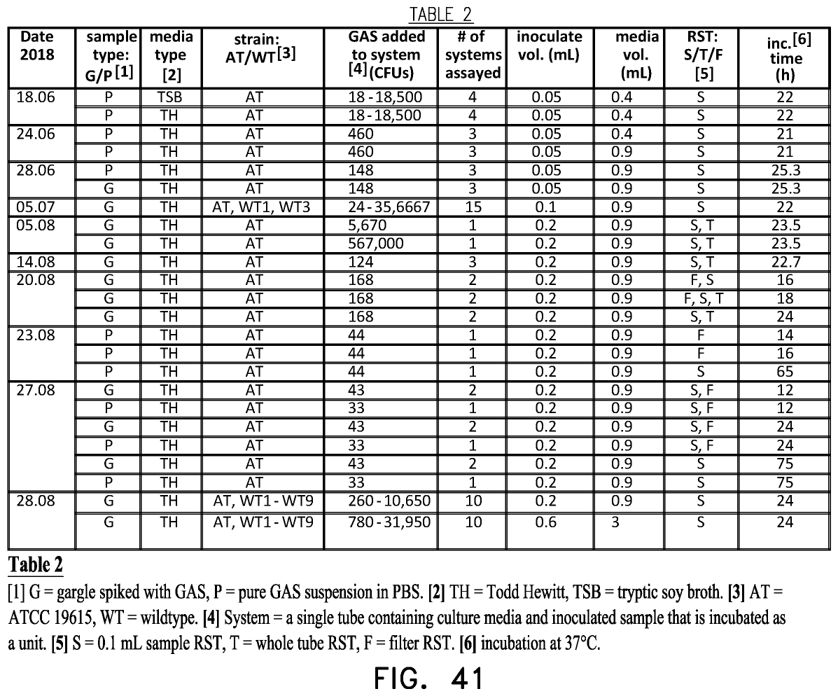

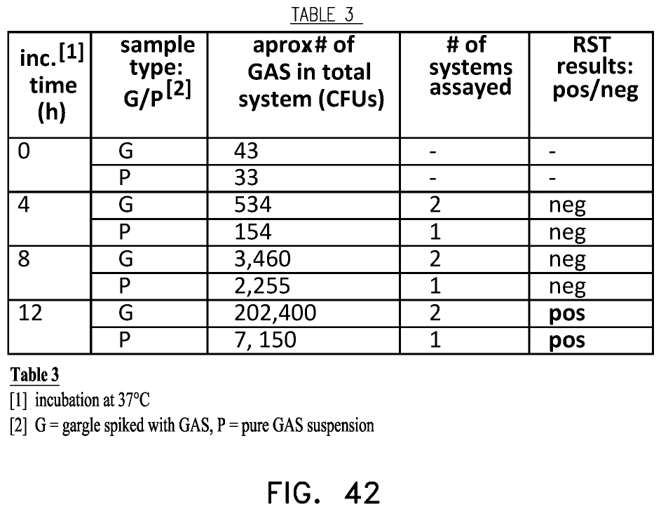

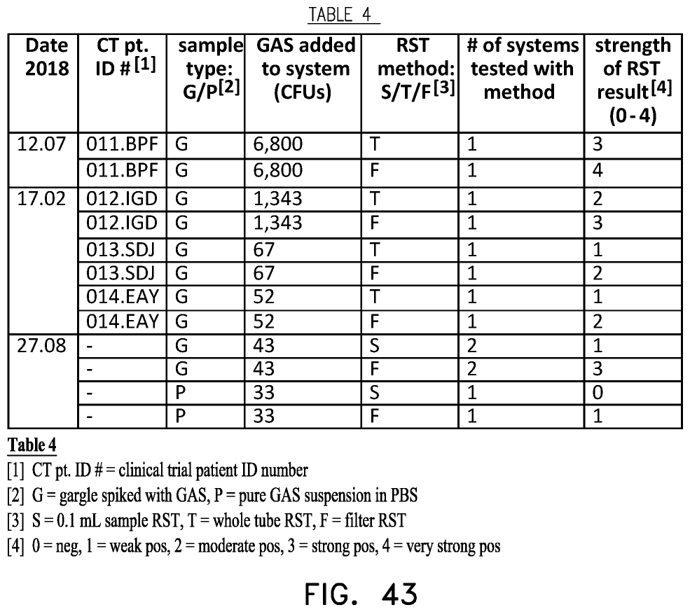

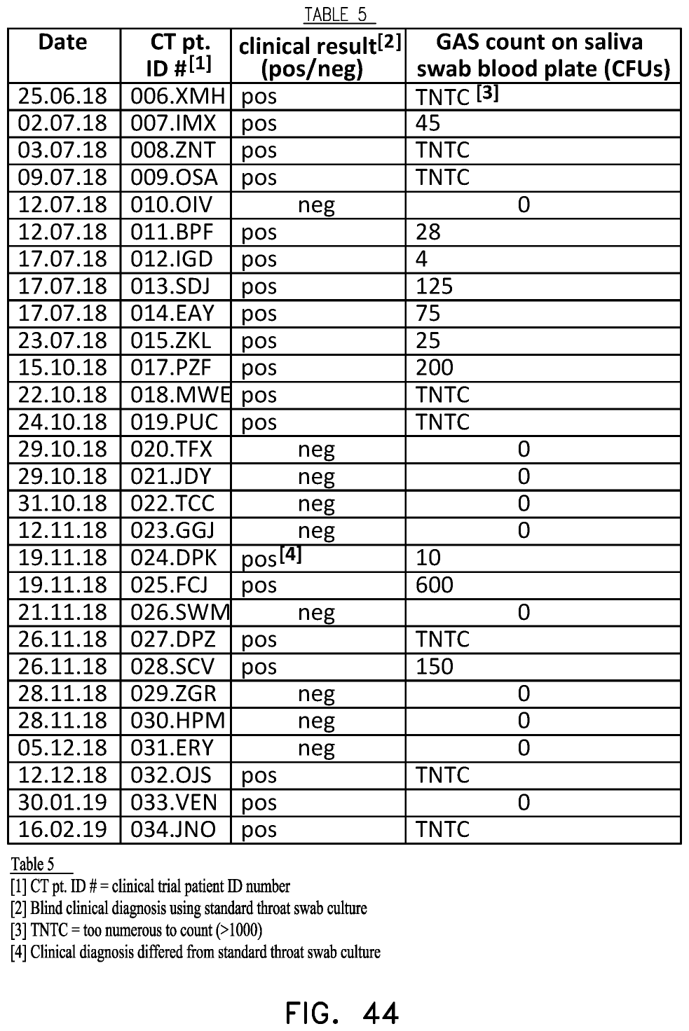

In a clinical trial performed by the inventors, 28 patients were asked to suck on a flocked swab for about ten seconds. The saliva swabs were then inoculated onto blood plates, and beta-hemolytic colonies were counted using a light table. As illustrated by Table 5 of FIG. 44 in the experimental data set forth hereinbelow in the section entitled, “Measuring Group A Beta-Hemolytic Streptococcus Bacteria in Saliva Sample: Results of Overnight Growth in Liquid Media, Assayed by Rapid Strep Test,” 18 out of 19 (94.7%) saliva swabs from subjects who were clinically positive for GAS pharyngitis were found to contain a number of colony-forming units (CFUs) of GAS ranging from four to “too numerous to count.” A false negative was obtained for 1 of the 19 saliva swabs.

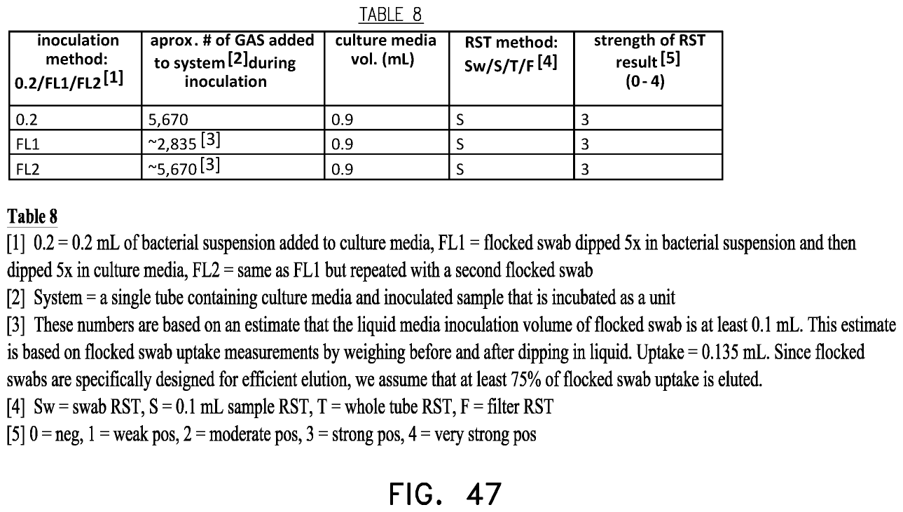

As illustrated by the experimental data, experiments were also carried out by the inventors using saliva swab simulations by dipping swabs into pure GAS bacteria suspensions (referred to as “saliva swab simulation 1” in the experimental data) or into gargled fluid that was spiked with GAS bacteria (referred to as “saliva swab simulation 2” in the experimental data). The data presented in Table 7 of FIG. 46 represent an experiment using “saliva swab simulation 1” which compared the total absorbance plus elution of GAS bacteria onto a plate from three different swab materials: cotton, polyester, and flocked. The flocked swabs appear to have the highest absorption plus elution efficiency, however the cotton and polyester swabs provide useful results as well. The data presented in Table 8 of FIG. 47 represent an experiment using “saliva swab simulation 2” showing that flocked swabs may be used as an efficient way of transferring liquid, e.g., saliva, or gargled fluid, into the culture medium.

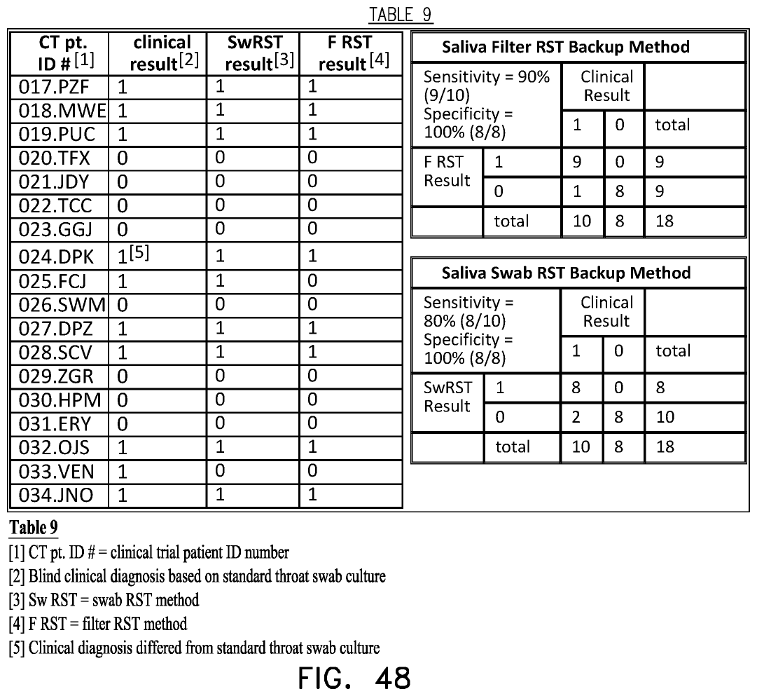

Almost all saliva swab clinical samples which were inoculated into Todd Hewitt (TH) broth and assayed by backup methods using RST methods yielded either true positive or true negative results for all subjects enrolled in phase 2 of the Proof of Concept Clinical Trial, seen in Table 9 of FIG. 48. The data presented in Table 9 describe Clinical Trial saliva swabs that were incubated in TH culture broth and were then assayed using backup test methods performed using RST methods. The Filter RST method had a sensitivity of 90% and the Swab RST method had a sensitivity of 80%.

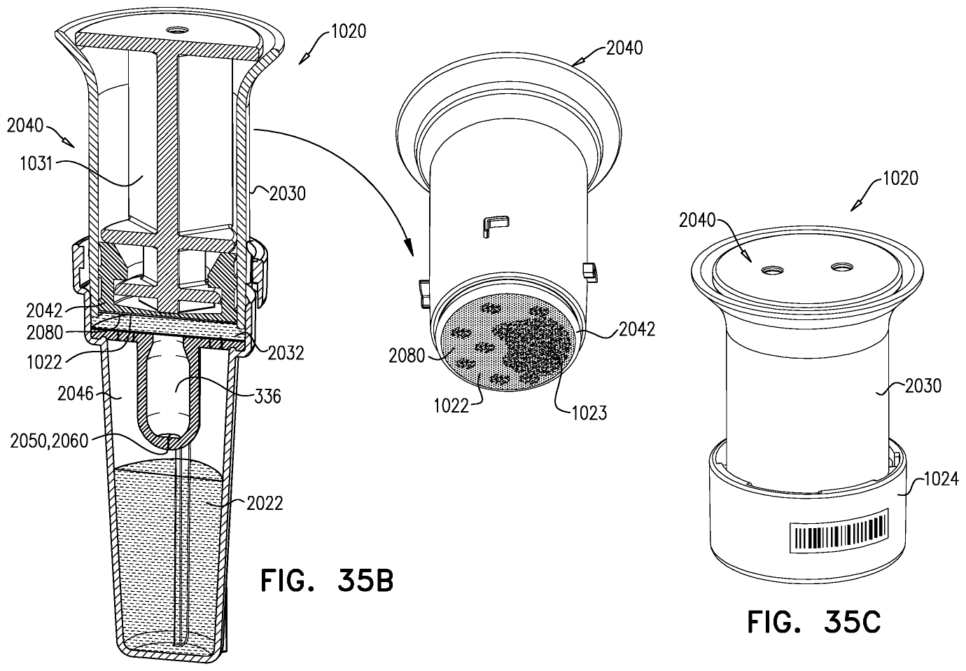

Reference is now made to FIGS. 35A-C, which are schematic illustrations of a testing device 1020 for testing for presence of particulate in liquid 2022, in accordance with an application of the present invention. Although testing device 1020 is illustrated as being similar to testing device 2020 described hereinabove with reference to FIGS. 22A-H, the techniques of testing device 1020 may also be combined with the other testing devices described herein, mutatis mulandis.

Testing device 1020 comprises:
liquid container 2030 for containing liquid 2022;
filter 2032, disposed in or downstream of liquid container 2030; and
plunger head 2042, which (a) is shaped so as to be insertable into liquid container 2030, (b) is configured to apply pressure to drive liquid 2022 from liquid container 2030 through filter 2032, and (c) has downstream surface 2080.

Downstream surface 2080 is at least partially coated with a solid (e.g., dehydrated and/or powdered) or semi-solid (e.g., gel and/or paste) growth medium 1022. For example, growth medium 1022 may comprise agar.

For some applications, a cap 1024 is provided that is configured to be coupled to and fully cover growth medium 1022 on downstream surface 2080 of plunger head 2042. For example, cap 1024 may be transparent to enable observation of the culture on downstream surface 2080 without removing the cap.

Typically, plunger head 2042 is shaped so as to be insertable into liquid container 2030 so as to form a movable seal with a wall of liquid container 2030. For some applications, testing device 1020 further comprises a plunger shaft 1031, and plunger head 2042 is disposed at a downstream end portion of plunger shaft 1031.

Plunger 2040 (including plunger head 2042 and plunger shaft 1031) may implement any of the configures of plunger 2040 described hereinabove with reference to FIGS. 22A-H. For some applications, an area of downstream surface 2080 of plunger head 2042 is between 0.3 and 100 cm2, such as between 0.3 and 30 cm2. For some applications, testing device 1020 further comprises waste liquid receptacle 2046, coupled to liquid container 2030 downstream of filter 2032.

For some applications, a method for using testing device 1020 comprises:
pushing plunger head 2042 to apply pressure to drive liquid 2022 from liquid container 2030 through filter 2032;
touching downstream surface 2080 of plunger head 2042 to filter 2032; particulate 1023, such as bacteria, on filter 2032 are captured by growth medium 1022 on downstream surface 2080; and
assessing downstream surface 2080 of plunger head 2042 for biological growth.

Typically, downstream surface 2080 is placed directly in an incubator before assessing, thereby obviating the need to use another device to take a backup sample and plate it onto agar. Downstream surface 2080 is optionally accessed by decoupling upstream component 2070 from downstream component 2072, such as described hereinabove with reference to FIGS. 22B-C.

For some applications, liquid 2022 includes at least one substance selected from the group of substances consisting of gargled fluid, saliva not swabbed from the throat of a patient, and an incubated culture medium containing a biological sample.

For some applications, downstream surface 2080 of plunger head 2042 is assessed for biological growth of a biological particulate selected from the group consisting of: a microorganism, a fungus, a bacterium, a spore, a virus, a mite, a biological cell, a biological antigen, a protein, a protein antigen, and a carbohydrate antigen. For some applications, plunger head 2042 is heated before downstream surface 2080 of plunger head 2042 is assessed for biological growth.

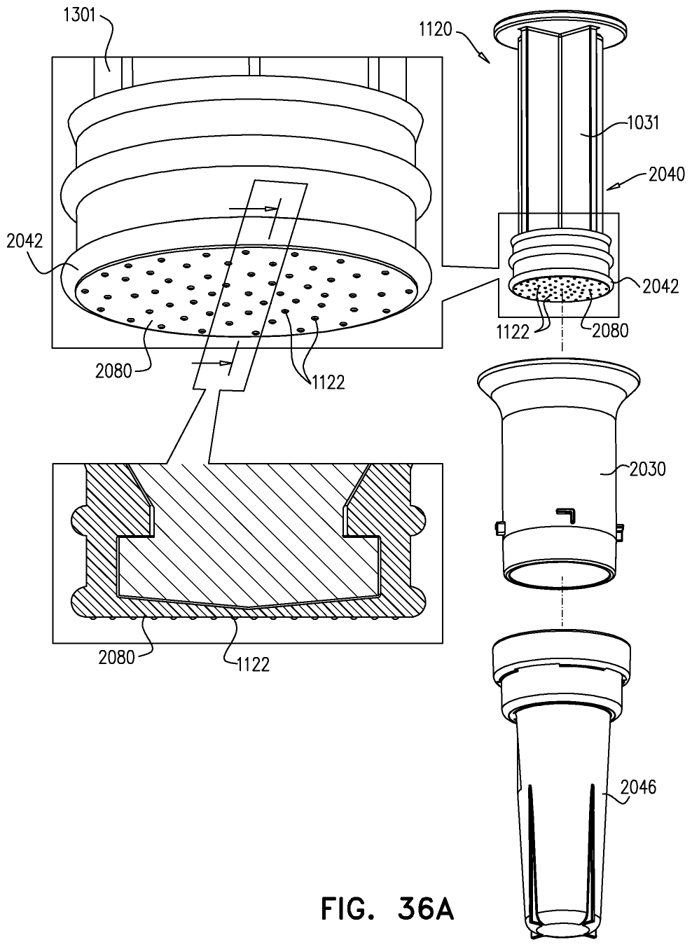

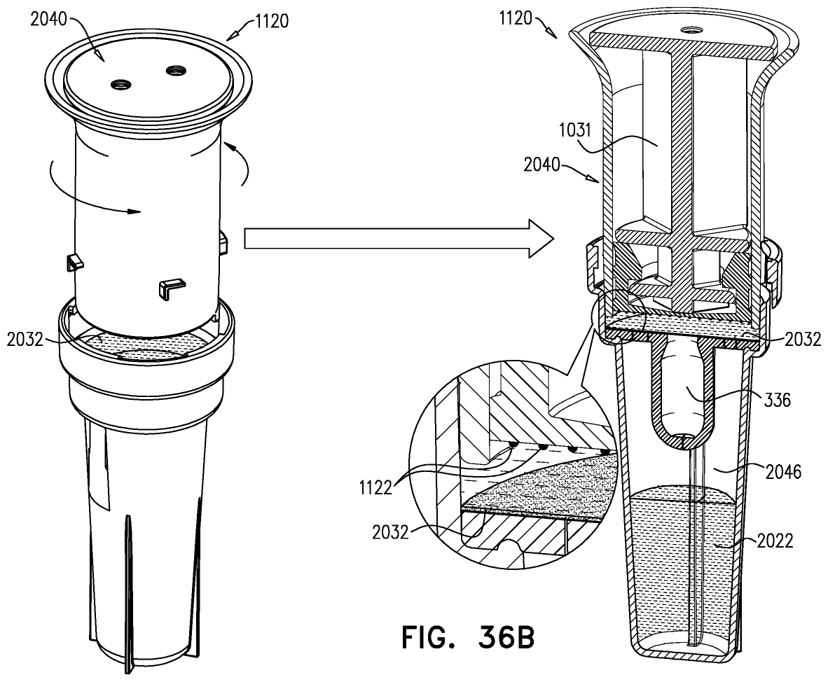

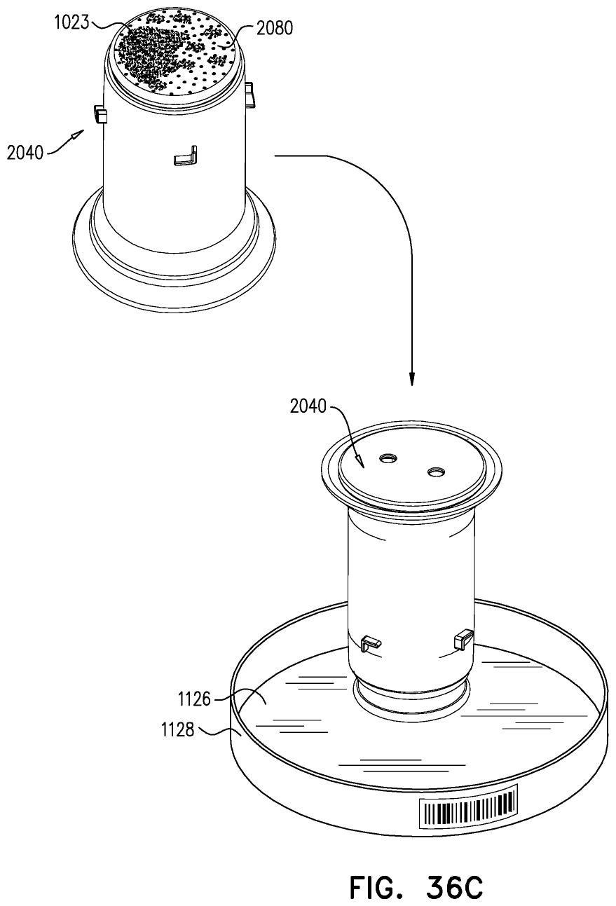

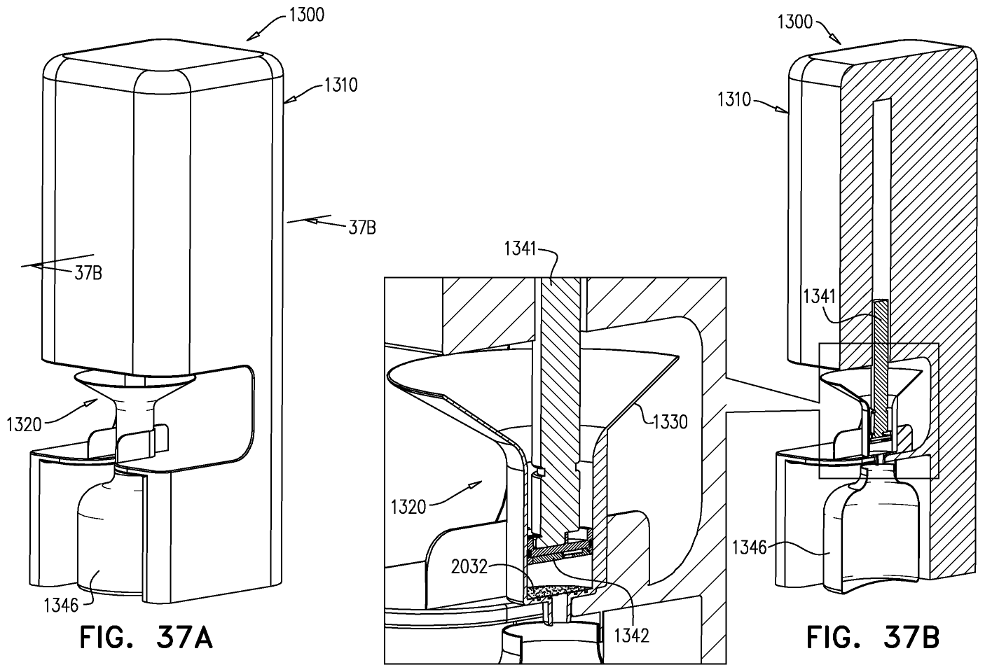

Reference is now made to FIGS. 36A-C, which are schematic illustrations of a method for using a testing device 1120 for testing for presence of particulate in liquid 2022, in accordance with an application of the present invention. Although testing device 1120 is illustrated as being similar to testing device 2020 described hereinabove with reference to FIGS. 22A-H, the method described with reference to testing device 1120 may also be combined with the other testing devices described herein, mutatis mulandis.

The method comprises:
pushing plunger head 2042 to apply pressure to drive liquid 2022 from liquid container 2030 of testing device 1120 through filter 2032;
touching downstream surface 2080 of plunger head 2042 to filter 2032;
thereafter, touching downstream surface 2080 of plunger head 2042 to culture medium 1126 contained in a culture-medium container 1128, such as a petri dish; for example, culture medium 1126 may include agar;
heating culture-medium container 1128; and
assessing culture-medium container 1128 for biological growth.

For some applications, liquid 2022 includes at least one substance selected from the group of substances consisting of gargled fluid, saliva not swabbed from the throat of a patient, and an incubated culture medium containing a biological sample.

For some applications, culture-medium container 1128 is assessed for biological growth of a biological particulate 1023 selected from the group consisting of: a microorganism, a fungus, a bacterium, a spore, a virus, a mite, a biological cell, a biological antigen, a protein, a protein antigen, and a carbohydrate antigen.

Downstream surface 2080 is optionally accessed by decoupling upstream component 2070 from downstream component 2072, such as described hereinabove with reference to FIGS. 22B-C.

S For some applications, plunger head 2042 is rotated while touching downstream surface 2080 of plunger head 2042 to filter 2032 to increase the sample taken from filter 2032, such as by macerating or grinding the filter. For applications in which downstream surface 2080 is decoupled from upstream component 2070 by rotation, this rotation may itself increase the sample taken from filter 2032.

For some applications, downstream surface 2080 of plunger head 2042 is rough, i.e., is shaped so as to define many small protrusions 1122, such as like sandpaper, or with plastic protrusions, in order to collect a better sample of particulate 1023 by macerating or grinding the filter.

For some applications, touching downstream surface 2080 of plunger head 2042 to filter 2032 comprises grinding filter 2032 with rough downstream surface 2080.

For some applications, the method further comprising testing, within testing device 1120, for the presence of biological particulate 1023 trapped by filter 2032, such as described hereinabove. In these applications, the sample taken from downstream surface 2080 of plunger head 2042 is used to perform a backup test, e.g., a backup strep test, for the rapid test performed inside testing device 1120, as described hereinabove.

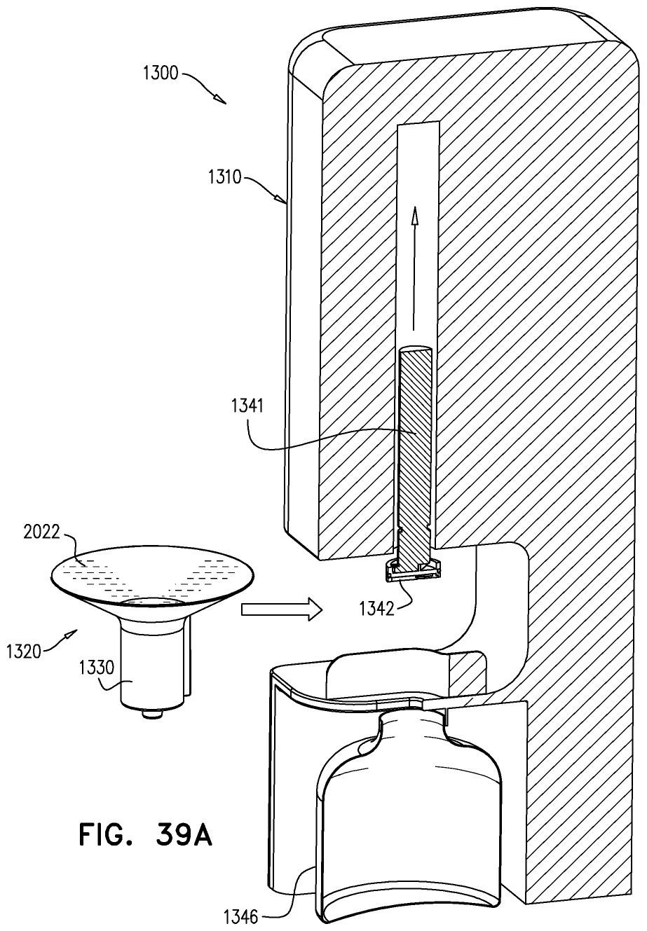

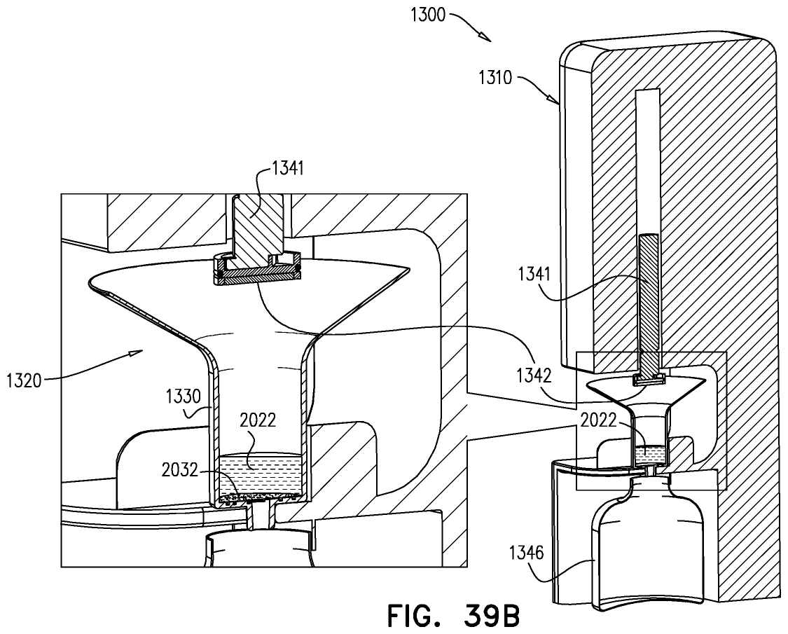

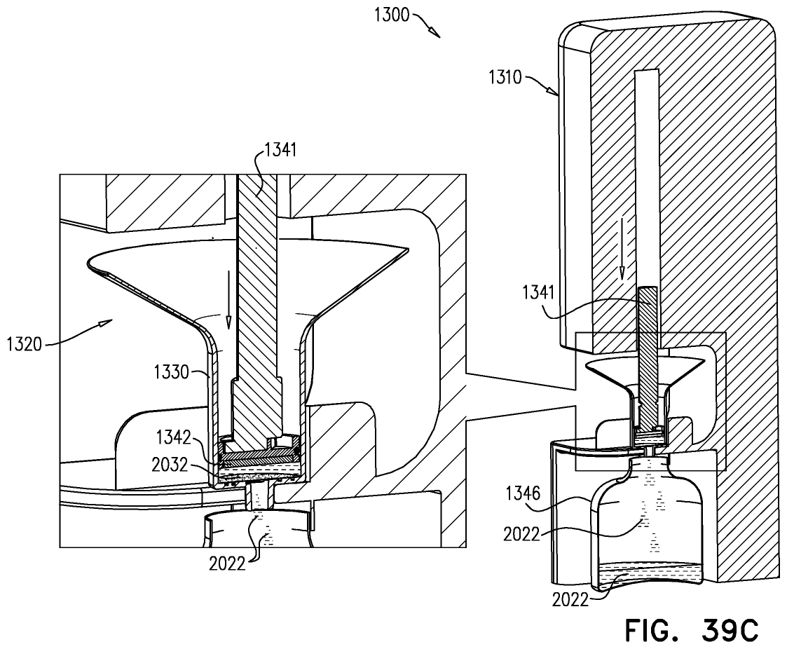

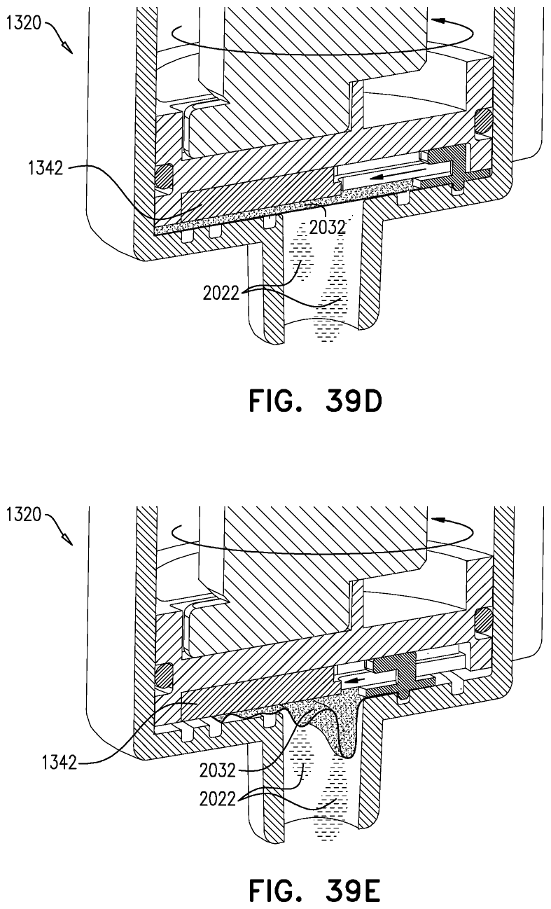

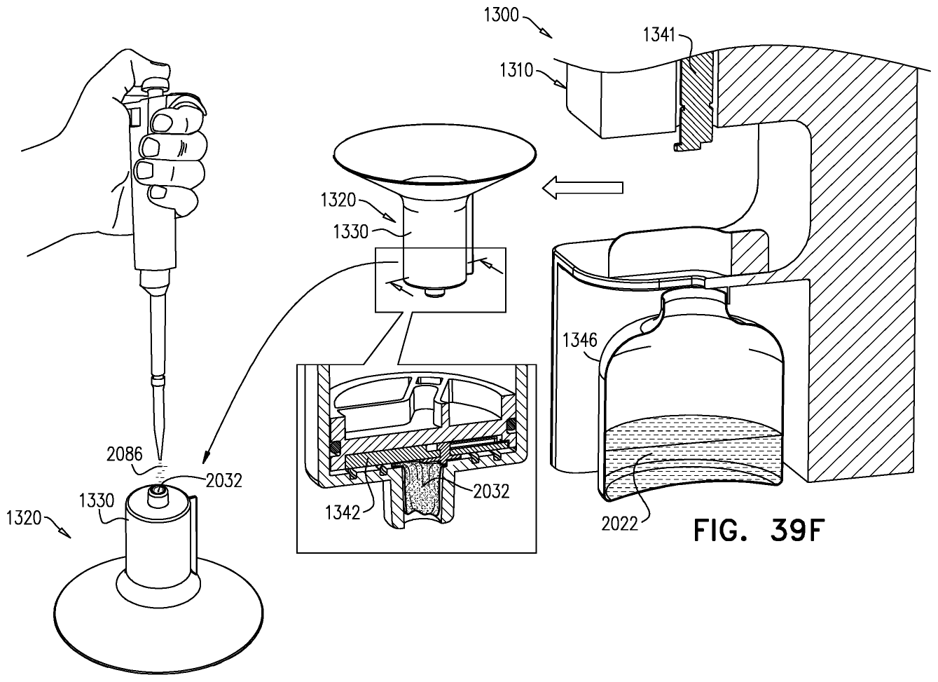

Reference is now made to FIGS. 37A-B, which are schematic illustrations of a testing system 1300, in accordance with an application of the present invention. Testing system 1300 comprises a testing machine 1310 and a testing device 1320 for testing for the presence of particulate in a liquid 2022 (shown in FIGS. 39B-F). Testing device 1320 is configured to be removably inserted into testing machine 1310 for performing a test. Testing device 1320 may be disposable, while testing machine 1310 is typically reused many times with separate testing devices 1320. Testing device may optionally implement any of the features of the other testing devices described herein, mutatis mulandis.

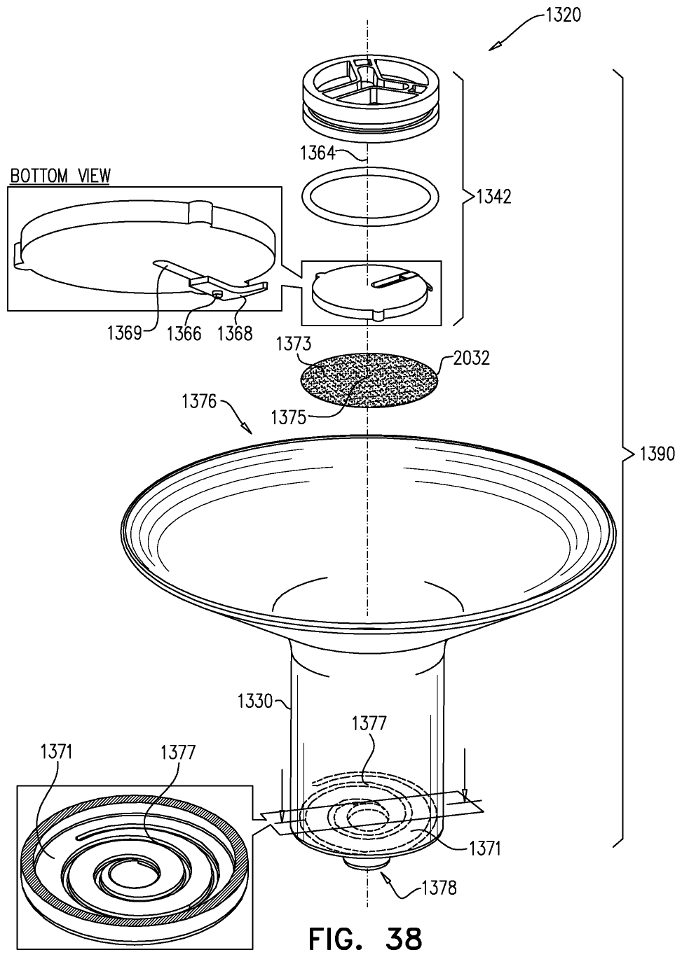

Reference is also made to FIG. 38, which is a schematic exploded view of testing device 1320, in accordance with an application of the present invention. Testing device 1320 comprises:
a liquid container 1330 for containing liquid 2022, liquid container 1330 shaped so as to define an upstream opening 1376 and a downstream opening 1378;
a filter 2032, removably disposed in liquid container 1330; and
a plunger head 1342 that (a) is shaped so as to be insertable into liquid container 1330 so as to form a movable seal with a wall of liquid container 1330, and (b) is arranged such that when pushed, plunger head 1342 applies pressure to drive liquid 2022 contained in liquid container 1330 through filter 2032 and then through downstream opening 1378.

Testing device 1320 is configured such that rotation of plunger head 1342 radially compresses filter 2032 toward a central longitudinal axis 1364 of plunger head 1342, as shown in FIG. 39F, described hereinbelow. This concentrates filter 2032 in a more compact volume to better enable the performance of a test for the particulate, as described hereinbelow with reference to FIG. 39F. For some applications, testing device 1320 is configured such that the rotation of plunger head 1342 crushes filter 2032, which may improve the sensitivity of the subsequent testing.

For some applications, plunger head 1342 comprises a protrusion 1366 (best seen in FIG. 38), and testing device 1320 is configured such that the rotation of plunger head 1342 causes protrusion 1366 to move radially toward central longitudinal axis 1364 of plunger head 1342. For example, protrusion 1366 may be coupled to a base 1368 that can slide radially within a track 1369, such as shown in the bottom view in FIG. 38, or protrusion 1366 may be directly slidable within a track.

For some applications, liquid container 1330 is shaped so as to define a filter-support surface 1371 surrounding downstream opening 1378. Filter-support surface 1371 supports a radial portion 1373 of filter 2032 excluding a central portion 1375 of filter 2032 (the central portion 1375 is typically removably disposed over downstream opening 1378). Filter-support surface 1371 is shaped so as to define a spiral groove 1377. Protrusion 1366 is configured to engage spiral groove 1377 through filter 2032.

Testing device 1320 is configured such that the rotation of plunger head 1342 (such as by between one-third of a turn to 10 turns) causes spiral groove 1377 to guide protrusion 1366 radially toward central longitudinal axis 1364 of plunger head 1342.

Reference is now made to FIGS. 39A-F, which are schematic illustrations of a method for using testing system 1300 to test for the presence of the particulate in liquid 2022, in accordance with an application of the present invention.

As shown in FIGS. 39A-B, plunger head 1342 of testing device 1320 is coupled to a plunger shaft 1341 of testing machine 1310. The other components of testing device 1320, including but not limited to liquid container 1330, are removably inserted into testing machine 1310, typically after liquid 2022 is contained in liquid container 1330.

As shown in FIG. 39C, plunger shaft 1341 pushes plunger head 1342 applies pressure to drive liquid 2022 contained in liquid container 1330 through filter 2032 and then through downstream opening 1378.

As shown in FIGS. 39D-E, plunger shaft 1341 rotates plunger head 1342 to radially compress filter 2032 toward central longitudinal axis 1364, shown in FIG. 39F.

Optionally, liquid container 1330 includes a narrower outlet portion, and radially compresses the filter also deposits all or a portion of the filter in the narrower outlet portion.

As shown in FIG. 39F, filter 2032 is tested for particulate trapped in filter 2032, including, for example, applying extraction reagent 2086, as described hereinabove.

For some applications, testing machine 1310 comprises a waste liquid receptacle 1346, into which liquid 2022 is driven ater passing through filter 2032.

Typically, waste liquid receptacle 1346 is large enough to accommodate tests performed using several testing devices 1320.

Reference is again made to FIG. 38. In an application of the present invention, a testing kit 1390 is provided for use with liquid 2022. Testing kit 1390 comprises:
liquid container 1330 for containing liquid 2022, liquid container 1330 shaped so as to define an upstream opening 1376 and a downstream opening 1378;
filter 2032, disposed in or downstream of liquid container 1330; and
plunger head 1342 that (a) is shaped so as to be insertable into liquid container 1330 so as to form a movable seal with a wall of liquid container 1330, and (b) is arranged such that when pushed, plunger head 1342 applies pressure to drive liquid 2022 contained in liquid container 1330 through filter 2032 and then through downstream opening 1378.

Testing kit 1390 does not comprise a plunger shaft. Instead, plunger head 1342 is removably coupled to plunger shaft 1341 of testing machine 1310, as described above with reference to FIGS. 39A-B.

Although testing kit 1390 has been described with reference to liquid container 1330 and plunger head 1342, testing kit 1390 may alternatively comprise any of the other liquid containers described herein or another liquid container known in the art, and/or plunger head 1342 may alternatively comprise any of the other plunger heads described herein or another plunger head known in the art.

For some applications, sterile packaging is provided, in which at least liquid container 1330, plunger head 1342, and filter 2032 are removably disposed. The sterile packaging comprises one or more sterile packages; for example, each element may be removably disposed in a separate one of the packages, and/or more than one the elements may be disposed in a single one of the packages.

Although techniques for testing, including rapid testing, are generally described herein as being performed for detecting strep, they may also be used to detect other biological particulate, such as a virus. For example, for detecting a virus, the filters described herein may capture epithelial cells that include the virus.

### Measuring Group A Beta-Hemolytic Streptococcus Bacteria in Throat Gargle: Results of Overnight Growth in Liquid Media, Assayed by Rapid Strep Test

In some applications of the present invention, group A streptococcus bacteria (GAS) can be detected in throat gargle by two primary method types: Direct (“Immediate”) methods and Indirect (“Backup”) methods. Immediate methods yield results faster than Backup methods (<20 minutes vs. 12-48 hours) but are not as sensitive (higher rate of false negatives).

In a clinical experiment performed by the inventors, Backup methods for GAS detection in throat gargle were tested. The experimental data is based on two types of GAS throat gargle simulations: pure GAS liquid suspension and throat gargle spiked with GAS. The Clinical Trial data is based on throat gargles obtained from patients with GAS pharyngitis who were enrolled in phase 2 of a Proof of Concept Clinical Trial (Protocol Number: STRP.P001, SNIH Clinical Trial Number: NCT03231098, Shaare Zedek Medical Center Helsinki IRB Number: SZMC-0181-17).

### Materials and Methods

Bacterial Culture: 10 GAS strains were used: 1 standard control strain and 9 wildtype strains. The control GAS strain was American Type Culture Collection (“ATCC”) 19615, a strain often used for quality control, and the wildtype GAS strains were isolated during Clinical Trials and labeled WT-1 through WT-9. All GAS bacteria used in experiments were taken from 1-7 days old cultures on blood agar plates stored at 4-8° C.

Growth conditions: The bacteria were routinely grown in a 37° C. incubator, without agitation. Liquid cultures were grown in 4 mL plastic test tubes, with liquid volumes of 0.45 mL, 0.6 mL, 1.0 mL, 1.1 mL, and 3.6 mL after inoculation. Cultures were incubated for 12-75 hours.

Growth media: Blood plate media: Standard 90 mm plate (Petri dishes) containing TSA+5% sheep blood. Blood plates were purchased from Hylabs (Rehovot, Israel, Cat. No. PD-049). Liquid media: Tryptic Soy Broth (“TSB”), which is a well-known general-purpose growth media. Sterile TSB tubes were purchased from Hylabs (Cat. No. TT139). Liquid media: Todd Hewitt broth (“TH”), which is a media specifically developed to grow Streptococci. TH powder was purchased from Sigma Aldrich (Missouri, USA, Cat. No. T1438-500g). The media was prepared and sterilized by filtration through 0.2 um filtration units. The liquid growth media was prepared with 4.5 times the concentration recommended in the instructions. At this higher concentration, the liquid growth media had the following concentrations: glucose: 9 g/L; nitrogen source: 135 g/L; inorganic molecules: 22.05 g/L; and total solids: 166.5 g/L (as shown in the 4.5 row of Table 11, described hereinbelow.

Bacterial suspensions: Pure GAS bacterial suspensions were made by transferring GAS colonies from culture into sterile Phosphate Buffer Saline (“PBS”).

Bacterial counts: 0.05 mL or 0.1 mL samples of bacterial suspensions or throat gargles were inoculated onto blood plates without dilution and with using the appropriate limiting dilutions (dilutions of 10-fold to 8000-fold) and beta-hemolytic colonies were counted using a light table.

Throat gargle: Throat gargles were obtained by gargling 10-1 mL PBS for approximately 10 seconds.

Gargle spiked with GAS: Pure GAS liquid suspensions were added to gargle and diluted with gargle as necessary.

RST methods: Lateral flow immuno-assay RST kits were purchased from Moore Medical (Connecticut, USA, Cat. No. 82792). Standard RST: Conducted according to manufacturers' instructions. Swab containing specimen sample was placed into a tube containing 8 drops of RST solutions, agitated slightly, and removed after 1-3 minutes. RST dipstick was then added to tube and removed after 5 minutes. 0.1 ml. Sample RST: Similar to standard RST. 0.1 mL of specimen sample was added to tube containing RST solutions instead of a swab. Whole tube RST: 8 drops of RST solutions were added directly into tubes containing liquid culture media (0.4 mL, 0.9 mL, or 3.0 mL) incubated with gargle or simulated gargle, and RST dipstick was added to tube 1-3 minutes after addition of RST solutions. Filter RST Culture media incubated with gargle or simulated gargle was filtered, membrane filter containing concentrated specimen sample was placed into a tube, 8 drops of RST solutions were added, and tube contents were mixed by a blunt tip for 30-45 seconds. The RST dipstick was added to the filter mixture approximately 3 minutes after addition of RST solutions.

### Summary of Results

Of the 28 patients enrolled in phase 2 of the Proof of Concept Clinical Trial, 19 patients had true positive results from Backup Test methods performed using RST methods and 9 patient had true negative results from Backup Test methods performed using RST methods, as displayed in Table 1A-ID of FIGS. 40A-D, respectively. The data in Table 1A of FIG. 40A presents the gargle Backup Test methods performed using RST results for 28 patient throat gargles. Each patient throat gargle was tested using one or two systems (total systems=53). Each system consisted of 0.2 mL inoculated gargle plus either 0.4 mL or 0.9 mL of TH culture media, for a total volume of either 0.6 mL or 1.1 mL. The calculated GAS within each inoculated gargle added to a system ranged from 36 CFU to 80,400 CFU. Systems were incubated for 21-25 hours at 37° C. and then processed using either the Filter RST method or Whole Tube RST method.

The data in Table 1B-1D of FIGS. 40B-D, respectively, present the sensitivity and specificity of the gargle Backup Test methods performed using RST results in general and separated by method. The data indicates that the Filter RST method has higher sensitivity (100%) than the Whole Tube RST method (95%).

Overall, 78 experimental systems containing simulated GAS gargles yielded true positive RST results, as detailed in Table 2 of FIG. 41. The data in Table 2 describes 78 systems which resulted in a positive lateral flow RST immunoassay after incubating a sample of simulated gargle containing GAS in culture media under a variety of conditions. Conditions include: incubating culture at 37° C. for 12 to 75 hours; using 2 different growth media; inoculation volumes of 0.05 mL, 0.1 mL, 0.2 mL, and 0.6 mL; overall volumes (inoculate+culture media) of 0.45 mL, 0.6 mL, 1.0 mL, 1.1 mL, and 3.6 mL; total number of starting GAS bacteria ranging between 18 to 567,000; and using 10 different GAS strains. All Backup method simulation tests using GAS bacteria suspension in a pure buffer, including tests which are not included in Table 2, consisted of at least 95% true positives. Backup method gargle simulation tests using gargle fluid spiked with GAS, including tests which are not included in Table 2, yielded a majority of true positive results, but were not as sensitive as pure GAS suspension due to inherent variability of the simulation and/or flaws in the model of spiking gargled fluid with GAS.

Minimum incubation time was 12 hours, as set forth in Table 3 of FIG. 42. The data in Table 3 describes an experiment where 9 systems were tested by lateral flow RST immunoassay after incubating a 0.2 mL sample of simulated gargle, either pure GAS suspension or gargle spiked with GAS, into 0.9 mL of Todd Hewitt culture media. Systems tested at 4 and 8 hours yielded negative RST, while systems tested at 12 hours yielded positive RST.

The data in Table 4 of FIG. 43 describe different lateral flow RST immunoassay methods and strength of RST results after incubating 0.2 mL of either a sample of simulated gargle containing GAS (four data points) or a sample of actual GAS pharyngitis patient gargle in 0.9 mL Todd Hewitt culture media (eight data points). The data suggest that of the three methods tested, the Filter RST method yields superior results and is the most sensitive method for detecting GAS using RST after incubation in culture media. The first eight rows of data from Table 4 (patient data) are also included in Table 1 (which also includes experimental collected performed after Table 4 was produced), while the last for rows of data (stimulations) are not included in Table 1 because the additional data in Table 1 rendered this data no longer relevant to support the conclusion because of the additional clinical data.

### Conclusions

These experimental data support the Backup method for GAS detection in throat gargle that involves the incubation of a sample of unfiltered throat gargle in liquid culture media for 12 to 75 hours followed by lateral flow RST immunoassay. A total of 53 systems from 28 patients enrolled in phase 2 of the Proof of Concept Clinical Trial all yielded either true positive or true negative results. A total of 78 experimental systems yielded positive Backup Test methods performed using RST results in multiple conditions. The Filter RST Backup method is presented as the most sensitive Backup Test method performed using a RST method, but all Backup Test methods performed using RST methods were satisfactory.

### Measuring Group A Beta-Hemolytic Streptococcus Bacteria in Saliva Sample: Results of Overnight Growth in Liquid Media, Assayed by Rapid Strep Test

In some applications of the present invention, group A streptococcus bacteria (GAS) can be detected from saliva swab by Indirect (“Backup”) methods. In a clinical experiment performed by the inventors, Backup methods for GAS detection from saliva swab were tested. The experimental data is based on GAS growth simulations and the Clinical Trial data is based on saliva swabs obtained from patients with GAS pharyngitis who were enrolled in phase 2 of a Proof of Concept Clinical Trial (Protocol Number: STRP.P001, SNIH Clinical Trial Number: NCT03231098, Shaare Zedek Medical Center Helsinki IRB Number: SZMC-0181-17).

### Materials and Methods

Bacterial Culture: 10 GAS strains were used: 1 standard control strain and 9 wildtype strains. The control GAS strain was American Type Culture Collection (“ATCC”) 19615, a strain often used for quality control, and the wildtype GAS strains were isolated during Clinical Trials and labeled WT-1 through WT-9. All GAS bacteria used in experiments were taken from 1-7 days old cultures on blood agar plates stored at 4-8° C.

Growth conditions: The bacteria were routinely grown in a 37° C. incubator, without agitation. Liquid cultures were grown in 4 mL plastic test tubes, with liquid volumes of 0.9-1.1 mL after inoculation. Cultures were incubated for 12-75 hours.

Growth media: Blood plate media: Standard 90 mm plate (Petri dishes) containing TSA+5% sheep blood. Blood plates were purchased from Hylabs (Rehovot, Israel, Cat. No. PD-049). Liquid media: Todd Hewitt broth (“TH”), which is a media specifically developed to grow Streptococci. TH powder was purchased from Sigma Aldrich (Missouri, USA, Cat. No. T1438-500g). The media was prepared and sterilized by filtration through 0.2 um filtration units. The liquid growth media was prepared with 4.5 times the concentration recommended in the instructions. At this higher concentration, the liquid growth media had the following concentrations: glucose: 9 g/L; nitrogen source: 135 g/L; inorganic molecules: 22.05 g/L; and total solids: 166.5 g/L (as shown in the 4.5 row of Table 11, described hereinbelow.

Swabs: Flocked Swabs: Swabs with a tip of short nylon brush-like fibers designed for efficient absorption and elution, purchased from Puritan Diagnostics (Maine, USA, Cat. No. 25-3306-H). Cotton Swabs: Swabs with a tip comprised of a cotton matrix, purchased from Kodan Medicam (Bet Shemesh, Israel, Cat. No. 1102245).

Polyeser Swabs: Swabs with a tip comprised of a polyester matrix, manufactured by Puritan Diagnostics (Guilford, Main, USA), Cat. No. 25-806 1PD SOLID).

Bacterial suspensions: Pure GAS bacterial suspensions were made by transferring GAS colonies from culture into sterile Phosphate Buffer Saline (“PBS”).

Gargle spiked with GAS: Throat gargles were obtained by gargling 10-1 mL PBS for approximately 10 seconds and pure GAS suspensions were added to gargle and diluted with gargle as necessary.

Saliva swabs: Saliva swabs were obtained from patients enrolled in the Clinical Trial. Patients were asked to suck on a flocked swab for approximately 10 seconds.

Saliva swabs obtained from Clinical Trial subjects were inoculated onto blood plates and beta-hemolytic colonies were counted using a light table. Some saliva swabs were then inoculated into TH culture media and swab was left in culture media during incubation. Both the culture media and the swab were later assayed by Backup methods performed using RST methods.

RST methods: Lateral flow immuno-assay RST kits were purchased from Moore Medical (Connecticut, USA, Cat. No. 82792). Swab sample RST: After incubation, saliva swab was removed from culture media and placed into a tube containing 8 drops of RST solutions, agitated slightly, and removed after 1-3 minutes. RST dipstick was then added to tube and removed after 5 minutes. 0.1 mL sample RST Similar to swab RST. 0.1 mL of specimen sample was added to tube containing RST solutions instead of a swab. Whole tube RST: In some cases, the 8 drops of RST solutions were added directly into tubes containing GAS in liquid culture media (0.9-1.1 mL) and RST dipstick was added to liquid culture media tube 1-3 minutes after addition of RST solutions. Filter RST: Culture media incubated with saliva swab was filtered, membrane filter was placed into a tube, 8 drops of RST solutions were added, and tube contents were mixed by a blunt tip for 30-45 seconds. The RST dipstick was added to the filter mixture approximately 3 minutes after addition of RST solutions.

Saliva swab simulation 1: Swabs were dipped into tubes containing pure GAS bacteria suspensions and agitated up and down 12-20 times before being removed for testing. Swabs were then inoculated onto blood plates and beta-hemolytic colonies were counted using a light table.

Saliva swab simulation 2: Swabs were dipped 5 times into tubes containing gargle spiked with GAS bacteria and then dipped 5 times into TH culture media to inoculate. Swabs were discarded prior to incubation of the culture media.

Bacterial counts: 0.05 mL or 0.1 mL samples of bacterial suspensions were inoculated onto blood plates using the appropriate limiting dilutions (dilutions of 8,000-fold or 30,000-fold) and beta-hemolytic colonies were counted using a light table.

### Summary of Results

GAS was successfully captured from almost all plated saliva swab samples of positive subjects enrolled in phase 2 of the Proof of Concept Clinical Trial, as seen in Table 5 of FIG. 44. The data in Table 5 describe the range of GAS CFU amounts observed on the blood plates inoculated with Clinical Trial phase 2 subject saliva swabs. Most plated saliva swabs from positive subjects successfully captured GAS with a range of 4 CFUs to TNTC, with only one case of false negative (pt. ID #033.VEN), yielding a capture rate of 94.7% (18/19). 84.2% of cases had greater than 20 CFUs, 73.7% of cases had greater than 40 CFUs, and 63.2% of cases had greater than 100 CFUs.

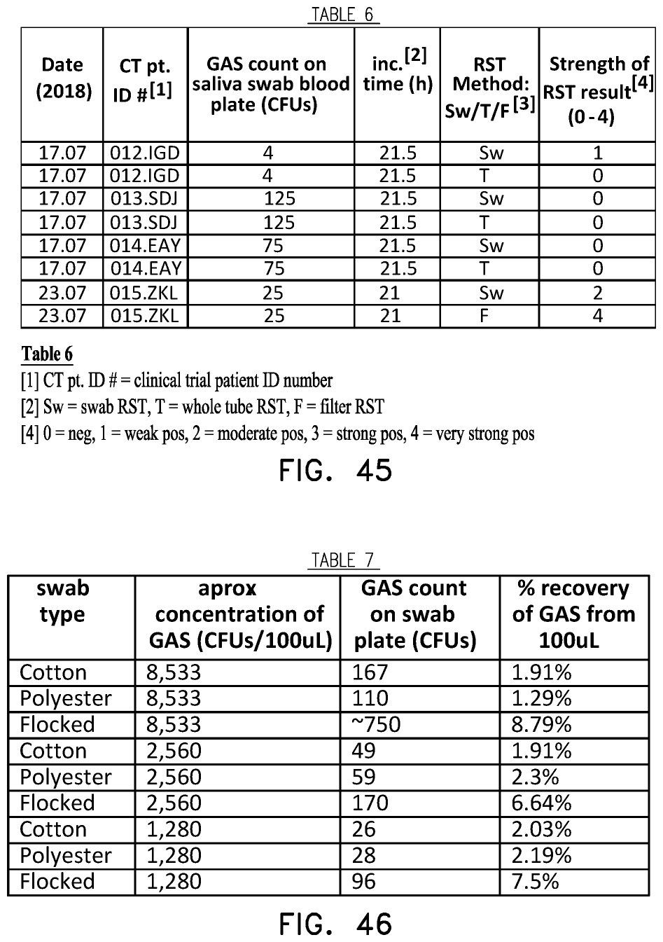

As presented in Table 6 of FIG. 45 (see also the next paragraph), four of the saliva swab samples (Patients 12-15) from the Clinical Trial, after being inoculated onto the blood plates, were also tested using Backup methods performed using RST methods, two of which yielded true positive results. The Filter RST method yielded the strongest RST result (RST 4), and the Whole Tube RST method yielded the weakest RST result (RST 0). The Swab RST method was sensitive enough to yield a positive result even for a patient with a very low amount of GAS in the saliva swab sample (4 CFUs).

After performing the experiment reflected in Table 6, the inventors appreciated that the data presented in Table 6 is invalid due to inaccurate testing. Due to the removal of some of the sample (for inoculating onto blood plates) prior to testing, as described above, the Backup methods using RST methods yielded inaccurate results, because the full sample was not tested. Subsequent clinical trial samples were tested properly utilizing the complete saliva sample (Patients 17-34), as described hereinbelow with reference to Table 8 of FIG. 47.

Saliva swab simulation 1 shows that flocked swabs are the preferred swab for obtaining a salvia swab sample, as can be seen in Table 7 of FIG. 46. The data in Table 7 present an experiment which compared the total absorbance plus elution of GAS onto a plate from three different swab materials: cotton, polyester, and flocked. Swabs were dipped into 0.6 mL of pure GAS liquid suspension and then inoculated onto a blood plate. The flocked swab showed 3 to 5 times more total absorbance plus elution efficiency using three different GAS concentrations.

Saliva swab simulation 2 shows that flocked swabs, when used to inoculate a sample into TH culture broth for a Backup Test performed using RST methods, are as efficient as direct liquid transfer, as can be seen in Table 8 of FIG. 47. The data in Table 8 present an experiment which compared methods for transferring gargle spiked with GAS into culture media (“inoculation methods”). Using a flocked swab for inoculation yielded a positive Backup Test performed using RST methods comparable to using a pipette to transfer 0.2 mL (RST 3).

Almost all saliva swab clinical samples which were inoculated into Todd Hewitt (TH) broth and assayed by Backup methods using RST methods yielded either true positive or true negative results for all subjects enrolled in phase 2 of the Proof of Concept Clinical Trial, seen in Table 9 of FIG. 48. The data presented in Table 9 describe Clinical Trial saliva swabs that were incubated in TH culture broth and were then assayed using Backup Test methods performed using RST methods. The Filter RST method had a sensitivity of 90% and the Swab RST method had a sensitivity of 80%.

### Conclusions

These experimental data support the Backup method for GAS detection using a saliva sample via the incubation of a saliva swab in liquid culture media followed by lateral flow RST immunoassay. Plated clinical saliva swab samples displayed a 94.7% successful capture rate of GAS which confirms that saliva samples are a viable alternative to gargling in cases where gargling is not possible. Saliva swab simulations support the concept that Backup Test methods performed using RST methods of saliva swabs incubated in liquid media is an efficient method for GAS detection. Additional saliva swab simulation data shows that flocked swabs increase the uptake and release of specimen samples compared to other swabs.

Clinical data from 18 samples demonstrates that a saliva swab Backup Test performed using RST methods in liquid media has high sensitivity (90%) and specificity (100%). Furthermore, the Filter Backup Test methods performed using RST methods yielded a higher sensitivity (90%) than the Swab Backup method performed using RST methods (80%).

A liquid growth medium and a method of using the liquid growth medium are provided for testing for the presence of group A streptococcus bacteria (GAS) in a sample of oral fluid obtained from a patient, in accordance with respective applications of the present invention. The liquid growth medium and/or the method may be used in combination with any of the techniques described hereinabove in which growth medium is used for testing for the presence of biological particulate, such as strep, e.g., GAS. Although the liquid growth medium and the method are generally described hereinbelow as being appropriate for testing for the presence of GAS, they may also be used to test for other types of streptococcus bacteria, other types of bacteria, a microorganism, a fungus, a spore, a virus, a mite, a biological cell, a biological antigen, a protein, a protein antigen, or a carbohydrate antigen.

The liquid growth medium has a substantially greater total nitrogen source concentration and a substantially greater total solids concentration than conventional liquid growth media used for incubating GAS. The liquid growth medium has a substantially greater osmotic value (indicative of the total concentration of molecules in the media) than conventional liquid growth media. In particular, the liquid growth medium typically has (a) a total nitrogen source concentration between 75 and 300 g/L and (b) a total solids concentration between 92.5 and 370 g/L.

The high-concentration liquid growth medium may be particularly useful for successfully growing low concentrations (typically between 100 and 500 CFU/ml) of GAS present in samples of oral fluid, such as gargled fluid gargled by the patient or saliva not swabbed from a throat of the patient. These samples of oral fluid typically contain many dozens of types (often over 100) of other types of interfering bacteria. As described below, the inventors have found that the use of conventional, lower concentration liquid growth media for testing for the presence of GAS in samples of oral fluid (rather than samples swabbed from the tonsils, as is conventional in strep testing) results in consumption of most of the nutrients in the liquid growth medium by the interfering bacteria, leaving insufficient nutrients to grow the GAS of interest to an extent adequate for accurate testing.

Many bacterial liquid growth media are used commercially to grow Group A streptococcus bacteria.

They all contain at least 2 of the following three types of components:

a. A sugar, usually glucose, as an energy source.

b. Nitrogen sources as building blocks for nitrogen and carbon.

c. Inorganic salts and molecules that serve as nutrients, as buffers to maintain pH during growth and to maintain osmotic balance.

The table below shows the respective concentrations of the abovementioned three components in some of the widely used, commercially available liquid growth media formulations (which can be obtained from many manufactures all over the world). The following are examples of typical formulations.

TABLE 10

| | | | Nitrogen | | Inorganic | | Total | |
|---|---|---|---|---|---|---|---|---|
| Formulation name | Glucose | | Source | | molecules | | solids | |
| Todd Hewitt Broth | 2 | g/L | 30 | g/L | 4.9 | g/L | 37 | g/L |
| Brain Heart Infusion | 2 | g/L | 27.5 | g/L | 7.5 | g/L | 37 | g/L |
| Tryptic Soy Broth | 2.5 | g/L | 20 | g/L | 7.5 | g/L | 30 | g/L |
| Columbia Broth | 2.5 | g/L | 23.1 | g/L | 9.41 | g/L | 35 | g/L |
| Nutrient Broth | None | | 20 | g/L | 5 | g/L | 25 | g/L |
| Thioglycollate broth | 5.5 | g/L | 20.5 | g/L | 3 | g/L | 29.75 | g/L |

Thus, a typical conventional liquid growth media for streptococcal growth will contain=<30 g/L of nitrogen sources, >10 g/L of Inorganic molecules, >40 g/L of total solids. These conventional liquid growth media, having the respective concentrations as shown in Table 10, all enable good growth of GAS in pure form.

Furthermore, the use of a high-concentration liquid growth medium is conventionally believed to depress the growth of GAS. See, for example, Bernheimer, A. W. and Pappenheimer A. M. Jr., “Factors necessary for massive growth of Group A hemolytic Streptococcus”. Journal of Bacteriology, Volume 43(4), pages 481-494 (1941).

As described hereinabove, the liquid growth medium of the present application has a substantially greater osmotic value (indicative of the total concentration of molecules in the media) than conventional liquid growth media. For some applications, the total nitrogen source concentration is between 105 and 180 g/L, such as between 120 and 160 g/L, and/or the total solids concentration is between 130 and 222 g/L, such as between 148 and 193 g/L.

Typically, the liquid growth medium has a pH of between 6 and 8.3, such as between 7.0 and 8.0.

For some applications, the liquid growth medium has a total sugar concentration of between 6 and 20, such as between 6 and 12. For some of these applications, the liquid growth medium has a glucose concentration of between 7 and 10, such as between 8 and 9.5.

For some applications, an assembly is provided that includes the liquid growth medium and a sealed sterile container that contains the liquid growth medium.

For some applications, an assembly is provided that includes the liquid growth medium and a container that contains the liquid growth medium and a sample of oral fluid obtained from a patient, such as described above.

For some applications, a kit is provided that includes the liquid growth medium and a lateral flow strep test strip, one or more extraction reagents, and/or a filter.

In an application of the present invention, a method of preparing the liquid growth medium includes adding a quantity of powdered growth medium to a volume of distilled water, and stirring until the powdered growth medium is dissolved in the distilled water to produce the liquid growth medium. The quantity of powdered growth medium and the volume of the distilled water are typically selected such that the liquid growth medium has (a) a total nitrogen source concentration between 75 and 300 g/L and (b) a total solids concentration between 92.5 and 370 g/L. The liquid growth medium may optionally have any of characteristics described above.

In an application of the present invention, a method is provided for testing for the presence of GAS in a sample of oral fluid obtained from a patient, the method including:
generating a biological product by incubating the sample of oral fluid for between 12 and 50 hours in a container that contains a liquid growth medium, the liquid growth medium having (a) a total nitrogen source concentration between 75 and 300 g/L and (b) a total solids concentration between 92.5 and 370 g/L; and
thereafter, performing a strep test using a rapid strep test (RST) technique on the biological product.

For some applications, incubating includes incubating for between 16 and 50 hours.

Typically, the container does not contain agar. Alternatively, the container does contain some agar, but it is typically a relatively small amount compared to conventional strep culturing techniques.

For some applications, performing the strep test using the RST technique includes performing a lateral flow test. For some applications, performing the strep test includes applying one or more extraction reagents to the biological product.

Alternatively, for some applications, performing the strep test using the RST technique includes performing an RST technique selected from the group consisting of: an ELISA-based RST, an antibody-coated-beads-based RST, a nucleic-acid-based RST, and a fluorescent immunoassaying (FIA) RST.

Typically, but not necessarily, the sample of oral fluid is selected from the group consisting of: gargled fluid gargled by the patient, and saliva not swabbed from a throat of the patient (e.g., spit by the patient, or sucked onto a swab by the patient).

Alternatively, the sample of oral fluid is saliva swabbed from a tonsil of the patient.

For some applications, generating the biological product further includes filtering the sample of oral fluid and the liquid growth medium after incubating. For some of these applications, performing the strep test using the RST technique includes performing the strep test using the RST technique on the filter. For some of these filtering applications, the sample of oral fluid is saliva swabbed from a tonsil of the patient.

Typically, RST values below 0.25 (average of S and 10 minutes readings) are considered to be negative results. An RST value of 0.5 is indicative of a bacteria concentration of at least 10,000 CFU/ml

The inventors performed experimentation where the lateral flow values of pure systems grown in TH-1 and TH-10 were compared with the lateral flow values of TH-1 systems with added salt (NaCl) or sugar (Glucose) at increasing concentrations. The Lateral flow value of TH-10 was similar to the values of TH-1+5% NaCl and 30% glucose.

5% Nacl and 30% glucose have similar Osmolarities, and inhibited the Strep to the same extent.

From this a value of about 180 MOsmomolar was calculated, and a demonstration of Osmolarity dependent Strep A growth inhibition was shown, in agreement with scientific literature.

A. The following is an experimental setup as performed by the inventors:

1. Each system consisted of a 5 ml test tube, containing 1.1 ml growth media.

2. 0.1 ml bacterial suspensions were added to each system and growth was started by incubation at 35.5° C., in air. Termination was done by withdrawal from the incubator and immediate processing, or by storage at 6-8° C. for up to 2 hours before processing.

3. The 0.1 ml bacterial suspensions were of 3 types:
(a) In “pure” systems the bacteria were suspended in sterile Phosphate-Buffered Saline (“PBS”).
(b) In “Gargle” systems the bacteria were suspended in a gargle solution. Gargle was obtained by gargling 10-11 ml of sterile PBS for about 10 seconds and then transferring the gargle to a collection cup.
(c) In “saliva” systems the bacteria were suspended in saliva. Saliva was obtained by spitting into a collection cup.

4. Incubation was for a period of at least 4 hours but up to 3 days, depending on the experiment.

5. Two strains of GAS were used: the well-known “ATCC 19615” strain, which is used as a control strain in many diagnostic applications, and a wild-type strain “WT-9,” which was isolated from a patient in a clinical trial performed on behalf of the inventors.

6. Bacterial stock suspensions were obtained by resuspending in PBS a 1-4-days-old bacterial colony, grown at 35.5° C. on a blood agar plate for 1-2 days and then stored at 6-8° C. till used. The stock was diluted 10-250,000 fold, depending on the experiment, in either PBS, gargle or saliva. Bacterial dilutions of 4,800-20,000 in PBS were plated (50 microliters), grown at least overnight at 35.5° C., and the beta-hemolytic colonies counted.

7. Processing the samples involved assaying 0.1 ml of sample in an antigen lateral flow, Rapid Strip Test, for Streptococcus Group A. Test strips were obtained from McKesson company, USA, and used in accordance with the manufacture instructions. Estimation of the strength of the positive line was done visually, by experience lab workers.

### Example 1

The following Gargle/Saliva growth Media [GSM] were prepared based on the Todd Hewitt formula, with successively increasing concentrations of Glucose, Nitrogen Sources, and Inorganic molecules, as indicated in Table 11 below:

TABLE 11

| | | | Nitrogen | | Inorganic | | Total | |
|---|---|---|---|---|---|---|---|---|
| Formulation name | Glucose | | Source | | molecules | | solids | |
| Todd Hewitt Broth X | 2 | g/L | 30 | g/L | 4.9 | g/L | 37 | g/L |
| 1 (TH-1) | | | | | | | | |
| Todd Hewitt Broth X | 5 | g/L | 75 | g/L | 12.25 | g/L | 92.5 | g/L |
| 2.5 (GSM -1) | | | | | | | | |
| Todd Hewitt Broth X | 9 | g/L | 135 | g/L | 22.05 | g/L | 166.5 | g/L |
| 4.5 (GSM-2) | | | | | | | | |
| Todd Hewitt Broth X | 14 | g/L | 210 | g/L | 34.3 | g/L | 259 | g/L |
| 7 (GSM-3) | | | | | | | | |
| Todd Hewitt Broth X | 20 | g/L | 300 | g/L | 49 | g/L | 370 | g/L |
| 10 (GSM-4) | | | | | | | | |

1.1 ml of each of the above sterile solutions was placed in a tube.

Bacterial suspensions were prepared by spiking even number of bacteria cells in PBS×1, Gargle fluid and saliva.

0.1 ml of bacterial suspension was added to each of the above growth media shown in Table 11 and incubated at 35.5° C., in air, for 16.5-17.5 hours to obtain cultures.

0.1 ml of each culture was transferred each to a new tube containing Solution A (2M Sodium nitrite) and Solution B (0.2M Acetic acid) followed by a short mix on a Vortex mixer.

McKesson RSTs were dipped into each solution and results were read after 5 and 10 minutes according to arbitrary test line intensity scale. Results are presented as the average between the 2 readings.

### Results

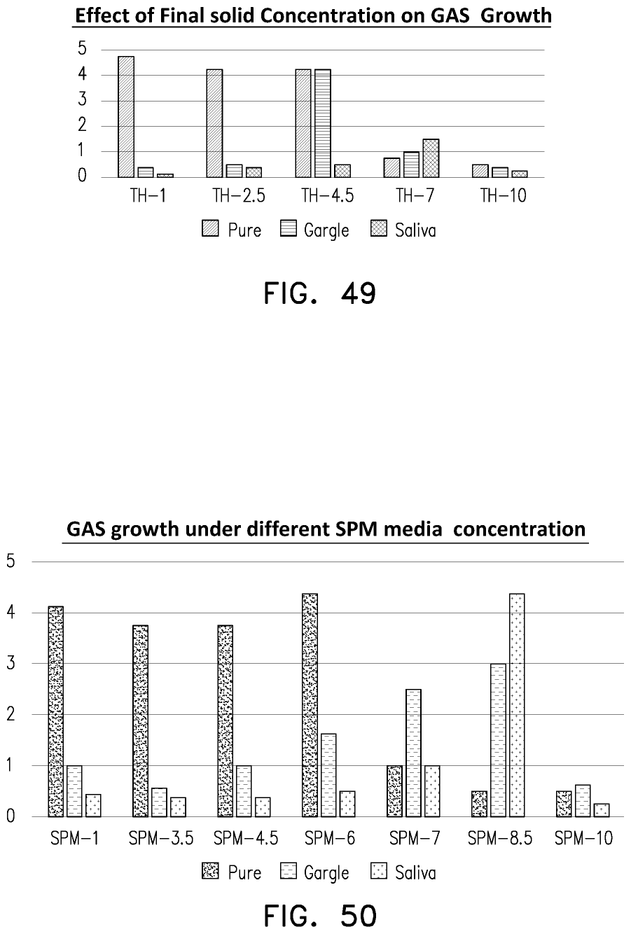

The following Table 12 summarizes test results, which are reflected as well in FIG. 49:

TABLE 12

| | Pure | Gargle | Saliva |
|---|---|---|---|
| Growth Media | Test Line intensity | | |
| TH-1 | 4.8 | 0.4 | 0.1 |
| GSM -1 | 4.3 | 0.5 | 0.4 |
| GSM- 2 | 4.3 | 4.3 | 0.5 |
| GSM - 3 | 0.75 | 1 | 1.5 |
| GSM-4 | 0.5 | 0.4 | 0.3 |

### Conclusions

Both gargle and saliva suspensions grow best in a GSM media having a high concentration of solids.

As per the above results, the highest RST readings for gargle suspension growth resulted when GSM-2 was used.

As per the above results, the highest RST readings for saliva suspension growth resulted when GSM-3 was used.

Thus, the inventors have realized that the optimal range of solids concentrations in liquid growth media for growing gargle and saliva suspensions should be between 4.5-7×TH, i.e., 4.5-7 times the solids concentration in conventional Todd Hewitt liquid growth medium.

In contrast to bacteria sourced from gargle fluid and saliva, the growth of pure GAS culture was inhibited by higher solids concentrations.

### Example 2

The following Gargle/Saliva growth Media [SPM] (shown in Table 14 below) were prepared based on a mix of several formulas as indicated in Table 13 below with successively increasing concentrations of Glucose, Nitrogen sources, and Inorganic molecules:

TABLE 13

| | Formulation name | | | | | |
|---|---|---|---|---|---|---|
| | | Brain | Tryptic | Beef | Yeast | |
| | Todd | Heart | Soy | Ex- | Ex- | Glu- |
| | Hewitt | Infusion | Broth | tract | tract | cose |
| Percentage of | 4.44 g/L | 6.66 g/L | 3.9 g/L | 10 g/L | 3 g/L | 2 g/L |
| total solids for | | | | | | |
| SPM X 1 | | | | | | |

TABLE 14

| | | | Nitrogen | Inorganic | | Total | |
|---|---|---|---|---|---|---|---|
| Formulation name | Glucose | | Source | molecules | | solids | |
| SPM X 1 (SPM-1) | 2.9 | g/L | 24.15 g/L | 2.9 | g/L | 30 | g/L |
| SPM X 3.5 (SPM-3.5 | 10.15 | | 85.525 | 10.15 | | 105 | g/L |
| SPM X 4.5 (SPM-4.5) | 13.05 | | 108.675 | 13.05 | | 135 | g/L |
| SPM X 6 (SPM-6) | 17.4 | | 144.9 | 17.4 | | 180 | g/L |
| SPM X 7 (SPM-7) | 20.3 | | 169.05 | 20.3 | | 210 | g/L |
| SPM X 8.5 (SPM-8.5) | 24.65 | | 205.275 | 24.65 | | 255 | g/L |
| SPM X 10 (SPM-10) | 29 | g/L | 241.5 g/L | 29 | g/L | 300 | g/L |

1.1 ml of each of the above sterile solutions was placed in a tube.

Bacterial suspensions were prepared by spiking even number of bacteria cells in PBS×1, Gargle fluid and saliva.

0.1 ml of bacterial suspension was added to each of the above growth media shown in Table 14 and incubated at 35.5° C., in air, for 22 hours to obtain cultures.

0.1 ml of each culture was transferred each to anew tube containing Solution A (2M Sodium nitrite) and Solution B (0.2M Acetic acid) followed by a short mix on a Vortex mixer.

McKesson RSTs were dipped into each solution and results were read after 5 and 10 minutes according to arbitrary test line intensity scale. Results are presented as the average between the 2 readings.

### Results

The following Table 15 summarizes test results, which are reflected as well in FIG. 50:

TABLE 15

| Growth Media | Pure | Gargle | Saliva |
|---|---|---|---|
| SPM-1 | 4.1 | 1 | 0.4 |
| SPM-3.5 | 3.8 | 0.6 | 0.4 |
| SPM-4.5 | 3.8 | 1 | 0.4 |
| SPM-6 | 4.4 | 1.6 | 0.5 |
| SPM-7 | 1 | 2.5 | 1 |
| SPM-8.5 | 0.5 | 3 | 4.4 |
| SPM-10 | 0.5 | 0.6 | 0.25 |

Thus, increasing the solids concentration in SPM growth media has a similar effect on GAS growth as shown using the GSM media of Example 1.

Solid concentration of growth media has the same effect in both cases (Example 1 and Example 2) regardless of the media nutrient composition.

### Example 3

1.1 ml of the TH-1 and 1.1 ml of GSM-1 growth media were each placed in respective tubes.

Bacterial suspensions were diluted to the final respective cell counts specified in Table 16 below for both growth media formulas. 0.1 ml of each diluted bacterial suspension was added to a tube of TH-1 growth medium and to a tube of GSM-1 growth medium and incubated at 35.5° C., in air, for 23 hours, to obtain cultures.

0.1 ml of each culture was each transferred to a new tube containing Solution A (2M Sodium nitrite) and Solution B (0.2M Acetic acid) followed by a short mix on a Vortex mixer.

McKesson RSTs were dipped in the solution and results were read after 5 and 10 minutes according to arbitrary test line intensity scale. Results are presented as the average between the 2 readings.

### Results

Lateral flow values of cultures grown at 35.5° C. for 23 h

TABLE 16

Test line intensity
CFU per ml

| Culture | CFU per tube | TH-1 | TH-4.5 |
|---|---|---|---|
| Pure | 180 | 4.3 | 4 |
| | 920 | 4 | 3.5 |
| | 4590 | 4.6 | 4.5 |
| | 45880 | 4 | 3.6 |
| | 458800 | 4.2 | 3.9 |
| Gargle | 180 | 0.1 | 4.4 |
| | 920 | 1 | 4.4 |
| | 4590 | 2.5 | 4.3 |
| | 45880 | 4 | 3.4 |
| | 458800 | 4.6 | 2.8 |

### Conclusions

The range of cell numbers per ml in the above experiment represents the variability of cell counts in gargle fluids that were collected during clinical study conducted by Hero Scientific.

GAS cells both from pure culture and gargle fluid grow well in TH×4.5 and can be easily detected by the RST.

TH×1 is inferior to the high-solids concentration broth when gargle fluid is present in the broth at lower cell numbers. At higher cell numbers, even though TH-1 resulted in higher RST readings, TH-4.5 still resulted in sufficiently high readings so as to provide unambiguous results.

In an embodiment, the techniques and apparatus described herein are combined with techniques and apparatus described in one or more of the following patent applications, which are assigned to the assignee of the present application and are incorporated herein by reference:
International Application PCT/IL2018/050225, filed Feb. 28, 2018, which published as WO 2018/158768 to Fruchter et al.;
U.S. Provisional Application 62/727,208, filed Sep. 5, 2018; and/or
   International Application PCT/IL2019/050994, filed Sep. 5, 2019, which published as WO 2020/049566 to Fruchter et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
collecting, from a patient, gargled fluid that potentially contains a particulate selected from the group consisting of: a bacterium and a virus;
passing the gargled fluid through a filter; and
subsequently, testing for the presence of the particulate trapped by the filter.

2. The method according to claim 1, wherein testing for the presence of the particulate trapped by the filter comprises testing the filter for the presence of the particulate.

3. The method according to claim 1, wherein the particulate is the bacterium, and wherein testing for the presence of the particulate trapped by the filter comprises testing for the presence of the bacterium trapped by the filter.

4. The method according to claim 3, wherein the particulate is Streptococcus bacterium, and wherein testing for the presence of the particulate trapped by the filter comprises testing for the presence of the Streptococcus bacterium trapped by the filter.

5. The method according to claim 4, wherein testing for the presence of the Streptococcus bacterium trapped by the filter comprises testing the filter for the presence of the Streptococcus bacterium.

6. The method according to claim 4, wherein testing for the presence of the Streptococcus bacterium trapped by the filter comprises testing for the presence of Streptococcus antigen trapped by the filter.

7. The method according to claim 6, wherein testing for the presence of the Streptococcus antigen trapped by the filter comprises adding a detergent to the gargled fluid.

8. The method according to claim 7, wherein adding the detergent to the gargled fluid comprises adding polysorbate to the gargled fluid.

9. The method according to claim 6, wherein testing for the presence of the Streptococcus antigen trapped by the filter comprises performing a rapid strep test using a dipstick.

10. The method according to claim 9, wherein performing the rapid strep test comprises performing the rapid strep test without first culturing the gargled fluid.

11. The method according to claim 9, wherein the dipstick is a lateral flow immunoassay test strip.

12. The method according to claim 6, wherein testing for the presence of the Streptococcus antigen trapped by the filter comprises applying, to the filter, an extraction solution configured to extract the Streptococcus antigen from the Streptococcus bacterium.

13. The method according to claim 12, wherein the extraction solution is configured to extract Strep A carbohydrate antigen from the Streptococcus bacterium, and wherein testing for the presence of the Streptococcus antigen trapped by the filter comprises testing for the presence of the Strep A carbohydrate antigen trapped by the filter.

14. The method according to claim 12, wherein testing for the presence of the Streptococcus bacterium trapped by the filter further comprises increasing a surface area of the filter that is exposed to the extraction solution before testing for the presence of the Streptococcus antigen trapped by the filter.

15. The method according to claim 12, wherein testing for the presence of the Streptococcus bacterium trapped by the filter further comprises mixing the filter after applying the extraction solution to the filter.

16. The method according to claim 1, wherein the particulate is the virus, and wherein testing for the presence of the particulate trapped by the filter comprises testing for the presence of the virus trapped by the filter.

17. The method according to claim 1, wherein testing for the presence of the particulate trapped by the filter further comprises tearing the filter before testing for the presence of the particulate trapped by the filter.

18. The method according to claim 1, further comprising puncturing the filter before testing for the presence of the particulate trapped by the filter.

19. The method according to claim 1, wherein testing for the presence of the particulate trapped by the filter further comprises crushing the filter before testing for the presence of the particulate trapped by the filter.

20. The method according to claim 1, wherein testing for the presence of the particulate trapped by the filter further comprises concentrating the filter into a more compact volume before testing for the presence of the particulate trapped by the filter.

21. The method according to claim 1, wherein collecting the gargled fluid comprises collecting a gargle fluid that the patient has gargled in his or her mouth and spit out, and wherein the gargle fluid includes a detergent.

22. The method according to claim 21, wherein the detergent includes polysorbate.

23. The method according to claim 1, wherein passing the gargled fluid through the filter comprises pushing the gargled fluid through the filter.

24. The method according to claim 1, further comprising, before passing the gargled fluid through the filter, culturing the particulate using a culture medium.

25. The method according to claim 1, further comprising, before passing the gargled fluid through the filter, preserving the particulate using a preserving medium.

26. The method according to claim 1, wherein passing the gargled fluid through the filter comprises passing the gargled fluid through the filter such that adhesive properties of the filter facilitate trapping of the particulate by the filter.

27. The method according to claim 1, wherein passing the gargled fluid through the filter comprises passing the gargled fluid through the filter such that the filter traps mucus.

28. The method according to claim 27, passing the gargled fluid through the filter comprises passing the gargled fluid through the filter such that the mucus adheres to the filter.

29. The method according to claim 1, wherein the filter includes first and second filters, and wherein passing the gargled fluid through the filter comprises passing the gargled fluid through the first and the second filters.

30. The method according to claim 29, wherein testing for the presence of the particulate comprises testing for the presence of the particulate captured by the first filter.

31. The method according to claim 30, wherein testing for the presence of the particulate comprises testing for the presence of the particulate captured by the first filter and testing for the presence of the particulate captured by the second filter.

32. The method according to claim 30,
wherein the particulate is the bacterium, and wherein testing for the presence of the particulate comprises testing for the presence of the bacterium captured by the first filter, and
wherein the method further comprises testing for the presence of a virus captured by the second filter.

33. The method according to claim 29, wherein passing the gargled fluid through the first and the second filters comprises passing the gargled fluid through the first and the second filters while the first filter is disposed within a tube.

34. The method according to claim 33, wherein passing the gargled fluid through the first and the second filters comprises advancing a plunger within the tube.

35. The method according to claim 29, wherein passing the gargled fluid through the first and the second filters comprises passing the gargled fluid through the first filter and then through the second filter, and wherein a pore size of the first filter is larger than a pore size of the second filter.

36. The method according to claim 35, wherein the pore size of the second filter is no more than 20 microns.

37. The method according to claim 36, wherein the pore size of the second filter is no more than 1 micron.

38. The method according to claim 37, wherein the pore size of the second filter is between 0.1 and 1 micron.

39. The method according to claim 37, wherein the pore size of the second filter is between 0.01 0.3 micron.

40. The method according to claim 35, wherein the pore size of the first filter is between 0.5 and 100 microns.

41. The method according to claim 40, wherein the pore size of the first filter is between 10 and 100 microns.

42. A kit for testing gargled fluid for the presence of a particulate selected from the group consisting of: a bacterium and a virus, the kit comprising:
a filtering apparatus, which comprises:
a tube having a proximal opening;
a first filter disposed within the tube, the first filter having a pore size of between 0.5 and 100 microns; and
a second filter having a pore size of no more than 20 microns, and less than the pore size of the first filter,
wherein the second filter is disposed distally to the first filter, such that the gargled fluid, when in the tube, passes through the first filter and then through the second filter; and
a lateral flow immunoassay test strip, which is configured to detect the presence of the particulate.

43. The kit according to claim 42, wherein the second filter has a pore size of no more than 1 micron.

44. The kit according to claim 42, wherein the second filter has a pore size of between 0.1 and 20 microns.

45. The kit according to claim 44, wherein the second filter has a pore size of between 0.1 microns and 1 micron.

46. The kit according to claim 44, wherein the second filter has a pore size of between 0.01 microns and 0.3 micron.

47. The kit according to claim 44, wherein the second filter has a pore size of between 1 micron and 10 microns.

48. The kit according to claim 42, wherein the first filter has a pore size of between 0.5 and 20 microns.

49. The kit according to claim 42, wherein the first filter has a pore size of between and 100 microns.

50. The kit according to claim 42, wherein the first filter has a pore size of between and 25 microns.

51. The kit according to claim 42, wherein the filtering apparatus further comprises a fluid-collection compartment distal to the first and the second filters.

52. The kit according to claim 51, wherein a wall of the fluid-collection compartment is shaped so as to define a pressure-release hole, such that air pressure in the fluid-collection compartment is released through the pressure-release hole.

53. The kit according to claim 42, wherein the filtering apparatus further comprises a plunger sized and shaped to be distally advanceable through the proximal opening of the tube and within the tube.

54. The kit according to claim 42, wherein the second filter is disposed within the tube.

55. A kit for testing gargled fluid for the presence of a particulate selected from the group consisting of: a bacterium and a virus, the kit comprising:
a filtering apparatus, which comprises:
a tube having a proximal opening;
a first filter disposed within the tube, the first filter having a pore size of between 0.5 and 100 microns; and
a second filter having a pore size of no more than 20 microns, and less than the pore size of the first filter,
wherein the second filter is disposed distally to the first filter, such that the gargled fluid, when in the tube, passes through the first filter and then through the second filter;
a vial; and
a liquid for bathing the particulate in the vial, the liquid selected from the group consisting of: a lysis buffer, saline solution, and transport medium.

56. The kit according to claim 55, wherein the first filter has a pore size of between 0.5 and 20 microns.

57. The kit according to claim 55, wherein the first filter has a pore size of between and 100 microns.

58. The kit according to claim 55, wherein the vial contains the liquid.

59. Apparatus comprising a testing device for testing for the presence of particulate in a liquid, the testing device comprising:
a liquid container for containing the liquid;
a filter, disposed in or downstream of the liquid container;
a liquid-pressure source, which is arranged to apply pressure to drive the liquid contained in the liquid container through the filter; and
a filter chamber that is (a) disposed downstream of the liquid container, (b) shaped so as to define an inlet, and (c) in fluid communication with the filter.

60. The apparatus according to claim 59, wherein the inlet of the filter chamber has an inlet area that is less than a greatest cross-sectional area of the filter chamber, the inlet area and the greatest cross-sectional area measured in respective planes parallel to each other.

61. The apparatus according to claim 59, wherein the filter chamber comprises one or more pressure-activated valves, not disposed at the inlet of the filter chamber.

62. The apparatus according to claim 59, wherein the filter is configured to trap at least 40% of the particulate to be tested and allow passage of the liquid.

63. The apparatus according to any one of claims 59-62, wherein the filter is removably disposed upstream of the filter chamber with the filter partially covering the inlet of the filter chamber.

64. The apparatus according to claim 63, wherein the apparatus further comprises an elongate member configured to push at least a portion of the filter into the filter chamber.

65. The apparatus according to claim 63,
wherein the liquid-pressure source comprises a plunger, which comprises a plunger head that is shaped so as to be insertable into the liquid container, and
wherein the plunger head is configured to push at least a portion of the filter into the filter chamber.

66. The apparatus according to claim 63, wherein the filter chamber comprises one or more valves, not disposed at the inlet of the filter chamber.

67. The apparatus according to claim 66, wherein the one or more valves comprise one or more pressure-activated valves.

68. The apparatus according to claim 67, wherein the one or more valves comprise one or more non-pressure-activated valves.

69. The apparatus according to claim 66,
wherein the liquid container is shaped so as to define one or more openings through a wall of the liquid container,
wherein the one or more openings are downstream of the filter when the filter is removably disposed upstream of the filter chamber with the filter partially covering the inlet of the filter chamber, and
wherein the filter chamber is not disposed so as to receive the liquid that is driven through the one or more openings.

70. The apparatus according to any one of claims 59-62, wherein the apparatus further comprises at least one container comprising an extraction reagent.

71. The apparatus according to claim 70, wherein the apparatus further comprises a test strip.

72. A method comprising:
applying pressure to drive liquid contained in a liquid container of a testing device through a filter of the testing device, wherein the filter is disposed in or downstream of the liquid container, and wherein the liquid includes at least one substance selected from the group of substances consisting of gargled fluid, saliva not swabbed from a throat of a patient, and an incubated culture medium containing a biological sample; and
thereafter, testing, within a filter chamber of the testing device, for the presence of particulate trapped by the filter while the filter is disposed at least partially in the filter chamber, wherein the filter chamber is (a) disposed downstream of the liquid container, (b) shaped so as to define an inlet, and (c) in fluid communication with the filter.

73. The method according to claim 72, wherein testing comprises applying an extraction reagent to the filter while the filter is in the filter chamber.

74. The method according to claim 72, wherein applying the pressure comprises applying positive pressure using a positive-pressure pump disposed upstream of the filter.

75. A method comprising:
applying pressure to drive liquid contained in a liquid container of a testing device (a) through a filter of the testing device and (b) then through one or more valves of the testing device, wherein the filter is disposed in or downstream of the liquid container, wherein the one or more valves are disposed downstream of the filter, and wherein the liquid includes at least one substance selected from the group of substances consisting of gargled fluid, saliva not swabbed from a throat of a patient, and an incubated culture medium containing a biological sample; and
thereafter, testing, within the testing device, for the presence of particulate trapped by the filter while the one or more valves are closed and the filter is disposed in the testing device.

76. The method according to claim 75, wherein testing comprises applying an extraction reagent to the filter.

77. The method according to claim 76, wherein testing further comprises after applying the extraction reagent, inserting a test strip into the testing device and examining the test strip to test for the presence of the particulate.

78. The method according to claim 75, wherein the liquid includes the saliva not swabbed from the throat of the patient.

79. The method according to claim 75, wherein the one or more valves include one or more pressure-activated valves.

80. The method according to claim 75, wherein the one or more valves include one or more non-pressure-activated valves.

81. The method according to claim 80, wherein the testing device is configured to automatically close the one or more non-pressure-activated valves after the pressure is applied to drive the liquid through the filter and then through the one or more non-pressure-activated valves.

82. The method according to claim 80, wherein the one or more non-pressure-activated valves include two discs that are shaped so as to define respective sets of openings, and wherein the one or more non-pressure-activated valves are configured to assume open and closed states when the two sets of openings are aligned and non-aligned with each other.

83. The method according to claim 80,
wherein applying the pressure comprises pushing a plunger including a plunger head inserted into the liquid container, and
wherein the testing device is configured to automatically close the one or more non-pressure-activated valves after the plunger applies the pressure to drive the liquid contained in the liquid container through the filter and then through the one or more non-pressure-activated valves.

84. The method according to claim 83, wherein the testing device is configured such that motion of the plunger automatically closes the one or more non-pressure-activated valves after the plunger applies the pressure to drive the liquid contained in the liquid container through the filter and then through the one or more non-pressure-activated valves.

85. The method according to claim 84,
wherein pushing the plunger comprise rotating the plunger, and
wherein the testing device is configured such that rotational motion of the plunger automatically closes the one or more non-pressure-activated valves after the plunger applies the pressure to drive the liquid contained in the liquid container through the filter and then through the one or more non-pressure-activated valves.

86. The method according to claim 85, wherein the plunger is shaped so as to define one or more plunger threads, and wherein an internal wall of the liquid container is shaped so as to define one or more liquid-container threads that engage the one or more plunger threads such that rotation of the plunger advances the plunger in a downstream direction within the liquid container.

87. The method according to claim 85,
wherein the one or more non-pressure-activated valves comprise two discs that are shaped so as to define respective sets of openings, and wherein the one or more non-pressure-activated valves are configured to assume open and closed states when the two sets of openings are aligned and non-aligned with each other,
wherein pushing the plunger comprise rotating the plunger, and
wherein the testing device is configured such that rotational motion of the plunger automatically closes the one or more non-pressure-activated valves by rotating at least one of the two discs with respect to the other of the discs, after the plunger applies the pressure to drive the liquid contained in the liquid container through the filter and then through the one or more non-pressure-activated valves.

88. The method according to claim 75, wherein the testing device further includes a filter chamber that is (a) disposed downstream of the liquid container, (b) shaped so as to define an inlet, and (c) in fluid communication with filter.

89. The method according to claim 88, wherein the filter chamber includes at least one of the one or more valves, not disposed at the inlet of the filter chamber.

90. The method according to claim 88, wherein applying the pressure comprises applying the pressure while the filter is removably disposed upstream of the filter chamber with the filter partially covering the inlet of the filter chamber.

91. The method according to claim 90, wherein the method further comprises, after applying the pressure and before testing for the presence of the particulate trapped by the filter, pushing at least a portion of the filter into the filter chamber.

92. The method according to claim 88, wherein the filter chamber includes at least one of the one or more valves, not disposed at the inlet of the filter chamber.

93. The method according to claim 92,
wherein the liquid container is shaped so as to define one or more openings through a wall of the liquid container,
wherein the one or more openings are downstream of the filter when the filter is removably disposed upstream of the filter chamber with the filter partially covering the inlet of the filter chamber,
wherein the filter chamber is not disposed so as to receive the liquid that is driven through the one or more openings, and
wherein applying the pressure comprises applying the pressure to drive the liquid (i) partially through (a) the filter and (b) one or more of the one or more valves of the testing device and (ii) partially through the one or more openings.

94. The method according to claim 75, wherein the filter is configured to trap at least 40% of the particulate.

95. The method according to claim 75, wherein the particulate comprises biological particulate.

96. The method according to claim 95, wherein the biological particulate is selected from the group consisting of: a microorganism, a fungus, a bacterium, a spore, a virus, a mite, a biological cell, a biological antigen, a protein, a protein antigen, and a carbohydrate antigen.

97. The method according to claim 75, wherein testing for the presence of the particulate comprises applying an extraction reagent to the filter after applying the pressure.

98. The method according to claim 97, wherein testing for the presence of the particulate comprises using a test strip.

99. Apparatus comprising a testing device for testing for the presence of particulate in a liquid, the testing device comprising:
a liquid container for containing the liquid, the liquid container shaped so as to define upstream and downstream openings;
a filter, removably disposed in the liquid container; and
a plunger head that (a) is shaped so as to be insertable into the liquid container so as to form a movable seal with a wall of the liquid container, and (b) is arranged such that when pushed, the plunger head applies pressure to drive the liquid contained in the liquid container through the filter and then through the downstream opening,
wherein the testing device is configured such that rotation of the plunger head radially compresses the filter toward a central longitudinal axis of the plunger head.

100. The apparatus according to claim 99, wherein the testing device is configured such that the rotation of the plunger head crushes the filter.

101. A method comprising:
inserting a plunger head into a liquid container of a testing device so as to form a movable seal with a wall of the liquid container;
pushing the plunger head to apply pressure to drive liquid contained in the liquid container through a filter of the testing device and then through a downstream opening of the liquid container, which also has an upstream opening, wherein the filter is removably disposed in the liquid container; and
rotating the plunger head to radially crush the filter toward a central longitudinal axis of the plunger head.

102. The method according to claim 101, further comprising, after rotating the plunger head, testing the filter for the presence of particulate trapped by the filter.

103. The method according to claim 101, wherein rotating the plunger head crushes the filter.

104. The method according to claim 103, wherein the plunger head includes a protrusion, and wherein rotating the plunger head causes the protrusion to move radially toward the central longitudinal axis of the plunger head.

105. Apparatus comprising a testing device for testing for the presence of particulate in a liquid, the testing device comprising:
a liquid container for containing the liquid, wherein the liquid container has an internal volume of between 0.5 and 500 ml;
one or more valves;
a filter, disposed in or downstream of the liquid container and upstream of the one or more valves; and
a plunger, which (a) comprises a plunger head that is shaped so as to be insertable into the liquid container, and (b) is arranged to apply pressure to drive the liquid contained in the liquid container through the filter and then through the one or more valves.

106. The apparatus according to claim 105, wherein the one or more valves comprise one or more pressure-activated valves.

107. The apparatus according to claim 105, wherein the one or more valves comprise one or more non-pressure-activated valves.

108. The apparatus according to claim 107, wherein the one or more non-pressure-activated valves comprise two discs that are shaped so as to define respective sets of openings, and wherein the one or more non-pressure-activated valves are configured to assume open and closed states when the two sets of openings are aligned and non-aligned with each other.

109. The apparatus according to claim 107, wherein the testing device is configured to automatically close the one or more non-pressure-activated valves after the plunger applies the pressure to drive the liquid contained in the liquid container through the filter and then through the one or more non-pressure-activated valves.

110. The apparatus according to claim 109, wherein the testing device is configured such that motion of the plunger automatically closes the one or more non-pressure-activated valves after the plunger applies the pressure to drive the liquid contained in the liquid container through the filter and then through the one or more non-pressure-activated valves.

111. The apparatus according to claim 110, wherein the testing device is configured such that rotational motion of the plunger automatically closes the one or more non-pressure-activated valves after the plunger applies the pressure to drive the liquid contained in the liquid container through the filter and then through the one or more non-pressure-activated valves.

112. The apparatus according to claim 111, wherein the plunger is shaped so as to define one or more plunger threads, and wherein an internal wall of the liquid container is shaped so as to define one or more liquid-container threads that engage the one or more plunger threads such that rotation of the plunger advances the plunger in a downstream direction within the liquid container.

113. The apparatus according to claim 111,
wherein the one or more non-pressure-activated valves comprise two discs that are shaped so as to define respective sets of openings, and wherein the one or more non-pressure-activated valves are configured to assume open and closed states when the two sets of openings are aligned and non-aligned with each other, and
wherein the testing device is configured such that rotational motion of the plunger automatically closes the one or more non-pressure-activated valves by rotating at least one of the two discs with respect to the other of the discs, after the plunger applies the pressure to drive the liquid contained in the liquid container through the filter and then through the one or more non-pressure-activated valves.

114. The apparatus according to claim 105, wherein the filter has a filter surface area of an upstream side of the filter that equals between 0.3 and 100 cm2.

115. The apparatus according to claim 114, wherein the filter surface area equals between 0.3 and 30 cm2.

116. The apparatus according to claim 105, wherein the filter is configured to trap at least 40% of the particulate to be tested and allow passage of the liquid.

117. The apparatus according to any one of claims 105-116, wherein the testing device further comprises a filter chamber that is (a) disposed downstream of the liquid container, (b) shaped so as to define an inlet, and (c) in fluid communication with the filter.

118. The apparatus according to claim 117, wherein the filter chamber comprises at least one of the one or more valves, not disposed at the inlet of the filter chamber.

119. The apparatus according to claim 117, wherein the filter is removably disposed upstream of the filter chamber with the filter partially covering the inlet of the filter chamber.

120. The apparatus according to claim 119, wherein the testing device further comprises a support for the filter, disposed at least partially between the inlet of the filter chamber and the filter.

121. The apparatus according to claim 119, wherein the apparatus further comprises an elongate member configured to push at least a portion of the filter into the filter chamber.

122. The apparatus according to any one of claims 105-116,
wherein the one or more valves are one or more first valves, and
wherein the testing device further comprises one or more second pressure relief valves, which are in fluid communication with the liquid container and are disposed upstream of the filter.

123. The apparatus according to any one of claims 105-116, wherein the apparatus further comprises at least one container containing an extraction reagent.

124. The apparatus according to claim 123, wherein the apparatus further comprises a test strip.

125. Apparatus comprising a testing device for testing for the presence of particulate in a liquid, the testing device comprising:
a liquid container for containing the liquid, wherein the liquid container has an internal volume of between 0.5 and 500 ml;
one or more non-pressure-activated valves;
a filter, disposed in or downstream of the liquid container and upstream of the one or more valves; and
a liquid-pressure source, which is arranged to apply pressure to drive the liquid contained in the liquid container through the filter and then through the one or more valves,
wherein the testing device is configured to automatically close the one or more non-pressure-activated valves after the liquid-pressure source applies the pressure to drive the liquid contained in the liquid container through the filter and then through the one or more non-pressure-activated valves.

126. The apparatus according to claim 125, wherein the testing device is configured such that motion of the liquid-pressure source automatically closes the one or more non-pressure-activated valves after the liquid-pressure source applies the pressure to drive the liquid contained in the liquid container through the filter and then through the one or more non-pressure-activated valves.

127. The apparatus according to claim 126, wherein the testing device is configured such that rotational motion of the liquid-pressure source automatically closes the one or more non-pressure-activated valves after the liquid-pressure source applies the pressure to drive the liquid contained in the liquid container through the filter and then through the one or more non-pressure-activated valves.

128. The apparatus according to claim 127,
wherein the one or more non-pressure-activated valves comprise two discs that are shaped so as to define respective sets of openings, and wherein the one or more non-pressure-activated valves are configured to assume open and closed states when the two sets of openings are aligned and non-aligned with each other, and
wherein the testing device is configured such that rotational motion of the liquid-pressure source automatically closes the one or more non-pressure-activated valves by rotating at least one of the two discs with respect to the other of the discs, after the liquid-pressure source applies the pressure to drive the liquid contained in the liquid container through the filter and then through the one or more non-pressure-activated valves.

129. A method comprising:
incubating gargled fluid for between 12 and 75 hours in a container that contains a liquid growth medium, a dehydrated growth medium, or a gel growth medium; and
thereafter, performing a strep test using a rapid strep test (RST) technique on the gargled fluid and growth medium.

130. The method according to claim 129, wherein performing the strep test using the RST technique comprises performing a lateral flow test.

131. The method according to claim 129, wherein performing the strep test using the RST technique comprises performing an RST technique selected from the group consisting of: an ELISA-based RST, an antibody-coated-beads-based RST, a nucleic-acid-based RST, and a fluorescent immunoassaying (FA) RST.

132. The method according to claim 129, wherein performing the strep test using the RST technique further comprises filtering the gargled fluid and the growth medium after incubating, and performing the strep test using the RST technique on the filter.

133. The method according to claim 132, wherein filtering the gargled fluid and the growth medium after incubating comprises:
placing the gargled fluid and the growth medium in a liquid container of a testing device, and
applying pressure to drive the gargled fluid and the growth medium contained in the liquid container (a) through a filter of the testing device and (b) then through one or more valves of the testing device, wherein the filter is disposed in or downstream of the liquid container, and wherein the one or more valves are disposed downstream of the filter.

134. A method for testing for the presence of particulate in gargled fluid, the method comprising:
incubating the gargled fluid for between 12 and 75 hours in a container that contains a liquid growth medium, a dehydrated growth medium, or a gel growth medium; and
thereafter, performing a test for the particulate using a rapid test technique on the gargled fluid and growth medium.

135. The method according to claim 134, wherein performing the test using the rapid test technique comprises performing a lateral flow test.

136. The method according to claim 134, wherein performing the test using the rapid test technique comprises performing a rapid test technique selected from the group consisting of an ELISA-based rapid test, an antibody-coated-beads-based rapid test, a nucleic-acid-based rapid test, and a fluorescent immunoassaying (FIA) rapid test.

137. A method for testing for the presence of particulate in gargled fluid, the method comprising:
incubating the gargled fluid for between 12 and 75 hours in a container that contains a liquid growth medium, a dehydrated growth medium, or a gel growth medium; and
thereafter, performing a lateral flow test for the particulate on the gargled fluid and growth medium.

138. The method according to claim 137, wherein the particulate is strep, and wherein performing the lateral flow test comprises performing the lateral flow test for the strep.

139. The method according to any one of claims 129, 134, and 79, wherein the container does not contain agar.

140. A method comprising:
incubating saliva not swabbed from a patient's throat for between 12 and 75 hours in a container that contains a liquid growth medium, a dehydrated growth medium, or a gel growth medium; and
thereafter, performing a strep test using a rapid strep test (RST) technique on the saliva and growth medium.

141. The method according to claim 140, wherein the saliva not swabbed from the throat of the patient is saliva spit by the patient.

142. The method according to claim 140, further comprising mixing the saliva with the growth medium before incubating.

143. The method according to claim 140, wherein performing the strep test using the RST technique comprises performing a lateral flow test.

144. The method according to claim 140, wherein performing the strep test using the RST technique comprises performing an RST technique selected from the group consisting of: an ELISA-based RST, an antibody-coated-beads-based RST, a nucleic-acid-based RST, and a fluorescent immunoassaying (FIA) RST.

145. The method according to claim 140, wherein incubating comprises:
receiving, on an absorbent element, saliva from the patient's mouth; and
thereafter, placing the absorbent element into the container that contains the liquid growth medium, dehydrated growth medium, or gel growth medium.

146. The method according to claim 145, wherein performing the strep test using the RST technique comprises performing the RST technique on the saliva and the growth medium while the saliva and the growth medium are in the container.

147. Apparatus comprising A testing kit for testing for the presence of particulate in a liquid, the testing kit comprising:
a liquid container for containing the liquid, the liquid container shaped so as to define upstream and downstream openings;
a filter, disposed in or downstream of the liquid container; and
a plunger head that (a) is shaped so as to be insertable into the liquid container so as to form a movable seal with a wall of the liquid container, and (b) is arranged such that when pushed, the plunger head applies pressure to drive the liquid contained in the liquid container through the filter and then through the downstream opening,
wherein the testing kit does not comprise a plunger shaft.

148. The apparatus according to claim 147, wherein the filter is configured to trap at least 40% of a particulate to be tested and allow passage of the liquid.

149. The apparatus according to claim 147, further comprising sterile packaging, in which at least the liquid container, plunger head, and the filter are removably disposed.

150. The apparatus according to claim 147, wherein the liquid container comprises a liquid-tight seal disposed downstream of the filter, and wherein the testing kit is arranged such that when the plunger head is pushed, the plunger head applies the pressure to break or open the seal and drive the liquid through the filter and then through the downstream opening.

151. A method comprising:
receiving a testing kit including (a) a liquid container, the liquid container shaped so as to define upstream and downstream openings, (b) a filter, disposed in or downstream of the liquid container, (c) and a plunger head;
coupling the plunger head to a plunger shaft;
receiving a liquid in the liquid container;
inserting the plunger head into the liquid container so as to form a movable seal with a wall of the liquid container; and
using the plunger shaft, pushing the plunger head to apply pressure to drive the liquid contained in the liquid container through the filter and then through the downstream opening,
wherein the testing kit does not include the plunger shaft.

152. The method according to claim 151, further comprising, after pushing the plunger head, testing for the presence of particulate trapped by the filter.
